(19) United States
(12) Patent Application Publication
(10) Pub. No.: US 2024/0141372 A1
(43) Pub. Date: May 2, 2024

(54) TRANSGENIC BANANA PLANTS HAVING INCREASED RESISTANCE TO FUSARIUM OXYSPORUM TROPICAL RACE 4 AND METHODS OF PRODUCING SAME

(71) Applicant: Elo Life Systems, Durham, NC (US)

(72) Inventors: Matthew DiLeo, Durham, NC (US); Jack Q. Wilkinson, Cary, NC (US); Daniel Stauffer, Raleigh, NC (US); Tomas Hasing, Durham, NC (US); Tengfang Huang, Cary, NC (US); Taylor Frazier-Douglas, Durham, NC (US); Maria Angels de Luis Balaguer, Redmond, WA (US); Fayaz Khazi, Chapel Hill, NC (US)

(21) Appl. No.: 18/470,048

(22) Filed: Sep. 19, 2023

Related U.S. Application Data
(60) Provisional application No. 63/376,188, filed on Sep. 19, 2022.

(51) Int. Cl. C12N 15/82; A01H 6/00

(52) U.S. Cl. CPC C12N 15/8282; A01H 6/00

(57) ABSTRACT

The present disclosure provides compositions and methods for producing transgenic banana plants that exhibit increased resistance to Fusarium oxysporum f.sp. cubense Tropical Race 4 (TR4), and the banana plants and bananas so produced.

### CROSS-REFERENCE TO RELATED APPLICATIONS

This application claims the benefit under 35 U.S.C. § 119(e) of U.S. provisional application Ser. No. 63/376,188, filed on Sep. 19, 2022, which is herein incorporated by reference in its entirety.

### INCORPORATION OF SEQUENCE LISTING

A sequence listing containing the file named “ELSS003US_ST26.xml” which is 470 kilobytes (measured in MS-Windows®) and created on Sep. 14, 2023, and comprises 224 sequences, is incorporated herein by reference in its entirety.

### FIELD OF THE INVENTION

The present disclosure relates to the field of genetically engineering banana plants, and more specifically to methods and compositions for producing banana plants exhibiting increased resistance to Fusarium oxysporum f.sp. cubense Tropical Race 4.

### BACKGROUND OF THE INVENTION

Bananas are one of the most popular fruits worldwide. They contain essential nutrients that can have a protective impact on health. Eating bananas can help lower blood pressure and may reduce the risk of cancer.

Most export bananas are a single variety class (Cavendish), which is highly susceptible to a relatively new race of an old banana pathogen, Fusarium oxysporum f.sp. cubense Tropical Race 4 (TR4). Fusarium oxysporum f.sp. cubense Tropical Race 4 (TR4) began affecting bananas in Taiwan in 1977, and has quickly spread throughout China, Southeast Asia, Australia, the Middle East, India, Peru, Venezuela and Colombia, and continues to spread to other major banana producing countries. There is no other variety of banana that is both resistant to this disease and that fits the agronomic, shipping and consumer expectation characteristics to allow a drop-in replacement for Cavendish. If no solution is found to this problem Cavendish bananas could be eliminated from the world market.

Therefore there is a need for banana plants that have increased resistance to Fusarium oxysporum f.sp. cubense Tropical Race 4 (TR4).

### SUMMARY

The present disclosure solves these and other problems in the art by providing banana plants exhibiting increased resistance to Fusarium oxysporum f.sp. cubense Tropical Race 4, and methods for making such banana plants.

The present disclosure provides a transgenic banana plant comprising a nucleic acid construct comprising: a) a first nucleic acid sequence encoding a Bcl-2 associated athanogene (BAG) family molecular chaperone regulator 1 protein; b) a second nucleic acid sequence encoding a nucleic acid molecule that inhibits cytochrome P450 lanosterol 14α-demethylase; c) a third nucleic acid sequence encoding at least a first antimicrobial peptide; d) a fourth nucleic acid sequence encoding a resistance gene analog 2 protein; e) a fifth nucleic acid sequence encoding at least a first betalain biosynthesis protein; f) a sixth nucleic acid sequence encoding a eugenol, limonene or geraniol biosynthesis gene; g) a seventh nucleic acid sequence encoding a UDP glycosyltransferase protein; h) an eighth nucleic acid sequence encoding a proteosome maturation factor; i) a ninth nucleic acid sequence encoding a disease resistance protein; or j) combinations thereof, wherein the first, second, third, fourth, fifth, sixth, seventh, eighth or ninth nucleic acid sequence is operably linked to a heterologous or gene edited promoter, and wherein the banana plant exhibits increased resistance to Fusarium oxysporum f.sp. cubense Tropical Race 4 (TR4) compared to a banana plant lacking the nucleic acid construct. In certain embodiments, the first nucleic acid sequence has at least 90% sequence identity to SEQ ID NO:1, or the complete complement thereof; the second nucleic acid sequence has at least 90% sequence identity to SEQ ID NO:3, or the complete complement thereof; the at least a third nucleic acid sequence has at least 90% sequence identity to SEQ ID NO:4, SEQ ID NO:106, SEQ ID NO: 125, SEQ ID NO:128, SEQ ID NO:131, SEQ ID NO:134, SEQ ID NO:137, SEQ ID NO:140, SEQ ID NO:143, SEQ ID NO:146, SEQ ID NO:149, SEQ ID NO:152, SEQ ID NO:155, SEQ ID NO:158, SEQ ID NO:161, SEQ ID NO:164, SEQ ID NO:167, SEQ ID NO:170, SEQ ID NO:173, SEQ ID NO:176, SEQ ID NO:179, SEQ ID NO:182, SEQ ID NO:185, SEQ ID NO:188, SEQ ID NO:191, SEQ ID NO:194, SEQ ID NO:197, SEQ ID NO:200, SEQ ID NO:203, SEQ ID NO:206, SEQ ID NO:209, SEQ ID NO:215, SEQ ID NO:216, SEQ ID NO:217, SEQ ID NO:218, or the complete complement thereof; the fourth nucleic acid sequence has at least 90% sequence identity to SEQ ID NO:6, or the complete complement thereof; the at least a fifth nucleic acid sequence has at least 90% sequence identity to SEQ ID NO:8, or the complete complement thereof; the sixth nucleic acid sequence has at least 90% sequence identity to SEQ ID NO:96, SEQ ID NO:98 or SEQ ID NO:114, or the complete complement thereof; the seventh nucleic acid sequence has at least 90% sequence identity to SEQ ID NO:110, or the complete complement thereof; the eighth nucleic acid sequence has at least 90% sequence identity to SEQ ID NO: 100, or the complete complement thereof; or the ninth nucleic acid sequence has at least 90% sequence identity to SEQ ID NO:102, or the complete complement thereof. In particular embodiments the banana plant is a Musa acuminata banana plant.

In certain embodiments the first nucleic acid sequence has at least 90% sequence identity to SEQ ID NO:14, or the complete complement thereof, the second nucleic acid sequence has at least 90% sequence identity to SEQ ID NO:13, or the complete complement thereof, the at least a third nucleic acid sequence has at least 90% sequence identity to SEQ ID NO:11 or SEQ ID NO:223, or the complete complement thereof, the fourth nucleic acid sequence has at least 90% sequence identity to SEQ ID NO:12, or the complete complement thereof, or the at least a fifth nucleic acid sequence has at least 90% sequence identity to SEQ ID NO:10, or the complete complement thereof.

In some embodiments the heterologous promoter is an inducible, plant, bacterial, viral, synthetic, constitutive, tissue specific, developmentally regulated, cell cycle regulated, temporally regulated, spatially regulated, and/or spatio-temporally regulated promoter. In other embodiments the heterologous promoter is a HLVH12 (SEQ ID NO:17), DCMV (SEQ ID NO:18), FSgt/PFLt (SEQ ID NO:19), dMMV (SEQ ID NO:20), CmYLCV (SEQ ID NO:21), e35S (SEQ ID NO:22), NOS (SEQ ID NO:23), ScBV (SEQ ID NO:24), CsVMV (SEQ ID NO:25), FMVSgt (SEQ ID NO:26), FS1_1 (SEQ ID NO:27), FE_3 (SEQ ID NO:28), ZmUbi1 (SEQ ID NO:116), OsAct1 (SEQ ID NO:117), VND7 (SEQ ID NO:118), Ma521 Ma09_g14890 (SEQ ID NO:119), Ma119 Ma08_g12140 (SEQ ID NO:120), MaM4A Ma01_g10480 (SEQ IS NO:121), MaBB Ma04_g25440 (SEQ ID NO:122), Ma40554 Ma09_g15840 (SEQ ID NO:123) or MaACT1 (SEQ ID NO:124) promoter. In certain embodiments the heterologous promoter is a root specific promoter. In some embodiments the root specific promoter is a Ma521 Ma09_g14890 (SEQ ID NO:119), Ma119 Ma08_g12140 (SEQ ID NO:120), MaM4A Ma01_g10480 (SEQ ID NO:121), MaBB Ma04_g25440 (SEQ ID NO:122) or Ma40554 Ma09_g15840 (SEQ ID NO:123) promoter.

In additional embodiments the transgenic banana plant further comprises a selectable marker sequence. In further embodiments the selectable marker sequence is a β glucuronidase, green fluorescent protein, or antibiotic resistance sequence. In yet further embodiments the selectable marker sequence is a kanamycin resistance sequence.

In certain embodiments the first, second, at least a third, fourth, at least a fifth, sixth, seventh, eighth or ninth nucleic acid sequence is operably linked to a terminator sequence. In particular embodiments the terminator sequence is a Pea3A (SEQ ID NO:29), AtUBQ3 (SEQ ID NO:30), GmaxMYB2 (SEQ ID NO:31), AtRBCS2b (SEQ ID NO:32), Pea E9 (SEQ ID NO:33), ATHSP18.2 (SEQ ID NO:34), potato Ubi3 (SEQ ID NO:35), AtTubB9 (SEQ ID NO:36) 35S (SEQ ID NO:37), CaMV 35S (SEQ ID NO:212), NOS (SEQ ID NO:213) or PBI synthetic (SEQ ID NO:214) terminator sequence.

In further embodiments the nucleic acid construct comprises two or more of the first, second, third, fourth, fifth, sixth, seventh, eighth or ninth nucleic acid sequences, or combinations of two or more of the first, second, third, fourth, fifth, sixth, seventh, eighth or ninth nucleic acid sequences. In some embodiments the two or more of the first, second, third, fourth, fifth, sixth, seventh, eighth or ninth nucleic acid sequences are operably linked to a single heterologous or gene edited promoter. In other embodiments the two or more of the first, second, third, fourth, fifth, sixth, seventh, eighth or ninth nucleic acid sequences are operably linked to different heterologous or gene edited promoters. In additional embodiments the first, second, third, fourth, fifth, sixth, seventh, eighth or ninth nucleic acid sequence further comprises a 2A self-cleaving peptide nucleic acid sequence.

The present disclosure also provides a plant part of a transgenic banana plant comprising a nucleic acid construct comprising: a) a first nucleic acid sequence encoding a Bcl-2 associated athanogene (BAG) family molecular chaperone regulator 1 protein; b) a second nucleic acid sequence encoding a nucleic acid molecule that inhibits cytochrome P450 lanosterol 14α-demethylase; c) at least a third nucleic acid sequence encoding an antimicrobial peptide; d) a fourth nucleic acid sequence encoding a resistance gene analog 2 protein; e) at least a fifth nucleic acid sequence encoding at least a first betalain biosynthesis protein; f) a sixth nucleic acid sequence encoding a eugenol, limonene or geraniol biosynthesis gene; g) a seventh nucleic acid sequence encoding a UDP glycosyltransferase protein; h) an eighth nucleic acid sequence encoding a proteosome maturation factor; i) a ninth nucleic acid sequence encoding a disease resistance protein; or j) combinations thereof, wherein the first, second, third, fourth, fifth, sixth, seventh, eighth or ninth nucleic acid sequence is operably linked to a heterologous or gene edited promoter, and wherein the banana plant exhibits increased resistance to Fusarium oxysporum f.sp. cubense Tropical Race 4 (TR4) compared to a banana plant lacking the nucleic acid construct. In particular embodiments the plant part is a fruit, seed, leaf, root, flower, shoot, cell, endosperm, banana pulp, banana peel, ovule, or pollen.

The present disclosure additionally provides a banana produced by a transgenic banana plant comprising a nucleic acid construct comprising: a) a first nucleic acid sequence encoding a Bcl-2 associated athanogene (BAG) family molecular chaperone regulator 1 protein; b) a second nucleic acid sequence encoding a nucleic acid molecule that inhibits cytochrome P450 lanosterol 14α-demethylase; c) at least a third nucleic acid sequence encoding an antimicrobial peptide; d) a fourth nucleic acid sequence encoding a resistance gene analog 2 protein; e) at least a fifth nucleic acid sequence encoding at least a first betalain biosynthesis protein; f) a sixth nucleic acid sequence encoding a eugenol, limonene or geraniol biosynthesis gene; g) a seventh nucleic acid sequence encoding a UDP glycosyltransferase protein; h) an eighth nucleic acid sequence encoding a proteosome maturation factor; i) a ninth nucleic acid sequence encoding a disease resistance protein; or j) combinations thereof, wherein the first, second, third, fourth, fifth, sixth, seventh, eighth or ninth nucleic acid sequence is operably linked to a heterologous or gene edited promoter, and wherein the banana plant exhibits increased resistance to Fusarium oxysporum f.sp. cubense Tropical Race 4 (TR4) compared to a banana plant lacking the nucleic acid construct, wherein the banana comprises a detectable amount of the first, second, third, fourth, fifth, sixth, seventh, eighth or ninth nucleic acid sequence.

The present disclosure also provides a banana comprising a nucleic acid construct comprising: a) a first nucleic acid sequence encoding a Bcl-2 associated athanogene (BAG) family molecular chaperone regulator 1 protein; b) a second nucleic acid sequence encoding a nucleic acid molecule that inhibits cytochrome P450 lanosterol 14α-demethylase; c) at least a third nucleic acid sequence encoding an antimicrobial peptide; d) a fourth nucleic acid sequence encoding a resistance gene analog 2 protein; e) at least a fifth nucleic acid sequence encoding at least a first betalain biosynthesis protein; f) a sixth nucleic acid sequence encoding a eugenol, limonene or geraniol biosynthesis gene; g) a seventh nucleic acid sequence encoding a UDP glycosyltransferase protein; h) an eighth nucleic acid sequence encoding a proteosome maturation factor; i) a ninth nucleic acid sequence encoding a disease resistance protein; or j) combinations thereof, wherein the first, second, third, fourth, fifth, sixth, seventh, eighth or ninth nucleic acid sequence is operably linked to a heterologous or gene edited promoter, and wherein the banana comprises a detectable amount of the first, second, third, fourth, fifth, sixth, seventh, eighth or ninth nucleic acid sequence.

The present disclosure further provides a banana product comprising a nucleic acid construct comprising: a) a first nucleic acid sequence encoding a Bcl-2 associated athanogene (BAG) family molecular chaperone regulator 1 protein; b) a second nucleic acid sequence encoding a nucleic acid molecule that inhibits cytochrome P450 lanosterol 14α-demethylase; c) at least a third nucleic acid sequence encoding an antimicrobial peptide; d) a fourth nucleic acid sequence encoding a resistance gene analog 2 protein; e) at least a fifth nucleic acid sequence encoding at least a first betalain biosynthesis protein; f) a sixth nucleic acid sequence encoding a eugenol, limonene or geraniol biosynthesis gene; g) a seventh nucleic acid sequence encoding a UDP glycosyltransferase protein; h) an eighth nucleic acid sequence encoding a proteosome maturation factor; i) a ninth nucleic acid sequence encoding a disease resistance protein; or j) combinations thereof, wherein the first, second, third, fourth, fifth, sixth, seventh, eighth or ninth nucleic acid sequence is operably linked to a heterologous or gene edited promoter, wherein the banana product comprises a detectable amount of the first, second, third, fourth, fifth, sixth, seventh, eighth or ninth nucleic acid sequence. In certain embodiments the banana product is banana puree, banana powder, banana pulp, banana peel, banana jam, banana sauce, a banana drink, pastillas de saging, a banana fig, banana vinegar, dried banana chips, fried banana chips, banana flour, banana flakes, banana peel pasta, banana bread, banana cake, banana cue, banana fritter, a banana pancake, banana pudding, banana roll, banana ice cream, or banana frozen yogurt.

The present disclosure further provides a method of producing a banana plant with increased resistance to Fusarium oxysporum f.sp. cubense Tropical Race 4 (TR4), comprising introducing into a banana plant a nucleic acid construct comprising: a) a first nucleic acid sequence encoding a Bcl-2 associated athanogene (BAG) family molecular chaperone regulator 1 protein; b) a second nucleic acid sequence encoding a nucleic acid molecule that inhibits cytochrome P450 lanosterol 14α-demethylase; c) at least a third nucleic acid sequence encoding an antimicrobial peptide; d) a fourth nucleic acid sequence encoding a resistance gene analog 2 protein; e) at least a fifth nucleic acid sequence encoding at least a first betalain biosynthesis protein; f) a sixth nucleic acid sequence encoding a eugenol, limonene or geraniol biosynthesis gene; g) a seventh nucleic acid sequence encoding a UDP glycosyltransferase protein; h) an eighth nucleic acid sequence encoding a proteosome maturation factor; i) a ninth nucleic acid sequence encoding a disease resistance protein; or j) combinations thereof, wherein the first, second, third, fourth, fifth, sixth, seventh, eighth or ninth nucleic acid sequence is operably linked to a heterologous or gene edited promoter. In certain embodiments the first nucleic acid sequence has at least 90% sequence identity to SEQ ID NO:1, or the complete complement thereof; the second nucleic acid sequence has at least 90% sequence identity to SEQ ID NO:3, or the complete complement thereof; the third nucleic acid sequence has at least 90% sequence identity to SEQ ID NO:4, SEQ ID NO:106, SEQ ID NO:125, SEQ ID NO:128, SEQ ID NO:131, SEQ ID NO:134, SEQ ID NO:137, SEQ ID NO:140, SEQ ID NO:143, SEQ ID NO:146, SEQ ID NO:149, SEQ ID NO: 152, SEQ ID NO:155, SEQ ID NO:158, SEQ ID NO:161, SEQ ID NO:164, SEQ ID NO:167, SEQ ID NO:170, SEQ ID NO:173, SEQ ID NO:176, SEQ ID NO:179, SEQ ID NO:182, SEQ ID NO:185, SEQ ID NO:188, SEQ ID NO:191, SEQ ID NO:194, SEQ ID NO:197, SEQ ID NO:200, SEQ ID NO:203, SEQ ID NO:206, SEQ ID NO:209, SEQ ID NO:215, SEQ ID NO:216, SEQ ID NO:217, SEQ ID NO:218, or the complete complement thereof; the fourth nucleic acid sequence has at least 90% sequence identity to SEQ ID NO:6, or the complete complement thereof; the fifth nucleic acid sequence has at least 90% sequence identity to SEQ ID NO:8, or the complete complement thereof, the sixth nucleic acid sequence has at least 90% sequence identity to SEQ ID NO:96, SEQ ID NO:98 or SEQ ID NO:114, or the complete complement thereof; the seventh nucleic acid sequence has at least 90% sequence identity to SEQ ID NO:110, or the complete complement thereof; the eighth nucleic acid sequence has at least 90% sequence identity to SEQ ID NO:100, or the complete complement thereof; or the ninth nucleic acid sequence has at least 90% sequence identity to SEQ ID NO:102, or the complete complement thereof. In particular embodiments the banana plant is a Musa acuminata banana plant.

In addition the present disclosure provides a nucleic acid construct comprising: a) a first nucleic acid sequence having at least 90% sequence identity to SEQ ID NO:1, or the complete complement thereof; b) a second nucleic acid sequence having at least 90% sequence identity to SEQ ID NO:3, or the complete complement thereof; c) at least a third nucleic acid sequence having at least 90% sequence identity to SEQ ID NO:4, SEQ ID NO:106, SEQ ID NO:125, SEQ ID NO:128, SEQ ID NO:131, SEQ ID NO:134, SEQ ID NO:137, SEQ ID NO:140, SEQ ID NO:143, SEQ ID NO:146, SEQ ID NO:149, SEQ ID NO:152, SEQ ID NO: 155, SEQ ID NO:158, SEQ ID NO:161, SEQ ID NO:164, SEQ ID NO:167, SEQ ID NO:170, SEQ ID NO:173, SEQ ID NO:176, SEQ ID NO:179, SEQ ID NO:182, SEQ ID NO:185, SEQ ID NO: 188, SEQ ID NO:191, SEQ ID NO:194, SEQ ID NO:197, SEQ ID NO:200, SEQ ID NO:203, SEQ ID NO:206, SEQ ID NO:209, SEQ ID NO:215, SEQ ID NO:216, SEQ ID NO:217, SEQ ID NO:218, or the complete complement thereof; d) a fourth nucleic acid sequence having at least 90% sequence identity to SEQ ID NO:6, or the complete complement thereof; e) at least a fifth nucleic acid sequence having at least 90% sequence identity to SEQ ID NO:8, or the complete complement thereof; f) a sixth nucleic acid sequence has at least 90% sequence identity to SEQ ID NO:96, SEQ ID NO:98 or SEQ ID NO:114, or the complete complement thereof; g) a seventh nucleic acid sequence has at least 90% sequence identity to SEQ ID NO:110, or the complete complement thereof; h) an eighth nucleic acid sequence has at least 90% sequence identity to SEQ ID NO:100, or the complete complement thereof; i) a ninth nucleic acid sequence has at least 90% sequence identity to SEQ ID NO:102, or the complete complement thereof. or j) combinations thereof, wherein the first, second, third, fourth, fifth, sixth, seventh, eighth or ninth nucleic acid sequence is operably linked to a heterologous or gene edited promoter. In some embodiments the first nucleic acid sequence has at least 90% sequence identity to SEQ ID NO:14, or the complete complement thereof; the second nucleic acid sequence has at least 90% sequence identity to SEQ ID NO:13, or the complete complement thereof; the third nucleic acid sequence has at least 90% sequence identity to SEQ ID NO:11 or SEQ ID NO:223, or the complete complement thereof; the fourth nucleic acid sequence has at least 90% sequence identity to SEQ ID NO: 12, or the complete complement thereof; or the fifth nucleic acid sequence has at least 90% sequence identity to SEQ ID NO:10, or the complete complement thereof.

### BRIEF DESCRIPTION OF THE DRAWINGS

The following drawings form part of the present specification and are included to further demonstrate certain aspects of the present disclosure. The present disclosure may be better understood by reference to one or more of these drawings in combination with the detailed description of specific embodiments presented herein.

FIG. 1. Map of expression vector SP0650.

FIG. 2. Map of expression vector SP1716.

FIG. 3. Map of expression vector SP2149.

FIG. 4. Map of expression vector SP4589.

FIG. 5. Map of expression vector SP4928.

FIG. 6. Agrobacterium-mediated transformations of embryogenic cell suspensions. Panel A: Agrobacterium-infected cells proliferating on selection medium with 100 mg/L Kanamycin; Panel B: embryos on embryo development medium (EDM); Panel C: embryos on embryo maturation medium (EMM); Panel D: shoot germination on embryo germination medium (EGM); Panel E: complete shoots in proliferation medium (PM), Panel F: potted transgenic plants in glasshouse.

FIG. 7. Diagram of injection sites of 2-chip disposal hemocytometer.

FIG. 8. Diagram of hemocytometer.

FIG. 9. Results of Rapid13 assay of Musa BAG1 events.

FIG. 10. Results of Rapid13 assay of ERG11 events.

FIG. 11. Results of Rapid13 assay of Smp-AMP-D1 events.

FIG. 12. Results of Rapid13 assay of RGA2 events.

FIG. 13. Results of ARSS assay of 12 different banana lines.

FIG. 14. Results of Rapid13 and ARSS assays of 12 different banana lines.

FIG. 15. Results of ARSS assay of 26 different banana lines.

FIG. 16. Alignment of amino acid sequences of certain defensin candidates, including signal peptide. Mba02_gl2080.1 (SEQ ID NO:210), Ma11_p12930.1 (SEQ ID NO:204), Ma08_p13660.1 (SEQ ID NO:183), Ma06_p21420.1 (SEQ ID NO:168), Ma04_p36140.1 (SEQ ID NO:153), Ma02_p12840.1 (SEQ ID NO:129), GB ID:RRT50697.1 (SEQ ID NO:126).

FIG. 17. Alignment of amino acid sequences of certain LTP candidates, including signal peptide. Ma09_p21930.1 (SEQ ID NO:192), Ma04_p17240.1 (SEQ ID NO:147), Ma04_p17190.1 (SEQ ID NO:144), Ma04_p30830.1 (SEQ ID NO:150), Ma04_p17200.1 (SEQ ID NO:141), Ma11_p18240.1 (SEQ ID NO:207).

FIG. 18. Alignment of amino acid sequences of certain snakin candidates, including signal peptide. Ma07_p21450.1 (SEQ ID NO:180), Ma10_p18110.1 (SEQ ID NO:201), Ma06_p09450.1 (SEQ ID NO:162), Ma09_p13940.1 (SEQ ID NO:189), Ma09_p27770.1 (SEQ ID NO:198), Ma06_p00870.1 (SEQ ID NO:159), Ma08_p22790.1 (SEQ ID NO:186), Ma06_p20150.1 (SEQ ID NO: 165).

FIG. 19. Alignment of amino acid sequences of certain TLP candidates, including signal peptide. Ma06_p33150.1 (SEQ ID NO:171), Ma06_p33170.1 (SEQ ID NO:174), Ma07_p17800.1 (SEQ ID NO:177), Ma02_p17990.1 (SEQ ID NO:135), Ma02_p13180.1 (SEQ ID NO:132), Ma03_p07220.1 (SEQ ID NO:138), Ma09_p26730.1 (SEQ ID NO:195), Ma04_p38470.1 (SEQ ID NO:156).

FIG. 20. Growth of Foc_TR4 with eugenol in DMSO solvent.

FIG. 21. Growth of Foc_TR4 with eugenol in EtOH/Tween-20 solvent.

FIG. 22. Percent of Foc_TR4 growth inhibition with eugenol in DMSO solvent.

FIG. 23. Percent of Foc_TR4 growth inhibition with eugenol in DMSO solvent.

FIG. 24. Growth of Foc_TR4 with geraniol in DMSO solvent.

FIG. 25. Growth of Foc_TR4 with geraniol in EtOH/Tween-20 solvent.

FIG. 26. Percent of Foc_TR4 growth inhibition with geraniol in DMSO solvent.

FIG. 27. Percent of Foc_TR4 growth inhibition with geraniol in EtOH/Tween-20 solvent.

FIG. 28. Growth of Foc_TR4 with limonene in DMSO solvent.

FIG. 29. Growth of Foc_TR4 with limonene in EtOH/Tween-20 solvent.

FIG. 30. Percent of Foc_TR4 growth inhibition with limonene in DMSO solvent.

FIG. 31. Percent of Foc_TR4 growth inhibition with limonene in EtOH/Tween-20 solvent.

FIG. 32. Map of expression vector SP0773.

### BRIEF DESCRIPTION OF THE SEQUENCES

SEQ ID NO:1: BAG1 (Musa BAG1) nucleic acid sequence.

SEQ ID NO:2: BAG1 (Musa BAG1) amino acid sequence.

SEQ ID NO:3: ERG11 RNAi nucleic acid sequence.

SEQ ID NO:4: Smp-AMP-D1 (also called Sm-AMP-D1) nucleic acid sequence.

SEQ ID NO:5: Smp-AMP-D1 (also called Sm-AMP-D1) amino acid sequence.

SEQ ID NO:6: RGA2 nucleic acid sequence.

SEQ ID NO:7: RGA2 amino acid sequence.

SEQ ID NO:8: RUBY nucleic acid sequence.

SEQ ID NO:9: RUBY amino acid sequence.

SEQ ID NO:10: SP0650, RUBY expression vector nucleic acid sequence.

SEQ ID NO:11: SP1716, Smp-AMP-D1 expression vector nucleic acid sequence.

SEQ ID NO:12: SP2149, RGA2 expression vector nucleic acid sequence.

SEQ ID NO:13: SP4589, ERG11 expression vector nucleic acid sequence.

SEQ ID NO:14: SP4928, BAG1 (Musa BAG1) expression vector nucleic acid sequence.

SEQ ID NO:15: 2A self-cleaving peptide nucleic acid sequence.

SEQ ID NO:16: 2A self-cleaving peptide amino acid sequence.

SEQ ID NO:17: HLVH12 promoter nucleic acid sequence.

SEQ ID NO:18: DCMV promoter nucleic acid sequence.

SEQ ID NO:19: FMVSgt:PCLSVFlt (also referred to as FSgt/PFLt) chimeric promoter nucleic acid sequence.

SEQ ID NO:20: Duplicated MMV (dMMV) promoter nucleic acid sequence.

SEQ ID NO:21: CmYLCV promoter nucleic acid sequence.

SEQ ID NO:22: CaMV e35S (e35S) promoter nucleic acid sequence.

SEQ ID NO:23: NOS promoter nucleic acid sequence.

SEQ ID NO:24: ScBV promoter nucleic acid sequence.

SEQ ID NO:25: CsVMV promoter nucleic acid sequence.

SEQ ID NO:26: FMVSgt promoter nucleic acid sequence.

SEQ ID NO:27: FS1_1 promoter nucleic acid sequence.

SEQ ID NO:28: FE_3 promoter nucleic acid sequence.

SEQ ID NO:29: Pea3A terminator nucleic acid sequence.

SEQ ID NO:30: At UBQ3 terminator nucleic acid sequence.

SEQ ID NO:31: Gmax MYB2 terminator nucleic acid sequence.

SEQ ID NO:32: AtRBCS2B terminator nucleic acid sequence.

SEQ ID NO:33: Pea E9 terminator nucleic acid sequence.

SEQ ID NO:34: AtHSP18.2 terminator nucleic acid sequence.

SEQ ID NO:35: Potato Ubi3 terminator nucleic acid sequence.

SEQ ID NO:36: At Tubulin B9 (AtTub) terminator nucleic acid sequence.

SEQ ID NO:37: 35S terminator nucleic acid sequence.

SEQ ID NO:38: Suberman MYB39 transcription factor (AtMYB39) nucleic acid sequence, from Arabidopsis thaliana, optimized for high GC content.

SEQ ID NO:39: Suberman MYB39 transcription factor (AtMYB39) from Arabidopsis thaliana, amino acid sequence.

SEQ ID NO:40: Blue copper-binding protein (GhUMC1) nucleic acid sequence, from Gossypium hirsutum, optimized for high GC content.

SEQ ID NO:41: Blue copper-binding protein (GhUMC1) from Gossypium hirsutum, amino acid sequence.

SEQ ID NO:42: I-3 R-gene receptor (I3) nucleic acid sequence, from Solanum pennellii, optimized for high GC content.

SEQ ID NO:43: I-3 R-gene receptor (I3) from Solanum pennellii, amino acid sequence.

SEQ ID NO:44: Ma02_g12980 from Musa acuminata, nucleic acid sequence.

SEQ ID NO:45: Ma02_g12980 from Musa acuminata, amino acid sequence.

SEQ ID NO:46: Ma03_g08560 from Musa acuminata, nucleic acid sequence.

SEQ ID NO:47: Ma03_g08560 from Musa acuminata, amino acid sequence.

SEQ ID NO:48: Ma03_g10750 from Musa acuminata, nucleic acid sequence.

SEQ ID NO:49: Ma03_g10750 from Musa acuminata, amino acid sequence.

SEQ ID NO:50: Ma03_g26280 (WRKY24 (PCD)) from Musa acuminata, nucleic acid sequence.

SEQ ID NO:51: Ma03_g26280 (WRKY24 (PCD)) from Musa acuminata, amino acid sequence.

SEQ ID NO:52: Ma04_g20880 (DMR6) from Musa acuminata, nucleic acid sequence.

SEQ ID NO:53: Ma04_g20880 (DMR6) from Musa acuminata, amino acid sequence.

SEQ ID NO:54: Ma04_g27910 (ATG8f (PCD)) from Musa acuminata, nucleic acid sequence.

SEQ ID NO:55: Ma04_g27910 (ATG8f (PCD)) from Musa acuminata, amino acid sequence.

SEQ ID NO:56: Ma05_g02830 (ATG8g (PCD)) from Musa acuminata, nucleic acid sequence.

SEQ ID NO:57: Ma05_g02830 (ATG8g (PCD)) from Musa acuminata, amino acid sequence.

SEQ ID NO:58: Ma05_g03720 nucleic acid sequence, from Musa acuminata, optimized for high GC content.

SEQ ID NO:59: Ma05_g03720 from Musa acuminata, amino acid sequence.

SEQ ID NO:60: Ma06_g00580 (NBS-LRR gene family member) nucleic acid sequence, from Musa acuminata, optimized for high GC content.

SEQ ID NO:61: Ma06_g00580 (NBS-LRR gene family member) from Musa acuminata, amino acid sequence.

SEQ ID NO:62: Ma06_g08420 nucleic acid sequence, from Musa acuminata, optimized for high GC content.

SEQ ID NO:63: Ma06_g08420 from Musa acuminata, amino acid sequence.

SEQ ID NO:64: Ma06_g31980 nucleic acid sequence, from Musa acuminata, optimized for high GC content.

SEQ ID NO:65: Ma06_g31980 from Musa acuminata, amino acid sequence.

SEQ ID NO:66: Ma06_g33150 antimicrobial protein, nucleic acid sequence, from Musa acuminata, optimized for high GC content.

SEQ ID NO:67: Ma06_g33150 antimicrobial protein, from Musa acuminata, amino acid sequence.

SEQ ID NO:68: Ma07_g03540 nucleic acid sequence, from Musa acuminata, optimized for high GC content.

SEQ ID NO:69: Ma07_g03540 from Musa acuminata, amino acid sequence.

SEQ ID NO:70: Ma07_g18150 from Musa acuminata, nucleic acid sequence.

SEQ ID NO:71: Ma07_g18150 from Musa acuminata, amino acid sequence.

SEQ ID NO:72: Ma08_g12090 (DMR6) from Musa acuminata, nucleic acid sequence.

SEQ ID NO:73: Ma08_g12090 (DMR6) from Musa acuminata, amino acid sequence.

SEQ ID NO:74: Ma08_g19730 from Musa acuminata, nucleic acid sequence.

SEQ ID NO:75: Ma08_g19730 from Musa acuminata, amino acid sequence.

SEQ ID NO:76: Ma09_g20240 from Musa acuminata, nucleic acid sequence.

SEQ ID NO:77: Ma09_g20240 from Musa acuminata, amino acid sequence.

SEQ ID NO:78: Ma09_g27170 (Bsr-d1 (P Barrier)) from Musa acuminata, nucleic acid sequence.

SEQ ID NO:79: Ma09_g27170 (Bsr-d1 (P Barrier)) from Musa acuminata, amino acid sequence.

SEQ ID NO:80: Ma09_g27770 nucleic acid sequence, from Musa acuminata, optimized for high GC content.

SEQ ID NO:81: Ma09_g27770 from Musa acuminata, amino acid sequence.

SEQ ID NO:82: Ma10_g02380 from Musa acuminata, nucleic acid sequence.

SEQ ID NO:83: Ma10_g02380 from Musa acuminata, amino acid sequence.

SEQ ID NO:84: Ma11_g02650 (DMR6) from Musa acuminata, nucleic acid sequence.

SEQ ID NO:85: Ma11_g02650 (DMR6) from Musa acuminata, amino acid sequence.

SEQ ID NO:86: Ma11_g07550 nucleic acid sequence, from Musa acuminata, optimized for high GC content.

SEQ ID NO:87: Ma11_g07550 from Musa acuminata, amino acid sequence.

SEQ ID NO:88: Ma11_gl4940 nucleic acid sequence, from Musa acuminata, optimized for high GC content.

SEQ ID NO:89: Ma11_g14940 from Musa acuminata, amino acid sequence.

SEQ ID NO:90: MaLYK1 (lysin-motif-containing receptor-like kinase 1) nucleic acid sequence, from Musa acuminata, G526A mutation to remove BsaI site.

SEQ ID NO:91: MaLYK1 (lysin-motif-containing receptor-like kinase 1) from Musa acuminata, amino acid sequence.

SEQ ID NO:92: MaRAR1 (co-chaperone of HSP90) nucleic acid sequence, from Musa acuminata, optimized for high GC content.

SEQ ID NO:93: MaRAR1 (co-chaperone of HSP90) from Musa acuminata, amino acid sequence.

SEQ ID NO:94: MpbHLH (ICE1-like transcription factor) nucleic acid sequence, from Musa acuminata×balbisiana, optimized for high GC content.

SEQ ID NO:95: MpbHLH (ICE1-like transcription factor) from Musa acuminata×balbisiana, amino acid sequence.

SEQ ID NO:96: ObEGS1 (eugenol biosynthesis) nucleic acid sequence, from Ocimum basilicum, optimized for high GC content.

SEQ ID NO:97: ObEGS1 (eugenol biosynthesis) from Ocimum basilicum, amino acid sequence.

SEQ ID NO:98: OsTPS19 (limonene biosynthesis) nucleic acid sequence, from Oryza sativa, optimized for high GC content.

SEQ ID NO:99: OsTPS19 (limonene biosynthesis) from Oryza sativa, amino acid sequence.

SEQ ID NO:100: OsUMP1 (proteosome maturation factor) nucleic acid sequence, from Oryza sativa, optimized for high GC content.

SEQ ID NO:101: OsUMP1 (proteosome maturation factor) from Oryza sativa, amino acid sequence.

SEQ ID NO:102: OsXa4 (disease resistance gene) nucleic acid sequence, from Oryza sativa, optimized for high GC content.

SEQ ID NO:103: OsXa4 (disease resistance gene) from Oryza sativa, amino acid sequence.

SEQ ID NO:104: PFLP, nucleic acid sequence.

SEQ ID NO:105: PFLP, amino acid sequence.

SEQ ID NO:106: Sm-AMP1-D1 alternate sequence, Stellaria media, nucleic acid sequence.

SEQ ID NO:107: Sm-AMP1-D1 alternate sequence, Stellaria media, amino acid sequence.

SEQ ID NO:108: SNC1-3 (R-gene receptor) nucleic acid sequence, from Arabidopsis thaliana, optimized for high GC content.

SEQ ID NO:109: SNC1-3 (R-gene receptor) Arabidopsis thaliana, amino acid sequence.

SEQ ID NO:110: UDP glycosyltransferase (SsGT1) nucleic acid sequence, from Solanum sogarandinum, optimized for high GC content.

SEQ ID NO:111: UDP glycosyltransferase (SsGT1) from Solanum sogarandinum, amino acid sequence.

SEQ ID NO:112: R-gene receptor (TPL) nucleic acid sequence, from Arabidopsis thaliana, optimized for high GC content.

SEQ ID NO:113: R-gene receptor (TPL) from Arabidopsis thaliana, amino acid sequence.

SEQ ID NO:114: Geraniol biosynthesis (VopGES) nucleic acid sequence, from Valeriana officinalis, optimized for high GC content.

SEQ ID NO:115: Geraniol biosynthesis (VopGES) from Valeriana officinalis, amino acid sequence.

SEQ ID NO:116: ZmUbi1 promoter, nucleic acid sequence.

SEQ ID NO:117: OsAct1 promoter, nucleic acid sequence.

SEQ ID NO:118: VND7 promoter, Musa acuminata, nucleic acid sequence.

SEQ ID NO:119: Ma521 Ma09_g14890 promoter, nucleic acid sequence.

SEQ ID NO:120: Ma119 Ma08_g12140 promoter, nucleic acid sequence.

SEQ ID NO:121: MaM4A Ma01_g10480 promoter, nucleic acid sequence.

SEQ ID NO:122: MaBB Ma04_g25440 promoter, nucleic acid sequence.

SEQ ID NO:123: Ma40554 Ma09_g15840 promoter, nucleic acid sequence.

SEQ ID NO:124: MaACT1 promoter, nucleic acid sequence.

SEQ ID NO:125: GB ID:RRT50697.1 defensin antimicrobial protein, Musa acuminata, nucleic acid sequence.

SEQ ID NO:126: GB ID:RRT50697.1 defensin antimicrobial protein, Musa acuminata amino acid sequence with yeast signal peptide.

SEQ ID NO:127: GB ID:RRT50697.1 defensin antimicrobial protein, Musa acuminata, amino acid sequence without yeast signal peptide.

SEQ ID NO:128: Ma02_g12840.1, defensin antimicrobial protein, Musa acuminata, nucleic acid sequence.

SEQ ID NO:129: Ma02_g12840.1, defensin antimicrobial protein, Musa acuminata, amino acid sequence with yeast signal peptide.

SEQ ID NO:130: Ma02_g12840.1, defensin antimicrobial protein, Musa acuminata, amino acid sequence without yeast signal peptide.

SEQ ID NO:131: Ma02_g13180.1, TLP antimicrobial protein, Musa acuminata, nucleic acid sequence.

SEQ ID NO:132: Ma02_g13180.1, TLP antimicrobial protein, Musa acuminata, amino acid sequence with yeast signal peptide.

SEQ ID NO:133: Ma02_1g3180.1, TLP antimicrobial protein, Musa acuminata, amino acid sequence without yeast signal peptide.

SEQ ID NO:134: Ma02_g17990.1, TLP antimicrobial protein, Musa acuminata, nucleic acid sequence.

SEQ ID NO:135: Ma02_g17990.1, TLP antimicrobial protein, Musa acuminata, amino acid sequence with yeast signal peptide.

SEQ ID NO:136: Ma02_g17990.1, TLP antimicrobial protein, Musa acuminata, amino acid sequence without yeast signal peptide.

SEQ ID NO:137: Ma03g07220.1, TLP antimicrobial protein, Musa acuminata, nucleic acid sequence.

SEQ ID NO:138: Ma03_g07220.1, TLP antimicrobial protein, Musa acuminata, amino acid sequence with yeast signal peptide.

SEQ ID NO:139: Ma03_g07220.1, TLP antimicrobial protein, Musa acuminata, amino acid sequence without yeast signal peptide.

SEQ ID NO:140: Ma04_g17200.1, LTP antimicrobial protein, Musa acuminata, nucleic acid sequence.

SEQ ID NO:141: Ma04_g17200.1, LTP antimicrobial protein, Musa acuminata, amino acid sequence with yeast signal peptide.

SEQ ID NO:142: Ma04_g17200.1, LTP antimicrobial protein, Musa acuminata, amino acid sequence without yeast signal peptide.

SEQ ID NO:143: Ma04_g17190.1, LTP antimicrobial protein, Musa acuminata, nucleic acid sequence.

SEQ ID NO:144: Ma04_g17190.1, LTP antimicrobial protein, Musa acuminata, amino acid sequence with yeast signal peptide.

SEQ ID NO:145: Ma04_g17190.1, LTP antimicrobial protein, Musa acuminata, amino acid sequence without yeast signal peptide.

SEQ ID NO:146: Ma04_g17240.1, LTP antimicrobial protein, Musa acuminata, nucleic acid sequence.

SEQ ID NO:147: Ma04_g17240.1, LTP antimicrobial protein, Musa acuminata, amino acid sequence with yeast signal peptide.

SEQ ID NO:148: Ma04_g17240.1, LTP antimicrobial protein, Musa acuminata, amino acid sequence without yeast signal peptide.

SEQ ID NO:149: Ma04_g30830.1, LTP antimicrobial protein, Musa acuminata, nucleic acid sequence.

SEQ ID NO:150: Ma04_g30830.1, LTP antimicrobial protein, Musa acuminata, amino acid sequence with yeast signal peptide.

SEQ ID NO:151: Ma04_g30830.1, LTP antimicrobial protein, Musa acuminata, amino acid sequence without yeast signal peptide.

SEQ ID NO:152: Ma04_g36140.1, defensin antimicrobial protein, Musa acuminata, nucleic acid sequence.

SEQ ID NO:153: Ma04_g36140.1, defensin antimicrobial protein, Musa acuminata, amino acid sequence with yeast signal peptide.

SEQ ID NO:154: Ma04_g36140.1, defensin antimicrobial protein, Musa acuminata, amino acid sequence without yeast signal peptide.

SEQ ID NO:155: Ma04_g38470.1, TLP antimicrobial protein, Musa acuminata, nucleic acid sequence.

SEQ ID NO:156: Ma04_g38470.1, TLP antimicrobial protein, Musa acuminata, amino acid sequence with yeast signal peptide.

SEQ ID NO:157: Ma04_g38470.1, TLP antimicrobial protein, Musa acuminata, amino acid sequence without yeast signal peptide.

SEQ ID NO:158: Ma06_g00870.1, snakin antimicrobial protein, Musa acuminata, nucleic acid sequence.

SEQ ID NO:159: Ma06_g00870.1, snakin antimicrobial protein, Musa acuminata, amino acid sequence with yeast signal peptide.

SEQ ID NO:160: Ma06_g00870.1, snakin antimicrobial protein, Musa acuminata, amino acid sequence without yeast signal peptide.

SEQ ID NO:161: Ma06_g09450.1, snakin antimicrobial protein, Musa acuminata, nucleic acid sequence.

SEQ ID NO:162: Ma06_g09450.1, snakin antimicrobial protein, Musa acuminata, amino acid sequence with yeast signal peptide.

SEQ ID NO:163: Ma06_g09450.1, snakin antimicrobial protein, Musa acuminata, amino acid sequence without yeast signal peptide.

SEQ ID NO:164: Ma06_g20150.1, snakin antimicrobial protein, Musa acuminata, nucleic acid sequence.

SEQ ID NO:165: Ma06_g20150.1, snakin antimicrobial protein, Musa acuminata, amino acid sequence with yeast signal peptide.

SEQ ID NO:166: Ma06_g20150.1, snakin antimicrobial protein, Musa acuminata, amino acid sequence without yeast signal peptide.

SEQ ID NO:167: Ma06_g21420.1, defensin antimicrobial protein, Musa acuminata, nucleic acid sequence.

SEQ ID NO:168: Ma06_g21420.1, defensin antimicrobial protein, Musa acuminata, amino acid sequence with yeast signal peptide.

SEQ ID NO:169: Ma06_g21420.1, defensin antimicrobial protein, Musa acuminata, amino acid sequence without yeast signal peptide.

SEQ ID NO:170: Ma06_g33150.1, TLP antimicrobial protein, Musa acuminata, nucleic acid sequence.

SEQ ID NO:171: Ma06_g33150.1, TLP antimicrobial protein, Musa acuminata, amino acid sequence with yeast signal peptide.

SEQ ID NO:172: Ma06_g33150.1, TLP antimicrobial protein, Musa acuminata, amino acid sequence without yeast signal peptide.

SEQ ID NO:173: Ma06_g33170.1, TLP antimicrobial protein, Musa acuminata, nucleic acid sequence.

SEQ ID NO:174: Ma06_g33170.1, TLP antimicrobial protein, Musa acuminata, amino acid sequence with yeast signal peptide.

SEQ ID NO:175: Ma06_g33170.1, TLP antimicrobial protein, Musa acuminata, amino acid sequence without yeast signal peptide.

SEQ ID NO:176: Ma07_g17800.1, TLP antimicrobial protein, Musa acuminata, nucleic acid sequence.

SEQ ID NO:177: Ma07_g17800.1, TLP antimicrobial protein, Musa acuminata, amino acid sequence with yeast signal peptide.

SEQ ID NO:178: Ma07_g17800.1, TLP antimicrobial protein, Musa acuminata, amino acid sequence without yeast signal peptide.

SEQ ID NO:179: Ma07_g21450.1, snakin antimicrobial protein, Musa acuminata, nucleic acid sequence.

SEQ ID NO:180: Ma07_g21450.1, snakin antimicrobial protein, Musa acuminata, amino acid sequence with yeast signal peptide.

SEQ ID NO:181: Ma07_g21450.1, snakin antimicrobial protein, Musa acuminata, amino acid sequence without yeast signal peptide.

SEQ ID NO:182: Ma08_g13660.1, defensin antimicrobial protein, Musa acuminata, nucleic acid sequence.

SEQ ID NO:183: Ma08_g13660.1, defensin antimicrobial protein, Musa acuminata, amino acid sequence with yeast signal peptide.

SEQ ID NO:184: Ma08_g13660.1, defensin antimicrobial protein, Musa acuminata, amino acid sequence without yeast signal peptide.

SEQ ID NO:185: Ma08_g22790.1, snakin antimicrobial protein, Musa acuminata, nucleic acid sequence.

SEQ ID NO:186: Ma08_g22790.1, snakin antimicrobial protein, Musa acuminata, amino acid sequence with yeast signal peptide.

SEQ ID NO:187: Ma08_g22790.1, snakin antimicrobial protein, Musa acuminata, amino acid sequence without yeast signal peptide.

SEQ ID NO:188: Ma09_g13940.1, snakin antimicrobial protein, Musa acuminata, nucleic acid sequence.

SEQ ID NO:189: Ma09_g13940.1, snakin antimicrobial protein, Musa acuminata, amino acid sequence with yeast signal peptide.

SEQ ID NO:190: Ma09_g13940.1, snakin antimicrobial protein, Musa acuminata, amino acid sequence without yeast signal peptide.

SEQ ID NO:191: Ma09_g21930.1, LTP antimicrobial protein, Musa acuminata, nucleic acid sequence.

SEQ ID NO:192: Ma09_g21930.1, LTP antimicrobial protein, Musa acuminata, amino acid sequence with yeast signal peptide.

SEQ ID NO:193: Ma09_g21930.1, LTP antimicrobial protein, Musa acuminata, amino acid sequence without yeast signal peptide.

SEQ ID NO:194: Ma09_g26730.1, TLP antimicrobial protein, Musa acuminata, nucleic acid sequence.

SEQ ID NO:195: Ma09_g26730.1, TLP antimicrobial protein, Musa acuminata, amino acid sequence with yeast signal peptide.

SEQ ID NO:196: Ma09_g26730.1, TLP antimicrobial protein, Musa acuminata, amino acid sequence without yeast signal peptide.

SEQ ID NO:197: Ma09_g27770.1, snakin antimicrobial protein, Musa acuminata, nucleic acid sequence.

SEQ ID NO:198: Ma09_g27770.1, snakin antimicrobial protein, Musa acuminata, amino acid sequence with yeast signal peptide.

SEQ ID NO:199: Ma09_g27770.1, snakin antimicrobial protein, Musa acuminata, amino acid sequence without yeast signal peptide.

SEQ ID NO:200: Ma10_g18110.1, snakin antimicrobial protein, Musa acuminata, nucleic acid sequence.

SEQ ID NO:201: Ma10_g18110.1, snakin antimicrobial protein, Musa acuminata, amino acid sequence with yeast signal peptide.

SEQ ID NO:202: Ma10_g18110.1, snakin antimicrobial protein, Musa acuminata, amino acid sequence without yeast signal peptide.

SEQ ID NO:203: Ma11_g12930.1, defensin antimicrobial protein, Musa acuminata, nucleic acid sequence.

SEQ ID NO:204: Ma11_g12930.1, defensin antimicrobial protein, Musa acuminata, amino acid sequence with yeast signal peptide.

SEQ ID NO:205: Ma11_g12930.1, defensin antimicrobial protein, Musa acuminata, amino acid sequence without yeast signal peptide.

SEQ ID NO:206: Ma11_g18240.1, LTP antimicrobial protein, Musa acuminata, nucleic acid sequence.

SEQ ID NO:207: Ma11_g18240.1, LTP antimicrobial protein, Musa acuminata, amino acid sequence with yeast signal peptide.

SEQ ID NO:208: Ma11_g18240.1, LTP antimicrobial protein, Musa acuminata, amino acid sequence without yeast signal peptide.

SEQ ID NO:209: Mba02_g12080.1, defensin antimicrobial protein, Musa acuminata, nucleic acid sequence.

SEQ ID NO:210: Mba02_g12080.1, defensin antimicrobial protein, Musa acuminata, amino acid sequence with yeast signal peptide.

SEQ ID NO:211: Mba02_g12080.1, defensin antimicrobial protein, Musa acuminata, amino acid sequence without yeast signal peptide.

SEQ ID NO:212: CaMV 35S terminator, nucleic acid sequence.

SEQ ID NO:213: NOS terminator, nucleic acid sequence.

SEQ ID NO:214: PBI synthetic terminator, nucleic acid sequence.

SEQ ID NO:215: GB ID:RRT50697.1 defensin antimicrobial protein, Musa acuminata, nucleic acid sequence.

SEQ ID NO:216: GB ID:RRT50697.1 defensin antimicrobial protein, Musa acuminata, nucleic acid sequence.

SEQ ID NO:217: Mba02_g12080.1, defensin antimicrobial protein, Musa acuminata, genomic DNA.

SEQ ID NO:218: Mba02_g12080.1, defensin antimicrobial protein, Musa acuminata, genomic DNA.

SEQ ID NO:219: MaRGA2 promoter, Musa acuminata, genomic DNA.

SEQ ID NO:220—MaRGA2 promoter edit (Ma03_g09130A)—nucleic acid sequence.

SEQ ID NO:221—MaRGA2 promoter edit (Ma03_g09130B)—nucleic acid sequence.

SEQ ID NO:222—MaRGA2 promoter edit (Ma03_g09130C)—nucleic acid sequence.

SEQ ID NO:223—SP0773, TLP/snakin antimicrobial peptide expression vector nucleic acid sequence.

SEQ ID NO:224—Bsr-kl (P Barrier), Musa acuminata, genomic nucleic acid sequence.

### DETAILED DESCRIPTION

The present disclosure generally describes transgenic banana plants having increased resistance to Fusarium oxysporum f.sp. cubense Tropical Race 4 (TR4), and methods for making such transgenic plants. The following sections provide embodiments that describe the subject matter in greater detail.

### I. Nucleic Acid and Polypeptide Sequences

Certain embodiments of the current disclosure concern nucleic acid sequences (polynucleotides) and the corresponding amino acid sequences (proteins or polypeptides) for increasing resistance of banana plants to Fusarium oxysporum f.sp. cubense Tropical Race 4 (TR4). Complements to any nucleic acid or protein sequences described herein are also provided.

“Identity,” as is well understood in the art, is a relationship between two or more polypeptide sequences or two or more polynucleotide sequences, as determined by comparing the sequences. In the art, “identity” also means the degree of sequence relatedness between polypeptide or polynucleotide sequences, as determined by the match between strings of such sequences. Methods to determine “identity” are designed to give the largest match between the sequences tested. Moreover, methods to determine identity are codified in publicly available programs. “Identity” can be readily calculated by any of the many methods known to those of skill in the art. Computer programs can be used to determine “identity” between two sequences these programs include but are not limited to, GCG; suite of five BLAST programs, three designed for nucleotide sequences queries (BLASTN, BLASTX, and TBLASTX) and two designed for protein sequence queries (BLASTP and TBLASTN). The BLASTX program is publicly available from NCBI and other sources (BLAST Manual, NCBI NLM NIH, Bethesda, Md. 20894). The well-known Smith Waterman algorithm can also be used to determine identity.

In accordance with the present disclosure, a polynucleotide or polypeptide sequence as described herein may exhibit at least from about 34%, 40%, 50%, 60%, 62% or 70% to about 100% sequence identity to at least one of the sequences set forth herein. For example, in one embodiment, a nucleic acid sequence as described herein may comprise, for example, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a sequence selected from SEQ ID NOs:1, 3, 4, 6, 8 or 9-15, or a complement thereof. In other embodiments, an amino acid sequence as described herein may comprise for example, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a sequence selected from SEQ ID NOs:2, 5, 7 or 9.

Parameters for polypeptide sequence comparison include the following: Algorithm: Needleman and Wunsch (J. Mol. Biol. 48:443-453, 1970); Comparison matrix: BLOSUM62 from Hentikoff and Hentikoff, (Proc. Natl. Acad. Sci. USA 89:10915-10919, 1992); Gap Penalty: 12; and Gap Length Penalty: 4. A program that can be used with these parameters is publicly available as the “gap” program from Genetics Computer Group, Madison WI. The above parameters along with no penalty for end gap may serve as default parameters for peptide comparisons.

Parameters for nucleic acid sequence comparison include the following: Algorithm: Needleman and Wunsch (supra); Comparison matrix: matches=+10; mismatches=0; Gap Penalty: 50; and Gap Length Penalty: 3. A program that can be used with these parameters is publicly available as the “gap” program from Genetics Computer Group, Madison Wis. The above parameters may serve as the default parameters for nucleic acid comparisons.

As used herein, “hybridization,” “hybridizes,” or “capable of hybridizing” is understood to mean the forming of a double- or triple-stranded molecule or a molecule with partial double- or triple-stranded nature. Such hybridization may take place under relatively high-stringency conditions, including low salt and/or high temperature conditions, such as provided by a wash in about 0.02 M to about 0.15 M NaCl at temperatures of about 50° C. to about 70° C. for 10 min. In one embodiment of the present disclosure, the conditions are 0.15 M NaCl and 70° C. Stringent conditions tolerate little mismatch between a nucleic acid and a target strand. Such conditions are well-known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like. Also included may be a protein or polypeptide, or fragment thereof, such as any of those set forth herein.

“Fragment”, with respect to the nucleic acid sequences disclosed herein, refers to any part of a polynucleotide molecule that retains a usable, functional characteristic. Useful fragments include oligonucleotides and polynucleotides that may be used as probes or primers in hybridization or amplification technologies or in the regulation of replication, transcription or translation. A polynucleotide fragment refers to any subsequence of a polynucleotide, typically, of at least about 15 consecutive nucleotides, at least about 16 consecutive nucleotides, at least about 17 consecutive nucleotides, at least about 18 consecutive nucleotides, at least about 19 consecutive nucleotides, at least about 20 consecutive nucleotides, at least about 21 consecutive nucleotides, at least about 22 consecutive nucleotides, at least about 23 consecutive nucleotides, at least about 24 consecutive nucleotides, at least about 25 consecutive nucleotides, at least about 30 consecutive nucleotides, at least about 35 nucleotides, at least about 40 consecutive nucleotides, at least about 45 consecutive nucleotides, or at least about 50 nucleotides or more, of any of the nucleic acid sequences provided herein.

Fragments may also include subsequences of polypeptides and protein molecules, or a subsequence of the polypeptide, as disclosed herein. Fragments may have antigenic potential, or may be a subsequence of the polypeptide that performs at least one biological function of the intact polypeptide in substantially the same manner, or to a similar extent, as does the intact polypeptide. Fragments can vary in size from as few as 5 amino acids to the full length of the intact polypeptide, but are preferably at least about 10 amino acids in length, at least about 15 amino acids in length, at least about 20 amino acids in length, at least about 25 amino acids in length, at least about 30 amino acids in length, at least about 35 amino acids in length, at least about 40 amino acids in length, at least about 45 amino acids in length, at least about 50 amino acids in length, at least about 55 amino acids in length, or at least about 60 amino acids in length or more, of any of the amino acid sequences provided herein.

The nucleic acids and amino acids provided herein may be from any source, e.g., identified as naturally occurring in a plant, or synthesized, e.g., by mutagenesis of the disclosed nucleic acid sequences, for example to create a coding sequence with a G/C content more like the G/C content of naturally occurring genes from a particular plant. The naturally occurring sequence may be from any plant or algal species, as described herein.

### II. Plant Transformation Constructs

Vectors used for plant transformation may include, for example, plasmids, cosmids, YACs (yeast artificial chromosomes), BACs (bacterial artificial chromosomes) or any other suitable cloning system, as well as fragments of DNA therefrom. Thus when the term “vector” or “expression vector” is used, all of the foregoing types of vectors, as well as nucleic acid sequences isolated therefrom, are included. It is contemplated that utilization of cloning systems with large insert capacities will allow introduction of large DNA sequences comprising more than one selected gene. In accordance with the present disclosure, this could be used to introduce genes corresponding to an entire biosynthetic pathway into a plant. Introduction of such sequences may be facilitated by use of bacterial or yeast artificial chromosomes (BACs or YACs, respectively), or even plant artificial chromosomes. For example, the use of BACs for Agrobacterium-mediated transformation was disclosed by Hamilton et al. (Proc. Natl. Acad. Sci. USA 93:9975-9979, 1996).

Particularly useful for transformation are expression cassettes that have been isolated from such vectors. DNA segments used for transforming plant cells will, of course, generally comprise the cDNA, gene or genes that one desires to introduce into and have expressed in the host cells. These DNA segments can further include structures such as promoters, enhancers, polylinkers, terminators or even regulatory genes as desired. The DNA segment or gene chosen for cellular introduction will often encode a protein that will be expressed in the resultant recombinant cells resulting in a screenable or selectable trait and/or that will impart an improved phenotype to the resulting transgenic plant. However, this may not always be the case, and the present disclosure also encompasses transgenic plants incorporating non-expressed transgenes. Components that may be included with vectors used in the current disclosure are as follows.

### A. Promoters and Other Regulatory Elements

In certain embodiments, the presently disclosed expression cassettes further comprise one or more promoters. In addition to the promoter sequences disclosed herein, other exemplary promoters for expression of a nucleic acid sequence include a plant promoter such as the CaMV 35S promoter (Odell et al., Nature 313:810-812, 1985), or others such as CaMV 19S (Lawton et al., Plant Mol. Biol. 9:315-324, 1987), nos (Ebert et al., Proc. Natl. Acad. Sci. USA 84:5745-5749, 1987), Adh (Walker et al., Proc. Natl. Acad. Sci. USA 84:6624-6628, 1987), sucrose synthase (Yang and Russell, Proc. Natl. Acad. Sci. USA 87:4144-4148, 1990), α-tubulin, actin (Wang et al., Mol. Cell Biol. 12:3399-3406, 1992), cab (Sullivan et al., Mol. Gen. Genet. 215:431-440, 1989), PEPCase (Hudspeth and Grula, Plant Mol. Biol. 12:579-589, 1989) or those associated with the R gene complex (Chandler et al., Plant Cell 1:1175-1183, 1989). Tissue specific promoters such as root cell promoters (Conkling et al., Plant Physiol. 93:1203-1211, 1990) and tissue specific enhancers are also contemplated to be useful, as are inducible promoters such as ABA- and turgor-inducible promoters. The PAL2 promoter may also be useful with the disclosure (U.S. Patent Application Publication No. 2004/0049802, the entire disclosure of which is specifically incorporated herein by reference). In one embodiment of the present disclosure, the native promoter associated with one or more of the nucleic acid sequences disclosed herein is used. In some embodiments, the promoter is a strong promoter or a weak promoter.

The DNA sequence between the transcription initiation site and the start of the coding sequence, i.e., the untranslated leader sequence, can also influence gene expression. One may thus wish to employ a particular leader sequence with a transformation construct of the present disclosure. Leader sequences are contemplated to include those that comprise sequences predicted to direct optimum expression of the attached gene, i.e., to include a consensus leader sequence that may increase or maintain mRNA stability and prevent inappropriate initiation of translation. The choice of such sequences will be known to those of skill in the art in light of the present disclosure. Sequences that are derived from genes that are highly expressed in plants may be desirable.

It is contemplated that vectors for use in accordance with the present disclosure may be constructed to include an ocs enhancer element. This element was first identified as a 16 bp palindromic enhancer from the octopine synthase (ocs) gene of Agrobacterium (Ellis et al., EMBO J. 6:3203-3208, 1987), and is present in at least 10 other promoters (Bouchez et al., EMBO J. 8:4197-4204, 1989). The use of an enhancer element, such as the ocs element and particularly multiple copies of the element, may act to increase the level of transcription from adjacent promoters when applied in the context of plant transformation.

It is envisioned that presently disclosed coding sequences may be introduced under the control of novel promoters or enhancers, etc., or homologous or tissue specific promoters or control elements. Vectors for use in tissue-specific targeting of genes in transgenic plants will typically include tissue-specific promoters and may also include other tissue-specific control elements such as enhancer sequences. Promoters that direct specific or enhanced expression in certain plant tissues will be known to those of skill in the art in light of the present disclosure. These include, for example, the rbcS promoter, specific for green tissue; the ocs, nos and mas promoters that have higher activity in roots or wounded leaf tissue.

### B. Terminators

In certain embodiments, the presently disclosed expression cassettes further comprise one or more terminators. Transformation constructs prepared in accordance with the present disclosure will typically include a 3′ end DNA sequence that acts as a signal to terminate transcription and allow for the polyadenylation of the mRNA produced by coding sequences operably linked to a promoter. In one embodiment of the present disclosure, the native terminator associated with a nucleic acid sequence disclosed herein is used. Alternatively, a heterologous 3′ end may enhance the expression of sense or antisense sequences. In addition to the terminator sequences disclosed herein, further examples of terminators that are deemed to be useful in this context include those from the nopaline synthase gene of Agrobacterium tumefaciens (nos 3′ end) (Bevan et al., Nucl. Acids Res. 11:369-385, 1983), the terminator for the T7 transcript from the octopine synthase gene of Agrobacterium tumefaciens, and the 3′ end of the protease inhibitor I or II genes from potato or tomato. Regulatory elements such as an Adh intron (Callis et al., Genes Dev. 1:1183-1200, 1987), sucrose synthase intron (Vasil et al., Plant Physiol. 91:1575-1579, 1989) or TMV omega element (Gallie and Kado, Proc. Natl. Acad. Sci. USA 86:129-132, 1989), may further be included where desired.

### C. Transit or Signal Peptides

In certain embodiments of the present disclosure transit or signal sequences may be incorporated into the presently disclosed coding sequences. Sequences that are joined to the coding sequence of an expressed gene, which are removed post-translationally from the initial translation product and that facilitate the transport of the protein into or through intracellular or extracellular membranes, are termed transit (usually into vacuoles, vesicles, plastids and other intracellular organelles) and signal sequences (usually to the endoplasmic reticulum, golgi apparatus and outside of the cellular membrane). By facilitating the transport of the protein into compartments inside and outside the cell, these sequences may increase the accumulation of gene product protecting them from proteolytic degradation. These sequences also allow for additional mRNA sequences from highly expressed genes to be attached to the coding sequence of the genes. Since mRNA being translated by ribosomes is more stable than naked mRNA, the presence of translatable mRNA in front of the gene may increase the overall stability of the mRNA transcript from the gene and thereby increase synthesis of the gene product. Since transit and signal sequences are usually post-translationally removed from the initial translation product, the use of these sequences allows for the addition of extra translated sequences that may not appear on the final polypeptide. It further is contemplated that targeting of certain proteins may be desirable in order to enhance the stability of the protein (U.S. Pat. No. 5,545,818, incorporated herein by reference in its entirety).

Additionally, vectors may be constructed and employed in the intracellular targeting of a specific gene product within the cells of a transgenic plant or in directing a protein to the extracellular environment. This generally will be achieved by joining a DNA sequence encoding a transit or signal peptide sequence to the coding sequence of a particular gene. The resultant transit, or signal, peptide will transport the protein to a particular intracellular, or extracellular destination, respectively, and will then be post-translationally removed.

### D. Marker Genes

By employing a selectable or screenable marker protein, one can provide or enhance the ability to identify transformants. “Marker genes” are genes that impart a distinct phenotype to cells expressing the marker protein and thus allow such transformed cells to be distinguished from cells that do not have the marker. Such genes may encode either a selectable or screenable marker, depending on whether the marker confers a trait that one can “select” for by chemical means, i.e., through the use of a selective agent (e.g., a herbicide, antibiotic, or the like), or whether it is simply a trait that one can identify through observation or testing, i.e., by “screening” (e.g., the green fluorescent protein). In addition to the marker genes disclosed in the Sequence Listing, many additional examples of suitable marker proteins are known to the art and can be employed in the practice of the present disclosure.

Included within the terms “selectable” or “screenable” markers also are genes that encode a “secretable marker” whose secretion can be detected as a means of identifying or selecting for transformed cells. Examples include markers that are secretable antigens that can be identified by antibody interaction, or even secretable enzymes that can be detected by their catalytic activity. Secretable proteins fall into a number of classes, including small, diffusible proteins detectable, e.g., by ELISA; small active enzymes detectable in extracellular solution (e.g., α-amylase, β-lactamase, phosphinothricin acetyltransferase); and proteins that are inserted or trapped in the cell wall (e.g., proteins that include a leader sequence such as that found in the expression unit of extensin or tobacco PR S).

Many selectable marker coding regions are known and could be used with the present disclosure including, but not limited to, neo (Potrykus et al., Mol. Gen. Genet. 199:169-177, 1985), which provides kanamycin resistance and can be selected for using kanamycin, G418, paromomycin, etc.; bar, which confers bialaphos or phosphinothricin resistance; a mutant EPSP synthase protein conferring glyphosate resistance; a nitrilase such as bxn from Klebsiella ozaenae, which confers resistance to bromoxynil (Stalker et al., J. Biol. Chem. 263:6310-6314, 1988); a mutant acetolactate synthase (ALS), which confers resistance to imidazolinone, sulfonylurea or other ALS inhibiting chemicals (European Patent Application 154,204, 1985); a methotrexate resistant DHFR (Thillet et al., J. Biol. Chem. 263:12500-12508, 1988), a dalapon dehalogenase that confers resistance to the herbicide dalapon; or a mutated anthranilate synthase that confers resistance to 5-methyl tryptophan.

An illustrative embodiment of selectable marker capable of being used in systems to select transformants are those that encode the enzyme phosphinothricin acetyltransferase, such as the bar gene from Streptomyces hygroscopicus or the pat gene from Streptomyces viridochromogenes. The enzyme phosphinothricin acetyl transferase (PAT) inactivates the active ingredient in the herbicide bialaphos, phosphinothricin (PPT). PPT inhibits glutamine synthetase, causing rapid accumulation of ammonia and cell death.

Screenable markers that may be employed include a β glucuronidase (GUS) or uidA gene, which encodes an enzyme for which various chromogenic substrates are known; an R-locus gene, which encodes a product that regulates the production of anthocyanin pigments (red color) in plant tissues; a β lactamase gene (Sutcliffe, Proc. Natl. Acad. Sci. USA 75:3737-3741, 1978), which encodes an enzyme for which various chromogenic substrates are known (e.g., PADAC, a chromogenic cephalosporin); a xylE gene (Zukowsky et al., Proc. Natl. Acad. Sci. USA 80:1101-1105, 1983), which encodes a catechol dioxygenase that can convert chromogenic catechols; an α-amylase gene (Ikuta et al., Biotechnology 8:241-242, 1990); a tyrosinase gene (Katz et al., J. Gen. Microbiol. 129:2703-2714, 1983), which encodes an enzyme capable of oxidizing tyrosine to DOPA and dopaquinone, which in turn condenses to form the easily-detectable compound melanin; a β galactosidase gene, which encodes an enzyme for which there are chromogenic substrates; a luciferase (lux) gene (Ow et al., Science 234:856-859, 1986), which allows for bioluminescence detection; an aequorin gene (Prasher et al., Biochem. Biophys. Res. Commun. 126:1259-1268, 1985), which may be employed in calcium-sensitive bioluminescence detection; or a gene encoding for green fluorescent protein (GFP; Sheen et al., Plant J. 8:777-784, 1995; Haseloff et al., Proc. Natl. Acad. Sci. USA 94:2122-2127, 1997; Reichel et al., Proc. Natl. Acad. Sci. USA 93:5888-5893, 1996; WO 97/41228) is also contemplated as a useful reporter gene. Expression of green fluorescent protein may be visualized in a cell or plant as fluorescence following illumination by particular wavelengths of light.

### III. Antisense and RNAi Constructs

Antisense and RNAi treatments represent one way of altering gene activity in accordance with the present disclosure (e.g., by down regulation of genes or transcription factors that inhibit expression of an ERG11 gene).

Techniques for RNAi are well known in the art and are described in, for example, Lehner et al., (Brief Funct. Genomic Proteomic 3:68-83, 2004) and Downward (BMJ 328:1245-1248, 2004). The technique is based on the fact that double stranded RNA is capable of directing the degradation of messenger RNA with sequence complementary to one or the other strand (Fire et al., Nature 391:806-811, 1998). Therefore, by expression of a particular coding sequence in sense and antisense orientation, either as a fragment or longer portion of the corresponding coding sequence, the expression of that coding sequence can be down-regulated.

Antisense, and in some aspects RNAi, methodology takes advantage of the fact that nucleic acids tend to pair with “complementary” sequences. By complementary, it is meant that polynucleotides are those that are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense oligonucleotides, when introduced into a target cell, specifically bind to their target polynucleotide and interfere with transcription, RNA processing, transport, translation and/or stability. Antisense and RNAi constructs, or DNA encoding such RNA's, may be employed to inhibit gene transcription or translation or both within a host cell, either in vitro or in vivo, such as within a host plant cell. In certain embodiments of the present disclosure, such an oligonucleotide may comprise any unique portion of a nucleic acid sequence provided herein. In certain embodiments of the present disclosure, such a sequence comprises at least 18, 20, 25, 30, 50, 75 or 100 or more contiguous nucleic acids of a nucleic acid sequence of interest, and/or complements thereof, which may be in sense and/or antisense orientation. By including sequences in both sense and antisense orientation, increased suppression of the corresponding coding sequence may be achieved.

Constructs may be designed that are complementary to all or part of the promoter and other control regions, exons, introns or even exon-intron boundaries of a gene. It is contemplated that the most effective constructs may include regions complementary to intron/exon splice junctions. Thus, it is proposed that one embodiment includes a construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences that are completely complementary will be sequences that are entirely complementary throughout their entire length and have no base mismatches. Other sequences with lower degrees of homology also are contemplated. For example, an RNAi or antisense construct that has limited regions of high homology, but also contains a non-homologous region (e.g., ribozyme; see above) could be designed. Methods for selection and design of sequences that generate RNAi are well known in the art (e.g., Reynolds et al., Nat. Biotechnol. 22:326-330, 2004). These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone may be used. The cDNA or a synthesized polynucleotide may provide more convenient restriction sites for the remaining portion of the construct and, therefore, would be used for the rest of the sequence. Constructs useful for generating RNAi may also comprise concatemers of sub-sequences that display gene regulating activity.

### IV. Gene Editing

One method for producing the transgenic plants of the present disclosure is through genome modification using site-specific integration or genome editing. Targeted modification of plant genomes through the use of genome editing methods can be used to create improved plant lines through modification of plant genomic DNA. As used herein “site-directed integration” refers to genome editing methods that enable targeted insertion of one or more nucleic acids of interest into a plant genome. Suitable methods for altering a wild-type DNA sequence or a preexisting transgenic sequence (for example altering a native promoter to increase expression of the associated gene) or for inserting DNA into a plant genome at a pre-determined chromosomal site include any method known in the art. Exemplary methods include the use of sequence specific nucleases, such as zinc-finger nucleases, engineered or native meganucleases, TALE-endonucleases, or an RNA-guided endonucleases (for example, a Clustered Regularly Interspersed Short Palindromic Repeat (CRISPR)/Cas9 system, a CRISPR/Cpf1 system, a CRISPR/CasX system, a CRISPR/CasY system, or a CRISPR/Cascade system). Several embodiments relate to methods of genome editing by using single-stranded oligonucleotides to introduce precise base pair modifications in a plant genome. Methods of genome editing to modify, delete, or insert nucleic acid sequences into genomic DNA are known in the art.

In certain embodiments, the present disclosure provides modification or replacement of an existing coding sequence, such as an existing transgenic insert, within a plant genome with a sequence encoding a different protein, or an expression cassette comprising such a protein. Several embodiments relate to the use of a known genome editing methods, such as zinc-finger nucleases, engineered or native meganucleases, TALE-endonucleases, or an RNA-guided endonucleases (for example, a Clustered Regularly Interspersed Short Palindromic Repeat (CRISPR)/Cas9 system, a CRISPR/Cpf1 system, a CRISPR/CasX system, a CRISPR/CasY system, or a CRISPR/Cascade system).

Several embodiments may therefore relate to a recombinant DNA construct comprising an expression cassette(s) encoding a site-specific nuclease and, optionally, any associated protein(s) to carry out genome modification. These nuclease-expressing cassette(s) may be present in the same molecule or vector as a donor template for templated editing. Several methods for site-directed integration are known in the art involving different sequence-specific nucleases (or complexes of proteins or guide RNA or both) that cut the genomic DNA to produce a double strand break (DSB) or nick at a desired genomic site or locus. As understood in the art, during the process of repairing the DSB or nick introduced by the nuclease enzyme, the donor template DNA, transgene, or expression cassette may become integrated into the genome at the site of the DSB or nick. The presence of the homology arm(s) in the DNA to be integrated may promote the adoption and targeting of the insertion sequence into the plant genome during the repair process through homologous recombination, although an insertion event may occur through non-homologous end joining (NHEJ). As used herein, the term “double-strand break inducing agent” refers to any agent that can induce a double-strand break (DSB) in a DNA molecule. In some embodiments, the double-strand break inducing agent is a site-specific genome modification enzyme.

As used herein, the term “site-specific genome modification enzyme” refers to any enzyme that can modify a nucleotide sequence in a sequence-specific manner. In some embodiments, a site-specific genome modification enzyme modifies the genome by inducing a single-strand break. In some embodiments, a site-specific genome modification enzyme modifies the genome by inducing a double-strand break. In some embodiments, a site-specific genome modification enzyme comprises a cytidine deaminase. In some embodiments, a site specific genome modification enzyme comprises an adenine deaminase. Site-specific genome modification enzymes include endonucleases, recombinases, transposases, deaminases, helicases and any combination thereof. In some embodiments, the site-specific genome modification enzyme is a sequence-specific nuclease.

In one aspect, the endonuclease is selected from a meganuclease, a zinc-finger nuclease (ZFN), a transcription activator-like effector nucleases (TALEN), an Argonaute (non-limiting examples of Argonaute proteins include Thermus thermophilus Argonaute (TtAgo), Pyrococcus furiosus Argonaute (PfAgo), and Natronobacterium gregoryi Argonaute (NgAgo)), an RNA-guided nuclease, such as a CRISPR associated nuclease (non-limiting examples of CRISPR associated nucleases include, but are not limited to, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, CasX, CasY, homologs thereof, or modified versions thereof).

In some embodiments, the site-specific genome modification enzyme is a recombinase. Non-limiting examples of recombinases include a tyrosine recombinase attached to a DNA recognition motif and is selected from the group consisting of a Cre recombinase, a Gin recombinase, a Flp recombinase, and a Tnp1 recombinase. In one aspect, a Cre recombinase or a Gin recombinase is tethered to a zinc-finger DNA-binding domain, or a TALE DNA binding domain, or a Cas9 nuclease. In another aspect, a serine recombinase attached to a DNA recognition motif is selected from the group consisting of a PhiC31 integrase, an R4 integrase, and a TP-901 integrase. In another aspect, a DNA transposase attached to a DNA binding domain provided herein is selected from the group consisting of a TALE-piggyBac and TALE-Mutator.

Any DNA of interest as provided herein can be integrated into a target site of a chromosome sequence by introducing the DNA of interest and the disclosed site-specific genome modification enzymes. Any method provided herein can utilize any site-specific genome modification enzyme disclosed herein.

### V. Transformation

In some embodiments, transgenic plants of the present disclosure are created by transforming the selected natural plants with one or more of the expression cassettes disclosed herein. The natural plants prior to transformation are not naturally resistant to Fusarium oxysporum f.sp. cubense Tropical Race 4 (TR4). In certain embodiments, the selected natural plants for transformation include wild-type, or untransformed, or non-transformed banana plants, which are not naturally resistant to Fusarium oxysporum f.sp. cubense Tropical Race 4 (TR4).

Suitable methods for transformation of plant or other cells for use with the current disclosure are believed to include virtually any method by which DNA can be introduced into a cell, such as by direct delivery of DNA such as by PEG-mediated transformation of protoplasts (Omirulleh et al., Plant. Mol. Biol. 21:414-428, 1993), by desiccation/inhibition-mediated DNA uptake (Potrykus et al., Mol. Gen. Genet. 199:169-177, 1985), by electroporation (U.S. Pat. No. 5,384,253, specifically incorporated herein by reference in its entirety), by agitation with silicon carbide fibers (U.S. Pat. Nos. 5,302,523 and 5,464,765, specifically incorporated herein by reference in their entirety), by Agrobacterium-mediated transformation (U.S. Pat. Nos. 5,591,616 and 5,563,055; both specifically incorporated herein by reference in their entirety) and by acceleration of DNA coated particles (U.S. Pat. Nos. 5,550,318; 5,538,877; and 5,538,880; each specifically incorporated herein by reference in their entirety), etc. Through the application of techniques such as these, the cells of virtually any plant species may be transiently transformed, or stably transformed, and these cells developed into transgenic plants.

### A. Agrobacterium-Mediated Transformation

Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells is well known in the art. See, for example, the methods described by Fraley et al., (Proc. Natl. Acad. Sci. USA 80:4803-4807, 1985), and U.S. Pat. No. 5,563,055, specifically incorporated herein by reference in its entirety.

Agrobacterium-mediated transformation is most efficient in dicotyledonous plants and is an efficient method for transformation of dicots, including Arabidopsis, tobacco, tomato, alfalfa and potato. Indeed, while Agrobacterium-mediated transformation has been routinely used with dicotyledonous plants for a number of years, it has only recently become applicable to monocotyledonous plants. Advances in Agrobacterium-mediated transformation techniques have now made the technique applicable to nearly all monocotyledonous plants. For example, Agrobacterium-mediated transformation techniques have now been applied to rice (Hiei et al., Plant Mol. Biol. 35:205-218, 1997; U.S. Pat. No. 5,591,616, specifically incorporated herein by reference in its entirety), wheat and barley (McCormac et al., Mol. Biotechnol. 9:155-159, 1998), alfalfa and maize (Ishida et al., Nat. Biotechnol. 14:745-750, 1996). Similarly, Agrobacterium-mediated transformation has also proven to be effective in switchgrass.

Modern Agrobacterium transformation vectors are capable of replication in E. coli as well as Agrobacterium, allowing for convenient manipulations. Moreover, recent technological advances in vectors for Agrobacterium-mediated gene transfer have improved the arrangement of genes and restriction sites in the vectors to facilitate the construction of vectors capable of expressing various polypeptide coding genes. The vectors have convenient multi-linker regions flanked by a promoter and a polyadenylation site for direct expression of inserted polypeptide coding genes and are suitable for present purposes. In addition, Agrobacterium containing both armed and disarmed Ti genes can be used for the transformations. In those plant strains where Agrobacterium-mediated transformation is efficient, it is the method of choice because of the facile and defined nature of the gene transfer.

### B. Electroporation

To effect transformation by electroporation, one may employ either friable tissues, such as a suspension culture of cells or embryogenic callus or alternatively one may transform immature embryos or other organized tissue directly. In this technique, one would partially degrade the cell walls of the chosen cells by exposing them to pectin-degrading enzymes (pectolyases) or mechanically wounding in a controlled manner. Examples of some species that have been transformed by electroporation of intact cells include maize (U.S. Pat. No. 5,384,253, incorporated herein by reference in its entirety; Rhodes et al., Methods Mol. Biol. 55:121-131, 1995; D'Halluin et al., Plant Cell 4:1495-1505, 1992), wheat (Zhou et al., Plant Cell Rep. 12:612-616, 1993), tomato (Tsukada et al., Plant Cell Physiol. 30:599-603, 1989), soybean (Christou et al., Proc. Natl. Acad. Sci. USA 84:3962-3966, 1987) and tobacco (Riggs and Bates, Proc. Natl. Acad. Sci. USA 83:5602-5606, 1986).

One also may employ protoplasts for electroporation transformation of plants (Bates, Mol. Biotechnol. 2:135-145, 1994; Lazerri, Methods Mol. Biol. 49:95-106, 1995). For example, the generation of transgenic soybean plants by electroporation of cotyledon-derived protoplasts is described in WO 9217598 (specifically incorporated herein by reference). Other examples of species for which protoplast transformation has been described include barley (Lazerri, supra), sorghum (Battraw et al., Theor. Appl. Genet. 82:161-168, 1991), maize (Rhodes et al., Science 240:204-207, 1988), wheat (He et al., Plant Cell Rep. 14:192-196, 1994) and tomato (Tsukada, supra).

### C. Microprojectile Bombardment

Another method for delivering transforming DNA segments to plant cells in accordance with the present disclosure is microprojectile bombardment (U.S. Pat. Nos. 5,550,318; 5,538,880; 5,610,042; and PCT Application WO 94/09699; each of which is specifically incorporated herein by reference in its entirety). In this method, particles may be coated with nucleic acids and delivered into cells by a propelling force. Exemplary particles include those comprised of tungsten, platinum, and often, gold. It is contemplated that in some instances DNA precipitation onto metal particles would not be necessary for DNA delivery to a recipient cell using microprojectile bombardment. However, it is contemplated that particles may contain DNA rather than be coated with DNA. Hence, it is proposed that DNA-coated particles may increase the level of DNA delivery via particle bombardment but are not, in and of themselves, necessary.

For the bombardment, cells in suspension are concentrated on filters or solid culture medium. Alternatively, immature embryos or other target cells may be arranged on solid culture medium. The cells to be bombarded are positioned at an appropriate distance below the macroprojectile stopping plate.

An illustrative embodiment of a method for delivering DNA into plant cells by acceleration is the Biolistics Particle Delivery System, which can be used to propel particles coated with DNA or cells through a screen, such as a stainless steel or Nytex screen, onto a filter surface covered with monocot plant cells cultured in suspension. The screen disperses the particles so that they are not delivered to the recipient cells in large aggregates. Microprojectile bombardment techniques are widely applicable, and may be used to transform virtually any plant species. Examples of species that have been transformed by microprojectile bombardment include monocot species such as maize (PCT Application WO 95/06128), barley (Ritala et al., Plant Mol. Biol. 24:317-325, 1994; Hensgens et al., Plant Mol. Biol. 22:1101-1127, 1993), wheat (U.S. Pat. No. 5,563,055, specifically incorporated herein by reference in its entirety), rice (Hensgens et al., supra), oat (Torbet et al., Crop Science 38:226-231, 1998), rye (Hensgens et al., supra), sugarcane (Bower et al., Plant J. 2:409-416, 1992), and sorghum (Casas et al., Proc. Natl. Acad. Sci. USA 90:11212-11216, 1993; Hagio et al., Plant Cell Rep. 10:260-264, 1991); as well as a number of dicots including tobacco (Tomes et al., Plant Mol. Biol. 14:261-268, 1990), soybean (U.S. Pat. No. 5,322,783, specifically incorporated herein by reference in its entirety), sunflower (Knittel et al., Plant Cell Rep. 14:81-86, 1994), peanut (Singsit et al., Transgenic Res. 6:169-176, 1997), cotton (McCabe and Martinell, Nat. Biotechnol. 11:596-598, 1993), tomato (VanEck et al., Plant Cell. Rep. 14:299-304, 1995), switchgrass (Richards et al., Plant Cell Rep. 20:48-54, 2001) and legumes in general (U.S. Pat. No. 5,563,055, specifically incorporated herein by reference in its entirety).

### D. Other Transformation Methods

Transformation of protoplasts can be achieved using methods based on calcium phosphate precipitation, polyethylene glycol treatment, electroporation, and combinations of these treatments (see, e.g., Potrykus et al., supra; Omirulleh et al., supra;). Application of these systems to different plant strains depends upon the ability to regenerate that particular plant strain from protoplasts. Illustrative methods for the regeneration of cereals from protoplasts have been described (Toriyama et al., Nat. Biotechnol. 6:1072-1074, 1988; Abdullah et al., Nat. Biotechnol. 4:1087-1090, 1986; Omirulleh et al., supra, and U.S. Pat. No. 5,508,184; each specifically incorporated herein by reference in its entirety). Examples of the use of direct uptake transformation of cereal protoplasts include transformation of rice (Ghosh-Biswas et al., J. Biotechnol. 32:1-10, 1994), sorghum (Battraw et al., supra), barley (Lazzeri, supra), oat, and maize (Omirulleh et al., supra).

To transform plant strains that cannot be successfully regenerated from protoplasts, other ways to introduce DNA into intact cells or tissues can be utilized. For example, regeneration of cereals from immature embryos or explants can be effected as described (Vasil, supra). Also, silicon carbide fiber-mediated transformation may be used with or without protoplasting (Kaeppler et al., Theor. Appl. Genet. 84:560-566, 1992; U.S. Pat. No. 5,563,055, specifically incorporated herein by reference in its entirety). Transformation with this technique is accomplished by agitating silicon carbide fibers together with cells in a DNA solution. DNA passively enters as the cells are punctured. This technique has been used successfully with, for example, the monocot cereals maize (PCT Application WO 95/06128, specifically incorporated herein by reference in its entirety) and rice (Nagatani et al., Biotechnol. Tech. 11:471-473, 1997).

### E. Tissue Cultures

Tissue cultures may be used in certain transformation techniques for the preparation of cells for transformation and for the regeneration of plants therefrom. Maintenance of tissue cultures requires use of media and controlled environments. “Media” refers to the numerous nutrient mixtures that are used to grow cells in vitro, that is, outside of the intact living organism. The medium usually is a suspension of various categories of ingredients (salts, amino acids, growth regulators, sugars, buffers) that are required for growth of most cell types. However, each specific cell type requires a specific range of ingredient proportions for growth, and an even more specific range of formulas for optimum growth. Rate of cell growth also will vary among cultures initiated with the array of media that permit growth of that cell type.

Nutrient media is prepared as a liquid, but this may be solidified by adding the liquid to materials capable of providing a solid support. Agar is most commonly used for this purpose. BACTO®AGAR, GELRITE®, and GELGRO® are specific types of solid support that are suitable for growth of plant cells in tissue culture.

Some cell types will grow and divide either in liquid suspension or on solid media. As disclosed herein, plant cells will grow in suspension or on solid medium, but regeneration of plants from suspension cultures typically requires transfer from liquid to solid media at some point in development. The type and extent of differentiation of cells in culture will be affected not only by the type of media used and by the environment, for example, pH, but also by whether media is solid or liquid.

Tissue that can be grown in a culture includes meristem cells, Type I, Type II, and Type III callus, immature embryos and gametic cells such as microspores, pollen, sperm and egg cells. Type I, Type II, and Type III callus may be initiated from tissue sources including, but not limited to, immature embryos, seedling apical meristems, root, leaf, microspores and the like. Those cells that are capable of proliferating as callus also are recipient cells for genetic transformation.

Somatic cells are of various types. Embryogenic cells are one example of somatic cells that may be induced to regenerate a plant through embryo formation. Non-embryogenic cells are those that typically will not respond in such a fashion. Certain techniques may be used that enrich recipient cells within a cell population. For example, Type II callus development, followed by manual selection and culture of friable, embryogenic tissue, generally results in an enrichment of cells. Manual selection techniques that can be employed to select target cells may include, e.g., assessing cell morphology and differentiation, or may use various physical or biological means. Cryopreservation also is a possible method of selecting for recipient cells.

Manual selection of recipient cells, e.g., by selecting embryogenic cells from the surface of a Type II callus, is one means that may be used in an attempt to enrich for particular cells prior to culturing (whether cultured on solid media or in suspension).

Where employed, cultured cells may be grown either on solid supports or in the form of liquid suspensions. In either instance, nutrients may be provided to the cells in the form of media, and environmental conditions controlled. There are many types of tissue culture media comprised of various amino acids, salts, sugars, growth regulators and vitamins. Most of the media employed in the practice of the present disclosure will have some similar components, but may differ in the composition and proportions of their ingredients depending on the particular application envisioned. For example, various cell types usually grow in more than one type of media, but will exhibit different growth rates and different morphologies, depending on the growth media. In some media, cells survive but do not divide. Various types of media suitable for culture of plant cells previously have been described. Examples of these media include, but are not limited to, the N6 medium described by Chu et al., (Sci. Sin. [Peking] 18:659-668, 1975) and MS media (Murashige and Skoog, Physiol. Plant 15:473-479, 1962).

### VI. Production and Characterization of Stably Transformed Plants

After effecting delivery of exogenous DNA to recipient cells, the next steps generally concern identifying the transformed cells for further culturing and plant regeneration. In order to improve the ability to identify transformants, one may desire to employ a selectable or screenable marker gene with a transformation vector prepared in accordance with the present disclosure. In this case, one would then generally assay the potentially transformed cell population by exposing the cells to a selective agent or agents, or one would screen the cells for the desired marker gene trait.

### A. Selection

It is believed that DNA is introduced into only a small percentage of target cells in any one study. In order to provide an efficient system for identification of those cells receiving DNA and integrating it into their genomes one may employ a means for selecting those cells that are stably transformed. One exemplary embodiment of such a method is to introduce into the host cell a marker gene that confers resistance to some normally inhibitory agent, such as an antibiotic or herbicide. Examples of antibiotics that may be used include the aminoglycoside antibiotics neomycin, kanamycin and paromomycin, or the antibiotic hygromycin. Resistance to the aminoglycoside antibiotics is conferred by aminoglycoside phosphotransferase enzymes such as neomycin phosphotransferase II (NPT II) or NPT I, whereas resistance to hygromycin is conferred by hygromycin phosphotransferase.

Potentially transformed cells then are exposed to the selective agent. In the population of surviving cells will be those cells where, generally, the resistance-conferring gene has been integrated and expressed at sufficient levels to permit cell survival. Cells may be tested further to confirm stable integration of the exogenous DNA.

One herbicide that constitutes a desirable selection agent is the broad spectrum herbicide bialaphos. Bialaphos is a tripeptide antibiotic produced by Streptomyces hygroscopicus and is composed of phosphinothricin (PPT), an analogue of L-glutamic acid, and two L-alanine residues. Upon removal of the L-alanine residues by intracellular peptidases, the PPT is released and is a potent inhibitor of glutamine synthetase (GS), a pivotal enzyme involved in ammonia assimilation and nitrogen metabolism (Ogawa et al., Sci. Rep. Meiji Seika 13:42-48, 1973). Synthetic PPT, the active ingredient in the herbicide Liberty™ also is effective as a selection agent. Inhibition of GS in plants by PPT causes the rapid accumulation of ammonia and death of the plant cells.

The organism producing bialaphos and other species of the genus Streptomyces also synthesizes an enzyme phosphinothricin acetyl transferase (PAT), which is encoded by the bar gene in Streptomyces hygroscopicus and the pat gene in Streptomyces viridochromogenes. The use of the herbicide resistance gene encoding phosphinothricin acetyl transferase (PAT) is referred to in DE 3642 829 A, wherein the gene is isolated from Streptomyces viridochromogenes. In the bacterial source organism, this enzyme acetylates the free amino group of PPT preventing auto-toxicity (Thompson et al., EMBO J. 6:2519-2523, 1987). The bar gene has been cloned (Thompson et al., supra) and expressed in transgenic tobacco, tomato, potato (De Block et al., EMBO J. 6:2513-2518, 1987) Brassica (De Block et al., Plant Physiol. 91:694-701, 1989) and maize (U.S. Pat. No. 5,550,318, incorporated herein by reference in its entirety).

Another example of a herbicide that is useful for selection of transformed cell lines in the practice of the present disclosure is the broad spectrum herbicide glyphosate. Glyphosate inhibits the action of the enzyme EPSPS, which is active in the aromatic amino acid biosynthetic pathway. Inhibition of this enzyme leads to starvation for the amino acids phenylalanine, tyrosine, and tryptophan and secondary metabolites derived thereof. U.S. Pat. No. 4,535,060 (incorporated herein by reference in its entirety) describes the isolation of EPSPS mutations that confer glyphosate resistance on the Salmonella typhimurium gene for EPSPS, aroA. The EPSPS gene was cloned from Zea mays and mutations similar to those found in a glyphosate resistant aroA gene were introduced in vitro. Mutant genes encoding glyphosate resistant EPSPS enzymes are described in, for example, International Patent WO 97/4103.

To use the bar-bialaphos or the EPSPS-glyphosate selective system, transformed tissue is cultured for 0-28 days on nonselective medium and subsequently transferred to medium containing from 1-3 mg/l bialaphos or 1-3 mM glyphosate as appropriate. While ranges of 1-3 mg/l bialaphos or 1-3 mM glyphosate may be beneficial, it is proposed that ranges of 0.1-50 mg/l bialaphos or 0.1-50 mM glyphosate will find utility.

An example of a screenable marker trait is the enzyme luciferase. In the presence of the substrate luciferin, cells expressing luciferase emit light that can be detected on photographic or x-ray film, in a luminometer (or liquid scintillation counter), by devices that enhance night vision, or by a highly light sensitive video camera, such as a photon counting camera. These assays are nondestructive and transformed cells may be cultured further following identification. The photon counting camera is especially valuable as it allows one to identify specific cells or groups of cells that are expressing luciferase and manipulate those in real time. Another screenable marker that may be used in a similar fashion is the gene coding for green fluorescent protein.

### B. Regeneration and Seed Production

Cells that survive the exposure to the selective agent, or cells that have been scored positive in a screening assay, may be cultured in media that supports regeneration of plants. In an exemplary embodiment, MS and N6 media may be modified by including further substances such as growth regulators. One such growth regulator is dicamba or 2,4-D. However, other growth regulators may be employed, including NAA, NAA+2,4-D or picloram. Media improvement in these and like ways has been found to facilitate the growth of cells at specific developmental stages. Tissue may be maintained on a basic media with growth regulators until sufficient tissue is available to begin plant regeneration efforts, or following repeated rounds of manual selection, until the morphology of the tissue is suitable for regeneration, at least 2 weeks, then transferred to media conducive to maturation of embryoids. Cultures are transferred every 2 weeks on this medium. Shoot development will signal the time to transfer to medium lacking growth regulators.

The transformed cells, identified by selection or screening and cultured in an appropriate medium that supports regeneration, will then be allowed to mature into plants. Developing plantlets are transferred to soilless plant growth mix, and hardened, e.g., in an environmentally controlled chamber, for example, at about 85% relative humidity, 600 ppm CO₂, and 25-250 microeinsteins m² s⁻¹ of light. Plants may be matured in a growth chamber or greenhouse. Plants can be regenerated from about 6 weeks to 10 months after a transformant is identified, depending on the initial tissue. During regeneration, cells are grown on solid media in tissue culture vessels. Illustrative embodiments of such vessels are petri dishes and Plant Cons. Regenerating plants can be grown at about 19 to 28° C. After the regenerating plants have reached the stage of shoot and root development, they may be transferred to a greenhouse for further growth and testing.

Seeds on transformed plants may occasionally require embryo rescue due to cessation of seed development and premature senescence of plants. To rescue developing embryos, they are excised from surface-disinfected seeds 10-20 days post-pollination and cultured. An embodiment of media used for culture at this stage comprises MS salts, 2% sucrose, and 5.5 g/l agarose. In embryo rescue, large embryos (defined as greater than 3 mm in length) are germinated directly on an appropriate media. Embryos smaller than that may be cultured for 1 week on media containing the above ingredients along with 10⁻⁵ M abscisic acid and then transferred to growth regulator-free medium for germination.

### C. Characterization

To confirm the presence of the exogenous DNA or “transgene(s)” in the regenerating plants, a variety of assays may be performed. Such assays include, for example, “molecular biological” assays, such as Southern and northern blotting and PCR™; “biochemical” assays, such as detecting the presence of a protein product, e.g., by immunological means (ELISAs and western blots) or by enzymatic function; plant part assays, such as leaf or root assays; and also, by analyzing the phenotype of the whole regenerated plant.

### D. DNA Integration, RNA Expression and Inheritance

Genomic DNA may be isolated from cell lines or any plant parts to determine the presence of the exogenous gene through the use of techniques well known to those skilled in the art. Note, that intact sequences will not always be present, presumably due to rearrangement or deletion of sequences in the cell. The presence of DNA elements introduced through the methods of this disclosure may be determined, for example, by polymerase chain reaction (PCR™). Using this technique, discreet fragments of DNA are amplified and detected by gel electrophoresis. This type of analysis permits one to determine whether a gene is present in a stable transformant, but does not prove integration of the introduced gene into the host cell genome. It is typically the case, however, that DNA has been integrated into the genome of all transformants that demonstrate the presence of the gene through PCR™ analysis. In addition, it is not typically possible using PCR™ techniques to determine whether transformants have exogenous genes introduced into different sites in the genome, i.e., whether transformants are of independent origin. It is contemplated that using PCR™ techniques it would be possible to clone fragments of the host genomic DNA adjacent to an introduced gene.

Positive proof of DNA integration into the host genome and the independent identities of transformants may be determined using the technique of Southern hybridization. Using this technique specific DNA sequences that were introduced into the host genome and flanking host DNA sequences can be identified. Hence the Southern hybridization pattern of a given transformant serves as an identifying characteristic of that transformant. In addition it is possible through Southern hybridization to demonstrate the presence of introduced genes in high molecular weight DNA, i.e., confirm that the introduced gene has been integrated into the host cell genome. The technique of Southern hybridization provides information that is obtained using PCR™, e.g., the presence of a gene, but also demonstrates integration into the genome and characterizes each individual transformant.

It is contemplated that using the techniques of dot or slot blot hybridization, which are modifications of Southern hybridization techniques, one could obtain the same information that is derived from PCR™, e.g., the presence of a gene.

Both PCR™ and Southern hybridization techniques can be used to demonstrate transmission of a transgene to progeny. In most instances the characteristic Southern hybridization pattern for a given transformant will segregate in progeny as one or more Mendelian genes (Spencer et al., 1992) indicating stable inheritance of the transgene.

Whereas DNA analysis techniques may be conducted using DNA isolated from any part of a plant, RNA will only be expressed in particular cells or tissue types and hence it will be necessary to prepare RNA for analysis from these tissues. PCR™ techniques also may be used for detection and quantitation of RNA produced from introduced genes. In this application of PCR™ it is first necessary to reverse transcribe RNA into DNA, using enzymes such as reverse transcriptase, and then through the use of conventional PCR™ techniques amplify the DNA. In most instances PCR™ techniques, while useful, will not demonstrate integrity of the RNA product. Further information about the nature of the RNA product may be obtained by northern blotting. This technique will demonstrate the presence of an RNA species and give information about the integrity of that RNA. The presence or absence of an RNA species also can be determined using dot or slot blot northern hybridizations. These techniques are modifications of northern blotting and will only demonstrate the presence or absence of an RNA species.

### E. Gene Expression

While Southern blotting and PCR™ may be used to detect the gene(s) in question, they do not provide information as to whether the corresponding protein is being expressed. Expression may be evaluated by specifically identifying the protein products of the introduced genes or evaluating the phenotypic changes brought about by their expression.

Assays for the production and identification of specific proteins may make use of physical-chemical, structural, functional, or other properties of the proteins. Unique physical-chemical or structural properties allow the proteins to be separated and identified by electrophoretic procedures, such as native or denaturing gel electrophoresis or isoelectric focusing, or by chromatographic techniques such as ion exchange or gel exclusion chromatography. The unique structures of individual proteins offer opportunities for use of specific antibodies to detect their presence in formats such as an ELISA assay. Combinations of approaches may be employed with even greater specificity such as western blotting in which antibodies are used to locate individual gene products that have been separated by electrophoretic techniques. Additional techniques may be employed to absolutely confirm the identity of the product of interest such as evaluation by amino acid sequencing following purification. Although these are among the most commonly employed, other procedures may be additionally used.

Assay procedures also may be used to identify the expression of proteins by their functionality, especially the ability of enzymes to catalyze specific chemical reactions involving specific substrates and products. These reactions may be followed by providing and quantifying the loss of substrates or the generation of products of the reactions by physical or chemical procedures. Examples are as varied as the enzyme to be analyzed and may include assays for PAT enzymatic activity by following production of radiolabeled acetylated phosphinothricin from phosphinothricin and ¹⁴C-acetyl CoA or for anthranilate synthase activity by following loss of fluorescence of anthranilate, to name two.

Very frequently the expression of a gene product is determined by evaluating the phenotypic results of its expression. These assays also may take many forms including, but not limited to, analyzing changes in the chemical composition, morphology, or physiological properties of the plant. Chemical composition may be altered by expression of genes encoding enzymes or storage proteins that change amino acid composition and may be detected by amino acid analysis, or by enzymes that change starch quantity, which may be analyzed by near infrared reflectance spectrometry. Morphological changes may include greater stature or thicker stalks. Most often changes in response of plants or plant parts to imposed treatments are evaluated under carefully controlled conditions termed bioassays.

### VII. Breeding Plants

In addition to direct transformation of a particular plant genotype with a construct prepared according to the current disclosure, transgenic plants may be made by crossing a plant having a selected DNA of the present disclosure to a second plant lacking the construct. For example, a selected coding sequence can be introduced into a particular plant variety by crossing, without the need for ever directly transforming a plant of that given variety. Therefore, the current disclosure not only encompasses a plant directly transformed or regenerated from cells that have been transformed in accordance with the current disclosure, but also the progeny of such plants.

As used herein the term “progeny” denotes the offspring of any generation of a parent plant prepared in accordance with the instant disclosure, wherein the progeny comprises a selected DNA construct. “Crossing” a plant to provide a plant line having one or more added transgenes relative to a starting plant line, as disclosed herein, is defined as the techniques that result in a transgene of the present disclosure being introduced into a plant line by crossing a starting line with a donor plant line that comprises a transgene of the present disclosure. To achieve this one could, for example, perform the following steps:
(a) plant seeds of the first (starting line) and second (donor plant line that comprises a transgene of the present disclosure) parent plants;
(b) grow the seeds of the first and second parent plants into plants that bear flowers;
(c) pollinate a flower from the first parent plant with pollen from the second parent plant; and
(d) harvest seeds produced on the parent plant bearing the fertilized flower.

Backcrossing is herein defined as the process including the steps of:
(a) crossing a plant of a first genotype containing a desired gene, DNA sequence or element to a plant of a second genotype lacking the desired gene, DNA sequence or element;
(b) selecting one or more progeny plant containing the desired gene, DNA sequence or element;
(c) crossing the progeny plant to a plant of the second genotype; and
(d) repeating steps (b) and (c) for the purpose of transferring a desired DNA sequence from a plant of a first genotype to a plant of a second genotype.

Introgression of a DNA element into a plant genotype is defined as the result of the process of backcross conversion. A plant genotype into which a DNA sequence has been introgressed may be referred to as a backcross converted genotype, line, inbred, or hybrid. Similarly a plant genotype lacking the desired DNA sequence may be referred to as an unconverted genotype, line, inbred, or hybrid.

### VIII—Other Improved Agronomic Traits

In addition to increased resistance to Fusarium oxysporum f.sp. cubense Tropical Race 4, the presently disclosed banana plants can possess one or more other improved agronomic trait relative to a wild-type banana plant, or a banana plant not comprising the recited transgene(s) or genome modification(s). Such improved agronomic trait can include, but are not limited to, increased resistance to other strains of Fusarium oxysporum f sp. cubense, Mycosphaerella fijiensis (black sigatoka, black leaf streak disease), increased resistance to other pathogens and pests, increased yield, phosphate uptake, drought resistance, disease resistance, fungal resistance, nutrient uptake, water uptake, average primary root length, average number of lateral roots, average root hair density and length, average number of seed pods, average seed pod size, average seed size, average seed weight, seed germination, seed survival, average number of siliques, average silique size, average leaf area, average leaf length, and average plant height. Additional modified traits of the banana plants disclosed herein include changes in the color of parts of the banana, including the fruit, changes in the flavor, sweetness, fiber, shelf life, storability, size, and/or shape of the banana, and changes in plant development and biotic/abiotic/environmental stress responses.

### IX. Additional Definitions

The following definitions or interpretations of technical terms will be used throughout the present disclosure. The technical terms used herein are generally to be given the meaning commonly applied to them in the pertinent art of plant biology, molecular biology, bioinformatics, and plant breeding. All of the following term definitions apply to the complete content of this application.

To facilitate the understanding of this disclosure, a number of terms are defined below. Terms defined herein have meanings as commonly understood by a person of ordinary skill in the areas relevant to the present disclosure. The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of.” As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about,” “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

The terms “peptides,” “oligopeptides,” “polypeptide,” “protein”, or “enzyme” are used interchangeably herein and refer to amino acids in a polymeric form of any length, linked together by peptide bonds, unless mentioned herein otherwise. The terms “gene sequence(s),” “polynucleotide(s),” “nucleic acid sequence(s),” “nucleotide sequence(s),” “nucleic acid(s),” “nucleic acid molecule” are used interchangeably herein and refer to nucleotides, either ribonucleotides or deoxyribonucleotides or a combination of both, in a polymeric unbranched form of any length.

Endogenous. An “endogenous” or “native” nucleic acid and/or protein refers to a nucleic acid and/or protein as found in a plant or other organism in its natural form (i.e., without there being any human intervention, such as recombinant DNA engineering technology),

Exogenous. The term “exogenous” (in contrast to “endogenous”) means a nucleic acid or protein that has been introduced in a plant or other organism by means of recombinant DNA technology. An “exogenous” nucleic acid or protein can either not occur in a plant in its natural form, be different from the nucleic acid or protein as found in a plant in its natural form, be present at a higher or lower level than the nucleic acid or protein naturally present in a plant, or in the case of a nucleic acid can be identical to a nucleic acid found in a plant in its natural form, but integrated at a location different that its natural genetic environment.

Expression: The combination of intracellular processes, including transcription and translation undergone by a coding DNA molecule such as a structural gene to produce a polypeptide.

Expression Cassette. A nucleic acid sequence of interest operably linked to one or more control sequences (at least to a promoter) as described herein. An expression cassette can also include additional transcriptional and/or translational enhancers. An expression cassette can also include terminator, silencer and enhancer sequences, intron sequences added to the 5′ untranslated region (UTR) or in the coding sequence of the nucleic acid sequence, and/or other control sequences such as protein and/or RNA stabilizing elements. An expression cassette may be integrated into the genome of a host cell and replicated together with the genome of the host cell, or transiently present in a host cell.

Genetic Engineering: A process of introducing a DNA sequence or construct into a cell or protoplast; also includes gene editing.

Genetic Transformation: A process of introducing a DNA sequence or construct (e.g., a vector or expression cassette) into a cell or protoplast in which that exogenous DNA is incorporated into a chromosome or is capable of autonomous replication.

Heterologous: A sequence that is not normally present in a given host genome in the genetic context in which the sequence is currently found In this respect, the sequence may be native to the host genome, but be rearranged with respect to other genetic sequences within the host sequence. For example, a regulatory sequence may be heterologous in that it is linked to a different coding sequence relative to the native regulatory sequence.

Modulation. The term modulation refers to when the expression level is changed in comparison to the expression seen in a control plant. Modulation refers to an expression level that is either increased or decreased.

Obtaining: When used in conjunction with a transgenic plant cell or transgenic plant, obtaining means either transforming a non-transgenic plant cell or plant to create the transgenic plant cell or plant, or planting transgenic plant seed to produce the transgenic plant cell or plant. Such a transgenic plant seed may be from an R0 transgenic plant or may be from a progeny of any generation thereof that inherits a given transgenic sequence from a starting transgenic parent plant.

Operably Linked. The term “operably linked” or “functionally linked” is used interchangeably and, as used herein, refers to a functional linkage between, for example, a promoter sequence and a nucleic acid sequence of interest, such that the promoter sequence is able to direct transcription of the nucleic acid sequence of interest, or a functional linkage between a terminator sequence and a nucleic acid sequence of interest, such that the terminator sequence is able to stop or terminate transcription of the nucleic acid sequence of interest.

Plant. The term “plant” as used herein encompasses whole plants, ancestors and progeny of the plants and plant parts, including fruits, seeds, shoots, stems, leaves, roots (including tubers), flowers, and tissues and organs, wherein each of the aforementioned comprise the gene/nucleic acid of interest. The term “plant” also encompasses plant cells, suspension cultures, callus tissue, embryos, meristematic regions, gametophytes, sporophytes, pollen and microspores, again wherein each of the aforementioned comprises the gene/nucleic acid of interest.

Ploidy. Ploidy or chromosomal ploidy refers the number of complete sets of chromosomes occurring in the nucleus of a cell. Somatic cells, tissues, and individual organisms can be described according to the number of sets of chromosomes present (the “ploidy level”): monoploid (1 set), diploid (2 sets), triploid (3 sets), tetraploid (4 sets), pentaploid (5 sets), hexaploid (6 sets), heptaploid or septaploid (7 sets), etc. The generic term polyploidy is used herein to describe cells with three or more chromosome sets.

Promoter: A recognition site on a DNA sequence or group of DNA sequences that provides an expression control element for a structural gene and to which RNA polymerase specifically binds and initiates RNA synthesis (transcription) of that gene.

R0 transgenic plant: A plant that has been genetically transformed or has been regenerated from a plant cell or cells that have been genetically transformed.

Recombinant. A nucleic acid sequence, expression cassette, genetic construct, or vector comprising a nucleic acid sequence as disclosed herein, or an organism transformed with such nucleic acid sequences, expression cassettes or vectors, created by genetic engineering techniques in which either (a) the sequences of the nucleic acids or a part thereof, or (b) genetic control sequence(s) that is operably linked with the nucleic acid sequence, for example a promoter or terminator, or (c) combinations of (a) and (b), are not located in their natural genetic environment or have been modified and/or inserted artificially by genetic engineering methods.

Regeneration: The process of growing a plant from a plant cell (e.g., plant protoplast, callus or explant).

Selected DNA: A DNA segment that one desires to introduce or has introduced into a plant genome by genetic transformation.

Terminator. A DNA control sequence at the end of a transcriptional unit that signals 3′ processing and polyadenylation of a primary transcript and termination of transcription.

Transformation construct: A chimeric DNA molecule that is designed for introduction into a host genome by genetic transformation. Transformation constructs will often comprise all of the genetic elements necessary to direct the expression of one or more exogenous genes. In particular embodiments of the instant disclosure, it may be desirable to introduce a transformation construct into a host cell in the form of an expression cassette.

Transformed cell: A cell the DNA complement of which has been altered by the introduction of an exogenous DNA molecule into that cell.

Transgene: A segment of DNA that has been incorporated into a host genome or is capable of autonomous replication in a host cell and is capable of causing the expression of one or more coding sequences. Exemplary transgenes will provide the host cell, or plants regenerated therefrom, with a novel phenotype relative to the corresponding non-transformed cell or plant. Transgenes may be directly introduced into a plant by genetic transformation, or may be inherited from a plant of any previous generation that was transformed with the DNA segment.

Transgenic plant: A plant or progeny plant of any subsequent generation derived therefrom, wherein the DNA of the plant or progeny thereof contains an introduced exogenous DNA segment not naturally present in a non-transgenic plant of the same strain. The transgenic plant may additionally contain sequences that are native to the plant being transformed, but wherein the “exogenous” gene has been altered in order to alter the level or pattern of expression of the gene, for example, by use of one or more heterologous regulatory or other elements.

Vector: A DNA molecule designed for transformation into a host cell. Some vectors may be capable of replication in a host cell. A plasmid is an exemplary vector, as are expression cassettes isolated therefrom.

### EXAMPLES

The following examples are included to demonstrate illustrative embodiments of the present disclosure. It should be appreciated by those of skill in the art that the techniques disclosed in the examples that follow represent techniques discovered by the inventor to function well in the practice of the present disclosure, and thus can be considered to constitute one embodiment of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1

### Construction of Expression Cassettes

Various expression cassettes having one or more nucleotide sequences as disclosed herein were constructed. Construction of these expression cassettes was carried out following standard genetic engineering methods. The following expression cassettes were constructed.

SP0650 (SEQ ID NO:10): This expression vector comprises a nucleic acid sequence (SEQ ID NO: 8; termed herein as RUBY) comprising three betalain biosynthetic genes linked with a 2A sequence, which allows the three genes to be expressed with a single promoter to produce a protein (SEQ ID NO:9) that is processed into three separate proteins. The detailed description of the components of the SP0650 expression vector is shown below in Table 1. In Table 1, the T-DNA payload is indicated in italics, the backbone is indicated in underlining, and the RUBY element is indicated in bold.

TABLE 1

| Description | Function | Position |
|---|---|---|
| Origin of replication, p15A | E. coli origin of replication | 1-546 |
| Backbone from pJ-Kan low | Spacer | 547-1368 |
| pCAMBIA1305.1 backbone | Spacer | 1369-1712 |
| pVS1 oriV | required for function in Agro | 1713-1907 |
| pCAMBIA1305.1 backbone | Spacer | 1908-1972 |
| pVS1 RepA replication protein | required for function in Agro | 3046-1973 |
| from Pseudomonas plasmid pVS1 | | |
| pCAMBIA1305.1 backbone | Spacer | 3047-3474 |
| pVS1 STA stability protein from | required for function in Agro | 4104-3475 |
| Pseudomonas plasmid pVS1 | | |
| pCAMBIA1305.1 backbone | Spacer | 4105-5403 |
| T DNA repeat | Right border | 5404-5428 |
| pCAMBIA1305.1 backbone | Spacer | 5429-5446 |
| Spacer Sequence | Spacer | 5547-5565 |
| PBI Synthetic Terminator | Transcriptional Terminator | 5566-5958 |
| Spacer Sequence | Spacer | 5819-5822 |
| mCherry | Florescent protein marker | 5963-6673 |
| Spacer Sequence | Spacer | 6671-6674 |
| SynJ 5UTR | 5UTR sequence | 6675-6701 |
| CsVMV Promoter | Promoter driving mCherry | 6702-7215 |
| Spacer Sequence | Spacer | 7216-7232 |
| 35S Terminator | Transcriptional Terminator | 7233-7407 |
| Spacer Sequence | Spacer | 7408-7411 |
| Dicot RUBY | Proposed TR4 Resistance | 7412-11362 |
| CaMV e35S Promoter | Promoter driving Dicot RUBY | 11364-12105 |
| Spacer Sequence | Spacer | 12106-12109 |
| pCAMBIA1305.1 backbone | Spacer | 12110-12272 |
| T DNA repeat | Left Border | 12273-12298 |
| pCAMBIA1305.1 backbone | Spacer | 12299-12659 |
| Backbone from pJ-Kan low | Spacer | 12660-12995 |
| AmpR promoter | Promoter driving KanR | 12996-13087 |
| KanR ORF | Bacterial Selection | 13088-13897 |
| Backbone from pJ-Kan low | Spacer | 13918-13989 |

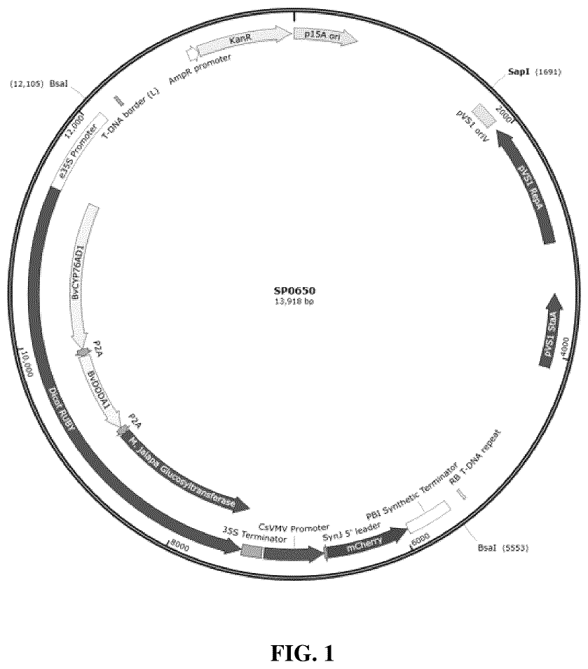

A map of the SP0650 expression vector is shown in FIG. 1.

SP1716 (SEQ ID NO:11): This expression vector comprises a nucleic acid sequence (SEQ ID NO:4) that encodes the Sm-AMP-D1 anti-microbial peptide (SEQ ID NO:5). The detailed description of the components of the SP1716 expression vector is shown below in Table 2. In Table 2, the T-DNA payload is indicated in italics, the backbone is indicated in underlining, and the Sm-AMP-D1 element is indicated in bold.

TABLE 2

| Description | Function | Position |
|---|---|---|
| Origin of replication, p15A | E. coli origin of replication | 1-546 |
| Backbone from pJ-Kan low | Spacer | 547-1368 |
| pCAMBIA1305.1 backbone | Spacer | 1369-1712 |
| pVS1 oriV | required for function in | 1713-1907 |
| | Agro | |
| pCAMBIA1305.1 backbone | Spacer | 1908-1972 |
| pVS1 RepA replication protein from | required for function in | 1973-3046 |
| Pseudomonas plasmid pVS1 | Agro | |
| pCAMBIA1305.1 backbone | Spacer | 3047-3474 |
| pVS1 STA stability protein from | required for function in | 3475-4104 |
| Pseudomonas plasmid pVS1 | Agro | |
| pCAMBIA1305.1 backbone | Spacer | 4105-5403 |
| T DNA repeat | Right border | 5404-5428 |
| pCAMBIA1305.1 backbone | Spacer | 5429-5446 |
| Spacer Sequence | Spacer | 5547-5565 |
| Nos Terminator | Transcriptional Terminator | 5566-5818 |
| Spacer Sequence | Spacer | 5819-5824 |
| Sm-AMP-D1 | Proposed TR4 Resistance | 5825-6070 |
| Spacer Sequence | Spacer | 6068-6072 |
| ZmUbi1 Promoter | Promoter driving | 6072-8063 |
| | MusaBAG1 | |
| Spacer Sequence | Spacer | 8064-8075 |
| PBI Synthetic Terminator | Transcriptional Terminator | 8076-8468 |
| Spacer Sequence | Spacer | 8469-8472 |
| EGFP with ER retention signal | Florescent protein marker | 8473-9291 |
| Spacer Sequence | Spacer | 9289-9292 |
| Duplicated MMV Promoter | Promoter driving EGFP | 9293-9913 |
| Spacer Sequence | Spacer | 9914-9931 |
| 35S Terminator | Transcriptional Terminator | 9931-10105 |
| Spacer Sequence | Spacer | 10106-10109 |
| Hygromycin B Phosphotransferase | Selectable Marker Gene | 10110-11138 |
| ORF | | |
| Spacer Sequence | Spacer | 11136-11139 |
| SynJ 5UTR | 5UTR sequence | 11140-11166 |
| CsVMV Promoter | Promoter driving HygR | 11167-11680 |
| Spacer Sequence | Spacer | 11681-11684 |
| pCAMBIA1305.1 backbone | Spacer | 11685-11847 |
| T DNA repeat | Left Border | 11848-11873 |
| pCAMBIA1305.1 backbone | Spacer | 11874-12235 |
| Backbone from pJ-Kan low | Spacer | 12236-12570 |
| AmpR promoter | Promoter driving KanR | 12571-12662 |
| KanR ORF | Bacterial Selection | 12663-13472 |
| Backbone from pJ-Kan low | Spacer | 13473-13493 |

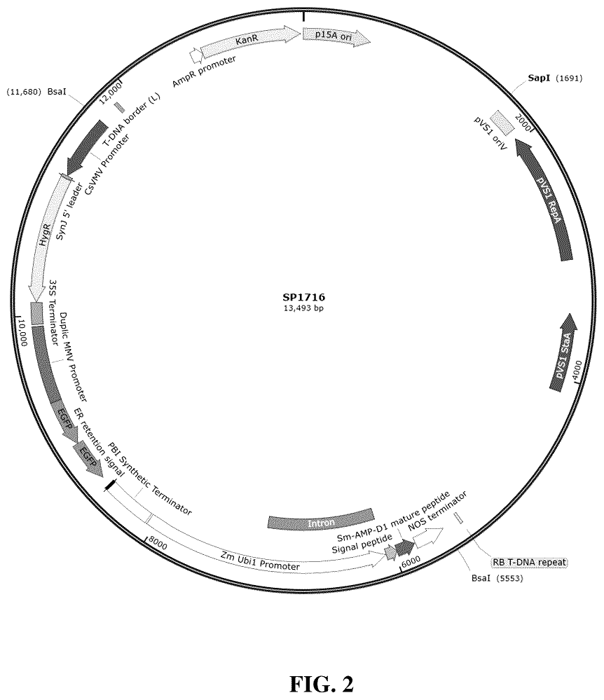

A map of the SP1716 expression vector is shown in FIG. 2.

SP2149 (SEQ ID NO:12): This expression vector comprises a nucleic acid sequence (SEQ ID NO:6) encoding the Musa acuminata subsp. malaccensis RGA2 protein (SEQ ID NO:7). The detailed description of the components of the SP2149 expression vector is shown below in Table 3. In Table 3, the T-DNA payload is indicated in italics, the backbone is indicated in underlining, and the RGA2 element is indicated in bold.

TABLE 3

| Description | Function | Position |
|---|---|---|
| Origin of replication, p15A | E. coli origin of replication | 1-546 |
| Backbone from pJ-Kan low | Spacer | 547-1368 |
| pCAMBIA1305.1 backbone | Spacer | 1369-1712 |
| pVS1 oriV | required for function in | 1713-1907 |
| | Agro | |
| pCAMBIA1305.1 backbone | Spacer | 1908-1972 |
| pVS1 RepA replication protein from | required for function in | 1973-3046 |
| Pseudomonas plasmid pVS1 | Agro | |
| pCAMBIA1305.1 backbone | Spacer | 3047-3474 |
| pVS1 STA stability protein from | required for function in | 3475-4104 |
| Pseudomonas plasmid pVS1 | Agro | |
| pCAMBIA1305.1 backbone | Spacer | 4105-5403 |
| T DNA repeat | Right border | 5404-5428 |
| pCAMBIA1305.1 backbone | Spacer | 5429-5446 |
| Spacer Sequence | Spacer | 5547-5565 |
| Nos Terminator | Transcriptional Terminator | 5566-5818 |
| Spacer Sequence | Spacer | 5819-5822 |
| M acuminata subsp. malaccensis | Proposed TR4 Resistance | 5823-9521 |
| RGA2 | | |
| Spacer Sequence | Spacer | 9519-9522 |
| ZmUbi1 Promoter | Promoter driving | 9523-11514 |
| | MusaBAG1 | |
| Spacer Sequence | Spacer | 11515-11526 |
| PBI Synthetic Terminator | Transcriptional Terminator | 11527-11919 |
| Spacer Sequence | Spacer | 11920-11923 |
| EGFP with ER retention signal | Florescent protein marker | 11924-12742 |
| Spacer Sequence | Spacer | 12740-12743 |
| Duplicated MMV Promoter | Promoter driving EGFP | 12744-13364 |
| Spacer Sequence | Spacer | 13365-13381 |
| 35S Terminator | Transcriptional Terminator | 13382-13556 |
| Spacer Sequence | Spacer | 13557-13560 |
| Hygromycin B Phosphotransferase | Selectable Marker Gene | 13561-14589 |
| ORF | | |
| Spacer Sequence | Spacer | 14587-14590 |
| SynJ 5UTR | 5UTR sequence | 14591-14617 |
| CsVMV Promoter | Promoter driving HygR | 14618-15131 |
| Spacer Sequence | Spacer | 15132-15135 |
| pCAMBIA1305.1 backbone | Spacer | 15136-15298 |
| T DNA repeat | Left Border | 15299-15324 |
| pCAMBIA1305.1 backbone | Spacer | 15325-15685 |
| Backbone from pJ-Kan low | Spacer | 15686-16021 |
| AmpR promoter | Promoter driving KanR | 16022-16113 |
| KanR ORF | Bacterial Selection | 16114-16923 |
| Backbone from pJ-Kan low | Spacer | 16924-16944 |

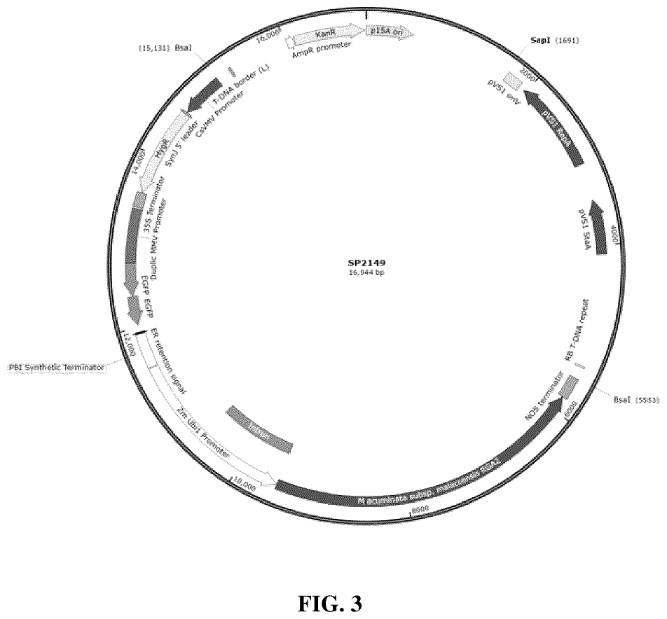

A map of the SP2149 expression vector is shown in FIG. 3.

SP4589 (SEQ ID NO:13): This expression vector comprises an ERG11 RNAi nucleic acid sequence (SEQ ID NO:3). The detailed description of the components of the SP4589 expression vector is shown below in Table 4. In Table 4, the T-DNA payload is indicated in italics, the backbone is indicated in underlining, and the ERG11 RNAi element is indicated in bold.

TABLE 4

| Description | Function | Position |
|---|---|---|
| Origin of replication, p15A | E. coli origin of replication | 1-546 |
| Backbone from pJ-Kan low | Spacer | 547-1368 |
| pCAMBIA1305.1 backbone | Spacer | 1369-1712 |
| pVS1 oriV | required for function in | 1713-1907 |
| | Agro | |
| pCAMBIA1305.1 backbone | Spacer | 1908-1972 |
| pVS1 RepA replication protein from | required for function in | 1973-3046 |
| Pseudomonas plasmid pVS1 | Agro | |
| pCAMBIA1305.1 backbone | Spacer | 3047-3474 |
| pVS1 STA stability protein from | required for function in | 3475-4104 |
| Pseudomonas plasmid pVS1 | Agro | |
| pCAMBIA1305.1 backbone | Spacer | 4105-5403 |
| T DNA repeat | Right border | 5404-5428 |
| pCAMBIA1305.1 backbone | Spacer | 5429-5446 |
| Spacer Sequence | Spacer | 5547-5565 |
| Nos Terminator | Transcriptional Terminator | 5566-5818 |
| Spacer Sequence | Spacer | 5819-5822 |
| ERG11 ihpRNAi | Proposed TR4 | 5823-8359 |
| | Resistance | |
| Spacer Sequence | Spacer | 8360-8363 |
| ZmUbi1 Promoter | Promoter driving | 8364-10355 |
| | MusaBAG1 | |
| Spacer Sequence | Spacer | 10356-10367 |
| PBI Synthetic Terminator | Transcriptional Terminator | 10368-10760 |
| Spacer Sequence | Spacer | 10761-10764 |
| EGFP with ER retention signal | Florescent protein marker | 10765-11583 |
| Spacer Sequence | Spacer | 11581-11584 |
| Duplicated MMV Promoter | Promoter driving EGFP | 11585-12205 |
| Spacer Sequence | Spacer | 12206-12222 |
| 35S Terminator | Transcriptional Terminator | 12223-12397 |
| Spacer Sequence | Spacer | 12398-12401 |
| Hygromycin B Phosphotransferase | Selectable Marker Gene | 12402-13430 |
| ORF | | |
| Spacer Sequence | Spacer | 13428-13431 |
| SynJ 5UTR | 5UTR sequence | 13432-13458 |
| CsVMV Promoter | Promoter driving HygR | 13459-13972 |
| Spacer Sequence | Spacer | 13973-13976 |
| pCAMBIA1305.1 backbone | Spacer | 13977-14139 |
| T DNA repeat | Left Border | 14140-14165 |
| pCAMBIA1305.1 backbone | Spacer | 14166-14526 |
| Backbone from pJ-Kan low | Spacer | 14527-14862 |
| AmpR promoter | Promoter driving KanR | 14863-14954 |
| KanR ORF | Bacterial Selection | 14955-15764 |
| Backbone from pJ-Kan low | Spacer | 15765-15785 |

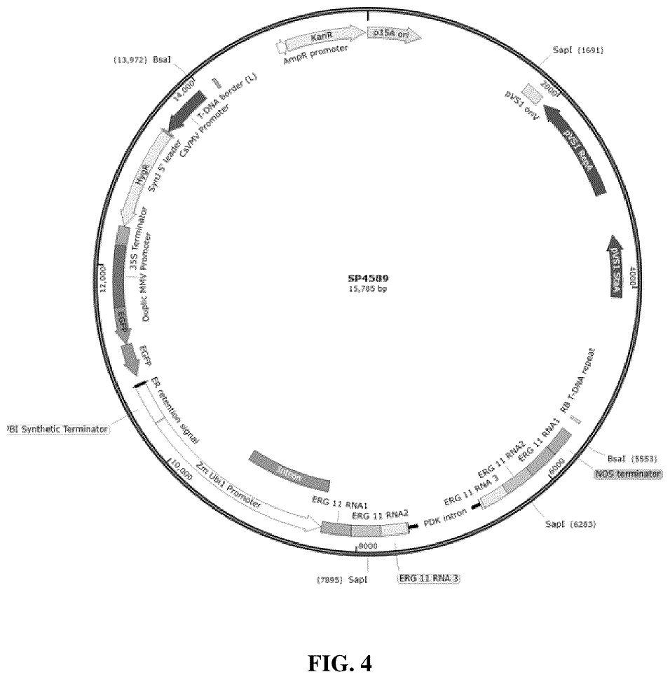

A map of the SP4589 expression vector is shown in FIG. 4.

SP4928 (SEQ ID NO:14): This expression vector comprises an nucleic acid sequence (SEQ ID NO:1) encoding the BAG1 protein from Musa acuminata (SEQ ID NO:2). The detailed description of the components of the SP4928 expression vector is shown below in Table 5. In Table 5, the T-DNA payload is indicated in italics, the backbone is indicated in underlining, and the BAG1 element is indicated in bold.

TABLE 5

| Description | Function | Position |
|---|---|---|
| Origin of replication, p15A | E. coli origin of replication | 1-546 |
| Backbone from pJ-Kan low | Spacer | 547-1368 |
| pCAMBIA1305.1 backbone | Spacer | 1369-1712 |
| pVS1 oriV | required for function in | 1713-1907 |
| | Agro | |
| pCAMBIA1305.1 backbone | Spacer | 1908-1972 |
| pVS1 RepA replication protein from | required for function in | 1973-3046 |
| Pseudomonas plasmid pVS1 | Agro | |
| pCAMBIA1305.1 backbone | Spacer | 3047-3474 |
| pVS1 STA stability protein from | required for function in | 3475-4104 |
| Pseudomonas plasmid pVS1 | Agro | |
| pCAMBIA1305.1 backbone | Spacer | 4105-5403 |
| T DNA repeat | Right border | 5404-5428 |
| pCAMBIA1305.1 backbone | Spacer | 5429-5446 |
| Spacer Sequence | Spacer | 5547-5565 |
| Nos Terminator | Transcriptional Terminator | 5566-5818 |
| Spacer Sequence | Spacer | 5819-5822 |
| MusaBAG1 | Proposed TR4 Resistance | 5823-6281 |
| Spacer Sequence | Spacer | 6282-6285 |
| ZmUbi1 Promoter | Promoter driving | 6286-8277 |
| | MusaBAG1 | |
| Spacer Sequence | Spacer | 8278-8289 |
| PBI Synthetic Terminator | Transcriptional Terminator | 8290-8682 |
| Spacer Sequence | Spacer | 8683-8686 |
| EGFP with ER retention signal | Florescent protein marker | 8687-9505 |
| Spacer Sequence | Spacer | 9503-9506 |
| Duplicated MMV Promoter | Promoter driving EGFP | 9507-10127 |
| Spacer Sequence | Spacer | 10128-10144 |
| 35S Terminator | Transcriptional Terminator | 10145-10319 |
| Spacer Sequence | Spacer | 10320-10323 |
| Hygromycin B Phosphotransferase | Selectable Marker Gene | 10324-11352 |
| ORF | | |
| Spacer Sequence | Spacer | 11350-11353 |
| SynJ 5UTR | 5UTR sequence | 11354-11380 |
| CsVMV Promoter | Promoter driving HygR | 11381-11894 |
| Spacer Sequence | Spacer | 11895-11898 |
| pCAMBIA1305.1 backbone | Spacer | 11899-12061 |
| T DNA repeat | Left Border | 12062-12087 |
| pCAMBIA1305.1 backbone | Spacer | 12088-12450 |
| Backbone from pJ-Kan low | Spacer | 12451-12784 |
| AmpR promoter | Promoter driving KanR | 12785-12876 |
| KanR ORF | Bacterial Selection | 12877-13686 |
| Backbone from pJ-Kan low | Spacer | 13687-13707 |

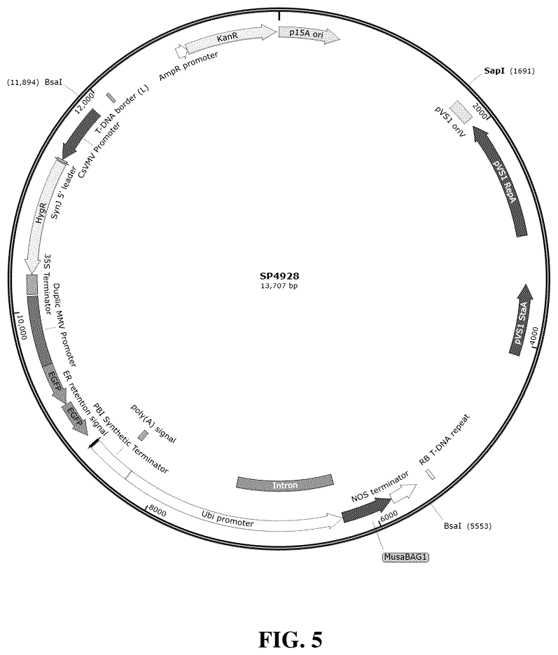

A map of the SP4928 expression vector is shown in FIG. 5.

SP0773 (SEQ ID NO:223): This expression vector comprises a nucleic acid sequence (Ma06g33150; SEQ ID NO:170) encoding a TLP antimicrobial peptide from Musa acuminata (SEQ ID NO:172), plus an nucleic acid sequence (Ma09g27770; SEQ ID NO:197) encoding a snakin antimicrobial peptide from Musa acuminata (SEQ ID NO:199). The detailed description of the components of the SP0773 expression vector is shown below in Table 6. In Table 6, the T-DNA payload is indicated in italics, the backbone is indicated in underlining, and the T4 resistance elements are indicated in bold.

TABLE 6

| Description | Function | Position |
|---|---|---|
| RB | T-DNA right border of | 1-25 |
| | Agrobacterium tumafaciens | |
| pCAMBIA1305.1 backbone | Spacer | 26-143 |
| Spacer Sequence | Spacer | 144-162 |
| AtHSP18.2 Terminator | Transcriptional Terminator | 648-163 |
| Spacer Sequence | Spacer | 649-652 |
| Ma06_{—}p33150.1 | Elo's proprietary | 1333-653 |
| | antifungal gene | |
| Spacer Sequence | Spacer | 1334-1334 |
| CmYLCV promoter | Promoter driving | 1799-1335 |
| | Ma06_{—}p33150.1 | |
| Spacer Sequence | Spacer | 1800-1811 |
| ZmUbi1 Promoter | Promoter driving Promoter | 1812-3803 |
| | driving Ma09_{—}p27770.1 | |
| Spacer Sequence | Spacer | 3804-3804 |
| Ma09_{—}p27770.1 | Elo's proprietary | 3805-4080 |
| | antifungal gene | |
| Spacer Sequence | Spacer | 4081-4084 |
| Gmax MYB2 Terminator | Transcriptional Terminator | 4085-4624 |
| Spacer Sequence | Spacer | 4625-4636 |
| CaMV e35S Promoter | Promoter driving | 4637-5378 |
| | NeoR/KanR | |
| Spacer Sequence | Spacer | 5379-5379 |
| NeoR/KanR | Plant selection marker | 5380-6174 |
| | gene | |
| Spacer Sequence | Spacer | 6175-6178 |
| CaMV 35S T | Transcriptional Terminator | 6179-6358 |
| Spacer Sequence | Spacer | 6359-6373 |
| pCAMBIA1305.1 backbone | Spacer | 6374-6530 |
| LB | T-DNA right border of | 6531-6555 |
| | Agrobacterium tumafaciens | |
| pCAMBIA1305.1 backbone | Spacer | 6556-6917 |
| Backbone from pJ-Kan low | Spacer | 6918-7252 |
| AmpR promoter | Promoter driving KanR | 7253-7344 |
| KanR | Bacterial Selection | 7345-8154 |
| Backbone from pJ-Kan low | Spacer | 8155-8175 |
| p15A ori | E. coli origin of replication | 8176-8721 |
| Backbone from pJ-Kan low | Spacer | 8722-9543 |
| pCAMBIA1305.1 backbone | Spacer | 9544-9887 |
| pVS1 oriV | required for function in | 9888-10082 |
| | Agro | |
| pCAMBIA1305.1 backbone | Spacer | 10083-10147 |
| pVS1 RepA | required for function in | 11221-10148 |
| | Agro | |
| pCAMBIA1305.1 backbone | Spacer | 11222-11649 |
| pVS1 StaA | required for function in | 12279-11650 |
| | Agro | |
| pCAMBIA1305.1 backbone | Spacer | 12280-13578 |

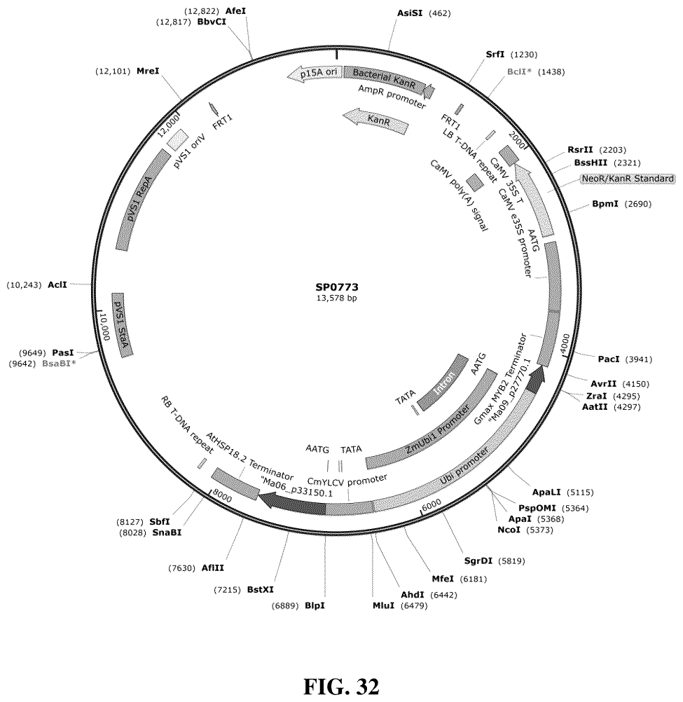

A map of the SP0773 expression vector is shown in FIG. 32.

SP1897: This expression vector comprises an OsXa4 (disease resistance gene) nucleic acid sequence, from Oryza sativa, optimized for high GC content (SEQ ID NO: 102), plus a UDP-glycosyltransferase (SsGT1) nucleic acid sequence, from Solanum sogarandinum, optimized for high GC content (SEQ ID NO:110). The detailed description of the components of the SP1897 expression vector is shown below in Table 7. In Table 7, the T-DNA payload is indicated in italics and backbone is indicated in underlining. The TR4 resistance elements are in bold.

TABLE 7

| Description | Function | Position |
|---|---|---|
| RB | T-DNA right border of | 1-25 |
| | Agrobacterium tumafaciens | |
| pCAMBIA1305.1 backbone | Spacer | 26-143 |
| Spacer Sequence | Spacer | 144-162 |
| AtHSP18.2 Terminator | Transcriptional terminator | 648-163 |
| Spacer Sequence | Spacer | 649-652 |
| OsXa4 GC | A cell wall-associated | 2776-653 |
| | kinase gene of Oryza | |
| | sativa, conferring | |
| | resistance to | |
| | Xanthomonas oryzae | |
| Spacer Sequence | Spacer | 2777-2777 |
| ZmUbi1 Promoter | Promoter driving OsXa4 | 4769-2778 |
| | GC | |
| Spacer Sequence | Spacer | 4770-4781 |
| CmYLCV promoter | Promoter driving SsGT1 | 4782-5246 |
| | GC | |
| Spacer Sequence | Spacer | 5247-5247 |
| SsGT1 GC | A glycosyltransferase | 5248-6669 |
| | gene of Solanum | |
| | sogarandinum, | |
| | conferring resistance to | |
| | fungal pathogens | |
| Spacer Sequence | Spacer | 6670-6673 |
| Gmax MYB2 Terminator | Transcriptional terminator | 6674-7213 |
| Spacer Sequence | Spacer | 7214-7225 |
| CaMV e35S promoter | Promoter driving | 7226-7967 |
| | NeoR/KanR | |
| Spacer Sequence | Spacer | 7968-7968 |
| NeoR/KanR | Plant selection marker | 7969-8763 |
| | gene | |
| Spacer Sequence | Spacer | 8764-8767 |
| CaMV 35S T | Transcriptional terminator | 8768-8947 |
| Spacer Sequence | Spacer | 8948-8962 |
| pCAMBIA1305.1 backbone | Spacer | 8963-9119 |
| LB | T-DNA right border of | 9120-9144 |
| | Agrobacterium tumafaciens | |
| pCAMBIA1305.1 backbone | Spacer | 9145-9506 |
| Backbone from pJ-Kan low | Spacer | 9507-9841 |
| AmpR promoter | Promoter driving KanR | 9842-9933 |
| KanR | Bacterial Selection | 9934-10743 |
| Backbone from pJ-Kan low | Spacer | 10744-10764 |
| p15A ori | E. coli origin of replication | 10765-11310 |
| Backbone from pJ-Kan low | Spacer | 11311-12132 |
| pCAMBIA1305.1 backbone | Spacer | 12133-12476 |
| pVS1 oriV | Origin of replication from | 12477-12671 |
| | Pseudomonas plasmid | |
| | pVS1, required for function | |
| | in Agrobacterium | |
| pCAMBIA1305.1 backbone | Spacer | 12670-12736 |
| pVS1 RepA | Replication protein from | 13810-12737 |
| | Pseudomonas plasmid | |
| | pVS1, required for function | |
| | in Agrobacterium | |
| pCAMBIA1305.1 backbone | Spacer | 13811-14238 |
| pVS1 StaA | Stability protein from | 14868-14239 |
| | Pseudomonas plasmid | |
| | pVS1, required for function | |
| | in Agrobacterium | |
| pCAMBIA1305.1 backbone | Spacer | 14869-16167 |

SP3941: This expression vector comprises a nucleic acid sequence (Ma06_g33150; SEQ ID NO: 170) encoding a TLP antimicrobial peptide from Musa acuminata (SEQ ID NO: 172), plus an OsUMP1 (proteosome maturation factor) nucleic acid sequence from Oryza sativa, optimized for high GC content (SEQ ID NO: 100). The detailed description of the components of the SP3941 expression vector is shown below in Table 8. In Table 8, the T-DNA payload is indicated in italics and backbone is indicated in underlining. The TR4 resistance elements are in bold.

TABLE 8

| Description | Function | Position |
|---|---|---|
| RB | T-DNA right border of | 1-25 |
| | Agrobacterium tumafaciens | |
| pCAMBIA1305.1 backbone | Spacer | 26-143 |
| Spacer Sequence | Spacer | 144-162 |
| AtHSP18.2 Terminator | Transcriptional terminator | 648-163 |
| Spacer Sequence | Spacer | 649-652 |
| Ma06_{—}p33150.1 | Elo's proprietary | 1333-653 |
| | antifungal gene | |
| Spacer Sequence | Spacer | 1334-1334 |
| CmYLCV promoter | Promoter driving | 1799-1335 |
| | Ma06_{—}p33150.1 | |
| Spacer Sequence | Spacer | 1800-1811 |
| ZmUbi1 Promoter | Promoter driving OsUMP1 | 1812-3803 |
| | variant R2115 | |
| Spacer Sequence | Spacer | 3804-3804 |
| OsUMP1 variant R2115 | A proteasome maturation | 3805-4296 |
| | factor gene of Oryza | |
| | sativa, conferring broad- | |
| | spectrum disease | |
| | resistance | |
| Spacer Sequence | Spacer | 4297-4300 |
| Gmax MYB2 Terminator | Transcriptional terminator | 4301-4840 |
| Spacer Sequence | Spacer | 4841-4852 |
| CaMV e35S promoter | Promoter driving | 4853-5594 |
| | NeoR/KanR | |
| Spacer Sequence | Spacer | 5595-5595 |
| NeoR/KanR | Plant selection marker | 5596-6390 |
| | gene | |
| Spacer Sequence | Spacer | 6391-6394 |
| CaMV 35S T | Transcriptional terminator | 6395-6574 |
| Spacer Sequence | Spacer | 6575-6589 |
| pCAMBIA1305.1 backbone | Spacer | 6590-6746 |
| LB | T-DNA right border of | 6747-6771 |
| | Agrobacterium tumafaciens | |
| pCAMBIA1305.1 backbone | Spacer | 6772-7133 |
| Backbone from pJ-Kan low | Spacer | 7134-7468 |
| AmpR promoter | Promoter driving KanR | 7469-7560 |
| KanR | Bacterial Selection | 7561-8370 |
| Backbone from pJ-Kan low | Spacer | 8371-8391 |
| p15A ori | E. coli origin of replication | 8392-8937 |
| Backbone from pJ-Kan low | Spacer | 8938-9759 |
| pCAMBIA1305.1 backbone | Spacer | 9760-10103 |
| pVS1 oriV | Origin of replication from | 10104-10298 |
| | Pseudomonas plasmid | |
| | pVS1, required for function | |
| | in Agrobacterium | |
| pCAMBIA1305.1 backbone | Spacer | 10299-10363 |
| pVS1 RepA | Replication protein from | 11437-10364 |
| | Pseudomonas plasmid | |
| | pVS1, required for function | |
| | in Agrobacterium | |
| pCAMBIA1305.1 backbone | Spacer | 11438-11865 |
| pVS1 StaA | Stability protein from | 12495-11866 |
| | Pseudomonas plasmid | |
| | pVS1, required for function | |
| | in Agrobacterium | |
| pCAMBIA1305.1 backbone | Spacer | 12496-13794 |

### Example 2

### Transgenic Musa acuminata Plants

Initiation and Maintenance of Banana Embryonic Cell Suspension (ECS) Cultures

This protocol details how to initiate embryogenic callus from male flowers (inflorescences) of banana and how to establish and maintain banana embryogenic cell suspension (ECS) cultures. The quantity and quality of these cultures is important for successful Agrobacterium-mediated transformation of banana.

Materials

Male banana flowers (inflorescences) 10 weeks after flowering initiation are used. Supplies used are: sharp scissors or knife for cutting; scalpel (#11, #20); scalpel handle; full-strength commercial bleach; sterile water; 15 mL Falcon tubes; 50 mL Falcon tubes; sterile 60×15 mm petri plates; sterile 150×15 mm petri plates; 100% ethanol; sterile 10 mL wide-mouth pipettes; sterile 25 mL pipettes; pipet aid; sterile 50 mL Erlenmeyer flasks; sterile 125 mL Erlenmeyer flasks; sterile 250 mL Erlenmeyer flasks; and 750 um PluriStrainer (Pluriselect: SKU 43-50750-03).

The media compositions used for ECS initiation, subculture, and maintenance are detailed below.

T5 Medium (Flower Buds)

Full strength MS salts and vitamins, sucrose: 30 g/L, biotin: 1 mg/L, pH: 5.6-5.8, Phytagel: 2.3 g/L, autoclave 500 mL on liquid 25 cycle. After autoclaving and cooling add: 2,4-D: 4 mg/L, NAA: 1 mg/L and IAA: 1 mg/L. Pour media into tall culture plates.

ECS Liquid Media (Initiation and Maintenance)

Full strength MS salts and vitamins, sucrose: 45 g/L, L-glutamine: 100 mg/L, malt extract: 100 mg/L, biotin: 1 mg/L, pH 5.3, autoclave 500 mL on liquid 25 cycle. After autoclaving and cooling add: 2,4-D: 1 mg/L and Picloram: 0.25 mg/L.

Embryo Development Medium (EDM)

Full strength MS salts and vitamins, sucrose: 45 g/L, L-glutamine: 100 mg/L, malt extract: 100 mg/L, proline: 230 mg/L, maltose: 10 g/L, pH: 5.8, Gelrite: 3 g/L. After autoclaving and cooling add: Zeatin: 0.05 mg/L, Kinetin: 0.1 mg/L, NAA: 0.2 mg/L and 2iP: 0.2 mg/L.

Germination Medium (GM)

Full strength MS salts and vitamins, sucrose: 30 g/L, pH 6.0, Phytagel: 3 g/L. After autoclaving and cooling add: BAP: 1 mg/L and GA3: 0.5 mg/L.

Methods

All procedures are performed in a sterile laminar flow hood unless otherwise specified. It takes anywhere from 4 to 6 months for embryogenic callus to develop from the male flowers. After embryogenic callus forms, it takes between 3 to 4 months for high quality cell suspensions to establish.

Disinfection of Male Flowers and Embryogenic Callus Establishment

1. Male flowers (inflorescence) were collected from the flower bud. The material was collected 10 weeks after flowering initiation. The culture of the flowers started the same day of the collection or 24 hours later. 2. Using sharp scissors or a knife, the inflorescences were trimmed to approximately 3.5 cm in length and the material was transferred to a sterile 50 mL Falcon tube. No more than 2 inflorescences were used per tube. 3. 40 mL of a freshly prepared 20% commercial bleach solution was added to the flowers in the tube and incubated for 2 minutes with gentle inversion. 4. The bleach solution was poured off and discarded. 5. The floral tips were washed twice with 40 mL of sterile distilled water. 6. The floral tips were transferred to a sterile 150 mm×15 mm petri plate. 7. Using a scalpel (#20), the size of the inflorescences were further reduced until they were approximately 1.5 cm long. 8. The explants were transferred back to the 50 mL Falcon tube, 40 mL of freshly prepared 20% ethanol was added and incubated for 1.5 minutes with gentle inversion. 9. The 20% ethanol was poured off and discarded. 10. The explants were washed three times with 40 mL of sterile distilled water. 11. The explants were transferred to a sterile petri plate placed on the stage of a dissecting microscope. 12. A scalpel (#11) was used to extract the flowers. Once extracted, the flowers were placed in a 60×15 mm petri plate containing sterile distilled water for 1 minute. The immature flowers were taken from positions 3-10 of the flower tip, taking as reference position 1, which corresponds to the meristematic dome. 13. After 1 minute, the flowers were transferred to T5 medium. No more than 5 flowers were used per plate. 14. The plates were sealed with plastic wrap, and the plates were placed inside a growth chamber with constant lighting (1750 Lux) at 24±1C until the appearance of different types of calli. The length of time for callus induction varied between 4 and 6 months depending on the quality of the culture.

Embryogenic Calli Selection and ECS Initiation

1. The flowers were observed on T5 once a month and checked for the appearance of embryogenic callus. After the first three months, the explants were checked every two weeks. The explant's evolution until the appearance of embryogenic callus was visible through the following sequence of events: a. during the first month, tissue oxidation and thickening of the explant was visible; b. after the first month, yellow callus (not embryogenic) developed (this callus had a nodular appearance and was not used for cell suspension. White crystalline compact callus may also develop); c. after four months, embryogenic callus began to develop depending on the culture. This callus was made up of white/translucent pro-embryogenic masses and was suitable to start an embryogenic cell suspension. 2. Once embryogenic callus developed that was suitable for a cell suspension, initiation of an ECS culture began. 3. 5 mL of ECS media was added to a sterile 50 mL Erlenmeyer flask. 4. 18-20 embryos (either in a cluster or individually) were placed into the flask. 5. The flask was sealed with aluminum foil and plastic wrap and placed on a rotary shaker at 110 rpm under constant lighting (1500 Lux) at 24±1° C. 6. After 2 weeks, a sterile wide-mouth 10 mL pipet was used to transfer the cells and media to a sterile 15 mL Falcon tube. 7. The cells were allowed to settle for approximately 2 minutes. 8. 50% of the original medium volume was removed and replaced with an equal volume of fresh medium. 9. The cells were transferred to a clean sterile 50 mL Erlenmeyer flask and the flask was sealed with aluminum foil and plastic wrap. 10. The flask was placed back on the shaker and cultured under the same conditions as above. 11. Every week, weekly refreshments were continued using the methods detailed in steps 6 to 10. 12. After 30 days, the types of cells in each suspension were observed with the help of an inverted microscope. At this point, the quality of the suspensions was evaluated on a weekly basis. If an increase in the packed cell volume (PCV) was observed, 1-2 mL of fresh medium was added in addition to the volume of fresh media used to replace the old media. Once the volume of the culture reached 0.4 ml SCV or at least 9 mL of culture media, the contents were transferred to a sterile 125 mL Erlenmeyer flask for growth. Table 9, below, lists the growth containers and ECS and media requirements. With the weekly evaluation, selection of the most suitable cells for establishing a homogenous cell suspension was done. During the evaluation, if undesirable material was observed, it was removed using a graduated pipet.

TABLE 9

| | Flask | Minimum Medium | Maximum Medium |
|---|---|---|---|
| SCV | Size | Volume | Volume |
| Up to 0.4 mL | 50 mL | 5 mL | 9 mL |
| 0.4 mL to 1.0 mL | 125 mL | 10 mL | 18 mL |
| 1.0 mL to 2.0 mL | 250 mL | 20 mL | 60 mL |

ECS Maintenance and Subculture (Performed Weekly)

This procedure was for suspension cultures that had been previously established and where the SCV of the suspension was approximately 1 mL or greater. If the SCV values of the culture was less than 1 mL, Table 1 was used to guide the flask size and the volume of old ECS media to new ECS media, maintaining a 1:2 ratio of old media to new media. 1. The flasks containing ECS cultures were removed from the growth chamber and brought into a sterile laminar flow hood. 2. Using a sterile 25 mL pipet, the contents of the culture were transferred to a sterile 50 mL Falcon tube, and the cells were allowed to settle by gravity for two minutes. If a high percentage of the cell population was large clusters/embryos, the cells were filtered through a 750 um PluriStrainer when transferred to the 50 mL Falcon tube. Transferring cells that have formed a ring on the side of the Erlenmeyer flask was avoided. Ideal ECS cultures quickly settled to the bottom of the Falcon tube. Dead and/or dying cells took longer to settle to the bottom. 3. The supernatant was removed with the 25 mL pipet and transferred back to the culture flask, leaving 10 mL of culture in the 50 mL Falcon tube. 4. The cells were resuspended in the 10 mL of remaining media and transferred to a sterile 15 mL Falcon tube. Sterile 15 mL Falcon tubes that have volume graduations on the conical tip were used. 5. The cells were allowed to settle for two minutes. 6. While the cells were settling in the 15 mL Falcon tube, the discarded media was poured from the flask back into the 50 mL Falcon tube, and the contents were allowed to settle. 7. The supernatant of the cells in the 15 mL Falcon tube was observed. If the supernatant was mostly clear, the culture was of sufficient quality that all media in the 15 mL tube was used for subculture. If a portion of cells had settled to the bottom but a good amount remained in the supernatant, as much of the supernatant as possible was removed (including the cells that have not yet settled) and discarded in the culture flask. Media was added from the 50 mL Falcon tube to the cells up to a volume of 10 ml. 8. The cells were resuspended in the media and transferred to a new flask. If large clusters/clumps/embryos were observed in the cell population, these were manually removed with a pipet. 9. 5 mL of old media from the 50 mL Falcon tube was added to the flask. 10. 30 mL of fresh media was added to the flask. The ratio of old media to fresh media should generally be 1 volume old media to 2 volumes fresh media. 11. The cap was secured on the flask and labeled with cell line ID, date, and the SCV amount. 12. The flask was placed back on the rotary shaker and continued culture at 110 rpm under constant lighting (1500 Lux) at 24±1° C.

ECS Quality Control and Regeneration Checks

1. As the ECS were actively growing, a small portion of the culture was taken and dyed with FITC to determine cell viability. Cell shape and cell contents (granules) were also observed. Cell clusters were small and tightly compact with very fine granules inside the cells. 2. When the ECS cultures reached 0.5 mL SCV and were increasing at a rate of 0.3×SCV every week, a small aliquot was taken to test for regeneration. 50 μl-100μ of SCV was plated on embryo development medium (EDM). The plate was sealed with plastic wrap and cultured in the dark at 28° C. Embryo masses were observed between 22 and 30 days after plating on EDM. It certain cases it took as long as 7 weeks to observe a response. If no response was seen after 7 weeks, that culture was discarded. A culture was still considered good if >65% of the cells formed embryo masses. After differential embryos were formed that contained a clearly defined notch on the upper part of the embryo, they were transferred to germination media (GM). It took between 1 and 3 months for embryos to germinate and form shoots.

Protoplast Regeneration from Banana ECS

This protocol describes generation of genome edited TR4 resistant plants from banana ECS protoplasts.

Materials

Supplies

Banana embryogenic cell suspension culture; Macerozyme RS (Goldbio Duchefa Biochemie); Pectolyase Y-23 (Sigma V2010-250ML); Sodium alginate (Phytotechlabs A108); 5 mg/ml Fluorescein Diacetate (FDA); 1 mg/ml Propidium iodide (PI); 0.45 μm Millipore Steriflip-GP Filter 50 ml (Millipore SE1M003M00); 0.22 μm Millipore Steriflip-GP Filter 50 ml (Millipore SE1M003M02); 70 μm nylon cell strainers; 40 μm nylon cell strainers; 500 μm nylon cell strainers; C-Chip™ Disposable Hemacytometers (82030-468); Pipettors; 25 ml serological pipettes; 200 μl wide mouth tips (46620-642); 1000 μl wide mouth tips (89049-168); Polyethylene glycol 4000 (Sigma 81240-1KG); 2 ml centrifuge tubes (round bottom); 14 ml culture tubes (round bottom); 50 ml sterile centrifuge tubes; 15 ml sterile centrifuge tubes; 6-well tissue culture plates; 60×15 mm petri dishes; 100×15 mm petri dishes; 100×25 mm petri dishes.

Media and Composition

S1 solution (1 L): CaCl₂·2H₂O 6.68 g, KCl 30 g, MES 0.25 g, adjust pH 5.7 with 1 N KOH, make up the volume to 1 L with water, sterilize by using 0.22 μM filter; Enzyme Solution (40 ml): Cellulase RS 0.6 g (1.5%), Pectolyase Y-23 0.08 g (0.2%), S1 solution 40 ml, dissolve enzymes and centrifuge at 4000 rpm for 10 minutes (the enzyme cannot be completely dissolved to clear, a little cloudy is fine as long as there are no clumps remaining), take supernatant and filter using 0.45 μM filter tube (do not autoclave); W58 Salt solution (500 ml): water 450 ml, CaCl₂·2H₂O 0.90 g (0.0367 osmol), NaCl 8.00 g (0.547 osmol), KCl 0.20 g (0.0107 osmol), MES 0.30 g (0.0037 osmol), glucose 0.50 g (0.0055 osmol), adjust pH 5.7 with 1 N KOH, make up the volume to 500 ml with water, sterilize by using 0.22 μM filter; MMG solution (100 ml): ˜0.55 osmol: water 50 ml, MES 0.0853 g (0.004 M, 0.004 osmol), mannitol 9.1085 g (0.500 M, 0.5 osmol), MgCl₂·7H₂O 0.3050 g (0.015 M, 0.045 osmol), adjust pH 5.7 with 1 N KOH, make up the volume to 100 ml with water, sterilize by using 0.22 μM filter; 50% PEG-CaCl₂ Transfection solution (50 ml): ˜0.55 osmol: water 10 ml, PEG 4000 25 g (50%), mannitol 1.822 g (0.2 M, 0.2 osmol), CaCl₂·2H₂O 0.735 g (0.1 M, 0.3 osmol), make up the volume to 50 ml with water (heat over 66 C to encourage dissolution), sterilize by filtering through a 0.45 μM filter tube, cool down to room temperature prior to use, PEG solution can be stored at room temperature and should be used within 30 days; 0.6M Mannitol solution (500 ml): water—500 ml, mannitol 54.6 g, sterilize by using 0.22 μM filter; 1.6% alginate solution (20 ml): 0.6M mannitol 20 ml, sodium alginate 0.32 g, sterilize by using 0.22 μM filter; Bead-forming solution 0.8M CaCl₂) (1 L): water 1 L, CaCl₂·2H₂O 117 g (0.8M), sterilize by using 0.22 μM filter; ECS medium (1 L): MS salts with vitamins 4.33 g, malt extract 100 mg, biotin 1 mg, L-glutamine 100 mg, sucrose 45 g, 2,4 D 1 mg, Picloram 0.25 mg, adjust pH 5.7 with 1 N KOH, autoclave to sterilize; Depolymerization solution (1 L): mannitol 54.6 g, sodium citrate 5.16 g, adjust pH 5.7 with 1 N KOH, make up the volume to 1 L with water, sterilize by using 0.22 μM filter; 0.8% agarose (100 ml): water 100 ml, agarose 0.8 g, adjust pH 5.7 with 1 N KOH, autoclave to sterilize; Feed layer 2×PCM (1 L): MS salts 8.66 g, glucose 500 mg, maltose 100 g, sucrose 40 g, meso-inositol 450 mg, 2,4 D 4 mg, adjust pH 5.7 with 1 N KOH, make up the volume to 1 L with water, sterilize by using 0.22 μM filter; EDM medium (1 L): MS salts with vitamins 4.33 g, malt extract 100 mg, L-glutamine 100 mg, proline 230 mg, sucrose 45 g, maltose 10 g, NAA 0.2 mg, zeatin 0.05 mg, kinetin 0.1 mg, 2iP 0.2 mg, melatonin 100 uM, Gelrite 3 g, adjust pH 5.7 with 1 N KOH, autoclave to sterilize; EMM medium (1 L): MS salts with vitamins 4.33 g, Myo-inositol 100 mg, ascorbic acid 100 mg, sucrose 30 g, Gelrite 3 g, adjust pH 5.8 with 1 N KOH, autoclave to sterilize; GM medium (1 L): MS salts and vitamins 4.33 g, sucrose 30 g, GA3 0.5 mg, BAP 1 mg, Gelrite 3 g, adjust pH 6.0 with 1 N KOH, autoclave to sterilize.

Methods

Enzyme Digestion

1. Take out a flask of 4-day-suculture ECS from shaker (make sure to prepare fresh ECS subculture 4 days before the day of enzyme digestion). 2. Sterilize the flask and the hood before opening the cap of flask. 3. Transfer all the ECS from the flask to 15 ml. If the volume is too large, transfer to 50 ml tube first and then move the pellet and less than 15 ml supernatant to 15 ml tubes. 4. Let the ECS to settle for 10 minutes before remove the supernatant (do not spin down as the floating junk cells will be included into protoplast digestion). 5. Read the SCV from the 15 ml tube and record. Make sure the SCV does not exceed to 1.5 ml as each of the 15 ml can only hold up to 8.5 ml enzyme/ECS mixture ensure proper shaking and therefore highest digestion efficiency. Usually, healthy ECS produce 10-20 million protoplasts per 1 ml SCV. 6. Remove supernatant as much as possible and add 7.5 ml enzyme solution. Lay down the tube and tap on a rotary shaker at 26° C. in dark for overnight digestion (make sure to life up the bottom of the tube so that pellet is properly shaking).

Protoplast Isolation

1. Place a 70 μm and a 40 μm cell strainer into two separate sterile 50 ml centrifuge tubes. Sequentially, sieve the protoplast through two cell strainers by pipetting very slowly to remove undigested tissue. 2. Spin down the solution at 100×g for 5 minutes. A white pellet should appear at the bottom. Aspirate out the supernatant without disturbing the protoplast pellet. 3. Wash the pellet twice more with W58 solution (15 ml/tube) and centrifuge again at 100×g for 3 minutes (repeat 1-2 more times if dead debris still can be seen—indicated by a swirl of debris in the supernatant when taking out the tubes). 4. Resuspend the protoplast pellet in MMG solution (5-10 ml depending on the yield.).

Cell Count and Viability

1. Take 20 μl protoplast solution and dilute with 80 MMG solution in a 2 ml microcentrifuge tube. 2 Add 2 μl PI (1 mg/ml) and 0.2 μl FDA (5 mg/ml) to the 100 μl diluted protoplast solution. 3. Gently mix the protoplast solution by tapping the side of the tube and/or inverting the tube 3 times. The protoplast is sensitive to PI and viability will decrease if exposed for too long. If more than 2 samples are to be counted, prepare 2 samples at a time for cell count. 4. Load 10 μl of protoplast solution into each counting grid of a C-chip disposable hemacytometer and count the number of viable and dead protoplasts (see bitesizebio.com/13687/cell-counting-with-a-hemocytometer-easy-as-1-2-3/for full instruction. Number of protoplast/ml=N (average number of protoplasts from 4 corner squares, each corner square has 4×4 small squares)×104). 5. Adjust the protoplast concentration to 5×10⁵ cells/ml by either adding an additional amount of MMG or by centrifuging the protoplasts at 100×g for 2 minutes, and resuspending in MMG solution. 6. Keep the protoplasts at 4° C. until transfection.

Bulk PEG-Mediated Transfection for Regeneration

1. Add proper amount of reagent (see Table 10) to each of round bottom culture tubes (14 ml), all the mRNAs are adjusted to 2 μg/μl after cleanup, all the crRNAs are ordered through IDT Cas12a crRNA platform for 10 nmol delivery. 40 μl RNase free water was used to resuspend dry pellet to final concentration 250 μM.

TABLE 10
mRNA delivery dosage for 500,000 protoplasts per sample

| Reagent | Dose | Goal |
|---|---|---|
| HEn | 200 μg (400 pmol) | Knockout |
| | [100 μl] | |
| MAD7 + | 80 μg (57 pmol) + 126 μg | Knockout |
| crRNA | (10000 pmol) | |
| | [40 μl + 40 μl] | |
| HEn + | 100 μg (200 pmol) + 300 μg | Targeted |
| oligo donor | (2500 pmol) | Insertion |
| | [50 μl + 25 μl] | |
| MAD7 + crRNA + | 80 μg (57 pmol) + 126 μg | Targeted |
| oligo donor | (10000 pmol) + 300 μg | Insertion |
| | (2500 pmol) | |
| | [40 μl + 40 μl +25 μl] | |

2. Add 1000 μl of protoplasts (5×10⁵ protoplasts) with wide-bore pipette tips to each culture tube and mix by pipetting up and down very gently (1000 μl protoplasts are the maximal volume recommended to put in a culture tube. Increase the number of tubes if more protoplasts are needed for regeneration). For each delivery design, prepare 10 tubes of delivery (5×10⁶ protoplasts) which will be enough for 5 wells in a 6-well culture plate. 3. Using wide-bore pipette tips, add 1100 μl of 50% PEG-CaCl₂ transfection solution to the appropriate tubes and mix completely by gently pipetting up and down approximately ten times or till the solution is homogenized. Incubate the tubes at room temperature for 20 minutes. Keep the protoplast:RNA:PEG ratio 10:1:11. RNA should be very pure. 4. After incubation, dilute the transfection mixture with 10 ml of W58 Salt solution and mix well by gently reverting tubes five times. 5. Centrifuge at 200×g for 5 minutes at room temperature using a bench-top centrifuge. 6. Carefully remove and discard 9.5 ml of the supernatant. Only remove as much as can be allowed for the aspirate to remain clear. Do not get too close to the pellet. 7. Add a fresh volume of 10 ml of W58 salt solution to the appropriate number of tubes and gently resuspend the protoplasts by reverting tubes five times to mix the contents. 8. Centrifuge at 100×g for 3 minutes at room temperature using a bench-top centrifuge. After this wash the protoplasts should be better pelleted at the bottom. 9. Carefully remove and discard 10.5 ml of the supernatant, control the pipette slowly and only remove as much as can be allowed for the aspirate to remain clear. Approximately 1.5 ml should remain in the tubes. 10. Resuspend protoplasts by gently swirling the tubes. Transfer protoplast solution to a sterile 2 ml round bottom tube. 11. Centrifuge at 100×g for 3 minutes at room temperature using a bench-top centrifuge. A nice pellet should form at the bottom of the tubes. 12. Remove supernatant as much as possible without disturbing the pellet. 13. Resuspend pellet with 250 μl 0.6 M mannitol (need to be left on ice if not proceeding immediately to the next step).

Alginate Beads Preparation

1. Add 250 μl 1.6% alginate to the mixture and mix well by gently pipette up and down 10 times. Transfected protoplasts are in 0.8% alginate solution with an estimated density of 6×10⁵ that are ready to be used for polymerization and alginate beads formation. Tubes with same transfection can be combined as needed. 2. Prepare 6-well plates filled with 4 ml 0.8M in CaCl₂) each well. Add 1 ml of protoplasts alginate solution with 200 μl wide-bore pipette tips to each well (it's ok if some of the beads fused together). A 6-well plate can hold approximately 3.6×10⁶ protoplasts for regeneration. 3. Wait for 10 minutes for polymerization to complete and remove CaCl₂) solution as much as possible without taking away alginate beads using 10 ml pipettes. 4. Wash alginate beads with 4 ml ECS medium once in each well and remove medium completely. 5. Prepare feeder layer solution same as ECS subculture 1:50 ratio, e.g., 1 ml SCV 5-day-old ECS+10 ml ECS old medium+40 ml ECS fresh medium will yield 50 ml feeder layer solution. No filtrate of ECS needed. 6. Add 4 ml feeder layer solution to each well. Make sure to thoroughly resuspend the ECS mixture each time as the cells settle down fast.

Protoplast Regeneration in Alginate Beads

1. Leave plates in 32° C. shaker for 5 days. 2. Take plates out on day 6 and check under microscope. Protoplast first division should be observed at this point. Transfer plates to 27° C. shaker. 3. On day 14, check protoplast samples under microscope. Small cell clusters should appear at this point. Feed 1 ml of fresh ECS medium before returning to shaker. Pay attention to the quality of ECS feeder layer. If the ECS clusters turned brown, replace the feeder layer with new feeder layer solution made of fresh ECS cultures: 1 ml SCV 5-day-old ECS+10 ml ECS old medium+40 ml ECS fresh medium will yield 50 ml feeder layer solution. No filtration of ECS is needed. 4. On day 21, check protoplast samples under microscope. Embryogenic structures should appear. The size of embryogenic calli varies from 100-200 μm. Pay attention to the quality of ECS feeder layer. If the ECS clusters turned brown, replace the feeder layer with new feeder layer solution made of fresh ECS cultures: 1 ml SCV 5-day-old ECS+10 ml ECS old medium+40 ml ECS fresh medium will yield 50 ml feeder layer solution. No filtration of ECS is needed. 5. On day 28 (week 4), check protoplast samples under microscope. The material should be ready to release from the alginate beads.

Prepare Agarose Feeder Layer

1. On day 26, prepare agarose feeder layer. 2. Melt 0.8% agarose and keep it warm in 37° C. beads tray until ready to use. 3. Filtrate 4-day-old ECS with 500 μM cell strainers. Approximately, 1 ml SCV filtrated ECS can be obtained from 2 ml SCV suspension culture (each feeder layer plate will need 0.5 ml SCV filtrated ECS cells. It is important to make sure to prepare enough ECS subculture ahead of time based on how many plates needed to be prepared). 4. Remove supernatant and resuspend ECS cells in 10× volume of PCM medium (to make 10% v/v ECS suspension). 5. Mix equal amount of agarose and ECS suspension thoroughly and immediately transfer 10 ml to 60×15 mm petri plate. Try to distribute ECS evenly in the plate. 6. Let it set for 15-20 minutes. Seal it until ready to use.

Release of Regenerated Cultures from Alginate Beads

1. Remove liquid feeder layer completely from 6-well plates containing alginate beads. Use fresh ECS medium when needed during feeder layer removal. Before adding depolymerization solution, check the plates under microscope to make sure all the ECS cells from feeder layer were completely removed from the wells. 2. Once feeder layer was completely removed, add 4 ml depolymerization solution to each well. Leave at room temperature for 30 minutes. 3. Pipette 20 times in each well to encourage dissolving process. Leave at room temperature for another 30 minutes. 4. Pipet until all the visible beads are dissolved (usually takes 3 times). 5. Transfer released cell clusters mixture to 15 ml conical tubes and let it set for 5 minutes. 6. Remove supernatant. Record the SCV in the tube. 7. Resuspend culture with ECS medium and let it set again for 5 minutes. 8. Remove supernatant and resuspend with ECS medium as 1:30 ratio (1 ml SCV culture in 30 ml ECS medium). 9. Transfer 4 ml resuspended cultures to a 35 mm plate containing agarose feeder layer. Continue until all the culture has been sub-cultured to agarose feeder layer plates. Make sure to resuspend the culture completely each time before transferring. 10. Leave all the plates in 27° C. shaker. 11. Check cultures under microscope weekly (Day 35, 42, 49, 56). Embryogenic clusters should continue to form and enlarge over time 400-500 μm. On day 42 (week 6) feed 1-2 ml of fresh ECS medium depending on the consumption by the culture. Do not let it dry up at any point as it might stress the culture and affect the growth. Be aware if the culture starts to turn dark that is the sign of stress.

Subculture Regenerated Microcalli to Solid Media

1. Day 56 (week 8), take the plates out of shaker and transfer the culture to 15 ml conical tubes. Wash the plate with 1 ml ECS medium for 3 times to remove all the culture from the plates. Measure the growth again. It should at least double the SCV. If not, transfer back to plates with feeder layer and culture for another week. If top of the culture is slightly blue, there is no need to try to remove them before subculture. These are the dead cells that didn't regenerate. They won't affect the rest of culture to regenerate. 2. Remove the supernatant until the volume is 6× of the SCV, e.g., for every 1 ml SCV add 6 ml ECS medium to resuspend. Take 600 μl and spread to a plate containing solid EDM medium. It is important to spread out the culture with liquid medium. When the culture is packed together, the regeneration efficiency will be affected. After spreading out the culture, try to remove access liquid as much as possible by gently tilting the plate. 3. Seal the plates and culture at 26° C. growth chamber. 4. Day 84 (week 12) Pick up pre-mature embryos and transfer to EMM for 2 weeks. 5. Week 14, check cultures under microscope. Subculture pre-mature embryo structures to fresh EMM and culture for another 2 weeks. Subculture mature embryos to GM for 2 weeks. 6. Week 16, at this point all the embryos should be matured. Subculture all embryos to fresh GM for 2 weeks. 7. Week 18, subculture embryos to fresh GM for 2 weeks. 8. Week 20, continue subculture culture to GM every 2 weeks. From this week on, pay attention to germination and transfer germinated plantlets to elongation medium under light.

Agrobacterium-Mediated Transformation of Banana Using ECS as Explant

Agrobacterium-mediated transformation offers several advantages over direct gene transfer methodologies, such as the possibility to transfer only one or few copies of DNA fragments carrying the genes of interest at higher efficiencies, with lower costs, and the transfer of large DNA fragments with minimal rearrangement. Transformation efficiency is also higher in Agrobacterium-mediated transformation in comparison to microprojectile bombardment. For banana, for example, in one study only ten individual transformation events were obtained from 90 bombardments (approximately 3 ml of settled cell volume), whereas another study generated up to 65 plants per 50 mg settled cell volume through Agrobacterium-mediated transformation of banana. At present, most of the reported transformation protocols for banana use embryogenic cell suspensions (ECSs) as the target tissue for transgene integration. The following protocol describes Agrobacterium-mediated transformation system for banana using embryogenic cell suspension (ECS) as explant.

Materials

Plant Material: Embryogenic cell suspension of banana (ECS) were developed from FEC. The age of the material was generally 7 days after subculture. High quality cells were creamy yellow in color and relatively uniform in size without large chunks of cells. The culture showed >65% regenerability to embryos within 7 weeks of plating on EDM media. Any volume of ECS up to 1 mL was used for each transformation.

Agrobacterium strain: A comparison of gene expression and cell death showed that EHA105 strain at OD₆₀₀ of 0.5-0.8 was the best suited for transformation of banana ECS. AGL1 strain at OD₆₀₀ of 0.5 was the second suited for transformation of ECS, and LBA4404 at OD₆₀₀ of 1.0 was the third best alternative.

Supplies: 2×YT liquid media (Teknova—Y0167); sterile 1.5 mL microcentrifuge tubes; sterile 200 μL tips; sterile 500 μL tips; sterile 10 mL plastic pipettes; sterile 10 mL wide-mouth pipets; pipet aid; sterile glass fiber filter circles 5.5 cm diameter (Fisherbrand 09-804-55A); sterile 2% Pluronic F-68 (Phytotechnology Labs—P770); Eppendorf 5810 centrifuge; sterile 125 mL Erlenmeyer flasks (preferably not beveled); plastic film for petri dish sealing; sterile forceps; media (see below); hormones (see below); sterile 15 mL Falcon tubes; sterile 50 mL Falcon tubes; rotary shaker; water bath pre-heated to 45° C.

Media and Composition: ECS liquid media: MS salts and vitamins, biotin 1 mg/L, L-glutamine 100 mg/L, malt extract 100 mg/L, sucrose 45 g/L, 2,4-D 1 mg/L, picloram 0.25 mg/L, pH 5.3. Embryo development medium (EDM): MS salts and vitamins, glutamine 100 mg/L, malt extract 100 mg/L, proline 230 mg/L, sucrose 45 g/L, maltose 10 g/L, Zeatin 0.05 mg/L, Kinetin 0.1 mg/L, NAA 0.2 mg/L, 2iP 0.2 mg/L, timentin 400 mg/L, melatonin 100 μM, Gelrite 3 g/L, pH 5.8. Embryo maturation medium (EMM): MS salts and vitamins, sucrose 30 g/L, ascorbic acid 100 mg/L, timentin 400 mg/L, Gelrite 3 g/L, pH 5.8. Germination medium (GM): MS salts and vitamins, sucrose 30 g/L, GA3 0.5 mg/L, BAP 1 mg/L, Gelrite 3 g/L, pH 6.0. Micropropagation medium (MP): MS salts and vitamins, sucrose 30 g/L, BAP 5 mg/L, L-cysteine 10 mg/L, Gelrite 2 g/L, pH 5.6. Rooting medium (RM): MS salts and vitamins, sucrose 30 g/L, IBA 1 mg/L, Gelrite 3 g/L, pH 5.8. TMA1: MS salts, MS vitamins, biotin 1 mg/L, malt extract 100 mg/L, glutamine 100 mg/L, proline 230 mg/L, ascorbic acid 40 mg/L, PVP 10 (5 g/L), cysteine 200 mg/L, IAA 1 mg/L, NAA 1 mg/L, 2,4-D 4 mg/L, sucrose 85.5 g/L, pH 5.3. Add melatonin 100 uM and acetosyringone 200 μM to solid TMA1 media.

Methods

Success rates for transformation depended largely upon the quality of the embryogenic cell suspension (ECS) and genotype of banana. Test the regeneration ability of ECS was tested before starting Agrobacterium-mediated transformation experiments. Any culture that regenerates less than 65% of embryo masses within 7 weeks of plating on EDM was not used for transformation. All steps listed below were done in a laminar flow hood unless specified otherwise.

Agrobacterium Preparation for Transformation (Performed One Day Before Agrobacterium Transformation)

1. Glycerol stocks were removed from −80° C. and placed on ice. 2. 25 mL of liquid 2×YT medium supplemented with appropriate antibiotics was added to a sterile 125 mL Erlenmeyer flask. Kanamycin 50 mg/L was the usual antibiotic for the binaries. Rif 25 mg/L was the usual antibiotic for EHA105 selection. Antibiotics varied among payloads and Agrobacterium strains. 3. 50 μl of 100 mM acetosyringone stock was added to the flask for a final concentration of 200 μM acetosyringone. 4. 500 μl of glycerol stock was added to the flask. 5. The flask was placed on a rotary shaker at 220 rpm and 28° C. for shaking overnight. The optical density (OD₆₀₀) reached 0.8 or more after overnight culture.

Agrobacterium Preparation for Transformation (this Step was Performed the Day of Agrobacterium Transformation)

1. A fresh glycerol stock of the transformation payload was prepared as follows: 800 μl of bacterial culture was aliquoted to a sterile 1.5 mL microcentrifuge tube. The tube was labeled and then 400 μl of sterile 50% glycerol was added to the tube. The bacteria and glycerol was mixed by inversion then placed at 80° C. for future transformations. 2. After making the glycerol stock, the bacterial culture was poured into a sterile 50 mL Falcon tube. 3. The bacterial cells were harvested by centrifugation at 4,150 rpm for 15 minutes at 25° C. 4. The supernatant was poured off (the flask was saved) and the cells were resuspended in 25 ml of TMA1 co-cultivation medium supplemented with 200 μM acetosyringone. Hormones and acetosyringone were added fresh each day of transformation. Effect of acetosyringone for banana Agrobacterium-mediated transformation was important. 5. The resuspended cells were poured back in the corresponding 125 ml Erlenmeyer flask (the 50 mL Falcon tubes were saved) and the lid was tightly closed. The flask was placed back on the rotary shaker, and the bacterial suspension was incubated at 28° C. for 3 hours with shaking at 220 rpm. 6. After 3 hours, the bacteria suspension was poured back into the corresponding 50 mL Falcon tube, and the bacterial cells were collected by centrifugation at 4,150 rpm for 15 minutes at 25° C. 7. The supernatant was poured off and the cells were resuspended in 5 mL of TMA1 medium. 8. 1 mL of resuspended bacterial cells was aliquoted to a new 50 mL Falcon tube and the optical density (OD₆₀₀) of the bacterial culture was adjusted to 0.9-1.0 with TMA1 medium. The volume of the culture was noted. 9. An equal volume in μl of sterile 2% Pluronic F-68 was added and the tubes were briefly vortexed to mix.

ECS Cell Preparation (this Step was Performed the Day of Agrobacterium Transformation, Preferably During Routine ECS Subculture)

1. 7 day old ECS cells were used the day of transformation. 2. A portion of ECS cells not to exceed 1 mL SCV was aliquoted to a sterile 15 mL Falcon tube. 3. The cells were allowed to settle and the SCV was recorded. 4. The volume of liquid in the 15 mL tube was adjusted to read 5 mL by either adding additional ECS liquid media or by removing excess media. 5. The above steps were repeated for each delivery payload. 6. The top and side of the 15 mL tube were labeled with the delivery payload ID. Cells were now ready for transformation.

Agrobacterium Transformation Procedure for Banana ECS

1. The 15 mL Falcon tubes containing up to 1 mL of ECS were placed in a 45° C. preheated water bath and incubated for 5 minutes. 2. The cells were removed from the water bath and excess water was wiped off with a kimwipe. 3. 5 ml of prepared Agrobacterium cell suspension was added to each designated tube. The total volume in each tube was 10 mL. 4. The tubes were inverted several times to mix the ECS cells with the Agrobacterium. 5. The tubes were placed horizontally on a rotary shaker and taped to the surface with lab tape to prevent them from rolling. 6. The tubes were gently shaken for 10 minutes on the shaker at 85 rpm. The tubes were periodically checked during incubation and each end of the tube was lifted to ensure cells remain resuspended and shaking. 7. The tubes were removed from the shaker and placed in the bench top centrifuge. 8. The cells were centrifuged for 10 minutes at 1,000 rpm. 9. The cells were removed from the centrifuge and each tube was inverted several times to resuspend the cells. 10. The tubes were placed horizontally on a rotary shaker and taped to the surface with lab tape to prevent them from rolling. 11. The tubes were gently shaken for 10 minutes on the shaker at 85 rpm. The tubes were periodically checked during incubation and each end of the tube was lifted to ensure cells remain resuspended and shaking. During this second round of shaking, TMA1 plates were prepared for co-cultivation. Sterile forceps were used to transfer sterile 5.5 cm glass fiber filters to the center of each plate. One TMA1 plate with filter was sufficient for each delivery that uses 500 μl of ECS cells or less. If greater than 500 μl of ECS cells was used per delivery, then two TMA1 plates with filters were needed for co-cultivation. 12. After the 10 minutes of shaking, the tubes were placed in a tube rack and the cells were allowed to settle to the bottom for approximately 5 minutes. 13. The majority of the supernatant was removed with a 10 mL wide mouth pipet and discarded. Enough of the supernatant was left so that the cells were easily resuspended. This volume varied depending on the amount of ECS used for transformation. In general, a volume that was 3× the amount of ECS cells used for transformation was left. 14. The cells were resuspended in the remaining liquid and transferred to the glass fiber filter on co-culture media TMA1. 15. The plate was tilted at a 450 angle to allow excess liquid to pool at the bottom of the plate. Sterile 200 μl tips were used to remove and discard the extra liquid from the plate. 16. The plates were sealed with plastic wrap and co-cultivation was carried out for 3-4 days at 25° C. in the dark. Transient expression of the reporter genes was be evaluated approximately 43 hours after inoculation. Cells were washed 3 to 4 days after transformation.

Washing Cells after Transformation (this Step was Performed 3 or 4 Days after Transformation)

1. The following materials that were needed for washing cells were gathered and brought into a laminar flow hood: 50 mL Falcon tubes, 10 mL wide mouth pipettes, pipet aid, liquid ECS media containing hormones and 400 mg/L timentin, EDM culture media plates containing 100 μM melatonin and 400 mg/L timentin, 200 μl pipet tips, and 200 μl pipettor. 2. The plates were taken out of the incubator and the plastic wrap was removed. 3. 50 mL of prepared liquid ECS media was poured into a clean, sterile 50 mL Falcon tube. 4. Approximately 10 mL of ECS media from the Falcon tube was poured onto the surface of the co-cultivation plate. 5. The liquid was pipetted up and down over the surface of the plate using the 10 mL wide mouth pipet to dislodge the cells from the glass fiber filter and the media. 6. 10 mL of media and cells was transferred from the plate to a new 50 mL Falcon tube. 7. Steps 4 to 6 were repeated with a fresh 10 mL of media from the 40 mL remaining in the Falcon tube. Approximately 30 mL of fresh ECS remained in the tube. 8. The cells were allowed to gradually settle in the tube for approximately 1 minute. 9. The supernatant was removed and discarded leaving 5 mL of supernatant covering the cells. 10. 10 mL of fresh media from the 30 mL remaining in the Falcon tube was added and pipetted up and down once to mix. 11. The cells were allowed to gradually settle in the tube for approximately 1 minute. 12. Steps 9 through 11 were repeated until the fresh media in the Falcon tube was used. The cells were washed three times at this point. 13. After the final settling, as much supernatant as possible was removed making sure to leave a supernatant volume sufficient for resuspending the cells. This was approximately 3× the volume of ECS that were in the tube. 14. The cells were resuspended in the remaining supernatant and transferred to an EDM culture plate containing 100 μM melatonin and 400 mg/L timentin. 15. The plate was tilted at a 450 angle to allow excess liquid to pool at the bottom of the plate. Sterile 200 μl tips were used to remove and discard the extra liquid from the plate. 16. The plate was sealed with plastic wrap and kept in the dark at 28° C. for two weeks.

Selection and Regeneration of Transgenic Banana Plants

1. After 2 weeks without selection, the ECS to were transferred to selective EDM medium containing 400 mg/L timentin and 50 mg/L kanamycin (nptII) or 25 mg/L hygromycin (hpt) for embryo development. The ECS were kept in the dark at 28° C. 2. The transformed ECS were subcultured every two weeks by moving the tissue to the same selective medium for two months. 3. After two months, putatively transformed embryos developed on selective embryo development medium (EDM) were transferred to selective embryo maturation medium (EMM) supplemented with 400 mg/L timentin and either kanamycin 100 mg/L or hygromycin 25 mg/L. The embryos were kept in the dark at 28° C. for 1 month. Putatively transformed embryos appeared cream/white in color. Material that is brown or black was not transferred. 4. Cream/white mature embryos were transferred to selective germination medium (GM) supplemented with 400 mg/L timentin and either kanamycin 100 mg/L or hygromycin 25 mg/L for germination. The plates were cultured in the dark at 28° C. until shoots appeared, usually one to three months. 5. Well-developed shoots (approximately 2 cm tall) were transferred to individual magenta boxes containing micropropagation medium without selection for multiplication of shoots. The plantlets were grown at 28° C. for 1 month on a 16 h/8 h light/dark cycle. 6. Transgenic shoots (at least 4 cm tall) were transferred to plastic Solo cups containing rooting medium without selection for rooting. The plants were grown for 2-4 weeks at 28° C. on a 16 h/8 h light/dark cycle. The inhibitory effect of kanamycin in the regeneration of plant transformation was previously reported for other crops. It was shown that kanamycin has a negative effect on the rooting of banana. Thus, kanamycin was omitted from banana shoot proliferation and rooting media. 7. Rooted plantlets were transferred to soil in pots and maintained in a growth chamber at 28° C. on a 16 h/8 h light/dark cycle. 8. The rate of regeneration and transformation was evaluated.

Control cultures did not show any embryo formation on selection media. Kanamycin and hygromycin proved to be equally effective as selection agents. Transformation efficiency was calculated as number of PCR positive transgenic lines regenerated on kanamycin or hygromycin selective medium per ml SCV of ECS of each cultivar. Approximately 20-70 transgenic lines per ml SCV were produced depending on the banana variety. For example, maximum number of transgenic lines (60-70) was obtained from ECS of “Sukali Ndiizi” and minimum number of lines (20-30) was obtained for “Gros Michel.” The transformation efficiency was well-correlated to regeneration efficiency of embryogenic cells of various varieties. Higher regeneration efficiency provided more independent transgenic shoots. About 25-65 plants per 50 mg of SCV of embryogenic suspension cells were previously reported for variety “Cavendish” and “Lady Finger”. Similarly, approximately 40 transgenic plants per 0.5 ml packed cell volume of ECS were reported for variety “Rasthali” (AAB). However, more than 600 independent transgenic lines from 50 mg of settled cells of Dwarf Cavendish was reported.

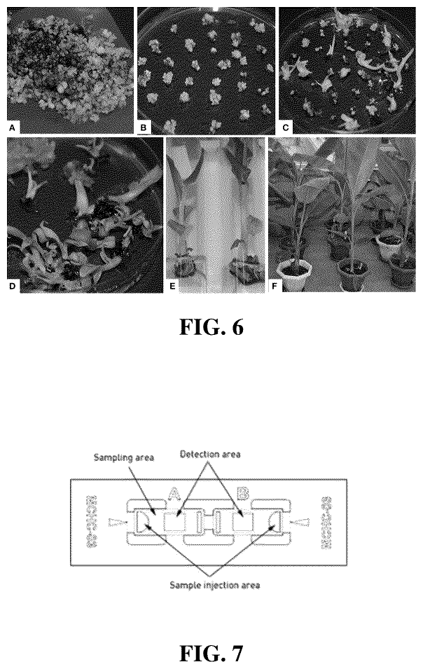

This protocol for Agrobacterium-mediated transformation of banana using embryogenic cell suspension (ECS) achieved rooted transgenic plants in 7-11 months (Table 11; FIG. 6).

TABLE 11

| Steps | Media | Time frame |
|---|---|---|
| Step 1 ECS | ECS media, TMA1 | 1 week (inoculation, co- |
| inoculation by | | cultivation, |
| Agrobacterium | | ECS preparation |
| | | before inoculation) |
| Step 2 Embryo | EDM + Selection | 1-2 months for |
| development | | white globular |
| medium (dark) | | embryos development |
| Step 3 Embryo | EMM + Selection | 1 month |
| maturation | | |
| medium (dark) | | |
| Step 4 Germination | GM + Selection | 1-2 months |
| medium | | |
| (dark) | | |
| Step 5 Micropropagation | MP No | 1-2 months |
| medium (light) | selection | |
| Step 6 Rooting | RM No | 2-4 weeks |
| medium | selection | |
| (Light) | | |

### Example 3

### Fusarium oxysporum TR4 Resistance Assays

Rapid ARSS (Rapid 13) Assays

This protocol is for the evaluation of small non-acclimated tissue culture (TC) plantlets against TR4 Fusarium oxysporum f sp. cubense (Foc).

Materials

Difco Potato dextrose broth (PDB) (cat. no. 254920, VWR); 1 cryotube of OR3-TR4 (Strain II-5); 1 mg/ml Propidium Iodide; 5 mg/ml Fluorescein Diacetate; 2-chip disposal hemocytometers (cat. no. DHC-N51, Bulldog Bio); BM6 growing medium (cat. no. 1012100, Hummert International); Osmocote (15-9-12; 5-6-month slow release) (cat. no. FE-OS15, Greenhouse Megastore); OHP Marathon 1% Granular (cat. no. CH-IN-MAR Greenhouse Megastore); 1020 trays with no drainage holes (cat. no. CN-FLHD, Greenhouse Megastore); Square black form 4-inch pots (cat. no. CN-SQV, Greenhouse Megastore); 1 L sterile Erlenmeyer vented flasks (cat. no. 89095-284, VWR); Peters 20-20-20 fertilizer (cat. no. FE-PE20-25); 38×48″ autoclave polyethylene double thick bags (cat. no. 14232-184, VWR).

Plant Acclimation

1. Removed 2 week-old, rooted TC plantlets from the root inducing media and rinsed in sterile distilled water. 2. Plantlets were placed into 4-inch pots containing BM6 growing medium (amended with the recommended rates Osmocote (15-9-12; 5-6-month slow release) and 1% OHP Marathon 1% Granular. The pots were filled to the line at the top of the 4 inch pot. Watered until saturated. The soil was not packed. 3. The pots were placed in 1020 trays with no drainage holes. 4. The pots were placed on shelf in chambers (irrigation valve is turned off). 5. Conditions in the Conviron walk in chamber were set to: Temperature 28° C./25° C. using a 12/12 cycle, Relative humidity to 80%. 6. Lighting system in the chambers were Fluence Bioengineering LED Physiospec Indoor (12/12 light/dark cycle, Light intensity 850 μmol m⁻² s⁻¹. 7. Plants were watered every other day with room temperature water, and fertilized weekly with Peters 20-20-20 fertilizer.

Inoculum Preparation

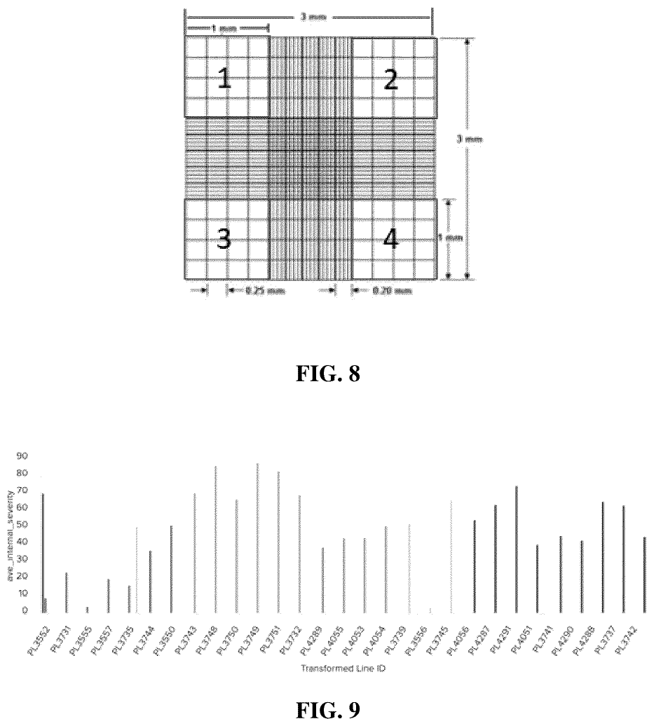

1. One week prior to inoculation and at the same time plants were placed in soil for acclimation, started to prepare the inoculum. 2. 1 cryotube of OR3-TR4 (Strain II-5) 15% glycerol stock was removed from −80° C. and placed on ice. 3. Once thawed, 100 μl of the spore suspension was placed into 500 ml vented flasks containing 100 ml ½ strength potato dextrose broth (12 g Difco PDB, 1 L water). 4. Flasks were labeled with strain ID, date, and media. 5. Flasks were placed on shaker set to 130 rpm at 28° C. 6. After 7-10 days, the spore suspension was filtered through 2 layers of sterile cheese cloth. 7. Determined percent viable vs non-viable spores using vital stains. 8. Generated a 1:3 dilution of the 7 dai inoculum. 9. 25 μl of the spore suspension and 75 μl of sterile distilled water was placed into a 1 ml centrifuge tube and added vital stain. 10. 2 μl of 1 mg/ml Propidium Iodide (PI) was added into 100 μl of solution 0.2 μl of 5 mg/ml Fluorescein Diacetate (FDA) into 100 μl 1:3 spore suspension. 11. 10 μl was injected to injection sites of the 2-chip disposal hemocytometer as shown in FIG. 7. 12 Slide was placed on stage of a Leica microscope. 13. The lamp was turned on. First the back switch was turned on and then the front switch was turned on. 14. Logged into the microscope computer. Typed in the username and password located on the side of the computer. 15. Opened LasX software. 16. Clicked on the live button. 17. Made sure the lever to switch between the eyepiece and camera was depressed. 18. Selected the 20× objective and focused on the edge of the detection area using the course and fine focus. Once in focus moved to 1 of the 4 squares of the hemocytometer. 19. Pulled out the lever to view the image on the monitor. Refocused the image using the fine focus knob. The image did not update in real time and was done in small increments. 20. Once in focus switched to the fluorescent light using the button on the lower left-hand side of the microscope. 21. Under users settings selected spore viability Foc. 22. Adjusted GFP exposure to 250 and gain to 1.3. 23. Adjusted TXR exposure to 100 and gain to 1. 24. Moved the slide to a corner that was not exposed. The spores with phot bleach very quickly, so it was important to make sure the settings and focus were correct before moving to a new location. 25. Once in the new square selected the capture button. 26. Image was saved under the projects tab. 27. Renamed image to Foc_viability_date. 28. Opened projects tab and exported photo to the spore viability folder. 29. Counted the number of spores in the squares in the 4 corners of the hemocytometer (see FIG. 8). 30. Entered the spore counts into the Batch registration table. Once entered went to the insight table>Spore counts and found the spore count associated with the batch. 31. Calculated how much of the spore suspension was needed to generate a final concentration of 1×10⁶ spores/ml using the formula C1V1=C2V2, where C1 is the original concentration, C2 is the desired concentration, V2 is the desired volume. 32. Adjusted the spore concentration to 1×10⁶ spores/ml using sterile distilled water (for example 50 ml of the spore suspension was needed to be added to 150 ml sterile distilled water to produce 200 ml of a 1×10⁶ spores/ml solution).

Plant Inoculations

1. Removed plants from soil and gently rinsed soil off roots in a beaker of room temperature water. 2. Fully submerged bare roots in the spore suspension (container varies depending on number of plants to be inoculated). 3. While plants were soaking filled 4-inch pots containing BM6 growing medium (amended with the recommended rates Osmocote (15-9-12; 5-6-month slow release) and 1% OHP Marathon 1% Granular. Pots were watered until the soil was saturated. 4. Placed pots in 1020 trays with no drainage holes. 5. After 3 hours, transferred plantlets to pots and labeled with appropriate pot tags. 6. Made sure APHIS notification sign was on the chamber door with current date, pathogen and contact information of PL. 7. Hand watered plants every other day. 8. Added enough water to saturate plants and avoid water collecting in trays.

Assessment of Symptoms

1. After 14 days evaluated plantlets for internal symptoms (rhizome discoloration). 2. Removed plant from the pot and trimmed off all roots to the base of the corm. 3. Cut off the pseudostem 2 inches from the base of the pseudostem. 4. Placed pot tag into the outside leaf of the trimmed plant. Placed plant with tag into a Ziplock bag labeled with the treatment code. All reps within a treatment were placed in one bag. 5. Once all the plants were harvested, the corms were taken to the photo station in the molecular assay room. 6. Using a large chef knife, the rhizome was vertically cut in half for the visual assessment of discoloration. 7. Lights were turned on and set at the 2 demarcations. 8. Secured camera to photo station and set at the 3rd demarcation. 9. Set camera to M setting, F 5.6, shutter speed 1/20, exposure −1.5. 10. Placed split rhizome on the matt black background and placed pot tag horizontally ½ inches from the base of the rhizome. 11. Took photo. Made sure camera is zoomed in to the edges of the black backdrop with 2 rows of 1 cm squares on either side. 12. Discarded plants into autoclave bins. 13. Transferred files to the appropriate folder. 14. Saved file as a JPGE and rename with PLID_treatment. 15. Transferred files to >TR4 disease assay>Rapid13. 16. Opened files in ASSESS software and using manual threshold option. 17. Used the free hand selection to circle the vascular steel. 18. Adjusted leaf color panel to Blue/Hue (H) to 191/31 and lesion panel to Red/Intensity (V) to 9/0. 19. Determined % Area and had spreadsheet selected. 20. Uploaded data to TR4 disease results table in Benching. 21. Calculated the average score and standard deviations to determine plant response (Benching did automatically in the insights tables).

Results

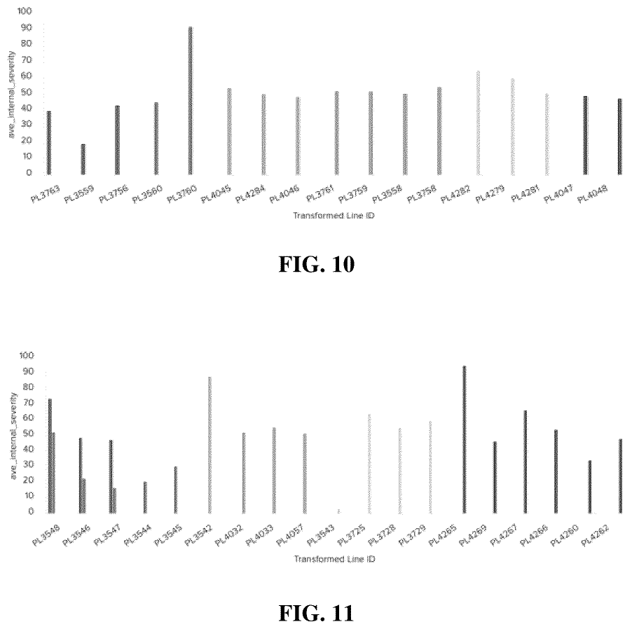

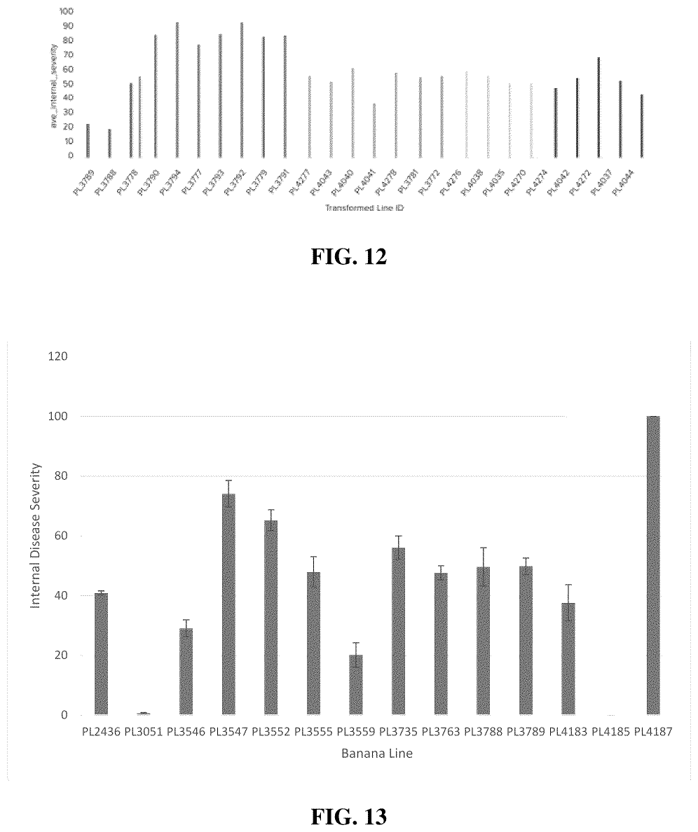

29 events of Musa BAG1 were screened, and 45% had less than 50% disease severity (FIG. 9). 17 events of ERG11 were screened, and 41% had less than 50% disease severity (FIG. 10). 22 events of Smp-AMP-D1 were screened, and 41% had less than 36% disease severity (FIG. 11). 17 events of RGA2 were screened, and 23% had less than 50% disease severity (FIG. 12).

ARSS Assays

This protocol is for the greenhouse evaluation of acclimated tissue culture (TC) plants against TR4 Fusarium oxysporum f sp. cubense (Foc).

Materials

Difco Potato dextrose broth; 2 litre vented flasks; 38×48″ autoclave polyethylene double thick bags (cat. no. 14232-184, VWR); Polypropylene containers (model 095/60=OD95) with #40 green filter plug (Microbox by Sac 02 saco2.com); BM6 growing medium (cat. no. 1012100, Hummert International); Osmocote (15-9-12; 5-6-month slow release) (cat. no. FE-OS15, Greenhouse Megastore); OHP Marathon 1% Granular (cat. no. CH-IN-MAR Greenhouse Megastore); 1020 trays with no drainage holes (cat. no. CN-FLHD, Greenhouse Megastore); Square black form 4-inch pots (cat. no. CN-SQV, Greenhouse Megastore); Elite Nursery 1-gallon pots (cat. no. CN-NCE, Greenhouse Megastore); Peters 20-20-20 fertilizer (cat. no. FE-PE20-25); Great River organic whole grain, hulled millet (Amazon).

Method

Inoculum

Fungal Strain Preparation

All Foc strains were stored as a spore suspension in 15% glycerol at −80° C. until needed. Removed cryotube of TR4 (Strain II-5) and placed on ice. Filled 2-liter vented flask with 1 liter of ½ strength PDB. Once slightly thawed, placed 500 μl of the spore suspension into ½ strength PDB. Labeled flask with strain ID and date. Incubated on orbital shaker at 28° C. and 130 rpm for 7 days.

Millet Preparation

Placed 1.5 kg of millet into a 38×48″ autoclave polyethylene double thick bag. Added 500 ml of distilled water. Sealed and autoclaved twice for 20 mins at 120° C. on consecutive days. After each round of autoclaving made sure to break up millet clumps and evenly distribute water (tried to flatten out millet in the bag). Once cooled, placed approximately 150 g of sterile millet into polypropylene containers with #40 green filter plug. There was 1 cm of head space in the containers. Added 10 ml (1 ml at a time, around the edge and on top) of 7-day spore suspension of Foc. Incubated containers on a Conviron shelf with the light off at 28° C. Inoculum was ready to use once the millet was fully colonized (˜2 weeks). Inoculum consisted of mycelia, chlamydospores, micro- and macroconidia.

Plant Material

Removed 10 cm rooted TC plantlets from the root inducing media and rinsed in sterile distilled water. Filled 4-inch pots with BM6 growing medium amended with the recommended rates of Osmocote (15-9-12; 5-6-month slow release) and OHP Marathon 1% Granular. Transplanted the rinsed plantlets into 4-inch pots. Placed plantlets on shelf in Conviron walk in chambers (made sure irrigation valve was turned on). Set chamber conditions to: Temperature 28° C./25° C. using a 12/12 cycle; Relative humidity to 80%. Lighting system in the chambers were Fluence Bioengineering LED Physiospec Indoor: 12/12 light/dark cycle; Light intensity 850 μmol m⁻² s⁻¹. Watered plants every other day with room temperature water. Fertilized weekly with Peters 20-20-20 fertilizer.

Plant Inoculations

Transferred the plants to the greenhouse once they reached 30 cm in height or had 5-6 true leaves. Filled 2-gallon pots half-way with BM6 growing medium amended with the recommended rates of Osmocote (15-9-12; 5-6-month slow release) and OHP Marathon 1% Granular. Added 10 g of infested millet to the first soil layer. Mixed into the top 1 inch of soil. Removed the plants from the soil and transferred to a 2-gallon pot. Topped with BM6 growing medium mix. Added an additional 10 g to the top layer and slightly mixed in.

Disease Assay Conditions

Placed 2 pots of the same line into 1020 trays with no drainage holes and placed on greenhouse bench. Set greenhouse conditions to: Temperature 328 C/25° C. using a 12/12 cycle; Supplemental lighting system for the greenhouse was high pressure sodium lights and metal halide, 12/12 light/dark cycle. Watered plants with room temperature water. Soil was moist but not wet. If soil was wet did not water. Fertilized weekly with Peters 20-20-20 fertilizer. Made sure APHIS notification sign was on the greenhouse door with current date, pathogen and contact information of PL.

Assessment of Symptoms

Evaluated plants for internal symptoms (rhizome discoloration) at 10-12 weeks or when Grand nain had greater than 50% external chlorosis. Removed plant from pot and cut off leaves ˜20 cm from the top of the rhizome. Cut off roots around the base of the rhizome. Harvested all lines and controls. Placed uncut rhizomes in Ziplock bags and transported to photo station. The rhizome was vertically cut in half for the visual assessment of discoloration. Photographed split rhizome using photo station and canon camera (set on manual) with a black background and included 2 rows (on either side of PVC black background square) of 1 cm squares in the field of view. Once photographed transferred JPEG files to box (TR4 Assays>ARSS trials). Opened file in Assess 2.0 software and opened the manual threshold panel. The leaf color panel was set to Blue/Hue 191/31 and the lesion panel to 90-100/0. Determined % Area and had spreadsheet selected. Based on Assess and visual ratings used ARSS rating scale (1-8) to determine ARSS score (Table 12). Uploaded data to TR4 disease results table in Benching. Calculated the average score and standard deviations to determine plant response (Benching did automatically in the insights tables). Genotypes/varieties that had an ARSS score between 1 and 3 (<5% rhizome discoloration) were categorized as resistant (R); score between 3 and 4 (>5 to <20%) were categorized as slightly susceptible (SS); score between 4 and 5 (>21 to <50%) were categorized as moderately susceptible (MS); score greater than 5 (>50%) was categorized as susceptible (S). Performed statistical analysis using one-way ANOVA and Duncan's multiple range test at the 5% level.

TABLE 12

| Discoloration Index | Description |
|---|---|
| 1 | No discoloration of the tissue in the stelar region |
| | of the rhizome and the surrounding region. |
| 2 | No discoloration of the stelar region of the rhizome. |
| | Discoloration at the junctions or root and rhizome. |
| 3 | Trace up to 5% of the stelar region discolored. |
| 4 | 6 to 20% of the stelar region discolored. |
| 5 | 21 to 50% of the stelar region discolored. |
| 6 | More than 50% of the stelar region discolored. |
| 7 | The entire rhizome stele is discolored. |
| 8 | The plant is completely dead. |

Results

Twelve lines were screened in the ARSS trial, in addition to positive and negative controls (PL2436—Grand Nain negative control; PL3051 (FHIA-25 (banana line known to be resistant to TR4 but lacking commercial qualities) positive resistance control), PL3546 (Sm-AMP-D1), PL3547 (Sm-AMP-D1), PL3552 (Musa BAG1), PL3555 (Musa BAG1), PL3559 (ERG11), PL3735 (Musa BAG1), PL3763 (ERG11), PL3788 (RGA2), PL3789 (RGA2), PL4183 (RUBY), PL4185 (RUBY), PL4187 (RUBY)), with events from all five payloads. Line PL3559 (ERG11) was the top candidate that had partial resistance and desired phenotypic characteristics. Line 4185 was the best RUBY line that had resistance equal to FHIA-25 (PL3051). PL3546 (Sm-AMP-D1) also had partial resistance but had an abnormal phenotype (stunted and corn-like leaves). PL2436 is Grand Nain—negative control (base germplasm for all transgenics). The results are shown in FIG. 13. In general lines with lower disease severity in ARSS assays had higher gene expression.

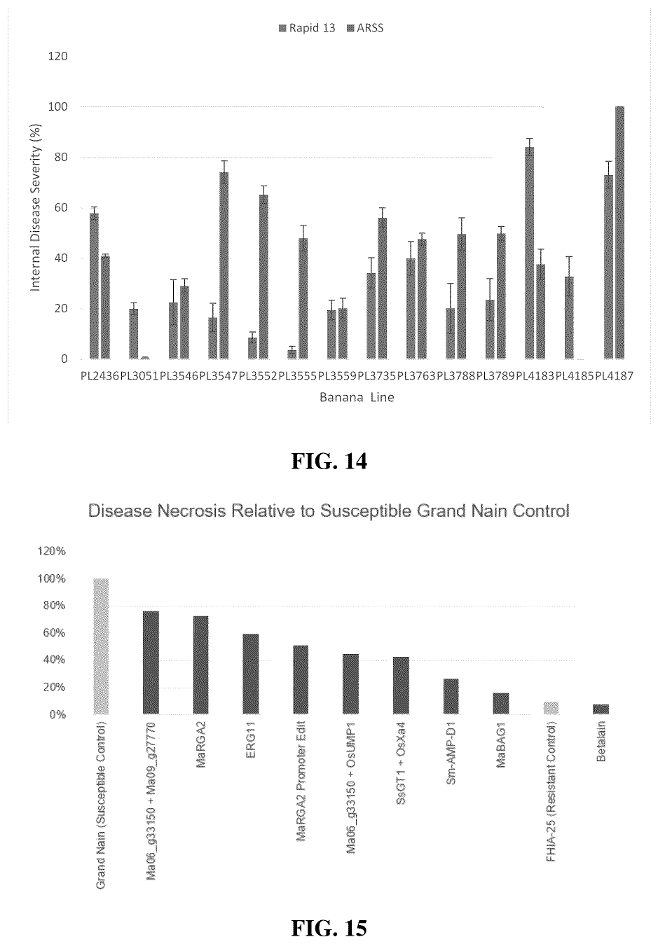

Results from the Rapid13 and ARSS assays are shown in FIG. 14. 66% of the lines that performed well in the Rapid13 assay did not perform well in the ARSS assay. 16% of the lines performed as expected base don he Rapid13 results. 16% of the lines that did not perform well in the Rapid13 assay performed better in the ARSS assay.

Additional ARSS studies were performed on additional lines: Sm-AMP-D1 (PL3544, PL3545, PL3546 and PL3547); Musa BAG1 (PL3552, PL3555, PL3557, PL4053, PL4055 and PL4289); ERG11 (PL3559, PL3560, PL3756, PL3763 and PL4046); RGA2 (PL3788 and PL3789), RUBY (PL4183, PL4184, PL4185, PL4186 and PL4189), an RGA2 promoter edit (PL4968), a TLP/snakin combination line (from vector SP0773), a combination line (from vector SP0773) of Ma06_g33150 and Ma09_g27770 (PL7921), a combination line (from vector SP1897) of SsGT1 and OsXa4 (PL7905), and a combination line (from vector SP3941) of Ma06_g33150 and OsUMP1 (PL7923), in addition to positive (PL3051 (FHIA-25 (banana line known to be resistant to TR4 but lacking commercial qualities) positive resistance control)) and negative (PL2436—Grand Nain negative control) controls. RUBY showed the best results (8% disease necrosis), which was in line with the positive control (FHIA-25, 9% disease necrosis), followed by MusaBAG1 (16% disease necrosis), Sm-AMP-D1 (27% disease necrosis), SsGT1+OsXa4 (43% disease necrosis), Ma06_g33150+OsUMP1 (46% disease necrosis), RGA2 promoter edit (51% disease necrosis), ERG11 (59% disease necrosis), RGA2 (72% disease necrosis) and TLP/snakin (76% disease necrosis). The negative control (Grand Nain) had 100% disease necrosis. The results are shown in FIG. 15.

### Example 4

### Testing Antifungal Proteins

This protocol describes the methods for testing antifungal proteins using an in vitro fungal growth inhibition assay. Four types of antifungal proteins (also called antimicrobial proteins or peptides herein) are tested: defensins, lipid transfer proteins (LTPs), snakins and thaumantin-like proteins (TLPs).

Defensins are small, cysteine-rich proteins found in plants that serve to defend them against pathogens and parasites. Their modes of action may vary; however, it is believed that many interact with the negatively charged cell membrane causing increased permeability and loss of ion gradients, or alteration of signaling cascades and production of reactive oxygen species.

LTPs, like other cationic membrane-active AMPs, are hypothesized to bind to the cell membrane of the phytopathogen through electrostatic interactions and cause destabilization and permeabilization of the membrane. A potential cause of the selective toxicity of plant LTPs is believed to be the differences in the lipid composition of the cell membranes of bacteria, fungi, plants, and mammals.

Snakins are antimicrobial peptides that play different roles in response to a variety of biotic (bacteria, fungi and nematode pathogens) and abiotic (salinity, drought and ROS) stresses. Their modes of action are not completely elucidated; however, it is believed that many interact with cell membranes to cause pore formation and cell leakage.

TLPs are known for their diverse roles in abiotic and biotic stress tolerance in plants. Overexpression of TLPs increases resistance against various fungus in both dicot and monocot plants. The mechanism of TLPs in fungal resistance is ambiguous; however, these are assumed to work by degradation and permeabilization of the fungal cell walls.

Methods

1. Antifungal protein candidates were initially identified from published reports. These served as previously described sequences possessing antifungal activity against certain pathogens, but most of these had not been tested for inhibition against the banana pathogen Foc_TR4. 2. Novel Musa antifungal protein candidates were identified by screening the banana proteome against various publicly available anti-microbial peptide databases and searching for specific motifs associated with defensins, lipid transfer proteins (LTPs), snakins, thaumatin-like proteins (TLPs), heveins, cyclotides, hairpinins, and thionins. This list was further trimmed using various criteria such as the presence of a signal peptide for secretion, relative RNA expression levels in various banana tissues and varieties, etc. 3. Genes for each candidate were codon-optimized for use in Pichia pastoris and ordered from a commercial vendor (Integrated DNA Technologies, Coralville, IA), cloned into the Pichia expression vector pD912 (ATUM, Newark, CA), and then transformed into Pichia strain BG10 (ATUM, Newark, CA) as described by the vendor. 4. Methanol-induced expression and secretion of each protein was carried out in Pichia as described in published protocols (ATUM, Newark, CA). 5. Secreted antifungal proteins were purified from the Pichia supernatants using Pierce™ Strong Cation or Anion Exchange Spin Columns according to the manufacturer's instructions (Thermo Scientific, Rockford, IL). 6. Final protein concentrations were calculated with a QuickDrop spectrophotometer (Molecular Devices, LLC, San Jose, CA) using the molecular weight and molar extinction coefficient of each secreted protein after entering their sequences into the website protparam.net/index.html. Additional details of the transformation, expression and purification of secreted proteins from Pichia pastoris are shown below. 7. The banana pathogen Foc_TR4 was grown on 0.5× Potato Dextrose Agar plates for at least 7 days at 28° C. to form a dense lawn. Sterile water was pipetted onto the plate, the surface was scraped using a plate spreader, and the loosened material was filtered through 4 layers of sterile cheesecloth to remove intact mycelial. Spores were collected by centrifuging the solution for 7 minutes at 2500 rpm, resuspending the pellet with water and spinning again, and then resuspending the final spore pellet in water. The spore concentration was determined under the microscope using a 2-Chip Hemocytometer (Bulldog Bio, Inc., Portsmouth, NH) as described by the vendor. 8. An in vitro spore germination and mycelial growth assay was carried out with Foc_TR4 spores and a dilution series of each purified antifungal protein using low ionic strength growth media (see below). 5 ng of Hygromycin B (Invitrogen) was used as a positive control, and 2 mM Potassium Phosphate Buffer, pH 5.0 was used as a negative control for growth inhibition. In some cases, Timentin antibiotic (200 ng/μl final concentration per well) was added to the growth media to prevent any bacterial contamination. 9. Germination and growth of the fungus at 25° C. in the dark was monitored by measuring absorbance at 595 nm using a SpectraMax iD3 plate reader at several time points over 48 hours. 10. Degree of fungal inhibition was determined by calculating the IC₅₀ value at 48 hours by entering the data into the website aatbio.com/tools/ic50-calculator/.

Pichia Competent Cell Production

1. Streak out the Pichia pastoris expression strain (wild-type BG10, ATUM cat. #PPS-9010) onto a YPD agar plate (1% Yeast Extract, 2% Peptone, 2% Glucose, 2% Bacto-agar) and incubate at 28-30° C. for at least 2 days. Note: alternative strains that are deficient in methanol metabolism such as aox1Δ (ATUM cat. #PPS-9011) or protease-deficient such as pep4Δprb1Δ (ATUM cat. #PPS-9019) can also be tested, although these strains tend to grow more slowly. The vendor's recommendation is to test the wild-type strain first. 2. Use a sterile loop to start a small culture (5-20 ml) of the host strain in YPD broth (1% Yeast Extract, 2% Peptone, 2% Glucose) and grow overnight at 28-30° C. with shaking at 250 rpm. 3. On day 2, transfer a small aliquot of the overnight culture (2 drops from a 5 ml pipette) into 50 ml of YPD broth in a 250 ml baffled flask with vented lid. The aliquot volume can be adjusted based on the time available for completion of the next steps. 4. Grow the culture to an OD₆₀₀ of 0.8-1.0 at 28-30° C. with shaking at 250 rpm (typical time is 4-5 hours). 5. Spin down the culture at 500×g for 15 minutes at room temperature and then carefully pour off the supernatant. 6. Gently resuspend the pellet in 9 ml of ice-cold BEDS solution (10 mM Bicine-NaOH, pH 8.3, 3% (v/v) ethylene glycol, 5% (v/v) DMSO (molecular biology grade), 1M Sorbitol. Filter-sterilize and store at 4° C.). Add 1 ml of 1M DTT and mix gently. 7. Incubate the cell suspension for 5 minutes at 100 rpm in the 28-30° C. incubator. 8. Centrifuge the cell suspension as in step 5, and then pour off the supernatant. 9. Gently resuspend the cell pellet in 1 ml of ice-cold BEDS solution without DTT. 10. The competent cells are ready for transformation, or can be stored at −80° C. by placing 200 μl aliquots into sterile microfuge tubes in a Styrofoam container and letting them slowly freeze overnight at −80° C. Competent cells can be stored at least 6 months at this temperature.

Cloning and Linearization of the Pichia Expression Vector

1. Clone the gene of interest/G-block flanked by the appropriate SapI restriction sites into the pD912 expression vector using SapI digestion and ligation. This vector will be linearized with PmeI restriction enzyme for integration into the Pichia AOX1 genomic location. Therefore, the gene of interest should not contain any internal PmeI sites. ATUM sells a variety of expression vectors that differ in the protein secretion signal leader, promoter, etc. The vendor recommendation is to start with the AOX1 methanol-inducible promoter, as it can provide better results for proteins that may cause toxicity to the cells. Cloning into this expression vector using SapI sites will add a Methionine at the N-terminus of the mature protein (i.e., after the signal peptide is cleaved off). Efficient secretion of a protein can be affected by the particular yeast signal sequence that is used. ATUM provides data for a variety of different leaders/vectors on their website as alternatives. Zeocin antibiotic is used for selection both in E. coli and Pichia. 2. Prepare an EF-Maxiprep of the sequence-confirmed vector using a Macherey-Nagel or similar kit. 3. Digest 20-40 μg of maxiprep DNA with PmeI to linearize the DNA. For example 20 μl EF-maxiprep DNA, 10 μl 10× Cutsmart Buffer, 5 μl PmeI, 65 μl dH₂O, Incubate at least 3 hours to overnight at 37° C. 4. Purify the DNA by adding 2 volumes of NTI Buffer and bind to a Macherey-Nagel NucleoSpin® PCR and Gel Purification Column, wash 2× with 650 μl Buffer A4, and elute the DNA with 20 μl of warm (65° C.) AE or TE Buffer.

Transformation of Electro-Competent Pichia Cells

1. On ice, mix 20 μl of PmeI-linearized, purified vector DNA with 60 μl of electro-competent Pichia cells prepared as detailed above (or frozen cells). Transfer the cells+DNA into a pre-chilled 2.0 mm electroporation cuvette and let sit on ice at least 2 minutes. 2. Add 0.5 ml of YPD Broth to a 14-ml round bottom culture tube and set aside in a rack. 3. Electroporate the DNA into the Pichia cells at 1500 V (time constant is typically 4.7-4.9). 4. Immediately add 0.5 ml of ice-cold 1M Sorbitol to the cuvette and transfer to the labeled culture tube containing YPD Broth. Use this YPD+Sorbitol mixture to wash out any remaining cells in the cuvette and transfer into the 14-ml culture tube. 5. Incubate the tubes at 28-30° C. for 1-2 hours with shaking at 200 rpm. 6. Spread out the transformation mixture onto YPDS+1000 ug/ml Zeocin plates (12.5 g YPD powder, 45.54 g Sorbitol, 5 g Bacto-agar, 2.5 ml 100 mg/ml Zeocin stock per 250 ml dH₂O, the mixture is typically boiled in the microwave to dissolve all the ingredients, cool to 50° C., add Zeocin, and pour 25 ml of media per plate) using glass beads (the volume can be adjusted to get the desired colony density). Using a high Zeocin concentration (such as 1000 μg/ml) favors transformation events with multiple DNA insertions, which in turn can give lines with higher protein expression. These are called “jackpot” clones. 7. Incubate the plates “face up” for 4-5 days at 28-30° C. until colonies reach a good size. 8. Optionally, use a pipette tip to pick 4-8 individual colonies and re-streak these onto a fresh YPD+1000 ug/ml Zeocin plate where the streaks are in separate ‘pie sections’ of a plate. Larger colonies are generally preferred, as these may have stronger antibiotic resistance due to multiple inserts. The general goal is to screen several colonies per vector, since the protein expression level may vary based on the number of DNA integrations or other factors. Sorbitol is not included in these plates. 9. Incubate the plates “face up” for 3-4 days at 28-30° C. Single colonies are not required, just re-streak to ensure good antibiotic resistance.

Expression of Secreted Proteins in Pichia Cultures

The next steps are performed in the sterile hood. 1. In the morning of Day 1, use a sterile loop to scrape a good amount of Pichia cells from the re-streaked ‘pie’ plate and inoculate into a 250 ml baffle-bottomed, vented lid flask containing 20 ml of BMGY media (1% Yeast Extract, 2% Peptone, 13.4 g/L Yeast Nitrogen Base (without amino acids), 100 mM Potassium Phosphate pH 6.0, 0.004 mg/L Biotin, 2% Glycerol)+250 ug/ml Zeocin. Keep the lid slightly loose and secure in place with air-pore tape. Place in the 28° C. incubator and shake at 250 rpm overnight. Typically between 4-8 clones per vector are screened to look for high expressing lines. 2. Late in the afternoon on Day 2, pour each of the 20 ml cultures into a sterile 50 ml tube and then spin the tubes at 3000 rpm for 10 minutes. The same flasks are reused for the next step so keep sterile in the hood. 3. While the tubes are spinning, add 15 ml of BMY media (1% Yeast Extract, 2% Peptone, 13.4 g/L Yeast Nitrogen Base (without amino acids), 0.004 mg/L Biotin, 100 mM Potassium Phosphate pH 6.0; methanol at the desired concentration is added directly to the flasks each day for induction) plus 62.5 μl of Zeocin stock (final concentration is 250 μg/ml) into each of the original “empty” flasks. 4. After the cells are pelleted, pour off the supernatants into a collection container for disposal. Resuspend each pellet in 20 ml of BMY media (no Zeocin). 5. Vortex the tubes until the cell pellets are completed resuspended. 6. Using a pipette, transfer 10 ml of the resuspended cells into the appropriate flask containing 15 ml BMY+Zeo250 media (so the final culture volume is 25 ml). Based on optimization experiments, transferring 1/2× volume of the resuspended cells seemed to produce the best results. This biomass seems to handle the subsequent methanol induction and recombinant protein secretion fairly well. 7. Add 500 μl of 100% methanol (final concentration=2%) to each flask, position the lid slightly loose on top and secure in place with air-pore tape, and shake in the 28° C. incubator at 250-275 rpm. Pichia requires very good aeration/oxygenation for optimal growth and productivity, so it is important to use baffled flasks, vented lids, a fast shaker speed, and a small culture volume to flask volume ratio. The percentage of methanol used for protein induction varies and may need to be optimized in certain cases. Based on the optimization experiments for Defensins, a 2% methanol concentration that is refreshed once per day seems to work best (methanol slowly gets metabolized). 8. At the end of each subsequent day, re-induce the cultures by adding 500 μl of 100% methanol to each flask, and continue this induction process for 48-96 hours. Based on the optimization experiments for Defensins, a longer induction period of 96 hours seems to work best, although 48-72 hours may be acceptable. 9. At the end of the induction process, pour the cultures into 50 ml tubes and spin down the pellets at 3000 rpm for 10 minutes. In most cases the supernatant will look clear, but for some AMPs the supernatant may look slightly cloudy (possibly due to toxicity and cell breakage). 10. Pour the supernatants into a fresh 50 ml tube, remove a 20 μl aliquot to a PCR tube for SDS-PAGE analysis, and store these tubes at −80° C. Pichia secretes very few proteins naturally, so the secreted recombinant protein should be the predominant band observed in the supernatant. Discard the pellets.

Evaluation of Secreted Proteins by SDS-PAGE

1. Remove pre-made gel(s) from the refrigerator to bring them to room temperature; microwave the 2× sample Buffer for 10 seconds or put in a heat block to dissolve the precipitated SDS. Novex 16% Tricine Gels, 1.0 mm×12 well, from ThermoFisher (cat. #EC66952BOX) can be used, as this high percentage gel provides good resolution for small proteins like Defensins. 2. Mix 20 μl of Pichia supernatant with 20 μl of Novex 2× Tricine SDS Sample Buffer (cat. #LC1676)+DTT (40 μl 1M DTT per 360 μl 2×SB) in PCR tubes. Make sure the Sample Buffer has no precipitate. 3. Denature the samples by heating at 95° C. for 10 minutes in the PCR machine, then cool to 50° C. before loading. If the samples are cooled below 50° C. or put on ice, the SDS may precipitate out. 4. Remove the white tape from the bottom of the gel plate, take out the gel comb, and rinse the wells 3× with 1× Novex Tricine SDS Running Buffer (cat. #LC1675). 5. Load 36 μl of each sample into the gel wells using a thin gel-loading pipette tip. Add 10-12 μl Novex Sharp Pre-stained Standards (cat. #LC5800) into the final well. Do not heat-denature or add sample buffer to the pre-stained protein standards. 6. Electrophorese the gel at 68V (or whatever fits into the schedule for timing) until the dye front migrates close to the bottom of the gel, or the 3.5 kDa standard is about 1/5 up from the bottom. 7. Turn off the power supply, remove the gel from the apparatus, split open the gel frame using a wedge knife, cut off the bottom of the gel that is not needed, and then stain the gel with InstantBlue® Coomasie Protein Stain (cat. #ISB1L) by slowly shaking in a square petri dish or similar tray overnight. InstantBlue® is a very good gel stain. It has great sensitivity, does not require pre-rinsing the gels with water to remove the SDS buffer, and does not stain the empty areas of the gel like other dyes.

Purification of Proteins from Pichia Supernatant

This cation exchange method works well for Defensins and other highly-charged proteins. 1. Thaw samples from −80° C. freezer and dilute each 20 ml aliquot of Pichia supernatant with 50 ml of 20 mM KPO₄, pH 5.0 Buffer. (the recombinant protein is typically purified from approximately 40 ml of supernatant). The buffer pH can be adjusted up or down based on the pI of the protein being purified. Typically stay two pH units below the protein's calculated pI for cation exchange. 2. Bring the table-top centrifuge down to 4° C. using the “Fast Temp” spin setting. 3. Filter-sterilize the diluted supernatants using a 0.2 μM or 0.45 μM filter flask to prevent clogging of the cation exchange column. Keep samples on ice. 4. Equilibrate the Pierce Strong Cation Exchange Spin Columns (Thermo Fisher cat. #9009) by adding 5 ml of 20 mM KPO₄, pH 5.0 Buffer and spin at 500×g, 5 minutes, 4° C. using a swinging bucket rotor. 5. Load ˜16 ml of diluted, filter-sterilized Pichia supernatant onto the column and spin at 500×g, 5 minutes, 4° C. using a swinging bucket rotor. Pour off flow-through and repeat this step as many times as necessary to process the entire sample. An aliquot of the flow-through can be saved to check by SDS-PAGE to be sure that the protein bound to the column under the conditions that were used. 6. Wash the column twice with 16 ml of 20 mM KPO₄, pH 5.0 Buffer, spinning as noted above. 7. Elute the proteins from the column by applying 4 ml of 20 mM KPO₄, pH 5.0 Buffer+0.5M NaCl, spin as noted above. Collect eluent to a new tube and save. 8. Repeat step 7 with 4 ml of 20 mM KPO₄, pH 5.0 Buffer+1.0M NaCl, spin as noted above. Collect eluent to a new tube and save. 9. Repeat step 7 with 4 ml of 20 mM KPO₄, pH 5.0 Buffer+2.0 M NaCl, spin as noted above. Collect eluent to a new tube and save. Using this 3-step method, it will be likely to see some protein eluted off the column with each NaCl concentration, but hopefully the protein will prefer to elute more significantly in one of the fractions. 10. Carry out buffer exchange (to reduce the high NaCl concentration) by transferring each of the 4 ml eluents to an Amicon Ultra-4 centrifugal filter 3K cutoff (cat. #UFC800396) and spin at 4000×g for 52 minutes at 4° C. (the volume should reduce from 4 ml to ˜250 μl). A 3K cutoff is used for small proteins like Defensins. A higher molecular weight cutoff, and shorter spin times, can be used for larger proteins. 11. Add 4 ml of 2 mM KPO₄, pH 5.0 Buffer to the sample tube, close the lid, mix the sample gently by inverting several times to displace any concentrated protein at the bottom, and then spin as in step 10. 12. Repeat step 11 two more times. Each buffer exchange in this scenario should reduce the salt concentration 10× for each wash (so if the initial NaCl is 2.0M, the final concentration after 3 exchanges should be roughly 2 mM). Occasionally the filter membrane may clog, and the sample volume does not reduce rapidly. In those cases, make sure to invert the sample with the fresh buffer and spin for a longer period of time until the final volume is 250-500 μl. 13. To ensure sterility for use in the in vitro assay, filter-sterilize the buffer-exchanged protein sample by passing it through a small (13 mm), 0.22 μm PVDF syringe filter (cat. #09-720-3), and rinse any remaining protein that may be trapped on the Amicon filter by adding more 2 mM KPO₄, pH 5.0 Buffer, pipetting up and down, and then pass this through the syringe filter until a final volume of ˜500 μl is obtained. 14. Determine the protein concentration of each sample using the QuickDrop spectrophotometer. Select Protein/Protein A280/Mode-Molar Extinction/pathlength-0.5 mm. Then type in the molar extinction coefficient of the protein into the AU 1/mol×1000 box (remember to divide the value by 1000 first), type in the molecular weight of the protein in kDa, blank with 2 mM KPO₄ Buffer, and then take 6-8 readings per sample and calculate the average concentration in μg/ml. Store samples at −80° C. until they can be evaluated by SDS-PAGE and used in the in vitro assay. The following website can be to calculate the molar extinction coefficient, molecular weight, and other parameters based on the sequence of each protein—protparam.net/index.html. The above website lists two molar extinction coefficients for each protein based on whether or not all of the disulfide bonds are formed. Typically the higher number is chosen, with the assumption that proteins expressed in Pichia are properly folded and all disulfide bonds have formed.

In Vitro Spore Germination and Mycelial Growth Assay

Preparation of Foc Spores

1. To a plate of Foc_TR4 mycelia that had grown for at least 8 days (older plates also worked) on 0.5×PDA media at 28° C., 10 mL of sterile water was added and the surface was scraped using a sterile plate spreader (VWR cat. #470236-284). 2. A pipette was used to collect the liquid, and the suspension was filtered through 4 layers of sterile cheesecloth into a 50 mL tube. 3. Repeated steps 1 and 2 with 6 mL of sterile water and collected flowthrough into the same 50 mL tube. The cheesecloth strained out any mycelial strands or mats; only spores passed through. 4. The spores were spun down at 2500 rpm for 7 minutes at room temperature, then the supernatant was poured off into a collection beaker with bleach for disposal. The pellet sometimes looked a little purple at this stage. 5. Washed the spore pellet 1× with 40 mL of sterile water and spun down the spores at 2500 rpm for 7 minutes. The pellet looked mostly tan at this point; if it still looked purple, then another wash step was carried out. 6. Poured off the supernatant and resuspended the final spore pellet in 10 mL of sterile water. Diluted a small aliquot of the resuspended spores 1:10 in water and then determined the spore concentration using a 2-Chip disposable hemocytometer (VWR cat. #102407-946). Counted the number of spores in one of the 4 corners of the slide (16 sub-squares). All 4 corners were sometimes counted for higher accuracy and then divided by 4. For example 210 spores×10-fold dilution factor×10⁴=2.1×10⁷ spores/mL, or 2.1×10⁴ spores/μl. 7. Diluted the spores to either 2×10² or 4×10² in 2× Low Ionic Strength (LIS) Media, since 100 μL of spore suspension was used per well, and 2-4×10⁴ spores per well were used for the assay. 4×10⁴ spores/well got slightly faster grow out. 2×LIS media (Table 4) was created to mimic the low ionic strength media used for fungal bioassays with Defensins. It was not an exact match, but pre-made micronutrient and vitamin reagents from PhytoTech were substituted to simplify the media preparation. In some studies the 2×LIS media was supplemented with Timentin antibiotic (200 ng/μl final concentration per well) to prevent bacterial contamination. LIS media was used in part because some Defensins (and possibly other proteins) are sensitive to the ionic strength of the growth media, and they can lose their ability to bind to microbial membranes in the presence of moderate to high levels of Ca²⁺, Mg²⁺, Na⁺, K⁺, or other ions. Also, with a synthetic media it was easier to control the concentrations of sugar and nitrogen that were present, and thus it was possible to slow down the growth of the fungus for this assay.

Preparation of In Vitro Assay Plates

1. Positive control: Diluted 50 mg/ml Hygromycin stock by adding 20 μL into 10 mL of sterile 2 mM KPO₄ pH 5.0 buffer, then diluted again 1:10 into KPO₄ buffer (to make 10 ng/μl stock). Used 100 μL per well (final concentration was 5 ng per well) as a ‘positive control’ that typically prevented all spore germination and mycelial growth. 2. Prepared a 2-fold dilution series of each purified protein sample based on the highest protein concentration being set to 20-30 μM as the 1× value. Dilutions were done in sterile 2 mM KPO₄ buffer, pH 5.0 buffer. The fastest way to prepare this dilution series was to use a multi-channel pipette and transfer 120 μL of each row into 120 μL of buffer for each dilution step. At the end, transferred 100 μL of each into a new plate, to which 100 μL of spore suspension was added. Thus, to have 20 μM in the 1× well, the starting protein sample was at 40 μM (since it got diluted in half by the spore solution). 3. Made sure to include the Hyg5 ‘positive control’ and Buffer only ‘negative control’ in a small set of wells to give the upper/lower baselines of growth and as an indicator that the assay plate worked properly. Ace-AMP1 was included as the ‘reference’ antifungal protein (AFP) for these assays. This protein had strong activity (approximately 0.2 μM) against Foc_TR4, and was the only clear example where there was bridging data to transgenic banana plants (approximately 0.3-0.4 μM Ace-AMP1 in leaves) that demonstrated enhanced Foc resistance. 4. Took a Time=0 reading of the plate using the SpectraMax iD3 plate reader. Protocol=JQW SPORE ASSAY v3, Mode: ABS well scan, wavelength: 595 nm, Method: Precise detection, shake 00:05 orbital, medium before reading, well scan: density 5. The full plate scan took 15 minutes. 5. Sealed the plate edges with saran film tape (or Parafilm), covered with Aluminum foil, and shook gently at speed 2 on a VWR shaker platform at 25° C. 6. Took plate readings at Time=24 hours, 48 hours, and 72 hours. 7. Processed the data and plotted the A₅₉₅ reading vs. time to generate the dose-response growth curves for each protein. These plots provided a good snapshot for the efficacy of the protein against Foc_TR4, and whether there was a nice dose-response effect. There was not significant fungal growth within the first 24 hours, but the cultures grew rapidly and had good absorbance by 48 hours. The 72 hour timepoint was helpful to delineate the dose-response for a protein with strong activity. 8. Created a table of % growth inhibition vs. buffer control for each protein concentration using the A₅₉₅ data at 48 hours. This information was used to determine the half maximal inhibitory concentration (IC₅₀) for that protein. 9. Used the following website to automatically calculate the IC₅₀ from the above data table: aatbio.com/tools/ic50-calculator/. The program re-plotted the data, created a slope equation, and then calculated an IC₅₀ value. 10. Disposed of the assay plate in an autoclave waste container. 11. Observed the plate wells under the microscope after 48 hours and took pictures to document interesting morphogenic phenotypes such as ‘hyperbranching’.

Results

The Sm-AMP-D1 had an IC₅₀ of 2.2 μM, a MW of 5.8 kDa, and a pI of 6.8-6.9.

Additional studies were performed on seven defensin candidates, and the results are shown in Table 13, below.

TABLE 13

| | IC₅₀ (μM, | MW | | # of Basic |
|---|---|---|---|---|
| Defensin | CE fraction) | (kDa) | pI | Amino Acids |
| Mba02_g12080.1 (SEQ ID NO: 211) | 6.00 ± 0.59 | 5.6 | 8.1-8.8 | 7 |
| Ma11_p12930.1 (SEQ ID NO: 205) | 0.17 ± 0.006 | 5.6 | 8.7-9.4 | 9 |
| Ma08_p13660.1 (SEQ ID NO: 184) | >25 | 5.8 | 9.5-9.6 | 11 |
| Ma06_p21420.1 (SEQ ID NO: 169) | 3.00 ± 0.17 | 5.3 | 7.9-8.5 | 8 |
| Ma04_p36140.1 (SEQ ID NO: 154) | 0.84 ± 0.06 | 5.3 | 8.5-9.4 | 10 |
| Ma02_p12840.1 (SEQ ID NO: 130) | 0.86 ± 0.07 | 5.5 | 8.5-9.1 | 8 |
| GB ID: RRT50697.1 (SEQ ID NO: 127) | 0.51 ± 0.01 | 5.3 | 8.5-9.2 | 10 |
| Ace-AMP1 (+LTP control) | 0.08 ± 0.01 | 11 | 11.8-12.3 | 19 |

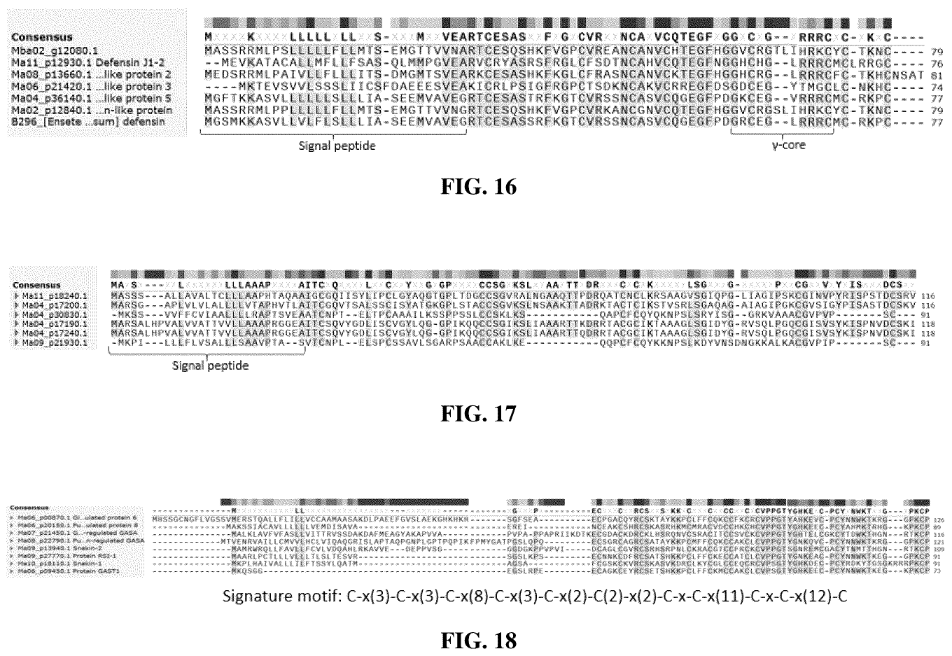

Ma11_p12930.1 and GB ID: RRT50697.1 (B296) are the best Musa Defensin leads based on the in vitro Foc-TR4 assay. Having more basic residues, like in the gamma core, has been linked to higher anti-microbial activity. Ma08_p13660.1 has a C-terminal extension that may negatively affect its activity. The alignment of these defensin candidates is shown in FIG. 16. The Mba02_gl2080.1, Ma08_p13660.1 and Ma02_p12840.1 candidates had high expression (no significant difference between FHIA-25 vs. Grand Nain), Ma11_p12930.1 and Ma04_p36140.1 candidates had low expression (no significant difference between FHIA-25 vs. Grand Nain), and the Ma06_p21420.1 candidate had no expression (no significant difference between FHIA-25 vs. Grand Nain).

Additional studies were performed on six LTP candidates, and the results are shown in Table 14, below.

TABLE 14

| | IC₅₀ (μM, | MW | | # of Basic |
|---|---|---|---|---|
| LTP | CE fraction) | (kDa) | pI | Amino Acids |
| Ma09_p21930.1 (SEQ ID NO: 193) | 1.86 ± 0.08 | 7.3 | 8.3-8.9 | 9 |
| Ma04_p17240.1 (SEQ ID NO: 148) | 1.92 ± 0.13 | 9.5 | 8.3-8.9 | 9 |
| Ma04_p17190.1 (SEQ ID NO: 145) | 3.47 ± 0.21 | 9.5 | 8.5-9.0 | 10 |
| Ma04_p30830.1 (SEQ ID NO: 151) | 6.34 ± 0.23 | 6.9 | 8.5-9.1 | 7 |
| Ma04_p17200.1 (SEQ ID NO: 142) | 2.09 ± 0.06 | 9.0 | 9.0-9.3 | 10 |
| Ma11_p18240.1 (SEQ ID NO: 208) | 2.25 ± 0.16 | 9.4 | 8.3-8.9 | 8 |
| Ace-AMP1 (+LTP control) | 0.08 ± 0.01 | 11 | 11.8-12.3 | 19 |

Most of these have IC₅₀ values around 2 μM or higher. The protein sequences are not that similar except for signature motif. The alignment of these LTP candidates is shown in FIG. 17. Ace-AMP1, an LTP from onion, has a consistently low IC₅₀ value. The Ma09_p21930.1, Ma04_p17190.1, Ma04_p17240.1 and Ma04_p30830.1 candidates had high expression (no significant difference between FHIA-25 vs. Grand Nain), and the Ma04_p17200.1 and Ma11_p18240.1 candidates had low expression (no significant difference between FHIA-25 vs. Grand Nain).

Additional studies were performed on eight snakin candidates, and the results are shown in Table 15, below.

TABLE 15

| | IC₅₀ (μM, | MW | | # of Basic |
|---|---|---|---|---|
| Snakin | CE fraction) | (kDa) | pI | Amino Acids |
| Ma07_p21450.1 (SEQ ID NO: 181) | 0.90 ± 0.05 | 10.1 | 8.2-8.9 | 15 |
| Ma10_p18110.1 (SEQ ID NO: 202) | 1.31 ± 0.03 | 7.5 | 8.9-9.3 | 16 |
| Ma06_p09450.1 (SEQ ID NO: 163) | 2.71 ± 0.53 | 7.5 | 8.1-8.7 | 15 |
| Ma09_p13940.1 (SEQ ID NO: 190) | 0.38 ± 0.01 | 9.1 | 8.3-8.9 | 13 |
| Ma09_p27770.1 (SEQ ID NO: 199) | 0.08 ± 0.003 | 7.4 | 8.0-8.6 | 10 |
| Ma06_p00870.1 (SEQ ID NO: 160) | >16 | 9.9 | 8.5-9.0 | 16 |
| Ma08_p22790.1 (SEQ ID NO: 187) | 0.52 ± 0.08 | 10.4 | 9.1-9.4 | 13 |
| Ma06_p20150.1 (SEQ ID NO: 166) | 0.53 ± 0.01 | 7.5 | 8.3-8.9 | 13 |
| Ace-AMP1 (+LTP control) | 0.08 ± 0.01 | 11 | 11.8-12.3 | 19 |

Ma09_p27770.1 and Ma09_p13940.1 are the best Musa Snakin leads based on the in vitro Foc-TR4 assay. Nothing obvious stands out for the sequence of Ma09_p27770.1 vs. others. The alignment of these LTP candidates is shown in FIG. 18. No clear correlation between activity and number of basic amino acids. These proteins are called ‘Snakins’ because there is a structure motif similarity to certain snake venoms. The Ma07_p21450.1, Ma06_p09450.1, Ma10_p18110.1 and Ma09_p27770.1 candidates had medium expression (no significant difference between FHIA-25 vs. Grand Nain), and the Ma09_p13940.1, Ma08_p22790.1, Ma06_p00870.1 and Ma06_p20150.1 candidates had low expression (no significant difference between FHIA-25 vs. Grand Nain).

Additional studies were performed on eight TLP candidates, and the results are shown in Table 16, below.

TABLE 16

| | IC₅₀ (μM, CE | MW | | # of Basic |
|---|---|---|---|---|
| TLP | fraction) | (kDa) | pI | Amino Acids |
| Ma06_p33150.1 (SEQ ID NO: 172) | 0.029 ± 0.001 | 21.3 | 7.0-7.5 | 15 |
| Ma06_p33170.1 (SEQ ID NO: 175) | 0.052 ± 0.002 | 21.3 | 7.3-7.9 | 16 |
| Ma07_p17800.1 (SEQ ID NO: 178) | >25 | 21.3 | 4.0-4.2 | 10 |
| Ma02_p17990.1 (SEQ ID NO: 136) | 0.64 | 24.5 | 7.4-7.9 | 17 |
| Ma02_p13180.1 (SEQ ID NO: 133) | >30 | 29.0 | 4.0-4.3 | 11 |
| Ma03_p07220.1 (SEQ ID NO: 139) | 0.009 ± 0.0005 | 23.6 | 8.2-8.9 | 21 |
| Ma09_p26730.1 (SEQ ID NO: 196) | >31 | 23.6 | 4.3-4.5 | 14 |
| Ma04_p38470.1 (SEQ ID NO: 157) | — | 21.3 | 4.0-4.2 | 8 |
| Ace-AMP1 (+LTP control) | 0.08 ± 0.01 | 11 | 11.8-12.3 | 19 |

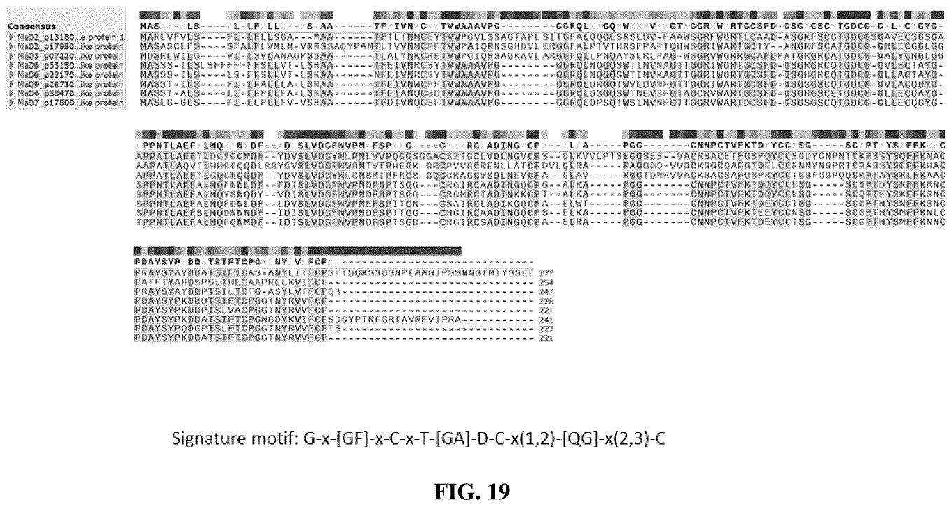

Ma06_p33150.1 and Ma06_p33170.1, and possibly Ma03_p07220.1, are the best Musa TLP leads based on the in vitro Foc-TR4 assay. TLPs with low PI's did not enrich well using anion exchange column. TLPs, in general, did not express or purify well using the Pichia system. The alignment of these TLP candidates is shown in FIG. 19. The Ma06_p33150.1 candidate had high expression (significant time-course differences for FHIA-25 vs. Grand Nain), Ma07_p17800.1 had high expression (reduced amplitude for FHIA-25 vs. Grand Nain), Ma06_p33170.1 had low expression (reduced amplitude for FHIA-25 vs. Grand Nain), Ma04_p38470.1 had low expression (higher and earlier for FHIA-25 vs. Grand Nain), and the Ma02_p17990.1, Ma02_p13180.1, Ma09_p26730.1 and Ma03_p07220.1 candidates had low expression (no significant difference between FHIA-25 vs. Grand Nain).

### Example 5

### Testing Metabolites

A number of metabolites were tested for antifungal properties using the same in vitro test described above for testing antifungal proteins. The metabolites tested were eugenol, geraniol and limonene.

Eugenol is a colorless to pale yellow, aromatic oily liquid extracted from certain essential oils, especially from clove oil, nutmeg, cinnamon, basil and bay leaf. It is known to have considerable antifungal activity. The structure for eugenol is shown below (1).

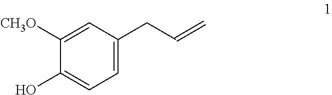

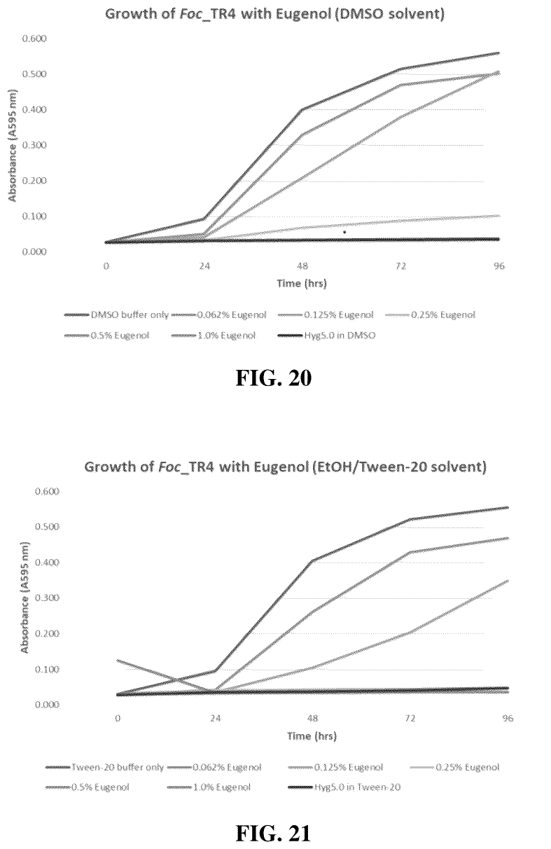

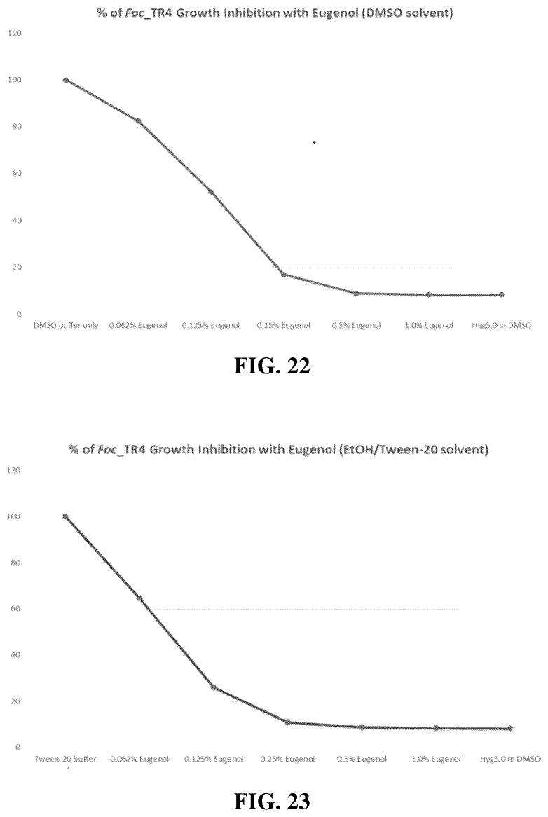

Different amounts of eugenol were tested with 1% DMSO or 1% EtOH/0.05% Tween-20 solvent against 2×10⁴ Foc_TR4 (OR3) spores per well in 0.5×PDB media. The growth of Foc_TR4 with eugenol in DMSO solvent is shown in FIG. 20, the growth of Foc_TR4 with eugenol in EtOH/Tween-20 solvent is shown in FIG. 21, the percent of Foc_TR4 growth inhibition with eugenol in DMSO solvent is shown in FIG. 22, and the percent of Foc_TR4 growth inhibition with eugenol in EtOH/Tween-20 solvent is shown in FIG. 23.

Geraniol is a monoterpenoid and an alcohol. It is the primary component of rose oil, palmarosa oil, and citronella oil. It is known to have antibacterial and antifungal activity. The structure for geraniol is shown below (2).

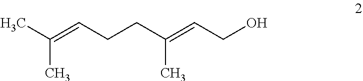

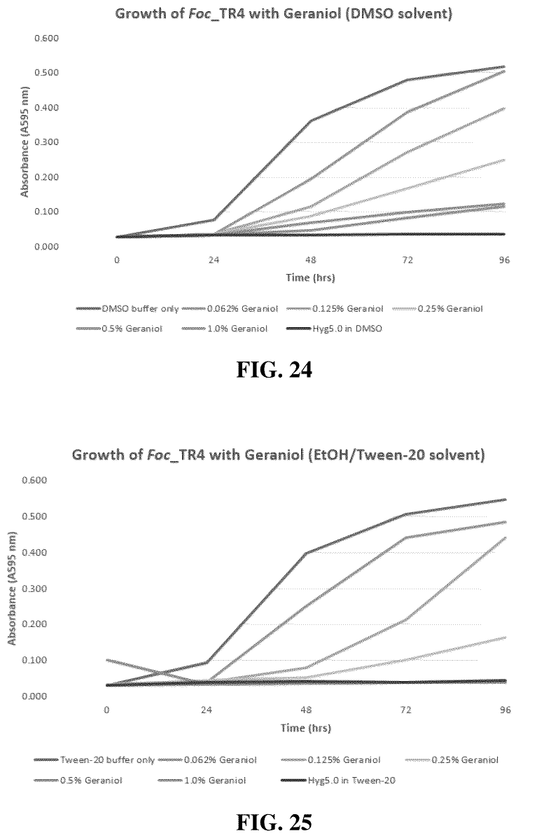

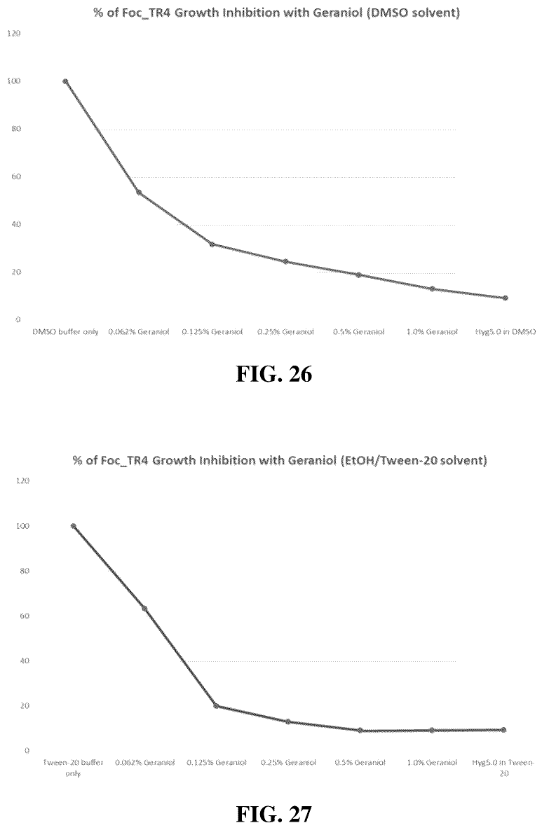

Different amounts of geraniol were tested with 1% DMSO or 1% EtOH/0.05% Tween-20 solvent against 2×10⁴ Foc_TR4 (OR3) spores per well in 0.5×PDB media. The growth of Foc_TR4 with geraniol in DMSO solvent is shown in FIG. 24, the growth of Foc_TR4 with geraniol in EtOH/Tween-20 solvent is shown in FIG. 25, the percent of Foc_TR4 growth inhibition with geraniol in DMSO solvent is shown in FIG. 26, and the percent of Foc_TR4 growth inhibition with geraniol in EtOH/Tween-20 solvent is shown in FIG. 27.

Limonene is an oil extracted from the peels of oranges and other citrus fruits. This cyclic monoterpene is known to have strong antifungal activity. The structure for geraniol is shown below (3).

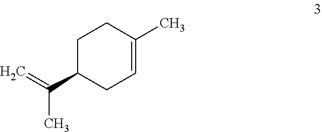

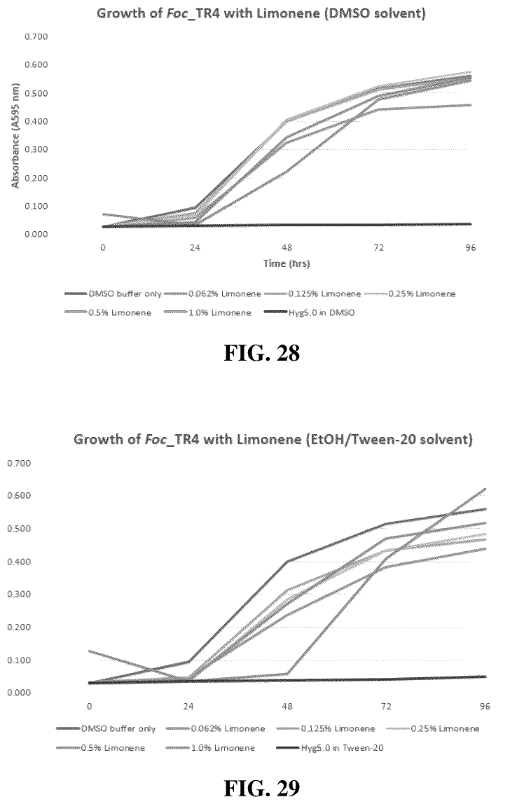

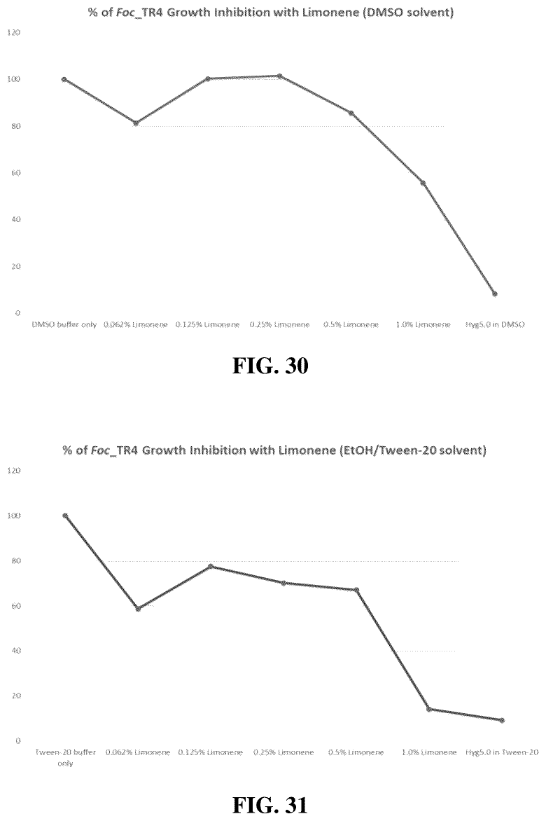

Different amounts of limonene were tested with 1% DMSO or 1% EtOH/0.05% Tween-20 solvent against 2×10⁴ Foc_TR4 (OR3) spores per well in 0.5×PDB media. The growth of Foc_TR4 with limonene in DMSO solvent is shown in FIG. 28, the growth of Foc_TR4 with limonene in EtOH/Tween-20 solvent is shown in FIG. 29, the percent of Foc_TR4 growth inhibition with limonene in DMSO solvent is shown in FIG. 30, and the percent of Foc_TR4 growth inhibition with limonene in EtOH/Tween-20 solvent is shown in FIG. 31.

### Example 6

### Transgenic Overexpression Stacks

A number of vectors were created to overexpress multiple genes. The vectors are described in greater detail below.

SP0773: This expression cassette was assembled using a CmYLCV promoter operably linked to a Musa06_g33150 nucleic acid sequence, which is operably linked to an AtHSP18.2 terminator, and a ZmUbi1 promoter operably linked to a Ma09_g27770 nucleic acid sequence, which is operably linked to a Gmax MYB2 terminator.

SP3941: This expression cassette was assembled using a CmYLCV promoter operably linked to a Musa06_g33150 nucleic acid sequence, which is operably linked to an AtHSP18.2 terminator, and a ZmUbi1 promoter operably linked to an OsUMP1 nucleic acid sequence, which is operably linked to a Gmax MYB2 terminator.

SP1897: This expression cassette was assembled using a CmYLCV promoter operably linked to a SsGT1 nucleic acid sequence, which is operably linked to a Gmax MYB2 terminator, and an ZmUbi1 promoter operably linked to an OsXa4 nucleic acid sequence, which is operably linked to an AtHSP18.2 terminator.

All of the compositions and/or methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. For example, all of the disclosed components of the preferred and alternative embodiments are interchangeable providing disclosure herein of many systems having combinations of all the preferred and alternative embodiment components. This application is therefore intended to cover any variations, uses, or adaptations of the disclosure using its general principles. Further, this application is intended to cover such departures from the present disclosure as come within known or customary practice in the art to which this disclosure pertains and which fall within the limits of the appended claims. All such similar substitutes and modifications apparent to those skilled in the art are deemed to be within the spirit, scope and concept of the disclosure as defined by the appended claims.

1. A transgenic banana plant comprising a nucleic acid construct comprising:
a) a first nucleic acid sequence encoding a Bcl-2 associated athanogene (BAG) family molecular chaperone regulator 1 protein;
b) a second nucleic acid sequence encoding a nucleic acid molecule that inhibits cytochrome P450 lanosterol 14α-demethylase;
c) a third nucleic acid sequence encoding at least a first antimicrobial peptide;
d) a fourth nucleic acid sequence encoding a resistance gene analog 2 protein;
e) a fifth nucleic acid sequence encoding at least a first betalain biosynthesis protein;
f) a sixth nucleic acid sequence encoding a eugenol, limonene or geraniol biosynthesis protein;
g) a seventh nucleic acid sequence encoding a UDP glycosyltransferase protein;
h) an eighth nucleic acid sequence encoding a proteosome maturation factor;
i) a ninth nucleic acid sequence encoding a disease resistance protein; or
j) combinations thereof,
wherein the first, second, third, fourth fifth, sixth, seventh, eighth or ninth nucleic acid sequence is operably linked to a heterologous or gene edited promoter, and wherein the banana plant exhibits increased resistance to Fusarium oxysporum f.sp. cubense Tropical Race 4 (TR4) compared to a banana plant lacking the nucleic acid construct.

2. The transgenic banana plant of claim 1, wherein:
a) the first nucleic acid sequence has at least 90% sequence identity to SEQ ID NO:1, or the complete complement thereof;
b) the second nucleic acid sequence has at least 90% sequence identity to SEQ ID NO:3, or the complete complement thereof;
c) the third nucleic acid sequence has at least 90% sequence identity to SEQ ID NO:4, SEQ ID NO:106, SEQ ID NO:125, SEQ ID NO:128, SEQ ID NO:131, SEQ ID NO:134, SEQ ID NO:137, SEQ ID NO:140, SEQ ID NO:143, SEQ ID NO:146, SEQ ID NO: 149, SEQ ID NO:152, SEQ ID NO:155, SEQ ID NO:158, SEQ ID NO:161, SEQ ID NO:164, SEQ ID NO:167, SEQ ID NO:170, SEQ ID NO:173, SEQ ID NO:176, SEQ ID NO:179, SEQ ID NO:182, SEQ ID NO:185, SEQ ID NO:188, SEQ ID NO:191, SEQ ID NO:194, SEQ ID NO:197, SEQ ID NO:200, SEQ ID NO:203, SEQ ID NO:206, SEQ ID NO:209, SEQ ID NO:215, SEQ ID NO:216, SEQ ID NO:217, SEQ ID NO:218, or the complete complement thereof;
d) the fourth nucleic acid sequence has at least 90% sequence identity to SEQ ID NO:6, or the complete complement thereof;
e) the fifth nucleic acid sequence has at least 90% sequence identity to SEQ ID NO:8, or the complete complement thereof;
f) the sixth nucleic acid sequence has at least 90% sequence identity to SEQ ID NO:96, SEQ ID NO:98 or SEQ ID NO:114, or the complete complement thereof;
g) the seventh nucleic acid sequence has at least 90% sequence identity to SEQ ID NO:110, or the complete complement thereof;
h) the eighth nucleic acid sequence has at least 90% sequence identity to SEQ ID NO:100, or the complete complement thereof; or
i) the ninth nucleic acid sequence has at least 90% sequence identity to SEQ ID NO:102, or the complete complement thereof.

3. The transgenic banana plant of claim 1, wherein the banana plant is a Musa acuminata banana plant.

4. The transgenic banana plant of claim 1, wherein the heterologous promoter is an inducible, plant, bacterial, viral, synthetic, constitutive, tissue specific, developmentally regulated, cell cycle regulated, temporally regulated, spatially regulated, gene edited, and/or spatio-temporally regulated promoter.

5. The transgenic banana plant of claim 4, wherein the heterologous promoter is a HLVH12 (SEQ ID NO:17), DCMV (SEQ ID NO:18), FSgt/PFLt (SEQ ID NO:19), dMMV (SEQ ID NO:20), CmYLCV (SEQ ID NO:21), e35S (SEQ ID NO:22), NOS (SEQ ID NO:23), ScBV (SEQ ID NO:24), CsVMV (SEQ ID NO:25), FMVSgt (SEQ ID NO:26), FS11 (SEQ ID NO:27), FE_3 (SEQ ID NO:28), ZmUbi1 (SEQ ID NO:116), OsAct1 (SEQ ID NO:117), VND7 (SEQ ID NO:118), Ma521 Ma09_g14890 (SEQ ID NO:119), Ma119 Ma08_g12140 (SEQ ID NO:120), MaM4A Ma01_g10480 (SEQ ID NO:121), MaBB Ma04_g25440 (SEQ ID NO:122), Ma40554 Ma09_g15840 (SEQ ID NO:123) or MaACT1 (SEQ ID NO:124) promoter.

6. The transgenic banana plant of claim 1, further comprising a selectable marker sequence.

7. The transgenic banana plant of claim 6, wherein the selectable marker sequence is a β glucuronidase, green fluorescent protein, or antibiotic resistance sequence.

8. The transgenic banana plant of claim 7, wherein the selectable marker sequence is a kanamycin resistance sequence.

9. The transgenic banana plant of claim 1, wherein the first, second, third, fourth, fifth, sixth, seventh, eighth or ninth nucleic acid sequence is operably linked to a terminator sequence.

10. The transgenic banana plant of claim 9, wherein the terminator sequence is a Pea3A (SEQ ID NO:29), AtUBQ3 (SEQ ID NO:30), GmaxMYB2 (SEQ ID NO:31), AtRBCS2b (SEQ ID NO:32), Pea E9 (SEQ ID NO:33), ATHSP18.2 (SEQ ID NO:34), potato Ubi3 (SEQ ID NO:35), AtTubB9 (SEQ ID NO:36) 35S (SEQ ID NO:37), CaMV 35S (SEQ ID NO:212), NOS (SEQ ID NO:213) or PBI synthetic (SEQ ID NO:214) terminator sequence.

11. The transgenic banana plant of claim 1, wherein the nucleic acid construct comprises two or more of the first, second, third, fourth, fifth, sixth, seventh, eighth or ninth nucleic acid sequences.

12. The transgenic banana plant of claim 11, wherein the two or more of the first, second, third, fourth, fifth, sixth, seventh, eighth or ninth nucleic acid sequences are operably linked to a single heterologous or gene edited promoter.

13. The transgenic banana plant of claim 11, wherein the two or more of the first, second, third, fourth, fifth, sixth, seventh, eighth or ninth nucleic acid sequences are operably linked to different heterologous or gene edited promoters.

14. The transgenic banana plant of claim 1, wherein the first, second, third, fourth, fifth, sixth, seventh, eighth or ninth nucleic acid sequence further comprises a 2A self-cleaving peptide nucleic acid sequence.

15. The transgenic banana plant of claim 1, wherein:
a) the first nucleic acid sequence has at least 90% sequence identity to SEQ ID NO:14, or the complete complement thereof;
b) the second nucleic acid sequence has at least 90% sequence identity to SEQ ID NO:13, or the complete complement thereof;
c) the third nucleic acid sequence has at least 90% sequence identity to SEQ ID NO:11 or SEQ ID NO:223, or the complete complement thereof;
d) the fourth nucleic acid sequence has at least 90% sequence identity to SEQ ID NO:12, or the complete complement thereof; or
e) the fifth nucleic acid sequence has at least 90% sequence identity to SEQ ID NO:10, or the complete complement thereof.

16. A plant part of the transgenic banana plant of claim 1.

17. The plant part of claim 16, wherein the plant part is a fruit, seed, leaf, root, flower, shoot, cell, endosperm, pseudostem, callus, cell culture, callus culture, ovule, or pollen.

18. A banana produced by the transgenic banana plant of claim 1, wherein said banana comprises a detectable amount of the first, second, third, fourth, fifth, sixth, seventh, eighth or ninth nucleic acid sequence.

19. A banana product comprising a nucleic acid construct comprising: a) a first nucleic acid sequence encoding a Bcl-2 associated athanogene (BAG) family molecular chaperone regulator 1 protein; b) a second nucleic acid sequence encoding a nucleic acid molecule that inhibits cytochrome P450 lanosterol 14α-demethylase; c) a third nucleic acid sequence encoding an antimicrobial peptide; d) a fourth nucleic acid sequence encoding a resistance gene analog 2 protein; e) a fifth nucleic acid sequence encoding at least a first betalain biosynthesis protein; f) a sixth nucleic acid sequence encoding a eugenol, limonene or geraniol biosynthesis protein; g) a seventh nucleic acid sequence encoding a UDP glycosyltransferase protein; h) an eighth nucleic acid sequence encoding a proteosome maturation factor; i) a ninth nucleic acid sequence encoding a disease resistance protein; or j) combinations thereof, wherein the first, second, third, fourth, fifth, sixth, seventh, eighth or ninth nucleic acid sequence is operably linked to a heterologous or gene edited promoter, and wherein the banana product comprises a detectable amount of the first, second, third, fourth, fifth, sixth, seventh, eighth or ninth nucleic acid sequence.

20. The banana product of claim 19, wherein the banana product is banana puree, banana powder, banana pulp, banana peel, banana jam, banana sauce, a banana drink, pastillas de saging, a banana fig, banana vinegar, dried banana chips, fried banana chips, banana flour, banana flakes, banana peel pasta, banana bread, banana cake, banana cue, banana fritter, a banana pancake, banana pudding, banana roll, banana ice cream, or banana frozen yogurt.

21. A method of producing a banana plant with increased resistance to Fusarium oxysporum f.sp. cubense Tropical Race 4 (TR4), comprising introducing into a banana plant a nucleic acid construct comprising:
a) a first nucleic acid sequence encoding a Bcl-2 associated athanogene (BAG) family molecular chaperone regulator 1 protein;
b) a second nucleic acid sequence encoding a nucleic acid molecule that inhibits cytochrome P450 lanosterol 14α-demethylase;
c) a third nucleic acid sequence encoding at least a first antimicrobial peptide;
d) a fourth nucleic acid sequence encoding a resistance gene analog 2 protein;
e) a fifth nucleic acid sequence encoding at least a first betalain biosynthesis protein;
f) a sixth nucleic acid sequence encoding a eugenol, limonene or geraniol biosynthesis protein;
g) a seventh nucleic acid sequence encoding a UDP glycosyltransferase protein;
h) an eighth nucleic acid sequence encoding a proteosome maturation factor;
i) a ninth nucleic acid sequence encoding a disease resistance protein; or
j) combinations thereof,
wherein the first, second, third, fourth, fifth, sixth, seventh, eighth or ninth nucleic acid sequence is operably linked to a heterologous or gene edited promoter.

22. The method of claim 21, wherein:
a) the first nucleic acid sequence has at least 90% sequence identity to SEQ ID NO:1, or the complete complement thereof;
b) the second nucleic acid sequence has at least 90% sequence identity to SEQ ID NO:3, or the complete complement thereof;
c) the third nucleic acid sequence has at least 90% sequence identity to SEQ ID NO:4, SEQ ID NO:106, SEQ ID NO:125, SEQ ID NO:128, SEQ ID NO:131, SEQ ID NO:134, SEQ ID NO:137, SEQ ID NO:140, SEQ ID NO:143, SEQ ID NO:146, SEQ ID NO: 149, SEQ ID NO:152, SEQ ID NO:155, SEQ ID NO:158, SEQ ID NO:161, SEQ ID NO:164, SEQ ID NO:167, SEQ ID NO:170, SEQ ID NO:173, SEQ ID NO:176, SEQ ID NO:179, SEQ ID NO:182, SEQ ID NO:185, SEQ ID NO:188, SEQ ID NO:191, SEQ ID NO:194, SEQ ID NO:197, SEQ ID NO:200, SEQ ID NO:203, SEQ ID NO:206, SEQ ID NO:209, SEQ ID NO:215, SEQ ID NO:216, SEQ ID NO:217, SEQ ID NO:218, or the complete complement thereof;
d) the fourth nucleic acid sequence has at least 90% sequence identity to SEQ ID NO:6, or the complete complement thereof;
e) the fifth nucleic acid sequence has at least 90% sequence identity to SEQ ID NO:8, or the complete complement thereof
f) the sixth nucleic acid sequence has at least 90% sequence identity to SEQ ID NO:96, SEQ ID NO:98 or SEQ ID NO:114, or the complete complement thereof;
g) the seventh nucleic acid sequence has at least 90% sequence identity to SEQ ID NO:110, or the complete complement thereof;
h) the eighth nucleic acid sequence has at least 90% sequence identity to SEQ ID NO:100, or the complete complement thereof; or
i) the ninth nucleic acid sequence has at least 90% sequence identity to SEQ ID NO:102, or the complete complement thereof.

23. The method of claim 21, wherein the banana plant is a Musa acuminata banana plant.

24. A nucleic acid construct comprising:
a) a first nucleic acid sequence having at least 90% sequence identity to SEQ ID NO:1, or the complete complement thereof;
b) a second nucleic acid sequence having at least 90% sequence identity to SEQ ID NO:3, or the complete complement thereof;
c) at least a third nucleic acid sequence having at least 90% sequence identity to SEQ ID NO:4, SEQ ID NO:106, SEQ ID NO:125, SEQ ID NO:128, SEQ ID NO:131, SEQ ID NO:134, SEQ ID NO:137, SEQ ID NO:140, SEQ ID NO:143, SEQ ID NO:146, SEQ ID NO:149, SEQ ID NO:152, SEQ ID NO:155, SEQ ID NO:158, SEQ ID NO:161, SEQ ID NO:164, SEQ ID NO:167, SEQ ID NO:170, SEQ ID NO:173, SEQ ID NO:176, SEQ ID NO:179, SEQ ID NO:182, SEQ ID NO:185, SEQ ID NO:188, SEQ ID NO:191, SEQ ID NO:194, SEQ ID NO: 197, SEQ ID NO:200, SEQ ID NO:203, SEQ ID NO:206, SEQ ID NO:209, SEQ ID NO:215, SEQ ID NO:216, SEQ ID NO:217, SEQ ID NO:218, or the complete complement thereof;
d) a fourth nucleic acid sequence having at least 90% sequence identity to SEQ ID NO:6, or the complete complement thereof;
e) at least a fifth nucleic acid sequence having at least 90% sequence identity to SEQ ID NO:8, or the complete complement thereof;
f) a sixth nucleic acid sequence having at least 90% sequence identity to SEQ ID NO:96, SEQ ID NO:98 or SEQ ID NO:114, or the complete complement thereof;
g) the seventh nucleic acid sequence has at least 90% sequence identity to SEQ ID NO:110, or the complete complement thereof;
h) the eighth nucleic acid sequence has at least 90% sequence identity to SEQ ID NO:100, or the complete complement thereof; or
i) the ninth nucleic acid sequence has at least 90% sequence identity to SEQ ID NO:102, or the complete complement thereof
j) combinations thereof,
wherein the first, second, third, fourth, fifth or sixth nucleic acid sequence is operably linked to a heterologous or gene edited promoter.

25. The nucleic acid construct of claim 24, wherein:
a) the first nucleic acid sequence has at least 90% sequence identity to SEQ ID NO:14, or the complete complement thereof;
b) the second nucleic acid sequence has at least 90% sequence identity to SEQ ID NO:13, or the complete complement thereof;
c) the third nucleic acid sequence has at least 90% sequence identity to SEQ ID NO:11 or SEQ ID NO:223, or the complete complement thereof;
d) the fourth nucleic acid sequence has at least 90% sequence identity to SEQ ID NO:12, or the complete complement thereof; or
e) the fifth nucleic acid sequence has at least 90% sequence identity to SEQ ID NO:10, or the complete complement thereof.


SEQUENCE LISTING (224 sequences)

SEQ ID NO: 1 - DNA, 459 bp, synthetic construct
  source 1..459: mol_type = other DNA
atgatcaggttgagatcaaagaagctattcaagaggagttcttccaaggccagtggcggcaatggtgggtgcagcaggaacggtgacatccagtgggagctgcggccaggaggcatgctggttcagaagagagaatgtggtgagggagctgatgaggtgatcacagtgagaatctctaccgggtcaagttggcatgagatatctattggagccactgccacatttggggagttgaaggtagttgtgggcatggtgacagggctggagccaagggagcagaggctactgttcaagggcaaggagagagaggatggtgatcatctccacatggtgggcgtgagggacaaggacaaggtgctggttttggaggatcctgcaatcaaggaaaggaagctgatgatggccatggcagagaggcaagtcatgagcagcccatgccacaccattattgaggtgtag

SEQ ID NO: 2 - protein, 152 aa, Musa acuminata
MIRLRSKKLFKRSSSKASGGNGGCSRNGDIQWELRPGGMLVQKRECGEGADEVITVRISTGSSWHEISIGATATFGELKVVVGMVTGLEPREQRLLFKGKEREDGDHLHMVGVRDKDKVLVLEDPAIKERKLMMAMAERQVMSSPCHTIIEV

SEQ ID NO: 3 - DNA, 870 bp, synthetic construct
  source 1..870: mol_type = other DNA
gcgctaaggacaacgatgacaccgagcacgatatgatgaagcatctcatgaactctacttacaagaacggcacccctgtccctgatcatgaggtcgcccacatgatgattgctctcctcatggctggccagcactcttcttcttctaccagctcttggatcatgctccgtctcgctcagtaccctcacacagagaactcggtgctgatttgcctcccctgacttacgacaaccttgccaagctgcccctcaaccaggccattatcaaggagactctccgccttcacgcccctatccactcactgacatgatcgccaacctgatgaattgcgagtacaaaaacgggcagccgattccggacaaagagatcgcgcacatgatgatcactcttctcatggctggacaacactcttcgtcatctgctagttcatggatcatactacatctggcttgtttctaaaggaacgaagagcccatacttaccctttggcgctggtcggcatcgctgcatcggggagaagtttgcttatgtcaacttgggcgttatcgttgcgactttggtgcgcaacttcagactgtcgactcttgatggcaagcctggaaggaattgtatgatgagcagattgaggttttggtatcaccgcaagagccgttgacgtgggagaatttggtgaagttgacgctgaatggacaggttatcaagaagaccttgcgtcttcatagtccaatccactcctttctccgacaggtcaagtcacctatgcgagttcccggcacgtattgggttgttccgccgtctcatacactcctcgcttcaccgggcacacaagcacgatccgaggaattctttcctcgacctatggaatgggat

SEQ ID NO: 4 - DNA, 249 bp, synthetic construct
  source 1..249: mol_type = other DNA
atggcaaaaactgtgttgggtatccatgttactttccttacactcttatttgccgtgatcctcctcaatgatgtcatgtatacacccgttgagggaaaaatatgcgaaagggcaagcggaacatggaagggaatatgtattcactcgaacgattgcaacaaccagtgcgttaaatgggagaatgctgggagtggctcttgccattatcagtttcctaactacatgtgcttctgttattttaactgttag

SEQ ID NO: 5 - protein, 82 aa, synthetic construct
MAKTVLGIHVTFLTLLFAVILLNDVMYTPVEGKICERASGTWKGICIHSNDCNNQCVKWENAGSGSCHYQFPNYMCFCYFNC

SEQ ID NO: 6 - DNA, 3699 bp, synthetic construct
  source 1..3699: mol_type = other DNA
atggctggtgtcacatcacaggcagcggcggtgttctccctggtgaatgaaatctttaaccggtccatcaatttgatcgtcgcggaactccggttgcagttgaatgcgagagccgagctgaacaatctgcagagaacactattgaggactcactctctgctcgaggaggcaaaggcgaggcggatgactgacaagtctctcgtgctgtggctgatggagctcaaggaatgggcctacgacgccgacgacatcctcgacgagtacgaggccgcagcaatccgactgaaggtaacacgctcgaccttcaaacgtcttatcgatcatgtgattataaatgttccattagcgcacaaagtagcagacatcaggaaaaggttgaacggggtcactcttgagagggagctaaatctgggtgcgctggaagggtcgcagccgcttgattccacgaaaagaggtgtgaccacttctcttctgactgaatcttgtattgtcgggcgagctcaagataaggagaatttgattcggttgctgttggagcccagcgatggggcggttcctgttgttcctatagttggattaggaggggcagggaagacgactctgtctcagcttatctttaatgacaagagagtggaggagcatttcccattgagaatgtgggtgtgtgtgtctgacgattttgatgtgaagagaattactagagagatcacagagtacgccaccaacggaaggttcatggatctcaccaacttgaatatgcttcaagttaatctgaaagaggagataagggggacgacatttttgcttgtgctggatgatgtgtggaacgaagaccccgtgaagtgggaaagcctgttagccccattagatgccggaggacggggaagcgtggtcattgtgacgacacagagcaaaaaggtcgccgatgtcaccggcacgatggagccatacgttctcgaggagttaacggaggatgacagttggtcactcatcgagagtcactccttcagggaggcgagctgctctagtacaaatcctagaatggaagagatcgggaggaagatagccaagaagatcagtggcctaccttacggagcaacagcaatggggagatatctaagatctaagcacggagaaagcagctggagagaagtcttggaaactgagacttgggagatgccaccggctgcaagtgatgtgttatccgctctaaggagaagttacgacaatctaccccctcagctgaagctctgttttgccttctgtgctctgtttacaaagggctacaggtttcgaaaggatacactgatccacatgtggatagctcaaaatttgattcaatcaacagagtcgaaaagatcggaggacatggcagaagaatgctttgatgatttggtgtgcagattcttctttcggtactcctggggcaactatgtgatgaatgactcagtccatgacctcgctcgatgggtttcattggatgaatattttcgagcagatgaagactcaccattgcatatttcaaagccaattcgtcatttgtcatggtgcagtgaaagaataaccaatgttcttgaggataataacactggtggagatgctgtcaatccgctcagcagtttgcgcactctccttttcttaggccaatctgagttccggtcgtatcatcttcttgatagaatgttcaggatgttgagccgaatccgtgttttggatttcagcaactgcgtcataagaaatttgccttcttcggttggaaatctgaaacatctgcgttacctgggcctgtctaatacgagaattcaaaggttgccggagtctgtaacacgtctttgcctccttcagacattgctactagagggctgtgaactgtgcaggttaccaagaagcatgagcaggctcgtcaaactgaggcagctcaaagcaaatccagatgtaattgccgacatagccaaagtcgggagattgatcgaacttcaagagctgaaagcctataatgttgacaagaaaaaaggacatgggattgcagagctaagtgcaatgaatcagcttcacggtgatctttccattagaaaccttcaaaatgtagagaaaacgcgagagtctcggaaggcgaggttggacgagaaacagaagcttaagctcttggatctgcgatgggctgacggtaggggtgccggagaatgtgatcgtgacaggaaagttcttaaaggcctccgaccacatccaaacctgagagaattgagtatcaaatactacggaggcacttcatctccgagttggatgacggatcagtatctgcccaacatggaaacgattcgcctgcgtagctgcgcaaggttgacggaactcccatgtctcggtcagctgcatatccttagacatttgcacatcgatgggatgtcccaagtgagacaaattaatctgcaattttatggcaccggagaagtttcaggttttccattgctggagctcctgaacatacgtcgcatgcccagtctggaggaatggtcggaaccacggagaaactgttgctacttccctcgcctccataaactgctgatcgaggattgtcccaggctcaggaatctgccctccctcccaccaacactggaagaactaaggatatcaagaacaggactagttgatcttccaggattccatggaaacggtgatgtgacgacgaatgtttccctttcttctttgcatgtttcggagtgtcgagaactgagatccctaagcgaaggattgttgcagcacaacctcgtcgccctcaagacagcggcatttaccgattgtgattctcttgagtttttgccggcggaaggattcagaacagccatttcacttgaatcattgataatgactaattgtccactgccttgcagttttcttttgccttcctctctcgagcatctaaagttgcagccatgcctctatccaaacaacaatgaggattcactgtcaacatgcttcgagaacctcacatctctttccttcttggacatcaaagattgtccaaatctgtcatcatttccaccgggtcctctatgtcagctatcagcactccaacatttgtccctcgtcaattgccagaggctacaatctattggcttccaggcactcacctccctcgaaagcttgacaattcagaactgccctcgcctcaccatgtcacacagtttggttgaggtgaataactcttccgatacagggctcgcgtttaatatcactcgatggatgcgcagacgaacaggtgacgacggcttgatgctcagacaccgagcacaaaatgattcatttttcgggggacttctgcaacacctcaccttcctccagtttctaaagatctgccagtgtccacaactcgtaaccttcaccggcgaagaggaagagaagtggagaaaccttacttctcttcaaattctgcacatcgttgattgtccaaacctggaggtactgcctgcaaacttgcaaagcctctgctccctcagcaccttgtacatcgtcagatgcccaagaatccatgcgtttcctcccggaggtgtcagcatgtccctggcacatttggtcatccatgaatgccctcagctgtgtcagcgatgtgatccaccgggaggtgatgattggcccttaatagctaatgtaccaagaatatgtcttggaaggactcatccatgtcgctgtagcaccacctga

SEQ ID NO: 7 - protein, 1232 aa, Musa acuminata
MAGVTSQAAAVFSLVNEIFNRSINLIVAELRLQLNARAELNNLQRTLLRTHSLLEEAKARRMTDKSLVLWLMELKEWAYDADDILDEYEAAAIRLKVTRSTFKRLIDHVIINVPLAHKVADIRKRLNGVTLERELNLGALEGSQPLDSTKRGVTTSLLTESCIVGRAQDKENLIRLLLEPSDGAVPVVPIVGLGGAGKTTLSQLIFNDKRVEEHFPLRMWVCVSDDFDVKRITREITEYATNGRFMDLTNLNMLQVNLKEEIRGTTFLLVLDDVWNEDPVKWESLLAPLDAGGRGSVVIVTTQSKKVADVTGTMEPYVLEELTEDDSWSLIESHSFREASCSSTNPRMEEIGRKIAKKISGLPYGATAMGRYLRSKHGESSWREVLETETWEMPPAASDVLSALRRSYDNLPPQLKLCFAFCALFTKGYRFRKDTLIHMWIAQNLIQSTESKRSEDMAEECFDDLVCRFFFRYSWGNYVMNDSVHDLARWVSLDEYFRADEDSPLHISKPIRHLSWCSERITNVLEDNNTGGDAVNPLSSLRTLLFLGQSEFRSYHLLDRMFRMLSRIRVLDFSNCVIRNLPSSVGNLKHLRYLGLSNTRIQRLPESVTRLCLLQTLLLEGCELCRLPRSMSRLVKLRQLKANPDVIADIAKVGRLIELQELKAYNVDKKKGHGIAELSAMNQLHGDLSIRNLQNVEKTRESRKARLDEKQKLKLLDLRWADGRGAGECDRDRKVLKGLRPHPNLRELSIKYYGGTSSPSWMTDQYLPNMETIRLRSCARLTELPCLGQLHILRHLHIDGMSQVRQINLQFYGTGEVSGFPLLELLNIRRMPSLEEWSEPRRNCCYFPRLHKLLIEDCPRLRNLPSLPPTLEELRISRTGLVDLPGFHGNGDVTTNVSLSSLHVSECRELRSLSEGLLQHNLVALKTAAFTDCDSLEFLPAEGFRTAISLESLIMTNCPLPCSFLLPSSLEHLKLQPCLYPNNNEDSLSTCFENLTSLSFLDIKDCPNLSSFPPGPLCQLSALQHLSLVNCQRLQSIGFQALTSLESLTIQNCPRLTMSHSLVEVNNSSDTGLAFNITRWMRRRTGDDGLMLRHRAQNDSFFGGLLQHLTFLQFLKICQCPQLVTFTGEEEEKWRNLTSLQILHIVDCPNLEVLPANLQSLCSLSTLYIVRCPRIHAFPPGGVSMSLAHLVIHECPQLCQRCDPPGGDDWPLIANVPRICLGRTHPCRCSTT

SEQ ID NO: 8 - DNA, 3951 bp, synthetic construct
  source 1..3951: mol_type = other DNA
atggatcatgcgaccctcgccatgatcctcgcgatctggttcatcagcttccacttcatcaagctgctgttctcccagcagaccaccaagctgcttccgccaggaccaaagccgcttccgatcatcggcaacatccttgaggtgggcaagaagccgcatcggtccttcgccaacctcgccaagattcacggcccactcatttccctcagactcggctctgtgaccaccatcgttgtgtcctctgccgacgtggccaaagagatgttcctcaagaaggatcacccgctctccaaccgcacgatcccgaatagtgttacagccggcgaccaccacaagctcaccatgtcttggctcccggtgtctccgaagtggcgcaacttccgcaagattaccgccgtgcatctgctctccccacagagactcgatgcctgccagacattcaggcacgccaaggtgcagcagctctacgagtacgttcaagagtgcgcccagaaaggccaggccgtggatattggcaaggccgcctttacgaccagcctcaacctcctcagcaagctgttcttcagcgtcgagctggcgcaccacaagtcccataccagccaagagttcaaagagctgatctggaacatcatggaagatataggcaagccgaactacgccgactacttcccgattctcggctgcgttgacccatctggcattagaagaaggctcgcctgctccttcgacaagctgatcgccgtgttccagggcatcatctgcgagagactcgccccagattcctccaccacaactaccaccaccaccgacgacgtgctcgatgtgctcctccagctgttcaagcagaacgagctgacgatgggcgagatcaaccacctcctcgtggacatcttcgacgccggcaccgataccacatcctccacattcgagtgggtgatgaccgagctgatccgcaatccagagatgatggaaaaggcccaagaggaaatcaagcaggtcctcggcaaggacaagcagatccaagagtccgacatcatcaacctgccgtacctccaggcgatcatcaaagagacactccgcctccatccgccgaccgtgttcttgctcccaagaaaggccgacaccgatgtcgagctgtacggctacatcgtgccgaaggatgcccagatcctcgtgaacctctgggccattggcagggacccaaacgcctggcagaacgccgatattttcagcccagagcgcttcatcggctgcgagatcgatgttaagggccgcgatttcggcctccttccatttggcgctggccgcagaatttgcccaggcatgaatctcgccatcaggatgctcaccctcatgctcgccacactcctccagttcttcaactggaagctcgaaggcgacatctccccgaaggacctcgacatggacgagaagttcggcattgcgctccaaaagaccaagccgctcaagctcatcccgattccgcgctacggaagcggagctactaacttcagcctgctgaagcaggctggagacgtggaggagaaccctggacctatgaagatgatgaacggcgaggacgccaacgaccagatgatcaaagagtccttcttcatcacccacggcaacccgatcctcaccgtcgaggatacacatccgctcaggccgttcttcgagacatggcgcgagaagattttctccaagaagccgaaggccatcctcatcatctccggccactgggagacagtgaagccaaccgtgaacgccgtgcacatcaacgacaccatccacgacttcgacgactacccagccgccatgtaccagttcaagtacccagctccaggcgagccagagcttgcgagaaaggtggaagagatcctcaagaagtccgggttcgagacagccgagacagaccaaaagaggggccttgatcacggcgcctgggttccactcatgctcatgtatccagaggcggacatcccggtgtgccagctctcagttcagccacatctcgacggcacctaccactacaatctcggcagagccctcgcgccgctcaagaatgatggcgtgctcattattggctccggcagcgccacacatccactcgatgagacaccgcactacttcgatggtgttgccccttgggccgctgccttcgattcttggcttaggaaggccctcatcaacggccgcttcgaggaagtgaacatctacgagagcaaggccccgaactggaagctcgcccatccatttccagagcacttctacccgctccacgttgtgctcggcgctgctggtgaaaagtggaaggccgagctgatccactcctcctgggatcatggcacactttgccacggctcctacaagttcacctccgccggttcaggtgccactaatttttctctcttgaaacaggccggtgacgttgaagagaacccaggtccaatgaccgccatcaagatgaacaccaacggcgagggcgagacacagcacatcctcatgatcccgttcatggcgcagggccacctcaggccatttctcgaactcgccatgttcctctacaagcgctcccacgtgatcatcaccctgctcacaactccgctcaacgccggcttcctcaggcacctccttcaccaccattcctactcctccagcggcatcaggatcgtcgagctgccattcaactccaccaaccacggactcccaccgggcatcgagaacaccgataagctcacactcccgctcgtggtgtccctcttccattccaccatcagcctcgatccgcacctccgcgattacatctccaggcatttcagcccagccaggccaccactctgcgtgatccatgatgtgttcctcggctgggttgaccaggtggccaaggatgtgggctctacaggcgtggtgttcacaacaggcggcgcttatggcacatccgcctacgtgtccatctggaacgatctcccgcaccagaactactccgacgaccaagagttcccgctgccaggcttcccagagaaccataagttccgcaggtcccagctccatcggttcctcagatatgccgacggctccgacgattggtccaagtatttccagccgcagctccgccagtccatgaagtcttttggctggctctgcaactccgtggaagagatcgagacactcggcttctccatcctccgcaactacaccaagctgccgatctggggcatcggcccacttattgcttccccagtgcagcactcctcctccgacaacaattcaacaggcgccgagttcgtgcagtggctcagcctcaaagagccggactccgtcctctacatctccttcggctcccagaacacgatcagcccgacgcagatgatggaactcgctgctggccttgagtcctccgagaagccattcctctgggtgatcagagccccgttcggcttcgacatcaacgaagagatgcgcccagagtggctgccagagggctttgaggaacgcatgaaggtgaagaaacagggcaagctcgtgtacaagctcggcccgcagcttgagatcctcaaccatgaatccatcggcggctttctcacccactgcggatggaacagcatccttgagtctcttcgcgagggcgttccgatgcttggatggccacttgctgccgagcaggcctacaacctcaagtacctcgaagatgagatgggcgtcgcggttgagcttgctagaggcctcgaaggcgagatctccaaagagaaggtcaagcgcatcgtcgagatgatccttgagcgcaacgagggctccaaaggctgggagatgaagaatcgcgccgtggaaatgggcaaaaagctcaaggacgccgtgaacgaggaaaaagagctgaagggctcctccgtgaaggcgatcgacgatttcctcgacgccgtcatgcaggccaaacttgagccaagcctccagtga

SEQ ID NO: 9 - protein, 518 aa, synthetic construct
MDHATLAMILAIWFISFHFIKLLFSQQTTKLLPPGPKPLPIIGNILEVGKKPHRSFANLAKIHGPLISLRLGSVTTIVVSSADVAKEMFLKKDHPLSNRTIPNSVTAGDHHKLTMSWLPVSPKWRNFRKITAVHLLSPQRLDACQTFRHAKVQQLYEYVQECAQKGQAVDIGKAAFTTSLNLLSKLFFSVELAHHKSHTSQEFKELIWNIMEDIGKPNYADYFPILGCVDPSGIRRRLACSFDKLIAVFQGIICERLAPDSSTTTTTTTDDVLDVLLQLFKQNELTMGEINHLLVDIFDAGTDTTSSTFEWVMTELIRNPEMMEKAQEEIKQVLGKDKQIQESDIINLPYLQAIIKETLRLHPPTVFLLPRKADTDVELYGYIVPKDAQILVNLWAIGRDPNAWQNADIFSPERFIGCEIDVKGRDFGLLPFGAGRRICPGMNLAIRMLTLMLATLLQFFNWKLEGDISPKDLDMDEKFGIALQKTKPLKLIPIPRYGSGATNFSLLKQAGDVEENPG

SEQ ID NO: 10 - DNA, 13918 bp, synthetic construct
  source 1..13918: mol_type = other DNA
ttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgaggtcccgcagccgaacgaccgagcgcagcggcgagagtagggaactgccaggcatcctgggcggttctgataacgagtaatcgttaatccgcaaataacgtaaaaacccgcttcggcgggtttttttatggggggagtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatgcatttagaataccttttgtgtcgcccttattcgactccctatagaagttcctattctctagaaagtataggaacttcccttcattttccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagacgttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaattcccaaataataaaaaagccggattaataatctggctttttatattctctctgcagcaccctgcaggggtttggccacgcagcacaccatccacctcatacggtctctagcgtgacagcgaaatgttctggcactgcacttgcactggggacagcctattttgctagtttgttttgtttcgttttgttttgatggagagcgtatgttagtactatcgattcacacaaaaaaccaacacacagatgtaatgaaaataaagatattttattcccgggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgaagctcacttgtacagctcgtccatgccgccggtggagtggcggccctcggcgcgttcgtactgttccacgatggtgtagtcctcgttgtgggaggtgatgtccaacttgatgttgacgttgtaggcgccgggcagctgcacgggcttcttggccttgtaggtggtcttgacctcagcgtcgtagtggccgccgtccttcagcttcagcctctgcttgatctcgcccttcagggcgccgtcctcggggtacatccgctcggaggaggcctcccagcccatggttttcttctgcattacggggccgtcggaggggaagttggtgccgcgcagcttcaccttgtagatgaactcgccgtcctgcagggaggagtcctgggtcacggtcaccacgccgccgtcctcgaagttcatcacgcgctcccacttgaagccctcggggaaggacagcttcaagtagtcggggatgtcggcggggtgcttcacgtaggccttggagccgtacatgaactgaggggacaggatgtcccaggcgaagggcagggggccacccttggtcaccttcagcttggcggtctgggtgccctcgtaggggcggccctcgccctcgccctcgatctcgaactcgtggccgttcacggagccctccatgtgcaccttgaagcgcatgaactccttgatgatggccatgttatcctcctcgcccttgctcaccattgtttagtatactagaattccagcgtgtcaaacttacaaatttctctgaagttgtatcctcagtacttcaaagaaaatagcttacaccaaattttttcttgttttcacaaatgccgaacttggttccttatataggaaaactcaagggcaaaaatgacacggaaaaatataaaaggataagtagtgggggataagattcctttgtgataaggttactttccgcccttacattttccaccttacatgtgtcctctatgtctctttcacaatcaccgaccttatcttcttcttttcattgttgtcgtcagtgcttacgtcttcaagattcttttcttcgcctggttcttctttttcaatttctatgtattcttcttcgtattctggcagtataggatcttgtatctgtacattcttcatttttgaacataggttgaatatgtgccgcatattgatctgcttcttgctgagttcacataatacttccatagtttttcccgtaaacattggattcttgatgctacatcttggataattaccttctgctcctctgagcgtgcaggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaaaagctcactggaggcttggctcaagtttggcctgcatgacggcgtcgaggaaatcgtcgatcgccttcacggaggagcccttcagctctttttcctcgttcacggcgtccttgagctttttgcccatttccacggcgcgattcttcatctcccagcctttggagccctcgttgcgctcaaggatcatctcgacgatgcgcttgaccttctctttggagatctcgccttcgaggcctctagcaagctcaaccgcgacgcccatctcatcttcgaggtacttgaggttgtaggcctgctcggcagcaagtggccatccaagcatcggaacgccctcgcgaagagactcaaggatgctgttccatccgcagtgggtgagaaagccgccgatggattcatggttgaggatctcaagctgcgggccgagcttgtacacgagcttgccctgtttcttcaccttcatgcgttcctcaaagccctctggcagccactctgggcgcatctcttcgttgatgtcgaagccgaacggggctctgatcacccagaggaatggcttctcggaggactcaaggccagcagcgagttccatcatctgcgtcgggctgatcgtgttctgggagccgaaggagatgtagaggacggagtccggctctttgaggctgagccactgcacgaactcggcgcctgttgaattgttgtcggaggaggagtgctgcactggggaagcaataagtgggccgatgccccagatcggcagcttggtgtagttgcggaggatggagaagccgagtgtctcgatctcttccacggagttgcagagccagccaaaagacttcatggactggcggagctgcggctggaaatacttggaccaatcgtcggagccgtcggcatatctgaggaaccgatggagctgggacctgcggaacttatggttctctgggaagcctggcagcgggaactcttggtcgtcggagtagttctggtgcgggagatcgttccagatggacacgtaggcggatgtgccataagcgccgcctgttgtgaacaccacgcctgtagagcccacatccttggccacctggtcaacccagccgaggaacacatcatggatcacgcagagtggtggcctggctgggctgaaatgcctggagatgtaatcgcggaggtgcggatcgaggctgatggtggaatggaagagggacaccacgagcgggagtgtgagcttatcggtgttctcgatgcccggtgggagtccgtggttggtggagttgaatggcagctcgacgatcctgatgccgctggaggagtaggaatggtggtgaaggaggtgcctgaggaagccggcgttgagcggagttgtgagcagggtgatgatcacgtgggagcgcttgtagaggaacatggcgagttcgagaaatggcctgaggtggccctgcgccatgaacgggatcatgaggatgtgctgtgtctcgccctcgccgttggtgttcatcttgatggcggtcattggacctgggttctcttcaacgtcaccggcctgtttcaagagagaaaaattagtggcacctgaaccggcggaggtgaacttgtaggagccgtggcaaagtgtgccatgatcccaggaggagtggatcagctcggccttccacttttcaccagcagcgccgagcacaacgtggagcgggtagaagtgctctggaaatggatgggcgagcttccagttcggggccttgctctcgtagatgttcacttcctcgaagcggccgttgatgagggccttcctaagccaagaatcgaaggcagcggcccaaggggcaacaccatcgaagtagtgcggtgtctcatcgagtggatgtgtggcgctgccggagccaataatgagcacgccatcattcttgagcggcgcgagggctctgccgagattgtagtggtaggtgccgtcgagatgtggctgaactgagagctggcacaccgggatgtccgcctctggatacatgagcatgagtggaacccaggcgccgtgatcaaggcccctcttttggtctgtctcggctgtctcgaacccggacttcttgaggatctcttccacctttctcgcaagctctggctcgcctggagctgggtacttgaactggtacatggcggctgggtagtcgtcgaagtcgtggatggtgtcgttgatgtgcacggcgttcacggttggcttcactgtctcccagtggccggagatgatgaggatggccttcggcttcttggagaaaatcttctcgcgccatgtctcgaagaacggcctgagcggatgtgtatcctcgacggtgaggatcgggttgccgtgggtgatgaagaaggactctttgatcatctggtcgttggcgtcctcgccgttcatcatcttcataggtccagggttctcctccacgtctccagcctgcttcagcaggctgaagttagtagctccgcttccgtagcgcggaatcgggatgagcttgagcggcttggtcttttggagcgcaatgccgaacttctcgtccatgtcgaggtccttcggggagatgtcgccttcgagcttccagttgaagaactggaggagtgtggcgagcatgagggtgagcatcctgatggcgagattcatgcctgggcaaattctgcggccagcgccaaatggaaggaggccgaaatcgcggcccttaacatcgatctcgcagccgatgaagcgctctgggctgaaaatatcggcgttctgccaggcgtttgggtccctgccaatggcccagaggttcacgaggatctgggcatccttcggcacgatgtagccgtacagctcgacatcggtgtcggcctttcttgggagcaagaacacggtcggcggatggaggcggagtgtctctttgatgatcgcctggaggtacggcaggttgatgatgtcggactcttggatctgcttgtccttgccgaggacctgcttgatttcctcttgggccttttccatcatctctggattgcggatcagctcggtcatcacccactcgaatgtggaggatgtggtatcggtgccggcgtcgaagatgtccacgaggaggtggttgatctcgcccatcgtcagctcgttctgcttgaacagctggaggagcacatcgagcacgtcgtcggtggtggtggtagttgtggtggaggaatctggggcgagtctctcgcagatgatgccctggaacacggcgatcagcttgtcgaaggagcaggcgagccttcttctaatgccagatgggtcaacgcagccgagaatcgggaagtagtcggcgtagttcggcttgcctatatcttccatgatgttccagatcagctctttgaactcttggctggtatgggacttgtggtgcgccagctcgacgctgaagaacagcttgctgaggaggttgaggctggtcgtaaaggcggccttgccaatatccacggcctggcctttctgggcgcactcttgaacgtactcgtagagctgctgcaccttggcgtgcctgaatgtctggcaggcatcgagtctctgtggggagagcagatgcacggcggtaatcttgcggaagttgcgccacttcggagacaccgggagccaagacatggtgagcttgtggtggtcgccggctgtaacactattcgggatcgtgcggttggagagcgggtgatccttcttgaggaacatctctttggccacgtcggcagaggacacaacgatggtggtcacagagccgagtctgagggaaatgagtgggccgtgaatcttggcgaggttggcgaaggaccgatgcggcttcttgcccacctcaaggatgttgccgatgatcggaagcggctttggtcctggcggaagcagcttggtggtctgctgggagaacagcagcttgatgaagtggaagctgatgaaccagatcgcgaggatcatggcgagggtcgcatgatccattgttggtcgatcgacagatctgcgaaagctcgagagagatagatttgtagagagagactggtgatttcagcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcactcctgagaccgaattccaaacgcgaagtaatcttttcggttttaaagaaaaagggcagggtggtgacaccttgcccgtttttttgccggaccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtccccccttcagaagttcctatactttctagagaataggaacttccctatagagtcgaataagggcgacaccccctaattagcccgggcgaaaggcccagtctttcgactgagcctttcgttttatttgatgcctggcagttccctactctcgcatggggagtccccacactaccatcggcgctacggcgtttcacttctgagttcggcatggggtcaggtgggaccaccgcgctactgccgccaggcaaacaaggggtgttatgagccatattcaggtataaatgggctcgcgataatgttcagaattggttaattggttgtaacactgacccctatttgtttatttttctaaatacattcaaatatgtatccgctcatgagacaataaccctgataaatgcttcaataatattgaaaaaggaagaatatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaaaagcttaataagatgatcttc

SEQ ID NO: 11 - DNA, 13493 bp, synthetic construct
  source 1..13493: mol_type = other DNA
ttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgaggtcccgcagccgaacgaccgagcgcagcggcgagagtagggaactgccaggcatcctgggcggttctgataacgagtaatcgttaatccgcaaataacgtaaaaacccgcttcggcgggtttttttatggggggagtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatgcatttagaataccttttgtgtcgcccttattcgactccctatagaagttcctattctctagaaagtataggaacttcccttcattttccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagacgttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaattcccaaataataaaaaagccggattaataatctggctttttatattctctctgcagcaccctgcaggggtttggccacgcagcacaccatccacctcatacggtctctagcgcaagagcggatctagtaacatagatgacaccgggcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcgtcgcaagaccggcaacaggattcaatcttaagaaactttattgccaaatgtttgaacgatcaagctgtcagcaatcaaagtaacagaaacacatataattcgggaactggtaatggcacgagcctgagcccgcattttcccatttcacgcattggttattgcagtcgttggagtgtatacagatccccttccaagtgccagatgcccgttcacatattttttccaccggcgtatacatcacatcattcaggagcaggacggcgaacaacaatgtcagaaaggtaacgtgtatacccaagactgtcttcgccattctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggggggaggacgagcggcgtaccttgaagcggaggtgccgacgggtggatttgggggagatctggttgtgtgtgtgtgcgctccgaacaacacgaggttggggaaagagggtgtggagggggtgtctatttattacggcgggcgaggaagggaaagcgaaggagcggtgggaaaggaatcccccgtagctgccgtgccgtgagaggaggaggaggccgcctgccgtgccggctcacgtctgccgctccgccacgcaatttctggatgccgacagcggagcaagtccaacggtggagcggaactctcgagaggggtccagaggcagcgacagagatgccgtgccgtctgcttcgcttggcccgacgcgacgctgctggttcgctggttggtgtccgttagactcgtcgacggcgtttaacaggctggcattatctactcgaaacaagaaaaatgtttccttagtttttttaatttcttaaagggtatttgtttaatttttagtcactttattttattctattttatatctaaattattaaataaaaaaactaaaatagagttttagttttcttaatttagaggctaaaatagaataaaatagatgtactaaaaaaattagtctataaaaaccattaaccctaaaccctaaatggatgtactaataaaatggatgaagtattatataggtgaagctatttgcaaaaaaaaaggagaacacatgcacactaaaaagataaaactgtagagtcctgttgtcaaaatactcaattgtcctttagaccatgtctaactgttcatttatatgattctctaaaacactgatattattgtagtactatagattatattattcgtagagtaaagtttaaatatatgtataaagatagataaactgcacttcaaacaagtgtgacaaaaaaaatatgtggtaattttttataacttagacatgcaatgctcattatctctagagaggggcacgaccgggtcacgctgcactgcagctcctgacagcgaaatgttctggcactgcacttgcactggggacagcctattttgctagtttgttttgtttcgttttgttttgatggagagcgtatgttagtactatcgattcacacaaaaaaccaacacacagatgtaatgaaaataaagatattttattcccgggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgaagctcagagttcatcatgttcagagttatcgagatccggtggatcacgggcccgcggaaccgtcgactggagaatacgcagcttgagttcgaggtctgagtacttatacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcactgccattagatgagagatttcgattccgattttgatttcgattccgattttgatttcgattgatctcttccttctgatttgtgttccttatataaggaaattcttgtgggatcctagacgtcatggcttacgtcatttccttcgtcctgttgctcactgattgagctgtgagtggagggaccactggaagatgcttcactaattttcttagtggagggaccggcttcacatgcttcacacaagtggctgtcgggcatcatcttttttagcttttgacaaagcaatgttttagtggtggctcccactcttatcttcaacattattatcttatcttcaaaggacgataagatgttgatgtctgtggacgaagatcctagacgtcatggcttacgtcatttccttcgtcctgttgctcactgattgagctgtgagtggagggaccactggaagatgcttcactaattttcttagtggagggaccggcttcacatgcttcacacaagtggctgtcgggcatcatcttttttagcttttgacaaagcaatgttttagtggtggctcccactcttatcttcaacattattatcttatcttcaaaggacgataagatgttgatgtctgtggacgactcctctgagcgtgcaggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaaaagcctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcagatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgaacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttggacattgtttagtatactagaattccagcgtgtcaaacttacaaatttctctgaagttgtatcctcagtacttcaaagaaaatagcttacaccaaattttttcttgttttcacaaatgccgaacttggttccttatataggaaaactcaagggcaaaaatgacacggaaaaatataaaaggataagtagtgggggataagattcctttgtgataaggttactttccgcccttacattttccaccttacatgtgtcctctatgtctctttcacaatcaccgaccttatcttcttcttttcattgttgtcgtcagtgcttacgtcttcaagattcttttcttcgcctggttcttctttttcaatttctatgtattcttcttcgtattctggcagtataggatcttgtatctgtacattcttcatttttgaacataggttgaatatgtgccgcatattgatctgcttcttgctgagttcacataatacttccatagtttttcccgtaaacattggattcttgatgctacatcttggataattaccttctgctcctgagaccgaattccaaacgcgaagtaatcttttcggttttaaagaaaaagggcagggtggtgacaccttgcccgtttttttgccggaccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtccccccttcagaagttcctatactttctagagaataggaacttccctatagagtcgaataagggcgacaccccctaattagcccgggcgaaaggcccagtctttcgactgagcctttcgttttatttgatgcctggcagttccctactctcgcatggggagtccccacactaccatcggcgctacggcgtttcacttctgagttcggcatggggtcaggtgggaccaccgcgctactgccgccaggcaaacaaggggtgttatgagccatattcaggtataaatgggctcgcgataatgttcagaattggttaattggttgtaacactgacccctatttgtttatttttctaaatacattcaaatatgtatccgctcatgagacaataaccctgataaatgcttcaataatattgaaaaaggaagaatatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaaaagcttaataagatgatcttc

SEQ ID NO: 12 - DNA, 16944 bp, synthetic construct
  source 1..16944: mol_type = other DNA
ttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgaggtcccgcagccgaacgaccgagcgcagcggcgagagtagggaactgccaggcatcctgggcggttctgataacgagtaatcgttaatccgcaaataacgtaaaaacccgcttcggcgggtttttttatggggggagtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatgcatttagaataccttttgtgtcgcccttattcgactccctatagaagttcctattctctagaaagtataggaacttcccttcattttccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagacgttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaattcccaaataataaaaaagccggattaataatctggctttttatattctctctgcagcaccctgcaggggtttggccacgcagcacaccatccacctcatacggtctctagcgcaagagcggatctagtaacatagatgacaccgggcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcgtcgcaagaccggcaacaggattcaatcttaagaaactttattgccaaatgtttgaacgatcaagctcaggtggtgctacagcgacatggatgagtccttccaagacatattcttggtacattagctattaagggccaatcatcacctcccggtggatcacatcgctgacacagctgagggcattcatggatgaccaaatgtgccagggacatgctgacacctccgggaggaaacgcatggattcttgggcatctgacgatgtacaaggtgctgagggagcagaggctttgcaagtttgcaggcagtacctccaggtttggacaatcaacgatgtgcagaatttgaagagaagtaaggtttctccacttctcttcctcttcgccggtgaaggttacgagttgtggacactggcagatctttagaaactggaggaaggtgaggtgttgcagaagtcccccgaaaaatgaatcattttgtgctcggtgtctgagcatcaagccgtcgtcacctgttcgtctgcgcatccatcgagtgatattaaacgcgagccctgtatcggaagagttattcacctcaaccaaactgtgtgacatggtgaggcgagggcagttctgaattgtcaagctttcgagggaggtgagtgcctggaagccaatagattgtagcctctggcaattgacgagggacaaatgttggagtgctgatagctgacatagaggacccggtggaaatgatgacagatttggacaatctttgatgtccaagaaggaaagagatgtgaggttctcgaagcatgttgacagtgaatcctcattgttgtttggatagaggcatggctgcaactttagatgctcgagagaggaaggcaaaagaaaactgcaaggcagtggacaattagtcattatcaatgattcaagtgaaatggctgttctgaatccttccgccggcaaaaactcaagagaatcacaatcggtaaatgccgctgtcttgagggcgacgaggttgtgctgcaacaatccttcgcttagggatctcagttctcgacactccgaaacatgcaaagaagaaagggaaacattcgtcgtcacatcaccgtttccatggaatcctggaagatcaactagtcctgttcttgatatccttagttcttccagtgttggtgggagggagggcagattcctgagcctgggacaatcctcgatcagcagtttatggaggcgagggaagtagcaacagtttctccgtggttccgaccattcctccagactgggcatgcgacgtatgttcaggagctccagcaatggaaaacctgaaacttctccggtgccataaaattgcagattaatttgtctcacttgggacatcccatcgatgtgcaaatgtctaaggatatgcagctgaccgagacatgggagttccgtcaaccttgcgcagctacgcaggcgaatcgtttccatgttgggcagatactgatccgtcatccaactcggagatgaagtgcctccgtagtatttgatactcaattctctcaggtttggatgtggtcggaggcctttaagaactttcctgtcacgatcacattctccggcacccctaccgtcagcccatcgcagatccaagagcttaagcttctgtttctcgtccaacctcgccttccgagactctcgcgttttctctacattttgaaggtttctaatggaaagatcaccgtgaagctgattcattgcacttagctctgcaatcccatgtccttttttcttgtcaacattataggctttcagctcttgaagttcgatcaatctcccgactttggctatgtcggcaattacatctggatttgctttgagctgcctcagtttgacgagcctgctcatgcttcttggtaacctgcacagttcacagccctctagtagcaatgtctgaaggaggcaaagacgtgttacagactccggcaacctttgaattctcgtattagacaggcccaggtaacgcagatgtttcagatttccaaccgaagaaggcaaatttcttatgacgcagttgctgaaatccaaaacacggattcggctcaacatcctgaacattctatcaagaagatgatacgaccggaactcagattggcctaagaaaaggagagtgcgcaaactgctgagcggattgacagcatctccaccagtgttattatcctcaagaacattggttattctttcactgcaccatgacaaatgacgaattggctttgaaatatgcaatggtgagtcttcatctgctcgaaaatattcatccaatgaaacccatcgagcgaggtcatggactgagtcattcatcacatagttgccccaggagtaccgaaagaagaatctgcacaccaaatcatcaaagcattcttctgccatgtcctccgatcttttcgactctgttgattgaatcaaattttgagctatccacatgtggatcagtgtatcctttcgaaacctgtagccctttgtaaacagagcacagaaggcaaaacagagcttcagctgagggggtagattgtcgtaacttctccttagagcggataacacatcacttgcagccggtggcatctcccaagtctcagtttccaagacttctctccagctgctttctccgtgcttagatcttagatatctccccattgctgttgctccgtaaggtaggccactgatcttcttggctatcttcctcccgatctcttccattctaggatttgtactagagcagctcgcctccctgaaggagtgactctcgatgagtgaccaactgtcatcctccgttaactcctcgagaacgtatggctccatcgtgccggtgacatcggcgacctttttgctctgtgtcgtcacaatgaccacgcttccccgtcctccggcatctaatggggctaacaggctttcccacttcacggggtcttcgttccacacatcatccagcacaagcaaaaatgtcgtcccccttatctcctctttcagattaacttgaagcatattcaagttggtgagatccatgaaccttccgttggtggcgtactctgtgatctctctagtaattctcttcacatcaaaatcgtcagacacacacacccacattctcaatgggaaatgctcctccactctcttgtcattaaagataagctgagacagagtcgtcttccctgcccctcctaatccaactataggaacaacaggaaccgccccatcgctgggctccaacagcaaccgaatcaaattctccttatcttgagctcgcccgacaatacaagattcagtcagaagagaagtggtcacacctcttttcgtggaatcaagcggctgcgacccttccagcgcacccagatttagctccctctcaagagtgaccccgttcaaccttttcctgatgtctgctactttgtgcgctaatggaacatttataatcacatgatcgataagacgtttgaaggtcgagcgtgttaccttcagtcggattgctgcggcctcgtactcgtcgaggatgtcgtcggcgtcgtaggcccattccttgagctccatcagccacagcacgagagacttgtcagtcatccgcctcgcctttgcctcctcgagcagagagtgagtcctcaatagtgttctctgcagattgttcagctcggctctcgcattcaactgcaaccggagttccgcgacgatcaaattgatggaccggttaaagatttcattcaccagggagaacaccgccgctgcctgtgatgtgacaccagccattctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggggggaggacgagcggcgtaccttgaagcggaggtgccgacgggtggatttgggggagatctggttgtgtgtgtgtgcgctccgaacaacacgaggttggggaaagagggtgtggagggggtgtctatttattacggcgggcgaggaagggaaagcgaaggagcggtgggaaaggaatcccccgtagctgccgtgccgtgagaggaggaggaggccgcctgccgtgccggctcacgtctgccgctccgccacgcaatttctggatgccgacagcggagcaagtccaacggtggagcggaactctcgagaggggtccagaggcagcgacagagatgccgtgccgtctgcttcgcttggcccgacgcgacgctgctggttcgctggttggtgtccgttagactcgtcgacggcgtttaacaggctggcattatctactcgaaacaagaaaaatgtttccttagtttttttaatttcttaaagggtatttgtttaatttttagtcactttattttattctattttatatctaaattattaaataaaaaaactaaaatagagttttagttttcttaatttagaggctaaaatagaataaaatagatgtactaaaaaaattagtctataaaaaccattaaccctaaaccctaaatggatgtactaataaaatggatgaagtattatataggtgaagctatttgcaaaaaaaaaggagaacacatgcacactaaaaagataaaactgtagagtcctgttgtcaaaatactcaattgtcctttagaccatgtctaactgttcatttatatgattctctaaaacactgatattattgtagtactatagattatattattcgtagagtaaagtttaaatatatgtataaagatagataaactgcacttcaaacaagtgtgacaaaaaaaatatgtggtaattttttataacttagacatgcaatgctcattatctctagagaggggcacgaccgggtcacgctgcactgcagctcctgacagcgaaatgttctggcactgcacttgcactggggacagcctattttgctagtttgttttgtttcgttttgttttgatggagagcgtatgttagtactatcgattcacacaaaaaaccaacacacagatgtaatgaaaataaagatattttattcccgggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgaagctcagagttcatcatgttcagagttatcgagatccggtggatcacgggcccgcggaaccgtcgactggagaatacgcagcttgagttcgaggtctgagtacttatacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcactgccattagatgagagatttcgattccgattttgatttcgattccgattttgatttcgattgatctcttccttctgatttgtgttccttatataaggaaattcttgtgggatcctagacgtcatggcttacgtcatttccttcgtcctgttgctcactgattgagctgtgagtggagggaccactggaagatgcttcactaattttcttagtggagggaccggcttcacatgcttcacacaagtggctgtcgggcatcatcttttttagcttttgacaaagcaatgttttagtggtggctcccactcttatcttcaacattattatcttatcttcaaaggacgataagatgttgatgtctgtggacgaagatcctagacgtcatggcttacgtcatttccttcgtcctgttgctcactgattgagctgtgagtggagggaccactggaagatgcttcactaattttcttagtggagggaccggcttcacatgcttcacacaagtggctgtcgggcatcatcttttttagcttttgacaaagcaatgttttagtggtggctcccactcttatcttcaacattattatcttatcttcaaaggacgataagatgttgatgtctgtggacgactcctctgagcgtgcaggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaaaagcctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcagatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgaacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttggacattgtttagtatactagaattccagcgtgtcaaacttacaaatttctctgaagttgtatcctcagtacttcaaagaaaatagcttacaccaaattttttcttgttttcacaaatgccgaacttggttccttatataggaaaactcaagggcaaaaatgacacggaaaaatataaaaggataagtagtgggggataagattcctttgtgataaggttactttccgcccttacattttccaccttacatgtgtcctctatgtctctttcacaatcaccgaccttatcttcttcttttcattgttgtcgtcagtgcttacgtcttcaagattcttttcttcgcctggttcttctttttcaatttctatgtattcttcttcgtattctggcagtataggatcttgtatctgtacattcttcatttttgaacataggttgaatatgtgccgcatattgatctgcttcttgctgagttcacataatacttccatagtttttcccgtaaacattggattcttgatgctacatcttggataattaccttctgctcctgagaccgaattccaaacgcgaagtaatcttttcggttttaaagaaaaagggcagggtggtgacaccttgcccgtttttttgccggaccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtccccccttcagaagttcctatactttctagagaataggaacttccctatagagtcgaataagggcgacaccccctaattagcccgggcgaaaggcccagtctttcgactgagcctttcgttttatttgatgcctggcagttccctactctcgcatggggagtccccacactaccatcggcgctacggcgtttcacttctgagttcggcatggggtcaggtgggaccaccgcgctactgccgccaggcaaacaaggggtgttatgagccatattcaggtataaatgggctcgcgataatgttcagaattggttaattggttgtaacactgacccctatttgtttatttttctaaatacattcaaatatgtatccgctcatgagacaataaccctgataaatgcttcaataatattgaaaaaggaagaatatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaaaagcttaataagatgatcttc

SEQ ID NO: 13 - DNA, 15785 bp, synthetic construct
  source 1..15785: mol_type = other DNA
ttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgaggtcccgcagccgaacgaccgagcgcagcggcgagagtagggaactgccaggcatcctgggcggttctgataacgagtaatcgttaatccgcaaataacgtaaaaacccgcttcggcgggtttttttatggggggagtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatgcatttagaataccttttgtgtcgcccttattcgactccctatagaagttcctattctctagaaagtataggaacttcccttcattttccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagacgttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaattcccaaataataaaaaagccggattaataatctggctttttatattctctctgcagcaccctgcaggggtttggccacgcagcacaccatccacctcatacggtctctagcgcaagagcggatctagtaacatagatgacaccgggcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcgtcgcaagaccggcaacaggattcaatcttaagaaactttattgccaaatgtttgaacgatcaagcgcgctaaggacaacgatgacaccgagcacgatatgatgaagcatctcatgaactctacttacaagaacggcacccctgtccctgatcatgaggtcgcccacatgatgattgctctcctcatggctggccagcactcttcttcttctaccagctcttggatcatgctccgtctcgctcagtaccctcacacagagaactcggtgctgatttgcctcccctgacttacgacaaccttgccaagctgcccctcaaccaggccattatcaagtagactctccgccttcacgcccctatccactcactgacatgatcgccaacctgatgaattgcgagtacaaaaacgggcagccgattccagacaaagagatcgcgcacatgatgatcactcttctcatggctggacaacactcttcgtcatctgctagttcatggatcatactacatctggcttgtttctaaaggaacgaagagcccatacttaccctttggcgctggtcggcatcgctgcatcggggagaagtttgcttatgtcaacttgggcgttatcgtcgcgactttggtgcgcaacttcagactgtcgactcttgatggcaagcctggaagaattgtatgatgagcagattgaggttttggtatcaccgcaagagccgttgacgtgggagaatttgcagaagttgacgctgaatggacaggttatcaaggagacttgcgtcttcatagtccaatccactccattctccgacaggtcaagtcacctatgcgagttcccggcacgtattgggttgttccgccgtctcatacactcctcgcttcaccgggcacacaagcacgatccgaggaattctttcctcgacctatggaatgggatctgcttcgaacccaatttcccaactgtaatcaatccaaatgtaagatcaatgataacacaatgacatgatctatcatgttaccttgtttattcatgttcgactaattcatttaattaatagtcaatccatttagaagttaataaaactacaagtattatttagaaattaataagaatgttgattgaaaataatactatataaaatgatagatcttgcgctttgttatattagcattagattatgttttgttacattagattactgtttctattagtttgatattatttgttactttagcttgttatttaatattttgtttattgataaattacaagcagattggaatttctaacaaaatatttattaacttttaaactaaaatatttagtaatggtatagatatttaattatataataaactattaatcataaaaaaatattattttaatttatttattcttatttttactatagtattttatcattgatatttaattcatcaaaccagctagaattactattatgattaaaacaaatattaatgctagtatatcatcttacatgttcgatcaaattcattaaaaataatatacttactctcaacttttatcttcttcgtcttacacatcacttgtcatatttttttacattactatgttgtttatgtaaacaatatatttataaattattttttcacaattataacaactatattattataatcatactaattaacatcacttaactattttatactaaaaggaaaaaagaaaataattatttccttaccaattggcgaaatcccattccataggtcgaggaaagaattcctcggatcgtgcttgtgtgcccggtgaagcgaggagtgtatgagacggcggaacaacccaatacgtgccgggaactcgcataggtgacttgacctgtcggagaatggagtggattggactatgaagacgcaagtctccttgataacctgtccattcagcgtcaacttctgcaaattctcccacgtcaacggctcttgcggtgataccaaaacctcaatctgctcatcatacaattcttccaggcttgccatcaagagtcgacagtctgaagttgcgcaccaaagtcgcgacgataacgcccaagttgacataagcaaacttctccccgatgcagcgatgccgaccagcgccaaagggtaagtatgggctcttcgttcctttagaaacaagccagatgtagtatgatccatgaactagcagatgacgaagagtgttgtccagccatgagaagagtgatcatcatgtgcgcgatctctttgtctggaatcggctgcccgtttttgtactcgcaattcatcaggttggcgatcatgtcagtgagtggataggggcgtgaaggcggagagtctacttgataatggcctggttgaggggcagcttggcaaggttgtcgtaagtcaggggaggcaaatcagcaccgagttctctgtgtgagggtactgagcgagacggagcatgatccaagagctggtagaagaagaagagtgctggccagccatgaggagagcaatcatcatgtgggcgacctcatgatcagggacaggggtgccgttcttgtaagtagagttcatgagatgcttcatcatatcgtgctcggtgtcatcgttgtccttagcgccattctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggggggaggacgagcggcgtaccttgaagcggaggtgccgacgggtggatttgggggagatctggttgtgtgtgtgtgcgctccgaacaacacgaggttggggaaagagggtgtggagggggtgtctatttattacggcgggcgaggaagggaaagcgaaggagcggtgggaaaggaatcccccgtagctgccgtgccgtgagaggaggaggaggccgcctgccgtgccggctcacgtctgccgctccgccacgcaatttctggatgccgacagcggagcaagtccaacggtggagcggaactctcgagaggggtccagaggcagcgacagagatgccgtgccgtctgcttcgcttggcccgacgcgacgctgctggttcgctggttggtgtccgttagactcgtcgacggcgtttaacaggctggcattatctactcgaaacaagaaaaatgtttccttagtttttttaatttcttaaagggtatttgtttaatttttagtcactttattttattctattttatatctaaattattaaataaaaaaactaaaatagagttttagttttcttaatttagaggctaaaatagaataaaatagatgtactaaaaaaattagtctataaaaaccattaaccctaaaccctaaatggatgtactaataaaatggatgaagtattatataggtgaagctatttgcaaaaaaaaaggagaacacatgcacactaaaaagataaaactgtagagtcctgttgtcaaaatactcaattgtcctttagaccatgtctaactgttcatttatatgattctctaaaacactgatattattgtagtactatagattatattattcgtagagtaaagtttaaatatatgtataaagatagataaactgcacttcaaacaagtgtgacaaaaaaaatatgtggtaattttttataacttagacatgcaatgctcattatctctagagaggggcacgaccgggtcacgctgcactgcagctcctgacagcgaaatgttctggcactgcacttgcactggggacagcctattttgctagtttgttttgtttcgttttgttttgatggagagcgtatgttagtactatcgattcacacaaaaaaccaacacacagatgtaatgaaaataaagatattttattcccgggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgaagctcagagttcatcatgttcagagttatcgagatccggtggatcacgggcccgcggaaccgtcgactggagaatacgcagcttgagttcgaggtctgagtacttatacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcactgccattagatgagagatttcgattccgattttgatttcgattccgattttgatttcgattgatctcttccttctgatttgtgttccttatataaggaaattcttgtgggatcctagacgtcatggcttacgtcatttccttcgtcctgttgctcactgattgagctgtgagtggagggaccactggaagatgcttcactaattttcttagtggagggaccggcttcacatgcttcacacaagtggctgtcgggcatcatcttttttagcttttgacaaagcaatgttttagtggtggctcccactcttatcttcaacattattatcttatcttcaaaggacgataagatgttgatgtctgtggacgaagatcctagacgtcatggcttacgtcatttccttcgtcctgttgctcactgattgagctgtgagtggagggaccactggaagatgcttcactaattttcttagtggagggaccggcttcacatgcttcacacaagtggctgtcgggcatcatcttttttagcttttgacaaagcaatgttttagtggtggctcccactcttatcttcaacattattatcttatcttcaaaggacgataagatgttgatgtctgtggacgactcctctgagcgtgcaggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaaaagcctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcagatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgaacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttggacattgtttagtatactagaattccagcgtgtcaaacttacaaatttctctgaagttgtatcctcagtacttcaaagaaaatagcttacaccaaattttttcttgttttcacaaatgccgaacttggttccttatataggaaaactcaagggcaaaaatgacacggaaaaatataaaaggataagtagtgggggataagattcctttgtgataaggttactttccgcccttacattttccaccttacatgtgtcctctatgtctctttcacaatcaccgaccttatcttcttcttttcattgttgtcgtcagtgcttacgtcttcaagattcttttcttcgcctggttcttctttttcaatttctatgtattcttcttcgtattctggcagtataggatcttgtatctgtacattcttcatttttgaacataggttgaatatgtgccgcatattgatctgcttcttgctgagttcacataatacttccatagtttttcccgtaaacattggattcttgatgctacatcttggataattaccttctgctcctgagaccgaattccaaacgcgaagtaatcttttcggttttaaagaaaaagggcagggtggtgacaccttgcccgtttttttgccggaccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtccccccttcagaagttcctatactttctagagaataggaacttccctatagagtcgaataagggcgacaccccctaattagcccgggcgaaaggcccagtctttcgactgagcctttcgttttatttgatgcctggcagttccctactctcgcatggggagtccccacactaccatcggcgctacggcgtttcacttctgagttcggcatggggtcaggtgggaccaccgcgctactgccgccaggcaaacaaggggtgttatgagccatattcaggtataaatgggctcgcgataatgttcagaattggttaattggttgtaacactgacccctatttgtttatttttctaaatacattcaaatatgtatccgctcatgagacaataaccctgataaatgcttcaataatattgaaaaaggaagaatatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaaaagcttaataagatgatcttc

SEQ ID NO: 14 - DNA, 13707 bp, synthetic construct
  source 1..13707: mol_type = other DNA
ttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgaggtcccgcagccgaacgaccgagcgcagcggcgagagtagggaactgccaggcatcctgggcggttctgataacgagtaatcgttaatccgcaaataacgtaaaaacccgcttcggcgggtttttttatggggggagtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatgcatttagaataccttttgtgtcgcccttattcgactccctatagaagttcctattctctagaaagtataggaacttcccttcattttccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagacgttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaattcccaaataataaaaaagccggattaataatctggctttttatattctctctgcagcaccctgcaggggtttggccacgcagcacaccatccacctcatacggtctctagcgcaagagcggatctagtaacatagatgacaccgggcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcgtcgcaagaccggcaacaggattcaatcttaagaaactttattgccaaatgtttgaacgatcaagcctacacctcaataatggtgtggcatgggctgctcatgacttgcctctctgccatggccatcatcagcttcctttccttgattgcaggatcctccaaaaccagcaccttgtccttgtccctcacgcccaccatgtggagatgatcaccatcctctctctccttgcccttgaacagtagcctctgctcccttggctccagccctgtcaccatgcccacaactaccttcaactccccaaatgtggcagtggctccaatagatatctcatgccaacttgacccggtagagattctcactgtgatcacctcatcagctccctcaccacattctctcttctgaaccagcatgcctcctggccgcagctcccactggatgtcaccgttcctgctgcacccaccattgccgccactggccttggaagaactcctcttgaatagcttctttgatctcaacctgatcatcattctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggggggaggacgagcggcgtaccttgaagcggaggtgccgacgggtggatttgggggagatctggttgtgtgtgtgtgcgctccgaacaacacgaggttggggaaagagggtgtggagggggtgtctatttattacggcgggcgaggaagggaaagcgaaggagcggtgggaaaggaatcccccgtagctgccgtgccgtgagaggaggaggaggccgcctgccgtgccggctcacgtctgccgctccgccacgcaatttctggatgccgacagcggagcaagtccaacggtggagcggaactctcgagaggggtccagaggcagcgacagagatgccgtgccgtctgcttcgcttggcccgacgcgacgctgctggttcgctggttggtgtccgttagactcgtcgacggcgtttaacaggctggcattatctactcgaaacaagaaaaatgtttccttagtttttttaatttcttaaagggtatttgtttaatttttagtcactttattttattctattttatatctaaattattaaataaaaaaactaaaatagagttttagttttcttaatttagaggctaaaatagaataaaatagatgtactaaaaaaattagtctataaaaaccattaaccctaaaccctaaatggatgtactaataaaatggatgaagtattatataggtgaagctatttgcaaaaaaaaaggagaacacatgcacactaaaaagataaaactgtagagtcctgttgtcaaaatactcaattgtcctttagaccatgtctaactgttcatttatatgattctctaaaacactgatattattgtagtactatagattatattattcgtagagtaaagtttaaatatatgtataaagatagataaactgcacttcaaacaagtgtgacaaaaaaaatatgtggtaattttttataacttagacatgcaatgctcattatctctagagaggggcacgaccgggtcacgctgcactgcagctcctgacagcgaaatgttctggcactgcacttgcactggggacagcctattttgctagtttgttttgtttcgttttgttttgatggagagcgtatgttagtactatcgattcacacaaaaaaccaacacacagatgtaatgaaaataaagatattttattcccgggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgaagctcagagttcatcatgttcagagttatcgagatccggtggatcacgggcccgcggaaccgtcgactggagaatacgcagcttgagttcgaggtctgagtacttatacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcactgccattagatgagagatttcgattccgattttgatttcgattccgattttgatttcgattgatctcttccttctgatttgtgttccttatataaggaaattcttgtgggatcctagacgtcatggcttacgtcatttccttcgtcctgttgctcactgattgagctgtgagtggagggaccactggaagatgcttcactaattttcttagtggagggaccggcttcacatgcttcacacaagtggctgtcgggcatcatcttttttagcttttgacaaagcaatgttttagtggtggctcccactcttatcttcaacattattatcttatcttcaaaggacgataagatgttgatgtctgtggacgaagatcctagacgtcatggcttacgtcatttccttcgtcctgttgctcactgattgagctgtgagtggagggaccactggaagatgcttcactaattttcttagtggagggaccggcttcacatgcttcacacaagtggctgtcgggcatcatcttttttagcttttgacaaagcaatgttttagtggtggctcccactcttatcttcaacattattatcttatcttcaaaggacgataagatgttgatgtctgtggacgactcctctgagcgtgcaggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaaaagcctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcagatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgaacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttggacattgtttagtatactagaattccagcgtgtcaaacttacaaatttctctgaagttgtatcctcagtacttcaaagaaaatagcttacaccaaattttttcttgttttcacaaatgccgaacttggttccttatataggaaaactcaagggcaaaaatgacacggaaaaatataaaaggataagtagtgggggataagattcctttgtgataaggttactttccgcccttacattttccaccttacatgtgtcctctatgtctctttcacaatcaccgaccttatcttcttcttttcattgttgtcgtcagtgcttacgtcttcaagattcttttcttcgcctggttcttctttttcaatttctatgtattcttcttcgtattctggcagtataggatcttgtatctgtacattcttcatttttgaacataggttgaatatgtgccgcatattgatctgcttcttgctgagttcacataatacttccatagtttttcccgtaaacattggattcttgatgctacatcttggataattaccttctgctcctgagaccgaattccaaacgcgaagtaatcttttcggttttaaagaaaaagggcagggtggtgacaccttgcccgtttttttgccggaccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtccccccttcagaagttcctatactttctagagaataggaacttccctatagagtcgaataagggcgacaccccctaattagcccgggcgaaaggcccagtctttcgactgagcctttcgttttatttgatgcctggcagttccctactctcgcatggggagtccccacactaccatcggcgctacggcgtttcacttctgagttcggcatggggtcaggtgggaccaccgcgctactgccgccaggcaaacaaggggtgttatgagccatattcaggtataaatgggctcgcgataatgttcagaattggttaattggttgtaacactgacccctatttgtttatttttctaaatacattcaaatatgtatccgctcatgagacaataaccctgataaatgcttcaataatattgaaaaaggaagaatatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaaaagcttaataagatgatcttc

SEQ ID NO: 15 - DNA, 66 bp, synthetic construct
  source 1..66: mol_type = other DNA
ggcagcggcgccacaaacttctctctgctaaagcaagcaggtgatgttgaagaaaaccccgggcct

SEQ ID NO: 16 - protein, 22 aa, synthetic construct
GSGATNFSLLKQAGDVEENPGP

SEQ ID NO: 17 - DNA, 500 bp, Horseradish latent virus
  source 1..500: mol_type = genomic DNA
tggtagactatgaaacactagtctactcaaagaacttgaagaagacgactcaggaagacaggagcgtcatcaacaagtttcagcaaaagctgattagtggaaaaatccttggattccactctccagcaatctgccagcacataaaggtgacagcagaaaaagaagattgtgactaccactgcaatcagtgcgaatcttcaaaaggaaaggctatcgtttgcgataagcctgccgacagtggtccagccgacaatggagggccccagacacgtgaagacgcgtctgccgacagtgggtctaggacaacagacaccacgcactcagcaagtggatgaaataattcatctgctgacgtaagggatgacgatcaatcccactatcccaagacccttcacttctatataagtgaagttgcttcatttggagaaggcatctcgaaatctcaacacaactcgagctctcccttctctcttctttatctctctaaatgtgtgagtaga

SEQ ID NO: 18 - DNA, 439 bp, Dahlia mosaic virus
  source 1..439: mol_type = genomic DNA
tcgacgggaagacaaaaatcaagatcaattacgcacaggaaaaagtattgatcatgagtcactacggagcaccaatctccagacaagatgtcgacatcttatcttcctttgaagaaagcattgcacatgccaaagttgatgtagggacatctacaaaattattgctttgcaaacagctgaagaaattattggccgacaaccataaatgctcagcatgtgaaagtggggcctccaccagcaataatacaagctttacagcttgcagtgactcatcctccaataatggagaaaaagacgtcagcagtgacgaacaagggtcgaaagacttgcctatataagagcattctcccctcagttgaagatcatcgaaagttggagcaataaactctctcttcaacaaatctatcttttgtctagaatcatgtgtgagtagtaaccc

SEQ ID NO: 19 - DNA, 585 bp, synthetic construct
  source 1..585: mol_type = other DNA
gtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccagagtttttacttcggacagtcaaaaatgagtttaacttctcagccgaggtaaaacaagaaatatgcttacgtctacagagggatttctctgaagatcatgtttgccagctatgcgaacaatcatcgggagatcctgagccaatcaaagaggagtgatgtagacctaaagcaataatggagccatgacgtaagggcttacgccattacgaaataattaaaggctgatgtgacctgtcggtctatcagaacctttactttttatatttggcgtgtatttttaaatttccacggcaatgacgatgtgacctgtgcatccgctttgcctataaataagttttagtttgtattgatcgacacgatcgagaagacacggc

SEQ ID NO: 20 - DNA, 621 bp, synthetic construct
  source 1..621: mol_type = other DNA
tcgtccacagacatcaacatcttatcgtcctttgaagataagataataatgttgaagataagagtgggagccaccactaaaacattgctttgtcaaaagctaaaaaagatgatgcccgacagccacttgtgtgaagcatgtgaagccggtccctccactaagaaaattagtgaagcatcttccagtggtccctccactcacagctcaatcagtgagcaacaggacgaaggaaatgacgtaagccatgacgtctaggatcttcgtccacagacatcaacatcttatcgtcctttgaagataagataataatgttgaagataagagtgggagccaccactaaaacattgctttgtcaaaagctaaaaaagatgatgcccgacagccacttgtgtgaagcatgtgaagccggtccctccactaagaaaattagtgaagcatcttccagtggtccctccactcacagctcaatcagtgagcaacaggacgaaggaaatgacgtaagccatgacgtctaggatcccacaagaatttccttatataaggaacacaaatcagaaggaagagatcaatcgaaatcaaaatcggaatcgaaatcaaaatcggaatcgaaatctctcatct

SEQ ID NO: 21 - DNA, 465 bp, Cestrum yellow leaf curling virus
  source 1..465: mol_type = genomic DNA
tggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagctt

SEQ ID NO: 22 - DNA, 742 bp, Cauliflower mosaic virus
  source 1..742: mol_type = genomic DNA
tgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagctttcgcagatctgtcgatcgaccaac

SEQ ID NO: 23 - DNA, 299 bp, Agrobacterium tumefaciens
  source 1..299: mol_type = genomic DNA
gatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgca

SEQ ID NO: 24 - DNA, 1369 bp, Sugarcane bacilliform badnavirus
  source 1..1369: mol_type = genomic DNA
atccattccccacattcaagaaggacatgttcgaaagaacagagcaagcttttatgctaacagaggaaccagttctactctgtgcatgcaggaagcctgcaattcagttagtgtccagaacatctgccaacccaggaaggaaattcttcaagtgcgcaatgaacaaatgccattgctggtactgggcagatctcattgaagaacacattcaagacagaattgatgaatttctcaagaatcttgaagttctgaagaccggtggcgtgcaaacaatggaggaggaacttatgaaggaagtcaccaagctgaagatagaagagcaggagttcgaggaataccaggccacaccaagggctatgtcgccagtagccgcagaagatgtgctagatctccaagacgtaagcaatgacgattgaggaggcattgacgtcagggatgaccgcagcggagagtactgggcccattcagtggatgctccactgagttgtattattgtgtgcttttcggacaagtgtgctgtccactttcttttggcacctgtgccactttattccttgtctgccacgatgcctttgcttagcttgtaagcaaggatcgcagtgcgtgtgtgacaccaccccccttccgacgctctgcctatataaggcaccgtctgtaagctcttacgatcatcggtagttcaccacatgatcatttgagcaagtttgcttgaataaaagaactatcattccgcatacctgatcctatagtcctagcttgagaacaagagcgaagtctatagttgagatcctaagagaaactcgaggtttttcgggtttcctgggcgcgttccctggtatcagagcgaggtatgatttctgtatccgctatgttctaaatttcttagataaggggccaagggctctgctgatgagtttaaggacaactacttgtgcaagttacatagcatgatacgtcgaaaggctgaaaatatccaaaatactgtctattgtttggaaaactaggttgttctaggggagaacgttaatgaggggtaagcttagttcattctgaaaatcaaggtctgtgattgtagttgagctcagtaattaagtgctgaaggaagtaagatctaggtaggacaaaagtacccgtcaaggcaggaggccgctaaggggaaaagagccagacgatcaaagctttttcagcacggttgttgagtttagctatcaagaaatagccttgagactaagattcatcactaagaactgcctactcaagcctccctgaatccgcctattagtacaaacgagacaacagtataaggagaaactatatgcctgtaagacttaagtgcaaaagtaacctctggaactgggtagaagtctag

SEQ ID NO: 25 - DNA, 514 bp, Cassava vein mosaic virus
  source 1..514: mol_type = genomic DNA
cagaaggtaattatccaagatgtagcatcaagaatccaatgtttacgggaaaaactatggaagtattatgtgaactcagcaagaagcagatcaatatgcggcacatattcaacctatgttcaaaaatgaagaatgtacagatacaagatcctatactgccagaatacgaagaagaatacatagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttg

SEQ ID NO: 26 - DNA, 585 bp, Figwort mosaic virus; identical to SEQ ID NO: 19
  source 1..585: mol_type = genomic DNA

SEQ ID NO: 27 - DNA, 1932 bp, Citrullus lanatus
  source 1..1932: mol_type = genomic DNA
tcaacataaacgcattagtgattaatacatttatttatgttttttgaagttagaaatttaaattcttaaatgtacctttgtaaaaaaaaatatatatatatatatatattgtgcccatgtttatatcctagagcgcattgtaatcacaaatcagataaatatatatcagattaaaataataataataataataataataatataatcccttataattatgcatttctaactttaaaaaataatccgtccataaacaacccattaatattaaccaagttgttgcaaattttgaaaacatatatatttgaaagccgtgtgaatgtaaaatactaaataaaatgatacatattttagtttttgtttgaacatatttcatagcctaacaaattcaaaagaaagaaaaaataattcttcccaaatttgaacaaagttttttctttattctttagatggagaaaatagtgtgagtaggttaaaaattttgggagagtgatattattattggggtcgataagatatttttgtggaattgtaataatttttcacatgttgaaatttttatggtctgtagttttcttttttctgaagtttttctatattaatgcttgtgtttctaattttattgctttctttcaatttctttgttgtttttcttctcattcatatagtagtaatgggaggtttttcccaaaagtatacacattattttttaattttattttctaatttttcacataacatttatatattatttttgttttcaatttttttagatggtgtaatatgtgtttatactattttaatttttagtacacttgaaatcaattttaaaaaattaattctaattttatttttctcacatatacatatgcatatacaaatatttttaatttttagtaaatgattttatgaaaattagttgtataaaaaacttctattttgaaatttagataaatatttcaagcttcattatttgcctatatattttggaaaaattatcttaaatgataaaactggtgaaatatttacaattaatagcaaaatacatagtctattgcaataaacaataaaattttgctatatttataaatatttgggtttcgtttgctatatttgaaaatagccttatattttgccatggataattgtgtaaagggttattatgattttaaaactaatatatgaatattagattaaagttgagttcctaaatttctaaatttatgtctaatagaatctctaaatttttaattttgtgtataattaagtcttttaaaaatgtctaatagatctataaacttttatctaatagtttcaaacatacttaatttttttttatttaaaaaaaaaaaaagggatgaactgttatattgggcataaaatttaattttaggtttaaatgaccctcacattttaaatcttctaactaataaatatattcattttttaaaatgtaacccgttagatacgtaaagttaaattttatatttaatataacatttaattttcaataattagataaaaattgaaactatatgaatctattaaatacaagattaagaacttagagacacattgaaaattaattttaaagtttatggacaatatatatcaacagaaatttaaaacttcataataagctaaacttgtaagctttatatggatttgtgtccctgtcatgaagttatggattcgtcactcaatgtctggtctaattaaaaagatgaatatgaaaaacgttttaacgattagcaaacaccaaaaccctaattaattatctctctctctcctccttagatttggctttttccttagcgtgaaaagcccaacttgttgtagtgaaactcagtcctataaatacaagaaagaaaaaaccctcttcaaaacccaaaccataaagaaaagaaaaaaaaaaaaaaaaaaaaaaagaga

SEQ ID NO: 28 - DNA, 2000 bp, Citrullus lanatus
  source 1..2000: mol_type = genomic DNA
tctctgtgtaaaaaaataaaaaataaaaaactatttttttaaatatttgtattaaaatacattaaaattatataagctttttactattattgtcatacattatttaatcaaatagaagcagggcaggctagaagagtagaagaattttggcgggaataagttcttgattcctcaaaataattcgtgatccgtgatccgtgaacatttaaaaatcaaattgaaataaaattcaaaacataaagactaaaagtgtaatattttaaaatacaatggttaaattaaaacatgtatatatatatttaatttttggaggaaaatagaaaaagaaaggaaaggtgtaacacacaacaaataataagaatttgaagtcaagaaatgagaacacacttccccgtttcgtgtgttttggttgaacaacttcaacctcaatctcccaagtcccaacatgtgaaatctcagttttaccccccaccgcctttgaattaaacgccgcgttttcttctttccttcttcaacccaaacgccaagctcctcctttcttttttcctttcaacagacgacagcccacaaacagacctatggttttaaaacttttacacatatgtatacttcattagatgtcatattaagcaaatcaacgattaaaattaatgcatagaggatttaatttccaaagtaattcttgactaataacaaaggctaaaaattaatacaacactcttaaacaaacatgaagtaatttagctttaaccatatcaattaaatttcaattttaacatcttttaagaacaaattttcttaccgcgtggagtctaacgctgtagaccttattggtttgcttagtttttaagtagtttgaacttactgaattagatatttttgttgtatccaccaaatgctaaatacttttgtttctctctcagaaaggagaataaagttgcttactctgttgcaactttcacatcatctcttaaggattcttttaggtggaaggataaattttataattgtattatctctatcattaatggagagttcgtgtcgtatttggggttggatgctctatttgtatctttgtgtcttttttttttttcaatctcaatgttgtctagaacatgtctctaatttttgtttttttaaaccataagatttctaaacattttgttctgtgtctaatagatttataacttctttttaaaaaaatgtttaacaagtccttgtaagttgttgttaatttaattttagacatttatttgacactataactatgatatatgatctttaaacgcaaaatgtcaatttgagcacaattcaattgattaaaatattattcgtaacattaataaaaagattaaactactaaaaaatgttaattttagcgactgtatagagcgttgaattaagttctaaaattatgtgagaatcatagtattaattttccgtcaacatctctatattttttttgtttccatagatttgatatcgacattagaggtgaatcgaaatctccattatatcgtagaaaaatccacgaaacataaaagataatcacgaaatgttactaatataaatataatttaaataatagacatgtcaacttgttcaaaaaatcataaaacatttatttactaatttaaatttattttttattttttaattttatcacttaaaataaaaaaatttaaaaaaaaattcttcaataaatatgttaacttcaattttcaacggtattgaaattgagaaaggaaaaaaaaaaaaaacacaatttgcagcgaacacaaataatttaattagtttttagtttttttgtattccacgtggccacaaatctcacgcgacttttcaccacgcaattggacgcctagtcccggtggggtccacacgccttccggccaccctataaataagctccgaatacaacgctctcatttcaccgactcacaatttccagaattcacacacattccttttcttctcttaagtaatttctttcactttca

SEQ ID NO: 29 - DNA, 561 bp, Pisum sativum
  source 1..561: mol_type = genomic DNA
gtttcattgcccacacaccagaatcctactaagtttgagtattatggcattggaaaagctgttttcttctatcatttgttctgcttgtaatttactgtgttctttcagtttttgttttcggacatcaaaatgcaaatggatggataagagttaataaatgatatggtccttttgttcattctcaaattattattatctgttgtttttactttaatgggttgaatttaagtaagaaaggaactaacagtgtgatattaaggtgcaatgttagacatataaaacagtctttcacctctctttggttatgtcttgaattggtttgtttcttcacttatctgtgtaatcaagtttactatgagtctatgatcaagtaattatgcaatcaagttaagtacagtataggctttttgtgtcaacatttattcactttcctttctgcaattttcaaatcatgtttgtagtttttattggacttgatgaatctctcttaccaccaaaatagaacattttttcttgatataccagaatctcaatatttgtctgataattattctagctgca

SEQ ID NO: 30 - DNA, 405 bp, Arabidopsis thaliana
  source 1..405: mol_type = genomic DNA
tttttgtgatctgatgataagtggttggttcgtgtctcatgcacttgggaggtgatctatttcacctggtgtagtttgtgtttccgtcagttggaaaaacttatccctatcgatttcgttttcattttctgcttttcttttatgtaccttcgtttgggcttgtaacgggcctttgtatttcaactctcaataataatccaagtgcatgttaaacaatttgtcatctgtttcggctttgatatactactggtgaagatgggccgtactactgcatcacaacgaaaaataataataagatgaaaaacttgaagtggaaaaaaaaaaaaacttgaatgttcactactactcattgaccataatgtttaacatacatagctcaatagtatttttgtgaatatggccaac

SEQ ID NO: 31 - DNA, 540 bp, Glycine max
  source 1..540: mol_type = genomic DNA
acgggaattaaacctataaacataaatataaataatatatataaacctaagtgtctaagttccataaattaagctgtagtctctggcttaaaacatgttaggtttgtttatacaagtagttggatgtttggagtacttcggtcttttgcgtaccatcaatatttaagaactaagttagttatgttccgtaacttatgggctcttaattaaactatatctgcacaaaattatatatatatcaaatgtgatggtatgtggactataaaaagatatggttgagaaccacaaactttgaaacttcgaataatatattgccagtgacagtcttgttgatttgttatagcaagtcctattttcttaatcattgctttgttttaacgtacctagatttcataacttttgtctttgtctcaagctgaacctaatgatgatagtaatattaacttattgtataggggtatttcataggataaaaaatgatgtgcaattacgtgtagaccaaatattacttgatgacagatggataaatattttaaaact

SEQ ID NO: 32 - DNA, 210 bp, Arabidopsis thaliana
  source 1..210: mol_type = genomic DNA
tcccctttctggaatattcagcgttgattattctggaacccatttctatgtggtcaatgcaaatttaagaaattatttgccgacttaacagttgaggaactattgtttgaaagtgaaaatgttattcctatcagtttctctataattatagttatcatttcatttcatttttgcccttaaatctttgaaatcttatttttcgtttagctc

SEQ ID NO: 33 - DNA, 295 bp, Pisum sativum
  source 1..295: mol_type = genomic DNA
attatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacatcccaaac

SEQ ID NO: 34 - DNA, 486 bp, Arabidopsis thaliana
  source 1..486: mol_type = genomic DNA
gaagatgaagatgaaatatttggtgtgtcaaataaaaagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttctgcagcatataactactgtatgtgctatggtatggactatggaatatgattaaagataagatgggctcatagagtaaaacgaggcgagggacctataaacctcccttcatcatgctatttcatgatctattttataaaataaagatgtagaaaaaagtaagcgtaataaccgcaaaacaaatgatttaaaacatggcacataatgaggagattaagttcggtttacgtttattttagtactaattgtaacgtgagactacgtatcgggaatcgcctaattaaagcattaatgcgaacctg

SEQ ID NO: 35 - DNA, 414 bp, Solanum tuberosum
  source 1..414: mol_type = genomic DNA
tttaaactgattttaatgtttagcaaatgtcttatcagttttctctttttgtcgaacggtaatttagagttttttttgctatatggattttcgtttttgatgtatgtgacaaccctcgggattgttgatttatttcaaaactaagagtttttgtcttattgttctcgtctattttggatatcaatcttagttttatatcttttctagttctctacgtgttaaatgttcaacacactagcaatttggcctgccagcgtatggattatggaactatcaagtgtgtgggatcgataaatatgcttctcaggaatttgagattttacagtctttatgctcattcccttgagtataatatagtaaaaaaatagtaaatttaagcaataatgttaggtgctatgtgtctgtcgagactat

SEQ ID NO: 36 - DNA, 220 bp, Arabidopsis thaliana
  source 1..220: mol_type = genomic DNA
agaaaagatgattgattattacttttgtaaggctgtaagttcgtcgcactcatttccttttacctctttttctgaattgggattctctctttttgtgctttgtgtgttttgttattgaatttctttcccaaacctaagatttgttccagagtgtaatcacatttgcctatgcttctttgttttgtctatgaaaatatttcaatgtcaaaagcctatacaa

SEQ ID NO: 37 - DNA, 175 bp, Cauliflower mosaic virus
  source 1..175: mol_type = genomic DNA
tttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatc

SEQ ID NO: 38 - DNA, 1503 bp, synthetic construct
  source 1..1503: mol_type = other DNA
atgggaagatcaccgtgttgcgatcaagacaaaggcgtgaagaaaggaccgtggctgccagaagaagatgataagctcactgcttatataaacgagaatggttatgggaattggcggtcgcttcctaagctcgctggacttaaccgctgtggcaagagctgtcggctccggtggatgaattatctccggcctgatatccggagaggcaaattttccgatggagaagagagtactatcgttagactccatgccctccttggcaacaagtaagactatttttttttcgttaaagcttttatgctattttgttagcactaaatgatttaggtcttcgcttgtttcagacgtaattatgtgttcctgtttacatgtttttttcataattttaaaatataaaccattttcagttcttttaaaattaatataaagtgcaaaaacaaatttacgtatggataccattgcatagtttattattgctattatagaattcacatcctcaagactatagtctaaactttagtttataattctctaaccatacaaaaggttaagttttcgttggttcattttactaacgtttgagtcgaatttttaccatacagatggtcgaaaattgcgggtcatcttccaggaagaacagataatgaaattaaaaactattggaacactcatatgaggaagaagttattgcaaatggggattgatccagtgacgcacgagccaagaaccaacgatcttagccctatcctcgatgtttctcaaatgctcgcggcagcaatcaacaatggccaatttggtaacaataacctcctcaacaataacaccgctttggaagatattcttaaactccaattgatccataaaatgcttcaaatcataacccccaaagccataccaaacatcagtagcttcaagaccaatttactgaatcctaaaccagaaccggtagtcaatagcttcaataccaattcagtgaatcctaaaccggatcccccggctggtcttttcataaaccaaagtggaatcactcctgaggccgcctcggatttcattccatcttatgaaaatgtttgggatggttttgaagataaccagcttcctggtttggttacggtttctcaagagagtttaaatacagcaaaaccgggtaccagtacgaccaccaaggtaaatgatcatatcagaaccggtatgatgccgtgttactatggtgatcaactactagaaaccccatctactggttcggtatcggtttctcccgagacaaccagtttgaaccatcccagtacagctcaacactcatccggttcagatttcctagaggactgggagaagtttcttgatgatgaaacaagtgattcttgctggaaaagtttcttagagtaagatctatctccgcattgattaacatagtatatgatttatatttatggtttattaacagaattgttttatctaatctgcagcttaacgtcacccacatcgtcacctgtcccatggtag

SEQ ID NO: 39 - protein, 360 aa, Arabidopsis thaliana
MGRSPCCDQDKGVKKGPWLPEEDDKLTAYINENGYGNWRSLPKLAGLNRCGKSCRLRWMNYLRPDIRRGKFSDGEESTIVRLHALLGNKWSKIAGHLPGRTDNEIKNYWNTHMRKKLLQMGIDPVTHEPRTNDLSPILDVSQMLAAAINNGQFGNNNLLNNNTALEDILKLQLIHKMLQIITPKAIPNISSFKTNLLNPKPEPVVNSFNTNSVNPKPDPPAGLFINQSGITPEAASDFIPSYENVWDGFEDNQLPGLVTVSQESLNTAKPGTSTTTKVNDHIRTGMMPCYYGDQLLETPSTGSVSVSPETTSLNHPSTAQHSSGSDFLEDWEKFLDDETSDSCWKSFLDLTSPTSSPVPW

SEQ ID NO: 40 - DNA, 510 bp, synthetic construct
  source 1..510: mol_type = other DNA
atggcgatgaagatcatcagcatggccgccctcgtggtcgtcgtgctcgcgtcctcggtgctccagtcgacgtacgctgcgaactacacggtgggcgacgcgaccgggtggacggtgccgaaccccaacaacaccgagttctacgacgactggacggacaacaagaacttcgtggtgggcgacgtcctcgagtttagcttcaccaccggccgccacgacgtggcggaggtgacggaggcggcctacgacaactgcaacacgaccaatcccatcgtccggtacaccaccggcccggcgagcatcacgctgaaccggaccggggagtaccacttcatctgcacgttcgacgggcactgcggcctcgggcagaagctctccatcgacgtccggacggggccgagcactgcgccgacccctggctccacccccaacccgtcgtccagcgcgtcggccctggtggccaccatctcgctcgtgttcatgtccatcgccctggcgctgttctgctaa

SEQ ID NO: 41 - protein, 169 aa, Gossypium hirsutum
MAMKIISMAALVVVVLASSVLQSTYAANYTVGDATGWTVPNPNNTEFYDDWTDNKNFVVGDVLEFSFTTGRHDVAEVTEAAYDNCNTTNPIVRYTTGPASITLNRTGEYHFICTFDGHCGLGQKLSIDVRTGPSTAPTPGSTPNPSSSASALVATISLVFMSIALALFC

SEQ ID NO: 42 - DNA, 2526 bp, synthetic construct
  source 1..2526: mol_type = other DNA
atgctggcggagcagaggctctacttcccgatcctgctcttcatctacctgctgttctgcagctcccggtttgcgatctcccagtccagctgggactacaacgccaccctgagcaactccatcgccggcttcgcgggcctgagcagcttctggatcaaccgcccgtcactgatcatcaacagcacgacggacagcttctacggctccacgaccccggtcctgcagagggggaacgcgggccctaggtttctgtgtggcttctactgtcgctacaacgtgacggagtgcctcctcggcatcctcctgtaccacaacaagtataacgagcagaacggcatgatcgacaagccgcagctggtgtggagcgcgaacaggaaccgcccggtgaagttcaacgcgaccctcgagctgggccaggacgggaacctggtcctcaccgacagcgacggcacgctggtgtggagcaccgacaccatcggcaagtccgtgagcggcctcaacctgaccgagatggggaacctggtgctgttcgacaagaggaagaggacgatctggcagtccttcgaccacccaaccgacagcctgctgccgggccaatccctcgtgtccggccgcaagctcgtggcctccgtcagcgccaccaaccacagccaggggctgctggcgctgacggtgctgaacgggagctgggccgcctacatggacacgaacccgccgcagtactactacaccagctactatgcggacagctcctactattccttcaacggccagaccttcacggtgctccactacccgaccaactccacggcgcagttcatgaagatcggcccggacggccacatcaaggtgttccagtggtccgtggtcgactggaacgaggtgtccgacatcctctccccgaacgtggacaactgcgcctacccgatggtgtgcggcagctacagcatctgcaccaacaacggccagtgcacgtgcccgagccaggagaacttcttccgccccttctccgagaggaagccggacctggggtgctcccagctcacctccgtcaactgcaactccagccagtaccacagcttcatcgagctgaagaacaccacctacttctccttcaacatcgaccaagagctgaacagcacgaaactgtggctcggccagaagaagctggaggactgcaagcgcgcgtgcctgtcccactgcagctgcaaggccgccgtcttcgagtacaactggaacggcgaccgccgcggcaactgcctgctgctgaacgaggtcttctccctgaaggacagcgaggaagtccgcggcgagacggtgttcctgaaggtccagaacacctccaaggcgcagacccagtccctgatcatcccggggggcaagaagagcaggccgttcaaggtgatcatcgggtccacgctggccgcgttcttcggcatcattctcagcatcgccacctgttttgtgatcttccgcaagcgcacccacgagtcccgcaaggcggccgacatcctcgacctggccccgatcctcccggggatcctgacccgcttctcctacaacgagctcaagatcatcacggacgacttctccagcaagctgggcgagggcggcttcggctgcgtgtacgagggcaccctgcgcaacggcaccaagatcgccgtgaagaacctggacggcgtggggcaggtcaaggagagcttcctgactgaggtgaaggcggtgggcggcatccaccacatcaacctggtcaagctgattggcttctgcgcggagaagacccaccgcctgctcatctacgagtacatggtgaatggcagcctggatcgctggatcacgcacgagaacagggagaacggcctgacctggagcaccaggcagaggatcatcagcgacatcgccaagggactggcgtatctgcacgaggactgcagccagaagatcatccacctggacatcaagccgcagaacatcctcctcgaccagtacctcaacgcgaagatcagcgacttcgggctcagcaagctcattgagaaggacaagagcaaggtggtcacccgcatgagggggacgaggggctacctcgccccagagtggctgtcctccgtgatcaccgagaaggtggacgtgtacgccttcggcatcgtgctcctggagatcctgtgcgggcgcaagaacctcgattggagccagccggacgaggaagacgtccacctcctgtccgtgttccgcaggaaagcggagcaggagcagctcatggacatggtggacaagaacaacgaggacatgcaactccacagggaggcggtgaccgagatgatgagcctcgccgcgtggtgcctgcagggcgacttctccaaaaggccatccatgtccctggtggtgaaagccctggagggcctggtgaccgtcgagacgaacctcaactacgacttcacccacgtgccggaggtcggcgcggggaaccaggagagggaggtgatcgtgtcctccatcttcccgtccatcctctccggcccgcgctag

SEQ ID NO: 43 - protein, 841 aa, Solanum pennellii
MLAEQRLYFPILLFIYLLFCSSRFAISQSSWDYNATLSNSIAGFAGLSSFWINRPSLIINSTTDSFYGSTTPVLQRGNAGPRFLCGFYCRYNVTECLLGILLYHNKYNEQNGMIDKPQLVWSANRNRPVKFNATLELGQDGNLVLTDSDGTLVWSTDTIGKSVSGLNLTEMGNLVLFDKRKRTIWQSFDHPTDSLLPGQSLVSGRKLVASVSATNHSQGLLALTVLNGSWAAYMDTNPPQYYYTSYYADSSYYSFNGQTFTVLHYPTNSTAQFMKIGPDGHIKVFQWSVVDWNEVSDILSPNVDNCAYPMVCGSYSICTNNGQCTCPSQENFFRPFSERKPDLGCSQLTSVNCNSSQYHSFIELKNTTYFSFNIDQELNSTKLWLGQKKLEDCKRACLSHCSCKAAVFEYNWNGDRRGNCLLLNEVFSLKDSEEVRGETVFLKVQNTSKAQTQSLIIPGGKKSRPFKVIIGSTLAAFFGIILSIATCFVIFRKRTHESRKAADILDLAPILPGILTRFSYNELKIITDDFSSKLGEGGFGCVYEGTLRNGTKIAVKNLDGVGQVKESFLTEVKAVGGIHHINLVKLIGFCAEKTHRLLIYEYMVNGSLDRWITHENRENGLTWSTRQRIISDIAKGLAYLHEDCSQKIIHLDIKPQNILLDQYLNAKISDFGLSKLIEKDKSKVVTRMRGTRGYLAPEWLSSVITEKVDVYAFGIVLLEILCGRKNLDWSQPDEEDVHLLSVFRRKAEQEQLMDMVDKNNEDMQLHREAVTEMMSLAAWCLQGDFSKRPSMSLVVKALEGLVTVETNLNYDFTHVPEVGAGNQEREVIVSSIFPSILSGPR

SEQ ID NO: 44 - DNA, 1068 bp, Musa acuminata
  source 1..1068: mol_type = genomic DNA
atggcgaagaagacgaccaccttcctcctcctcaacctgttcttggttgctctctccacgctaacgacagctgcagtttcacctgcggtctttgttttcggagactccctcgtcgacgtcgggaacaacaacttcttgcccagcgatgcaccgaaggtcaacttccctccctggggaatcgattaccctggacgaactcccaccgggaggttcaccaatggcttcaactacgccgattacgtcgccaaagcagtaggcttagcgatgagcccgccacctttcctttctctttccaatgggaatcagatgctgagaggagtaaacttcgcatctggtgggacagggattctcgataccacaggcggagatgtgatcgccatggcgacacagattgaagacttcgagcaggttgcggcgaacctcacggaaagattagggaagaagtctgctgctgtcttcctggagaagtctctcgttcacttgatcgttgggagcaatgacatctacgcgctgtattctcttctcagtccaggaaacagcactcagaaggatgaagtcgtcgttcttctactcgacaagttcaagcatcaaatagagaggctatatgatctcggagcacgaaaatttgcagtcctgggtgttccaccgattgggtgtgttccgatggtcagagtcgcaatttcttcgtatggctgcaacgaagatttaaatgatctttgtctacgcttcaagactgccacaaaggctctcctgaaggacctcagcatgtcatttaagggattccagtactcgttcggagacttatatgaaatgttcacccaaatcttctctgatccccaacaatatgggtttacggagctcaaagctgcgtgctgtggaggtgggagattgaacgccgagtctgcctgcctgccgaactccacatactgcagcaaccgtgaccaatacgccttctgggacttgagtcatccctcccaggctttgcacaaaacgatggctcagctggcactctatggaccgccattgatagccaaccccatcaacattcatcagttggtgaagagttag

SEQ ID NO: 45 - protein, 355 aa, Musa acuminata
MAKKTTTFLLLNLFLVALSTLTTAAVSPAVFVFGDSLVDVGNNNFLPSDAPKVNFPPWGIDYPGRTPTGRFTNGFNYADYVAKAVGLAMSPPPFLSLSNGNQMLRGVNFASGGTGILDTTGGDVIAMATQIEDFEQVAANLTERLGKKSAAVFLEKSLVHLIVGSNDIYALYSLLSPGNSTQKDEVVVLLLDKFKHQIERLYDLGARKFAVLGVPPIGCVPMVRVAISSYGCNEDLNDLCLRFKTATKALLKDLSMSFKGFQYSFGDLYEMFTQIFSDPQQYGFTELKAACCGGGRLNAESACLPNSTYCSNRDQYAFWDLSHPSQALHKTMAQLALYGPPLIANPINIHQLVKS

SEQ ID NO: 46 - DNA, 597 bp, Musa acuminata
  source 1..597: mol_type = genomic DNA
atggaagcggcggctttggcgacgagaccgattgtgaaggtggcagcgctctgcggttccctccgcaaggcttccatgaactgcggcctcatccgctccgccattcagctatgcgacgagtccatcgagggcatgacgatcgagtacgtggacatcgcgccgctcccattcgtcaacaccgacctcgaggtcgacggcacgttccccgagcccgtggaggccttccggcagacgatccgcggagccgacgccatcctatttgcttcgcccgagtacaactactccttcacgggtcctttaaagaatgctattgactgggcatcaagacctccaaacgtaatggcagacaagcctgctgctattataagcgctggtggtggttttggaggaggaaactctcagctccatcttcgccaggttggagtttttctggatcttcatttcatcaacaagccggaactctttgtccatgcattccagcctccaccaaaatttgacagagatggaaatcttattgatcctgagattaaagaacggctaaagcaactacttttatcgctcaaggctttcacccttcggcttcagaacaaatgctga

SEQ ID NO: 47 - protein, 198 aa, Musa acuminata
MEAAALATRPIVKVAALCGSLRKASMNCGLIRSAIQLCDESIEGMTIEYVDIAPLPFVNTDLEVDGTFPEPVEAFRQTIRGADAILFASPEYNYSFTGPLKNAIDWASRPPNVMADKPAAIISAGGGFGGGNSQLHLRQVGVFLDLHFINKPELFVHAFQPPPKFDRDGNLIDPEIKERLKQLLLSLKAFTLRLQNKC

SEQ ID NO: 48 - DNA, 906 bp, Musa acuminata
  source 1..906: mol_type = genomic DNA
atggcggagacagtggtcccctgtgttctccagaagcctcggcatgtcgtcagaaagtttctggccaagccgcaacacgaaggagacggcgccgtcgtcaggagaagcattgggaggtttgagctgaggtactttgatcctttcctcgttctggatgaattctcagtgactgctcctgcaggatttcctgatcatcctcacagaggttttgagacagtcacctacatgctagagggtgctgtgactcacgaagactttgaaggccacaagggtacaataaaggcgggcgacctgcaatggatgacggcaggaaggggaattgtgcactccgagatgccggcagggaaaggcacatccaaaggcctgcagctctgggttaatctgtcctccaagaacaaaatgatcgagccgaggtaccaagagatgcagagccaggacatagcttgtgcttccgggaacggcgtcgaggttcgagtcatcgccggcgaatcgatgggagtccgatccccggtctacactcgcaccccaaccatgtacttggacttcacgctgaagccccgggcgcacctccggcaacccatcccatccgcctggaacgcattcgtgtacgtgcttgaaggcgagggggcattcggcggcgacaagtccgccccgatcggcccgcacaatcttctcctgctggggcagggtgatggggttgacgtgtggaacaagtcggcgaagcccctccggttcgtgctcgtcggcggggagccgctgggcgagccggtagcccaactggggccgttcgtgatgaacaccgacgaggagatcgatcggaccatcgacgacttccagtactgcatcaacgggtttgagaaggccaagtactggaggtccgaggcgatggtgggaattgaaggctga

SEQ ID NO: 49 - protein, 301 aa, Musa acuminata
MAETVVPCVLQKPRHVVRKFLAKPQHEGDGAVVRRSIGRFELRYFDPFLVLDEFSVTAPAGFPDHPHRGFETVTYMLEGAVTHEDFEGHKGTIKAGDLQWMTAGRGIVHSEMPAGKGTSKGLQLWVNLSSKNKMIEPRYQEMQSQDIACASGNGVEVRVIAGESMGVRSPVYTRTPTMYLDFTLKPRAHLRQPIPSAWNAFVYVLEGEGAFGGDKSAPIGPHNLLLLGQGDGVDVWNKSAKPLRFVLVGGEPLGEPVAQLGPFVMNTDEEIDRTIDDFQYCINGFEKAKYWRSEAMVGIEG

SEQ ID NO: 50 - DNA, 1008 bp, Musa acuminata
  source 1..1008: mol_type = genomic DNA
atgccggacatggagaaaggcaaagcaagcgaacaaccagggcttctgctggctgagctagctcgagtgcaggagctggcgagagagatggaagctgaacatggccggcactctacagatgaattccgcagatcgctgctgcacgaattgctcgtgtcgatcgagaaggccatttgcatggccaagtctaccgcaccggacgtcaacccgcagcctgcggatggagactctccgcgctcctccggcgagagctcccggagcgagacatccaagctgaccttcagggagcagatgtccaagaagaggaagaaactgcccaggtggaccagggaggtgcgagtgagctcaggcgccggcggcggggtccatggccctgtcgatgacggctatagctggaggaagtacggccaaaaggatatcctcgggtccaaacatccaagaggctactacaggtgcacacatcgcatcatgcagggttgcccggcgacgaagcaagtgcagcgatcagacgaggacccgctgctgtttcatgtgacctatcatggggcgcacacctgccttccgaagagccctgcggcaccagcatcagcatcagcatggctgtcgcaggagcagcaacagcaggaagagaaggagcccgtggatcggcagcagcagaaccaagacttgcttttgagattccaaacggggctccgagtgaagacggaagacttggagttgggcggccatggccagaactcgctttccttctccttcgcgtcgccttccacgacgaccgacagtggcttcattagcagagcctcgcctgccttcctctccccaacatcagactcgacctacttctcgttctcgccatacagcttgaaaggaggaggaatcaagcttcggagccctgagtctggcatcacaggagctaattcagcttcagattcacctgctgctgcggacatggatttaatgctcgaagagctagacttcgaatcggacttctccagcttcttctcatga

SEQ ID NO: 51 - protein, 335 aa, Musa acuminata
MPDMEKGKASEQPGLLLAELARVQELAREMEAEHGRHSTDEFRRSLLHELLVSIEKAICMAKSTAPDVNPQPADGDSPRSSGESSRSETSKLTFREQMSKKRKKLPRWTREVRVSSGAGGGVHGPVDDGYSWRKYGQKDILGSKHPRGYYRCTHRIMQGCPATKQVQRSDEDPLLFHVTYHGAHTCLPKSPAAPASASAWLSQEQQQQEEKEPVDRQQQNQDLLLRFQTGLRVKTEDLELGGHGQNSLSFSFASPSTTTDSGFISRASPAFLSPTSDSTYFSFSPYSLKGGGIKLRSPESGITGANSASDSPAAADMDLMLEELDFESDFSSFFS

SEQ ID NO: 52 - DNA, 1014 bp, Musa acuminata
  source 1..1014: mol_type = genomic DNA
atggcaagtcaactgctctccgcggtcggcaaccacatctccctccccgagagctacgtccggccagagtcacaaaggcctcgtctccacgacgtgatcaacgacaccaacatccccatcatcgatctcggctcacccgataagcagcagatcgtcgcgcaagtcgccgacgcctgcaggtctttcggcttcttccaggtagtgaaccacggagtgacggtggattcgatgcagaagatgatgggagtggcttcggaattctttcacctcccaccggaagagaaggagaagtactactccgacgacaccacgaagaagatgaggctgtcgacgagcttcaacatcaggaaggagtcggtccgcaactggagggactatctccgcctccactgctatccgcttgaggagctcgtgcccggttggccttcaaaccccgcttcgttcaaggaagtggtgagcgcttactgcagggaagtccgtcaactagggtttcggctgctgggagcaatctctctgagcttggaactggaagaggactacctggaaaagatgctcggcgagcaggaacaacatatggcagtgaactactacccgaagtgccccgatcccgagctcacgtatggcttgccggctcacaccgaccccaacgtcctaaccattcttctccaggatccaaatgtggcggggttgcaggtcctcaaggacggcagatggatggccgttcgtcccctacccaacgccttggtcgtcaacgtcgctgaccaactgcaggcattgagcaatggaaggtataggagcgtgcggcatcgagcggtggtcaactccgacaaggagaggatatcggtggcgtcattcctttgccccggcaacagcgtggtgatcagccccccggagaagctcgtgggcggcgacgggtgtcgggccatgtacagaagctacacttacgaacagtactacgacaagttctggagcagagccctggacgagcattgcttggacttcttcaaacgctga

SEQ ID NO: 53 - protein, 337 aa, Musa acuminata
MASQLLSAVGNHISLPESYVRPESQRPRLHDVINDTNIPIIDLGSPDKQQIVAQVADACRSFGFFQVVNHGVTVDSMQKMMGVASEFFHLPPEEKEKYYSDDTTKKMRLSTSFNIRKESVRNWRDYLRLHCYPLEELVPGWPSNPASFKEVVSAYCREVRQLGFRLLGAISLSLELEEDYLEKMLGEQEQHMAVNYYPKCPDPELTYGLPAHTDPNVLTILLQDPNVAGLQVLKDGRWMAVRPLPNALVVNVADQLQALSNGRYRSVRHRAVVNSDKERISVASFLCPGNSVVISPPEKLVGGDGCRAMYRSYTYEQYYDKFWSRALDEHCLDFFKR

SEQ ID NO: 54 - DNA, 360 bp, Musa acuminata
  source 1..360: mol_type = genomic DNA
atggcgaagagttccttcaagttggagcatcccctcgaaaggaggcaagcagaggctgctcgtatcagggagaagtatcctgataggattcctgtaattgtggagaaggctgaaagaagtgacattccagatattgacaagaagaagtacctggttcctgctgatctgacagtaggacaatttgtttatgtggtccgcaagaggattaagctcagtgccgagaaggccatcttcatctttgtgaagaattctctgccacccacagctgctatgatgtctgcaatctacgaggagaacaaggatgaggatggttttctctacatgacttacagcggggagaacacatttggatctgtttaa

SEQ ID NO: 55 - protein, 119 aa, Musa acuminata
MAKSSFKLEHPLERRQAEAARIREKYPDRIPVIVEKAERSDIPDIDKKKYLVPADLTVGQFVYVVRKRIKLSAEKAIFIFVKNSLPPTAAMMSAIYEENKDEDGFLYMTYSGENTFGSV

SEQ ID NO: 56 - DNA, 360 bp, Musa acuminata
  source 1..360: mol_type = genomic DNA
atggcgaagaattccttcaagttggataatcccctcgaaaggaggcaagctgaggctgctcgtatccgggagaaatatcctgataggattcctgtgattgtagagaaggctgaaagaagtgatataccagatattgacaagaagaagtacctggttcctgccgatctcacagtgggacaatttgtttatgtggtgcgcaaaaggattaagctcggtgctgaaaaggccatcttcgtctttgtgaagaattcactaccgccgactgcttccctgatgtctgcgatctacgaggaaaacaaggatgaggatggttttctgtacatgagttacagtggggagaacacatttgggtctgcgtag

SEQ ID NO: 57 - protein, 119 aa, Musa acuminata
MAKNSFKLDNPLERRQAEAARIREKYPDRIPVIVEKAERSDIPDIDKKKYLVPADLTVGQFVYVVRKRIKLGAEKAIFVFVKNSLPPTASLMSAIYEENKDEDGFLYMSYSGENTFGSA

SEQ ID NO: 58 - DNA, 2130 bp, synthetic construct
  source 1..2130: mol_type = other DNA
atggagttcgagaggaacggccaccacgccaacggcggcctgtgcatcaaggacccgctcaactgggccgccgccgccgaggccctgaccggctcccacctggacgaggtgaagaagatggtggccgagtccaggcagccgctggtatccctggaggggaccaccctgaagatcgcccacgtggccgccgtcgccggccagtccggcgcccgcgtggagctctccgagtccgcgaggggcagggtgagggcctccagcgattgggtgatggactccatgaccaagggcaccgacagctacggcgtgaccacggggttcggcgcgacgtcccaccgccgcacgaagcaggggggcgccctgcagaaggagctgatccgcttcctgaacgcgggcatcttcggcagcggctccgaccagtccggccacaccctgccgccgccggccacccgcgccgccatgctggtgaggatcaacaccctcctgcagggctactccggcatcaggtttgagatcctggaggccatcacctccctcctcaactccaacattacgccgtgcctcccgctcaggggcaccatcaccgcctccggcgatctcgtgccgctgtcctatattgccggcattctgaccggcaggccgaactccaaagcgatctccccggccggcgaggccatcgacgccgccgaggccttccgcctggccgggatcccgcacggcttcttcgagctccagccgaaggagggcctggcgctggtgaacggcaccgcggtgggcagcgggctggcgtccatggtgctgttcgacgccaacatcctggccgtgctgtccgaggtcctgtccgccatcttctgcgaggtgatgcaggggaagccggagttcaccgaccacctgacccacaagctgaagcaccacccgggccagatcgaagccgccgccatcatggagcacgtgctggagggctccagctacatgaagatggccaaaaaactccacgagcaggacccgctgcagaagccgaagcaggacaggtacgcgctgaggacctccccgcagtggctgggcccgcagatcgaggtgatcaggtcctccacgaagtccatcgagagggagatcaacagcgtgaacgataacccgctgatcgacgtgagcaggaacaaggccctgcacggcggcaacttccagggcaccccaatcggcgtgtccatggacaacaccaggctcgccattgccgccatcgggaaactgatgttcgcgcagttctccgagctggtgaacgacttctacaacaacggcctgccgagcaacctctccggggggaggaacccgtccctcgactacggcttcaagggcgccgagatcgccatggccgcctactgctccgagctccagttcctggccaacccggtgaccaaccacgtgcagtccgccgaacagcacaaccaggacgtgaactccctgggcctgatttcctccaggaagaccgccgaggccgtggacattctgaagctgatgtcctccacctacctggtggcgctctgtcaggccatcgacctcaggcacctggaggagaacctgaaaaacgccgtgaagaacaccgtgattcaggtggccaagaagaccctgaccaccggggtgaacggcgagctgcacccgtccaggttctgtgagaaagacctgatcaccatcatcgatagggagcacgtgttctcctacgtggacgatccatgcagctccacctacccgctgatgcagaagctgaggcaagtgctggtggagcacgccctgaataatggcgagaaggagaaggacccgaataccagcatcttccagaagatcaccgccttcgaggaggaactgaaggccgccctcccgaaggaggtggaggccgccagggccgcggtggagaacggcaacgcggccaccgcgaacaggatcgaggagtgtaggtcctacccgctctaccgcttcgtcagggccgagctgagcaccggctacctgacgggcgagaaggtccgctccccgggcgaggatttcgacaagatcttcgcggccatcaacaagggtatggtgatcgacccgctgctggagtgtctcaaagagtggaatggcgccccgctgccgatctgctga

SEQ ID NO: 59 - protein, 709 aa, Musa acuminata
MEFERNGHHANGGLCIKDPLNWAAAAEALTGSHLDEVKKMVAESRQPLVSLEGTTLKIAHVAAVAGQSGARVELSESARGRVRASSDWVMDSMTKGTDSYGVTTGFGATSHRRTKQGGALQKELIRFLNAGIFGSGSDQSGHTLPPPATRAAMLVRINTLLQGYSGIRFEILEAITSLLNSNITPCLPLRGTITASGDLVPLSYIAGILTGRPNSKAISPAGEAIDAAEAFRLAGIPHGFFELQPKEGLALVNGTAVGSGLASMVLFDANILAVLSEVLSAIFCEVMQGKPEFTDHLTHKLKHHPGQIEAAAIMEHVLEGSSYMKMAKKLHEQDPLQKPKQDRYALRTSPQWLGPQIEVIRSSTKSIEREINSVNDNPLIDVSRNKALHGGNFQGTPIGVSMDNTRLAIAAIGKLMFAQFSELVNDFYNNGLPSNLSGGRNPSLDYGFKGAEIAMAAYCSELQFLANPVTNHVQSAEQHNQDVNSLGLISSRKTAEAVDILKLMSSTYLVALCQAIDLRHLEENLKNAVKNTVIQVAKKTLTTGVNGELHPSRFCEKDLITIIDREHVFSYVDDPCSSTYPLMQKLRQVLVEHALNNGEKEKDPNTSIFQKITAFEEELKAALPKEVEAARAAVENGNAATANRIEECRSYPLYRFVRAELSTGYLTGEKVRSPGEDFDKIFAAINKGMVIDPLLECLKEWNGAPLPIC

SEQ ID NO: 60 - DNA, 2568 bp, synthetic construct
  source 1..2568: mol_type = other DNA
atggcggccgtggacctgcgccacatcctgctggacctcctcccgagcttcgacatgaaggagaggtgccgcgagctcggcatccagcaccacctggagaagatcctcccggacctctgggccatcaacgccgtcatcagggacgcgatgatccgcgccttcacccagccggacgtcgagatgtggaccgccgacgcgggcgccgcgatcgccgacgtgcagaacctgctggacaggatcctcgagtggaccggcagggccgcggcgccgagcaacccactcctgcgcagctgcctgggcatccgcgtggcgttccgcctcagcatcctgcaggagctcaaggagatggggctgcgcctcaaggagctcgtgaggaggggcccagccctggacctcaggaaggagatgatggacgccatggacccgtgcgacgaggagtacttctacgtgctcggcgacgaggtcgtcggcagggacgaggaccgcgacaacatcgtcgagattctgcagcagaatcagtcctcctccaatggcgagccgtttgtgatccagatccacgacgagtggccgttcggctccctgtccaccggcaagaccaccctggcgaggatgatctaccaccacccatgggtgaggcagcacttccaccacaggatctgggtggacgtgagcttcgacctgtcctgggaccaggtgagcatcggccgcgaattcgcgaggtgcatcaccggcgagtcctgcgaccacctccagagccaccaggccatctggctgctcgtgaacgagcgcctgggccagcgcaggtacctgctcatcctgaacgatatcctgtactacgacgacaccgaggaggggctgaaggacaagtgggaccagctcaagcacaacctgctgcacgtgggtggcatcggcagcacggtgatcatcacgaccaagagggcctcccagtactccaggtccctctacatcagccgctccaaggagtatatgctggcggccctgtccgaggacgcgtggatcaagctggtgatgagggacaccttcatcggctccgcgcaggacaaggagaacacatgcaccatcaacctgctcctgaggttcgccgagcaccagtacgagacgatcaaggggagcccgctcctcgccaagaccctgggctccatcttccgcatccagcagctcccgcacaccatcgccaagctcgtgtccctgcgctacctcaacctgtcctacacggagatccaggccctcccgaagtacctcagccacctccagaacctgaagatcctgaagctcacccactgcgagaacttcaggaagctgccgggctggatccacaagctggagaagctgctgatcctgaagctggcctactgccagaagctgcagatgctgccggactccatcaccgccctgtcaaacctccaggagctggacgtggagggctgccagagcttcatgaagctgcccgaggggctggtgtccatgaagaagctgaccatgctgaacgtcgacaagtgcgtgtccctgaccaggctgccgcacggcatcgggcagctgaccaatctgcagaaactgtccgtgcacgccacggtcgactccctggcgagcgtcatcctggagctccagtccctggtcaacctgaaggagctcaggctgaagaagctcaacgggctctcctccacggaggacgccagggccctgaagctcgaggacaagatcctcctcgagtgcctgaccctctcctgggagtggtgggacatcgaggtggccctggtgacggacacgaccctgctgagggagcaggtcatggagaacctgcagccgaacctggccctggagaagctggagatcgtgtcctacatgggcaagaaactgccgtcctggatggcctgcaaggagggcaacctgtggcatctgcgcgagatcaagctggtgaacttgagaaaatgcgaaaggctgccaccagtgggccaactgccgggcctgaagaacgtggagatttctggcatggactccatcagcgccgtcgatgacgccttttacggggacggaaatggcgacacttttcctagattggaaaccttgatcttctccgagatgccactcctggagcgctggctgaaggccaagggggagggcgacatgttcccgatgctggagaggttgatactgatccagtgcccaaagctgaaggagctgcacgtgaggccgagctcctcaacccgcaagcttctccggttggagctctggctcaataacgacaagctcctgagctccgaattcgtcgggtggcaaaatctgataaacgtgtacgtcctcgagatcattggctgcgaggagctccgctgtctgccgcagggcattaagtacctcaaacatcttttccggctgaaaatcattaggtgcaacaacctcatcagcctcccggactggctcgccgagctgaagagcctggagtacctcatcgtccaggactgcgccatgctgagcttcatcccggagcgcctgaagcagtccccgcgcttccacatctggaacgagggctgcccaaagctgcagctgtga

SEQ ID NO: 61 - protein, 855 aa, Musa acuminata
MAAVDLRHILLDLLPSFDMKERCRELGIQHHLEKILPDLWAINAVIRDAMIRAFTQPDVEMWTADAGAAIADVQNLLDRILEWTGRAAAPSNPLLRSCLGIRVAFRLSILQELKEMGLRLKELVRRGPALDLRKEMMDAMDPCDEEYFYVLGDEVVGRDEDRDNIVEILQQNQSSSNGEPFVIQIHDEWPFGSLSTGKTTLARMIYHHPWVRQHFHHRIWVDVSFDLSWDQVSIGREFARCITGESCDHLQSHQAIWLLVNERLGQRRYLLILNDILYYDDTEEGLKDKWDQLKHNLLHVGGIGSTVIITTKRASQYSRSLYISRSKEYMLAALSEDAWIKLVMRDTFIGSAQDKENTCTINLLLRFAEHQYETIKGSPLLAKTLGSIFRIQQLPHTIAKLVSLRYLNLSYTEIQALPKYLSHLQNLKILKLTHCENFRKLPGWIHKLEKLLILKLAYCQKLQMLPDSITALSNLQELDVEGCQSFMKLPEGLVSMKKLTMLNVDKCVSLTRLPHGIGQLTNLQKLSVHATVDSLASVILELQSLVNLKELRLKKLNGLSSTEDARALKLEDKILLECLTLSWEWWDIEVALVTDTTLLREQVMENLQPNLALEKLEIVSYMGKKLPSWMACKEGNLWHLREIKLVNLRKCERLPPVGQLPGLKNVEISGMDSISAVDDAFYGDGNGDTFPRLETLIFSEMPLLERWLKAKGEGDMFPMLERLILIQCPKLKELHVRPSSSTRKLLRLELWLNNDKLLSSEFVGWQNLINVYVLEIIGCEELRCLPQGIKYLKHLFRLKIIRCNNLISLPDWLAELKSLEYLIVQDCAMLSFIPERLKQSPRFHIWNEGCPKLQL

SEQ ID NO: 62 - DNA, 459 bp, synthetic construct
  source 1..459: mol_type = other DNA
atgatcaggctgcgctccaagaagctgttcaagaggagctccagcaaggcgtccggcgggaacggcgggtgctcccgcaacggcgacatccagtgggagctccgcccaggcggcatgctggtccagaagcgcgagtgcggcgagggcgccgacgaggtgatcaccgtgcgcatcagcaccggcagctcctggcacgagatcagcatcggcgccaccgccaccttcggcgagctcaagatcgtggtgggcatggtgacggggctggagccaagggagcagcgcctgctcttcaagggcaaggagagggaggacggggaccatctgcacatggtgggcgtgagggataaggacaaggtgctcgtgctggaggacccagccatcaaggagcgcaagctgatgatggccatggcggagaggcaagtgatgtccagcccgtgccacaccatcatcgaggtgtga

SEQ ID NO: 63 - protein, 152 aa, Musa acuminata
MIRLRSKKLFKRSSSKASGGNGGCSRNGDIQWELRPGGMLVQKRECGEGADEVITVRISTGSSWHEISIGATATFGELKIVVGMVTGLEPREQRLLFKGKEREDGDHLHMVGVRDKDKVLVLEDPAIKERKLMMAMAERQVMSSPCHTIIEV

SEQ ID NO: 64 - DNA, 1155 bp, synthetic construct
  source 1..1155: mol_type = other DNA
atggcctggcgcgagcacatcgccttcctgctgctcatcacgctgttcttcccgtccatcttcagcctgtccaactgcgccgtcgccgcccgcaggaccctcatgggccagccggccgtgaaggcgggctactggccgacgtggaccttctcctactccccgccatcctccgtgcagttctcctacttcacgcacgtgttctacgcgttcgtgcaggtcgactccgccaccttcgggctggcgatcgcggccgacgacgaccgcatgctgaggaacttcaccgccgcggtgcacgcccacccgccagtgaagagcctcctgtccatcgggggcggggactccaacagcgccttcgccgccctggccgccgatccggccagccgctccgccttcatcaactccaccatcacggtggccagggagtacggcctcgacggcctggacctggactgggagttcccgaaggacgccaaggagatggacgatttcagcgcgctcctgctcgagtggcaggcggcggtggtgtccgaggccgcggcgacgggcaggccgaggctcctgctgaccgccgccgtgtacttcgcgccgcgcttcttcctgtccgacgatcagaggagctacccgatcgagcagatggccacggccctggactggatcaatgccatgtgctacgacttccatggctcctgggacacgaccgcgaccggcgagcacgccgcgctgtatgacccgaacagcaatattagcacctcctacgggctggagtcctgggtggccgcgggcatgccgaagaagaaggtcgccatgggcatgccgctgtacggcaagacctggaagctgaaggacccgagccagaacgggatcggcgccccggccgtgggcgtcggcccaggcgagaatggcgtcctggtgtattccgaggtggtcgccttcaacaagaacacgtccgccaccgtggtgcacgatgaggtcacgaccgccgcctattcctacgcgggcacgtcctgggtcgggtacgacgacccgtggagcgtcacgaggaaaatcaattacgcccaaaggctgggcattgggggctacttcttttgggccatcgggtatgataaggactggtccgtgtcctccacggcctggagggcctggaagaacaagtga

SEQ ID NO: 65 - protein, 384 aa, Musa acuminata
MAWREHIAFLLLITLFFPSIFSLSNCAVAARRTLMGQPAVKAGYWPTWTFSYSPPSSVQFSYFTHVFYAFVQVDSATFGLAIAADDDRMLRNFTAAVHAHPPVKSLLSIGGGDSNSAFAALAADPASRSAFINSTITVAREYGLDGLDLDWEFPKDAKEMDDFSALLLEWQAAVVSEAAATGRPRLLLTAAVYFAPRFFLSDDQRSYPIEQMATALDWINAMCYDFHGSWDTTATGEHAALYDPNSNISTSYGLESWVAAGMPKKKVAMGMPLYGKTWKLKDPSQNGIGAPAVGVGPGENGVLVYSEVVAFNKNTSATVVHDEVTTAAYSYAGTSWVGYDDPWSVTRKINYAQRLGIGGYFFWAIGYDKDWSVSSTAWRAWKNK

SEQ ID NO: 66 - DNA, 681 bp, synthetic construct
  source 1..681: mol_type = other DNA
atggcttcgtccagcattctctccctctccttcttcttcttcttcttcttctcccttcttgtcaccctctcccacgccgccaccttcgagatcgtgaacaggtgctcgtacacagtgtgggccgcggccgtccccggcggcggacgccagctcaaccaggggcagtcgtggaccatcaacgtgaacgcggggaccacgggcggccgcatctggggccgcaccggctgctccttcgacgggagcgggcgcgggcggtgccagacgggcgactgcggcggcgtgctctcatgcacggcgtacggcagcccgcccaacaccctggcggagttcgccctcaaccagttcaacaacctggacttcttcgatatctccctggtcgacggcttcaacgtgccaatggacttctcgcccacgagcggcggctgccgcggcatccggtgcgcggcggacatcaacgggcagtgccccggggcgctgaaggcgccgggcggctgcaacaacccctgcaccgtgttcaagacggaccagtactgttgcaactccggcagctgcagcccgaccgactactcccggttcttcaagcgcaactgccccgacgcctacagctatccgaaggacgatcagacgagcaccttcacttgccccggcgggaccaactaccgcgttgtcttctgcccttga

SEQ ID NO: 67 - protein, 226 aa, Musa acuminata
MASSSILSLSFFFFFFFSLLVTLSHAATFEIVNRCSYTVWAAAVPGGGRQLNQGQSWTINVNAGTTGGRIWGRTGCSFDGSGRGRCQTGDCGGVLSCTAYGSPPNTLAEFALNQFNNLDFFDISLVDGFNVPMDFSPTSGGCRGIRCAADINGQCPGALKAPGGCNNPCTVFKTDQYCCNSGSCSPTDYSRFFKRNCPDAYSYPKDDQTSTFTCPGGTNYRVVFCP

SEQ ID NO: 68 - DNA, 1763 bp, synthetic construct
  source 1..1763: mol_type = other DNA
atggccggcctgccatacagcgccccgcacccagccacgatcagcgcctcctccaacagcttcgcgtgcccattcaggagcaaggggctggtcttcccgtacccgacccgcagggcgctgcatgtgaggccgaacatcgcgtgcaaagccggggaggagcacgagatcgcggcgaaggtcgaccgcagggacgtcctggtgggcctgggcggcctgtgcggggcggccgcgggcctcggcggcttcgacaaggccgcgctcgccaacccgatccaggccccggacctgtccaagtgcggcccggccgacctgccaaccggggtgccggtggtgaactgctgcccaccgtacaggccgggcgcgaagatcgtggacttcaagcgcccgagcccctccagcccgctgagggtcaggccagcggcccacctcgtggacccggagtatctggcgaagtacaagaaggccatcgagctgatgaaggccctgccggccgacgacccgaggaacttcatgcagcaggcggacgtccactgtgcctactgcgacggcgcgtacgaccagatcggcttcccgaacctggagatccaggtgcacaatagctggctgttctttccgtggcacaggctctacctgtacttcaacgagcgcatcctgggcaagctgatcggggacgacacctttagcctgccattctggaactgggatgccccgggcgggatgatgctgccatccatctacgcggatccgagctccccgctgtacgacaaactgagggacgccaagcaccagccgccagtgctcgtcgatctggactataatgggaccgacccaaccttcccggacgcccagcagatcgaccacaatctgaagatcatgtataggcaagtgttcagcaatggcaaaaccccactgctcttcctggggtctgcctacagggccggcgagcagccgaatccaggcgcggggtccgtggagaacatgccgcacaacaacgtgcacctgtggaccggcgatcgcacccagccgaacttcgagaatatggggaccttctacgccgccgccagggatccaatcttcttcgcccaccacgccaacattgatcgcatgtggtacctgtggaagaaactgagcaggaagcaccaggactttaacgatagcgactggctgaaggcctccttcctgttctacgacgagaacgccgacctcgtgcgcgtgacggtgaaggactgcctggaaaccgactggctgaggtacacctaccaggacgtcaagatcccgtgggtgaatgccaggccaaccccaaagctggccaaagcccggaaggccgcctcctcctctctgaaagccaccgccgaggtccagttcccggtcacgctcgagtccccagttaaagccacggtcaaaaggccgaaggtggggaggagcgggaaggagaaggaggacgaggaagagatcctcatcgtggagggcatcgagttcgatagggactactttatcaaattcgacgttttcgtgaacgccaccgagggggacggcatcaccgcgggcgcctcagagttcgcgggctccttcgtcaacgtgccacacaagcacaagcacaggaaggacgagaacaaactgaagaccaggctctgtctcggcattaccgacctcctggaggacattggcgccgaggacgacgactccgtgctggtgacgatcgtgccaaaggcgggcaaaggaaaggtgtccgtgggtgggctgaggatcgacttcagcaagtg

SEQ ID NO: 69 - protein, 587 aa, Musa acuminata
MAGLPYSAPHPATISASSNSFACPFRSKGLVFPYPTRRALHVRPNIACKAGEEHEIAAKVDRRDVLVGLGGLCGAAAGLGGFDKAALANPIQAPDLSKCGPADLPTGVPVVNCCPPYRPGAKIVDFKRPSPSSPLRVRPAAHLVDPEYLAKYKKAIELMKALPADDPRNFMQQADVHCAYCDGAYDQIGFPNLEIQVHNSWLFFPWHRLYLYFNERILGKLIGDDTFSLPFWNWDAPGGMMLPSIYADPSSPLYDKLRDAKHQPPVLVDLDYNGTDPTFPDAQQIDHNLKIMYRQVFSNGKTPLLFLGSAYRAGEQPNPGAGSVENMPHNNVHLWTGDRTQPNFENMGTFYAAARDPIFFAHHANIDRMWYLWKKLSRKHQDFNDSDWLKASFLFYDENADLVRVTVKDCLETDWLRYTYQDVKIPWVNARPTPKLAKARKAASSSLKATAEVQFPVTLESPVKATVKRPKVGRSGKEKEDEEEILIVEGIEFDRDYFIKFDVFVNATEGDGITAGASEFAGSFVNVPHKHKHRKDENKLKTRLCLGITDLLEDIGAEDDDSVLVTIVPKAGKGKVSVGGLRIDFSK

SEQ ID NO: 70 - DNA, 1164 bp, Musa acuminata
  source 1..1164: mol_type = genomic DNA
atggccgatgtcgatgccaagaaagtatgcggcgtgccggagagggtgcagctgcatgtggccatgctggccttgcagtttgggtacgccggattccatgtcgtctcccgggcagcccttaacatggggatcagcaaggtggtgttccccgtctaccgcaacattatcgccttgatcctcctcgttccatttgcctacttcctcgagaaaaaggacagaccggctttgacactatcgttcgttgttcaattcttcctcctcgctctgtgtggtataactgcaaaccagggtttctaccttctcggcctcgataacacctctccaactttcgcctccgccatccaaaactccgtcccggccatcaccttcctcatggccgccattctcaggatagagaaggtgcggatcgaccgacgcgacgggatcgcgaagctgatgggcaccctggcctgcgtcggcggcgccaccatcataaccttgtacaaggggccgaccatcttcggcccctcgcgcgccctgaacgacgcgagccagtccaccatgctctggctgggggacgccaaggggaagaactggacgctggggtgcctgtacctgatcggccattgcttgtcgtggtccggctggctcgtgcttcaggcgccggtgctgaagaaatacccggcgcgactgtccgtgacctcctacacctgcttcttcggggtgatccagttcttggtcatcgccgcgttcatcgagagggacgctgaggcctggaagttccactccggcagcgagctcttcaccatcctctacgcggggttcgtggcgtcgggcatcgccttcgcggttcagatatggtgcatcgacaggggcggccctgtgttcgtggccgtgtaccagccagtccagactctagtcgtcgccatcatggcctccatcgccttgcgagaggagttctacctcggcggtatcatcggtgccgtattcatcatagccggactgtacctggtgctgtgggggaagagcgaagagagagccttcgctgcaaaggaagcggccctcacggcctcatccacccccgaccacgatggtctcagagctactaccggtgctgcttccttcaaggcttcctctctgaagcagccactcctgccatcatcgacaaccccggagaatgtctga

SEQ ID NO: 71 - protein, 387 aa, Musa acuminata
MADVDAKKVCGVPERVQLHVAMLALQFGYAGFHVVSRAALNMGISKVVFPVYRNIIALILLVPFAYFLEKKDRPALTLSFVVQFFLLALCGITANQGFYLLGLDNTSPTFASAIQNSVPAITFLMAAILRIEKVRIDRRDGIAKLMGTLACVGGATIITLYKGPTIFGPSRALNDASQSTMLWLGDAKGKNWTLGCLYLIGHCLSWSGWLVLQAPVLKKYPARLSVTSYTCFFGVIQFLVIAAFIERDAEAWKFHSGSELFTILYAGFVASGIAFAVQIWCIDRGGPVFVAVYQPVQTLVVAIMASIALREEFYLGGIIGAVFIIAGLYLVLWGKSEERAFAAKEAALTASSTPDHDGLRATTGAASFKASSLKQPLLPSSTTPENV

SEQ ID NO: 72 - DNA, 1026 bp, Musa acuminata
  source 1..1026: mol_type = genomic DNA
atggcggatcagcttctctccaccgcaacacaccaccatacccttccagagaactacgtaagaccagagtctcaaagacctcgcctcacacaagtgatcaccgacgcaagcatccccatcatcgatctcggctcccccgacaaggcccagatcatctctcggataggccaagcctgccaatcctacggcttcttccaggttgtgaaccatggaattgacactgaactgatggtgaagatgatgactactggtctggagttcttccgtcttccttccgaggagaaggcgaagctatattctgatgacccgtcgaagaaaattagactttcgacgagctttaatgtaaaaaaggagttggttcacaattggagggattacctccggcttcactgctatcctctcgaggagtatgttcccggttggccttctaatccctcctcatttaaggaagtggttagcacttactgtcagcaagttcgtggactgggattccgactccttggagcaatatcattgagcttaggtctggaggaggacttcatcgagagggcgctgggcgagcaagagcaacatatggccatcaactactacccgaagtgcccggaacctgagctcacatatggcctgccggcccacactgatccaaatgccctcactgttcttctccaggattctgatgtggctggcttgcaggttcttagggacggcaaatggatggcagtggatcccaagccaagcgcgttggtcatcaacatcggtgaccagctacaggcattaagcaacggtaggtacaacagcgtttggcatcgagctgtcgtcaactcgaacaaggaaaggatgtctgtggcatccttcctctgtccttgcaacaacgtgatcattagccctccggaggagctcgtcagcgagggatctccagccacttataggcgctacacatacgacgagtactacaagaagttttggagcagaaacttggacggcgaacactgcctggagcttttcaaaagggagaaagcattttag

SEQ ID NO: 73 - protein, 341 aa, Musa acuminata
MADQLLSTATHHHTLPENYVRPESQRPRLTQVITDASIPIIDLGSPDKAQIISRIGQACQSYGFFQVVNHGIDTELMVKMMTTGLEFFRLPSEEKAKLYSDDPSKKIRLSTSFNVKKELVHNWRDYLRLHCYPLEEYVPGWPSNPSSFKEVVSTYCQQVRGLGFRLLGAISLSLGLEEDFIERALGEQEQHMAINYYPKCPEPELTYGLPAHTDPNALTVLLQDSDVAGLQVLRDGKWMAVDPKPSALVINIGDQLQALSNGRYNSVWHRAVVNSNKERMSVASFLCPCNNVIISPPEELVSEGSPATYRRYTYDEYYKKFWSRNLDGEHCLELFKREKAF

SEQ ID NO: 74 - DNA, 561 bp, Musa acuminata
  source 1..561: mol_type = genomic DNA
atgattcctgcaagtgtctgcatggcctcatctgcatactttcttttcctcctccacctctttgccttcgccaacgttgcccctgcaagcggacatgtatatgctgcacccaaagagaagatgacccacttgcacttctacttccacgagatctacggcggccccaacacgaccaccatcaccgtcgtcaaccctcccggcaacaactcgttcaacaacttcgggagcatcggggtgggcgacaacatgcttagggaagggcccaacccgagctccaagctcatcgggagagcgcaggagctggccgttcaggcgtccttggagagcccggcctacctctccgcgttcaacttcgtgttcaccgccggggagtacaacggcagcagcatctccatcttcggccgggcggtgctcacagagacgatggagcggggtatcatcggagggaccggcaagttccgaatggcccggggctacaccattagtaggctcatcaggagaactgggactaccgaaatcgtgcttgtggtggagtacgatgcatacatctttcactactga

SEQ ID NO: 75 - protein, 186 aa, Musa acuminata
MIPASVCMASSAYFLFLLHLFAFANVAPASGHVYAAPKEKMTHLHFYFHEIYGGPNTTTITVVNPPGNNSFNNFGSIGVGDNMLREGPNPSSKLIGRAQELAVQASLESPAYLSAFNFVFTAGEYNGSSISIFGRAVLTETMERGIIGGTGKFRMARGYTISRLIRRTGTTEIVLVVEYDAYIFHY

SEQ ID NO: 76 - DNA, 3579 bp, Musa acuminata
  source 1..3579: mol_type = genomic DNA
atgcacccagagatgtctcgaccgggcgacgaagatctccttcggcagctgcaggaaaccctagattccgatcccgataatccgtcgcaccattataacatcggcctcttcttgtggaaaaagggcgaagcagtagatgatcaagctgaccaatcgaagcaattaagggagagagcagccgagcattttttggcctctgcgaagctgaaccctagtgacggcgtcgcattccgctttctggggcattactacagcacggtttccgtcgatgcacagaggtctgcaaagtgctaccagagggctgtcgcccttaatccccatgattctgaagctggcgaggcgctgtgtgatctgctggatggagaaggaaaggaaagcttggagattgcggtgtgtagagaagcatctgagaagtctcccagagctttctgggcattccagagattgggatacttgcaggtccaccagaggagatggtctgaggcagtacagagccttcagcatgctatacgaggattcccggcatgtgctgacttgtgggaagcgcttggtcttgcctaccatcgcttgggcatgtttactgcagctgttaagtcgtatgggcgagccattgagttggagaactcaagaatatttgcattggtggagagtgggcatattcagctaatgcttggttctttcagaaaaggagttgaacaatttcgatgtgctatggaacttgcaccacacaatatatctgcacaccttgggcttgcttctgggctgcttgattggtcaaaagattgcattaaatctggagcatttggttgggcagctgatctcttacaagaagcatcagaagctgcaaaagtcagtacctgtttgtctggaaatttatattctgcttggaagttgcatggggacatcaagattgcatatgcaaaatgctatccatggggaactggaaggattggatatgaaatagatgaggatctcttgaaatcttccatcatcagctggaaaaaaacatgctattcagctgcaattagtgctaaacactcgtatcagcgggctttacacttggctccatggcaggctaatatttacactgatattgcaatctctgttgattttattggctatctagaagagagaaacaacaatgatttggaaatttggcagctacctgagaggatggccctaggtggcttgttgcttgaaggagttaataaggagttctgggttttgttgggatgtctggctagcagtgatgctctgaagcaacatgctttgattagagcgttgcagttggatgtgtctctttcctcatcatgggcatatctgggaatgctctatagaaactcgggagaaaagcaattggcatgtcaagcatttgatcgtgcaagaagcatagatccttctttggcattaccatgggctggcatgtcagctagttatcaggatgggctttgctcgacaaatgaagcttttgaaagctgtctgtgggctgtgcagacactgcctcttgctgaatttcaagttggtctttgtgcacttgcagttctttctggtcatctgtcatccccgctggtaattggagccatagggcaggccgttcaaagggctccgtacttacccgagtcccataatttacatggcttagtatgtgaatcacggactgattatcagtctgcaattgttgcttatcagaaggcacgatgtgctttgagaatgtttcccaacttcaagagtgatcttcaatcttccttcactgatgtatcagttaaccttgctagatcactttgtaaggccggacatgcaattgatgctgcacaagagtgtgaaaatttgaagaaagaaggattccttgatagtaagggcttgcaaatatatgctgttgcattgtggaaacttggacaatacaacttagctcttactgtggcgagaaagttggctgagaatgtctcaaccatgaaacagacatgtgcagctgcttcccttgggttgatatgcacactaatgtacaacatttcaggatatgactctgtagttagaacaattagaaaatttccaagtgagtttttgcaaaatacaagaatgagtctcatcgtctgtgcattaaatgctttagacacaaataaacaactgcagtcgcttctgccaaccatttctcaagctgctgcttctcatggtattgccattgaaatacattccattaccgctattaacaaattgataactcaggagtctcctcaaactgtagccattgacaggggagtagattacctaagaaaggcccttcatatgtatcctaacagtcttttaataagaagccatcttggtttgcttctactgtctagtggagattggatggctcttcagaaagcagccaggtgtgctgtcattccaactggtcaccggcaccctgtcaagaaaggatttagactaccatatgaaattcatggtgctatggcagttgcttgcggttccatctgttccaccaacccgaagtcttctttccctacctgcaatgatgggcttatgcatggagctagacacttgcacttgcttcaaagatggttgcatcaagaaccatggaaccaaaatgctcgttacttacttgctcttaatgttttccagaaagctcatgaagagaacttcccccaacaactttgcattatttcaaaacggttggttttagatgctctctctacagaagagtttctgggggacaataagctatctcagtctcggagtctcctactcctctgtgcatcagaaataagtttgcagagtggtgactctgttggatgcatacatcatgcatctaatgcattaggtcttcttcctaccaatagtgatatgttttttgcacacttgcaattatgccgtggatatgcagtgcaggaggactattcaaaccttaggaatgagtacaccaagtgcttgcagatgaagaccattcatccgatttgttggatattactaaaatattttgaatctagatacagtttgcagaataatttggacataatacataccaactttcaggcatgtgcagccaggaaaggaagttcctccaacaattggtctgctaactttgaattagtttgtgctcaatgttatttgtgggatcaggactacttccacgcagagcaaaccttggcacgtgcttgtgtagcaaatatggacagctgccttctactttgtcatggtgccatctgtatggaacttgttaggcagcaggcaggtcttcagtttctgtctcgtgcaattagcagcctgacaaaagctcagaagtgttcgcctatgttcttgcccattgtttcactgttgttggctcaagcagaagcaagccttggagcaaaagccaagtgggagaagaaccttagccttgaatggtttgcctggccagcagaaaggaggccagctgagttgtatttccaaatgcacttgcttgcaagtcaatccagtacagtctccaaccaagattctggcgtagggtcgactcaaagcccagagaggtggatactacgagccatacatttgaacccctcatgtttgagatactggagaatacttcacaagtgttacagctag

SEQ ID NO: 77 - protein, 1192 aa, Musa acuminata
MHPEMSRPGDEDLLRQLQETLDSDPDNPSHHYNIGLFLWKKGEAVDDQADQSKQLRERAAEHFLASAKLNPSDGVAFRFLGHYYSTVSVDAQRSAKCYQRAVALNPHDSEAGEALCDLLDGEGKESLEIAVCREASEKSPRAFWAFQRLGYLQVHQRRWSEAVQSLQHAIRGFPACADLWEALGLAYHRLGMFTAAVKSYGRAIELENSRIFALVESGHIQLMLGSFRKGVEQFRCAMELAPHNISAHLGLASGLLDWSKDCIKSGAFGWAADLLQEASEAAKVSTCLSGNLYSAWKLHGDIKIAYAKCYPWGTGRIGYEIDEDLLKSSIISWKKTCYSAAISAKHSYQRALHLAPWQANIYTDIAISVDFIGYLEERNNNDLEIWQLPERMALGGLLLEGVNKEFWVLLGCLASSDALKQHALIRALQLDVSLSSSWAYLGMLYRNSGEKQLACQAFDRARSIDPSLALPWAGMSASYQDGLCSTNEAFESCLWAVQTLPLAEFQVGLCALAVLSGHLSSPLVIGAIGQAVQRAPYLPESHNLHGLVCESRTDYQSAIVAYQKARCALRMFPNFKSDLQSSFTDVSVNLARSLCKAGHAIDAAQECENLKKEGFLDSKGLQIYAVALWKLGQYNLALTVARKLAENVSTMKQTCAAASLGLICTLMYNISGYDSVVRTIRKFPSEFLQNTRMSLIVCALNALDTNKQLQSLLPTISQAAASHGIAIEIHSITAINKLITQESPQTVAIDRGVDYLRKALHMYPNSLLIRSHLGLLLLSSGDWMALQKAARCAVIPTGHRHPVKKGFRLPYEIHGAMAVACGSICSTNPKSSFPTCNDGLMHGARHLHLLQRWLHQEPWNQNARYLLALNVFQKAHEENFPQQLCIISKRLVLDALSTEEFLGDNKLSQSRSLLLLCASEISLQSGDSVGCIHHASNALGLLPTNSDMFFAHLQLCRGYAVQEDYSNLRNEYTKCLQMKTIHPICWILLKYFESRYSLQNNLDIIHTNFQACAARKGSSSNNWSANFELVCAQCYLWDQDYFHAEQTLARACVANMDSCLLLCHGAICMELVRQQAGLQFLSRAISSLTKAQKCSPMFLPIVSLLLAQAEASLGAKAKWEKNLSLEWFAWPAERRPAELYFQMHLLASQSSTVSNQDSGVGSTQSPERWILRAIHLNPSCLRYWRILHKCYS

SEQ ID NO: 78 - DNA, 738 bp, Musa acuminata
  source 1..738: mol_type = genomic DNA
atggccgtggacactgtcgagactgccgtcgctttggttccgccgcagccgccgccgagatcggagatgagcgaggacgagctcccgcccgtcgaggggtgggcgaagcggaagcgctcgaagcgccaccaccgtttcttcgaccacccgccgactgaggaggagtacctggccctctgcctcatgatgctcgcccgcggcggctccggccaccacctcccgtccgtctccaccgcctctgccgcggcgactcctacgcccaccgcgaaggtcgggttcaagtgctccgtctgcggcaaggccttcggttcctaccaagcgctaggagggcacaaggccagccaccgcaagctcaacgccagcagcggcggggaggagccctccgccgccgcgtccccggccgcttccgcttccggctcctccaccgccgcagcaggcgccaccaccaccggcggcaaagtgcaccagtgctccgtgtgcttcaagacgttcccgacggggcaggcgctgggcgggcacaagaggagacactacgatgggagcctcggcagcgggtcggccaccgcgtcgacggcgtcagagggcgccagctcgagccgcaaggggttcgatctgaacctaccggcgacggcggagttcgcgttggatgtcagccggctgtgcgtggccatggcggaggaggaggaagtgcagagcccgatggcgttcaagaaggccagacttctgattccggcataa

SEQ ID NO: 79 - protein, 245 aa, Musa acuminata
MAVDTVETAVALVPPQPPPRSEMSEDELPPVEGWAKRKRSKRHHRFFDHPPTEEEYLALCLMMLARGGSGHHLPSVSTASAAATPTPTAKVGFKCSVCGKAFGSYQALGGHKASHRKLNASSGGEEPSAAASPAASASGSSTAAAGATTTGGKVHQCSVCFKTFPTGQALGGHKRRHYDGSLGSGSATASTASEGASSSRKGFDLNLPATAEFALDVSRLCVAMAEEEEVQSPMAFKKARLLIPA

SEQ ID NO: 80 - DNA, 276 bp, synthetic construct
  source 1..276: mol_type = other DNA
atggccgcccgccttccgtgcacgctgctcctggtgctgctcctcaccctctccctcaccgagtcagtcaggtccggcagcctcaagcccagcgagtgcaacaacaagtgcgacttccggtgctcggcgacgtcgcacaagaagccctgcctcttcttctgcaaggagtgctgcgcgacctgcctgtgcgtgccgcccggcacctacggcaacaaggaggcgtgcccgtgctacaacaactggaagacgaaggagggggggcccaagtgcccctag

SEQ ID NO: 81 - protein, 91 aa, Musa acuminata
MAARLPCTLLLVLLLTLSLTESVRSGSLKPSECNNKCDFRCSATSHKKPCLFFCKECCATCLCVPPGTYGNKEACPCYNNWKTKEGGPKCP

SEQ ID NO: 82 - DNA, 357 bp, Musa acuminata
  source 1..357: mol_type = genomic DNA
atggaggtcaccgtgaagtgtctctacgtgcttgcgttgttgctgttgatctgcaacggcgggacggagagggtcgacggggcgggtgaatgcgggagggtgccggtgaggaggatggctctgcaaatggctccctgcgcgtcagctgcgcaggacgcccggctcccggtctcggctggctgctgcgcggcggtgaagaagatgggccgcaacccgagctgcctgtgcgccgtcatgctctccgacaccgccaagagcgtcggcgtgaagccggacgtggccatgacgatccccaagcggtgcaaccttgcagaccgccccgtgggttacaagtgcggcgggtatacattgccgtga

SEQ ID NO: 83 - protein, 118 aa, Musa acuminata
MEVTVKCLYVLALLLLICNGGTERVDGAGECGRVPVRRMALQMAPCASAAQDARLPVSAGCCAAVKKMGRNPSCLCAVMLSDTAKSVGVKPDVAMTIPKRCNLADRPVGYKCGGYTLP

SEQ ID NO: 84 - DNA, 1062 bp, Musa acuminata
  source 1..1062: mol_type = genomic DNA
atggcggatcagctcctctccaccgtaacttaccacgagacccttccggagaactacgtaaggccagagtctcaaagacctcgtctcacacaggtcatcagcgacgcaaacatccccatcatcgatctcggttcaccggataagtcccgaatcatctcccagatagggaaagcctgccaatcctatggcttcttccaggttgtgaaccatggaatcgatactgaattgatggtgaagatgatggctattagtctggaattcttccgtctacctcccgaggagaaggcgaagctctactccgatgacccggccaagaaaatgaggctctccacgagctttaatgtcagaaaggaggcggtacacaactggagggactacctccggcttcactgctatcctctggaggaatacgttcccggctggccttctaatccctcttcattcaaggaagtggtgagcacctactgtaaggaagttcgtcgactgggatttcggctccttggagcattatcattgagcttaggtttggaggaggagtacatcgaacgggtgctgggacagcaggagcagcatatggccatcaactactacccaaggtgcccggaaccggagctcacatatggcctgcccgcacacactgatccaaatgccctcaccattcttctccagcagcccaacgtggctggcttgcaggttctcaaggacggcaaatggatcgcagtggaacccagaccgaatgcgtttgtgatcaacatcggtgaccaactacaggcattaagcaacgccagattcaagagcgtttggcaccgagctgtagtcaactcggacaccgaaaggatgtctgtggcgtccttcctctgtccatgcaacaccgcgatcattagccctccggagaagctccttgccgagggatcaccagcggtctacaggagctacacatacgacgagtactacaacaagttttggagcagaaacctggatgacgaacattgcttggagcttttcaaaggggagaagagccaatcaggtggactgagcggcccctgcaaaagctcgacatga

SEQ ID NO: 85 - protein, 353 aa, Musa acuminata
MADQLLSTVTYHETLPENYVRPESQRPRLTQVISDANIPIIDLGSPDKSRIISQIGKACQSYGFFQVVNHGIDTELMVKMMAISLEFFRLPPEEKAKLYSDDPAKKMRLSTSFNVRKEAVHNWRDYLRLHCYPLEEYVPGWPSNPSSFKEVVSTYCKEVRRLGFRLLGALSLSLGLEEEYIERVLGQQEQHMAINYYPRCPEPELTYGLPAHTDPNALTILLQQPNVAGLQVLKDGKWIAVEPRPNAFVINIGDQLQALSNARFKSVWHRAVVNSDTERMSVASFLCPCNTAIISPPEKLLAEGSPAVYRSYTYDEYYNKFWSRNLDDEHCLELFKGEKSQSGGLSGPCKSST

SEQ ID NO: 86 - DNA, 348 bp, synthetic construct
  source 1..348: mol_type = other DNA
atggccaagagcaacgcctccgacgcccaggccctgatccgctccctgaggtccgcctacgccgccaccccgaccaacctgaagatcatcgacctgtatgtggtgttcgccatcgccacggccatcatccaggtggtgtacatgggcatcgtgggctccttcccgttcaactccttcctgtccggcgtcctgtcctgtgtgggcaccgcggtgctggccgtgtgcctcaggtcccaggtgaacaaggagaacaaggagttcaaggacctcccaccagagagggccttcgcggacttcgtgctgtgcaacctggtgctgcacctcgtgatcatgaatttcctgggctga

SEQ ID NO: 87 - protein, 115 aa, Musa acuminata
MAKSNASDAQALIRSLRSAYAATPTNLKIIDLYVVFAIATAIIQVVYMGIVGSFPFNSFLSGVLSCVGTAVLAVCLRSQVNKENKEFKDLPPERAFADFVLCNLVLHLVIMNFLG

SEQ ID NO: 88 - DNA, 2103 bp, synthetic construct
  source 1..2103: mol_type = other DNA
atggaggtggagaacggggcggccggcgggctgagggtcgaaaccgacccgctgaactgggcggccgccgccgaggctctggccggctcccacctggacgaggtgaggcgcatgacgtgggagttccgccagccacttgtgaagctggagggctccacgctgaggatctcccaggtcgccgcgattgccgccggggcccattccgcggtgagggtcgagctgagcgagtccgcccgcgacgaggtgtgcgcgtcctcccgctgggtcgtggacagcaccgcgaaggggaccaacagcaacggcatgacggccgggttcggcgcggcgtcccaccgcaggacgaaggagggcggggccctccaaaaggagctgattaggttcctgaacgtcgggatcttcggctccggggcggagagcggccatacgctgccgccatccgccacccgcgcggccatgctcgttcgcataaacacgcttcttcaggggtactctggcatccgcttcgagatcctcgaggcgatggcgtcactgcttaactccgggatcacgccatgcctcccgctgcgcggcacgatcacggcgagcggcgatctggtgccgctgagctacatcgcgggggtcctcacgggccgcccgaacgccaaggcgtgcgacccggcgggcgaaaccatggacgcggccgaggccttcaggcgcgcgggaatcccgcacgagttcttcgagctgcagccaaaggaggggctcgccctcgtgaacggcaccaccgtgggctccgggctcgcctccgtggtgctgtacgaggccaacgtgctggcgctgctggcggaggtgctgagcgccgtgttctgcgaggtcatgcaggggaacccggagttcacagaccacctgacccaccgcctgaagcaccacccaggccagatcgaggcggccgcgatcatggagcacatcctggatggctcctcctacatgaaaatggccaaaaaactgcacgaacaggacccactgcaaaagccaaagaaggacaggtacgccctgcgcacttccccacagtggctgggcccgcagattgaggtcatccgctccagcaccaagtccatagagagggagatcaatagcgtcaatgacaaccctttgatcgacgtcgccaggaacaagaccatatatggcggcaacttccagggcaccccgatcggcgtctccatggacaatgccaggctggccctggccgcggtgggcaaactgatgttcgcccagttcagcgagctggtcaacgacctgtacaacaacggcctgccgtccaacctgtccgggggccgcaacccgtccctcgactacggcttcaagggcgccgaaatcgccatggccgcgtactgctccgagttgcagttcctcgcgaaccccgtcacgaaccacgtccagtccgcggagcagcacaaccaggacgtgaactcactcggcttcatctctagccgcaaggccgcggaggcggtggacattctgaaactgatgtctgccacctacctcctggccctgtgccaggccattgacctgaggcacctcgaagagaacctgaagaacgctgtgaagaacaccgtgagccaggtggccaagagggtgctgaccaccggcgccttctgcgagaagaacctgatcaccgtggccgaccgggagcacatcttcggctacattgatgacccgtgctcctccacctacgccctgatgccgaagctgaggatggcgctggtggagcacgcccttaacaacggggagaaggagaaggacgaaaccacctccgtgttccagaaaatcaccgcgttcgaggaggaactgaaggccgtgcttccgaaggaggccgaggccgccagggccgccgtcgagtccggtaacccggccatggtgaacaggatcaaggagtgcaggtcctacccgttgtacaggttcgtgagggaggagctggggacggaatacctcaccggcgagaaggtcaggagcccgggcgaggagttcgacaaggtgttcgtggccatcgacagggggctggtgatcgacccgctgctggagtgtctcaaggagtggaacggcgtgccgctcccgatgtgctaa

SEQ ID NO: 89 - protein, 700 aa, Musa acuminata
MEVENGAAGGLRVETDPLNWAAAAEALAGSHLDEVRRMTWEFRQPLVKLEGSTLRISQVAAIAAGAHSAVRVELSESARDEVCASSRWVVDSTAKGTNSNGMTAGFGAASHRRTKEGGALQKELIRFLNVGIFGSGAESGHTLPPSATRAAMLVRINTLLQGYSGIRFEILEAMASLLNSGITPCLPLRGTITASGDLVPLSYIAGVLTGRPNAKACDPAGETMDAAEAFRRAGIPHEFFELQPKEGLALVNGTTVGSGLASVVLYEANVLALLAEVLSAVFCEVMQGNPEFTDHLTHRLKHHPGQIEAAAIMEHILDGSSYMKMAKKLHEQDPLQKPKKDRYALRTSPQWLGPQIEVIRSSTKSIEREINSVNDNPLIDVARNKTIYGGNFQGTPIGVSMDNARLALAAVGKLMFAQFSELVNDLYNNGLPSNLSGGRNPSLDYGFKGAEIAMAAYCSELQFLANPVTNHVQSAEQHNQDVNSLGFISSRKAAEAVDILKLMSATYLLALCQAIDLRHLEENLKNAVKNTVSQVAKRVLTTGAFCEKNLITVADREHIFGYIDDPCSSTYALMPKLRMALVEHALNNGEKEKDETTSVFQKITAFEEELKAVLPKEAEAARAAVESGNPAMVNRIKECRSYPLYRFVREELGTEYLTGEKVRSPGEEFDKVFVAIDRGLVIDPLLECLKEWNGVPLPMC

SEQ ID NO: 90 - DNA, 1821 bp, synthetic construct
  source 1..1821: mol_type = other DNA
atgatacgatcgagctccatcaccttcgtcctcctaattgtcgcttcgctcctcctcggacgatcggaatcgtcctgccaaacaggctgcaacctcgccctgggatcctactacctcacctccaatgacaacctcacctacatctccaacctcttcagtgccggtggcgccaacaatctcctcccttacaatcccaacatcacgaacgtcaacagcatcaagaccggaggccgcgtcaatgtcttcttctcttgcgactgcctcaatggcgatttcctcggccacaatttctcctacgtcgtcgttcaaggcgacacctacacttcgatcgcccaaactgtgtataaaaatttgacaacggttgacatgctgtctacgttcaatagctttccggcgaccaacgtgccgactggagcgaccatcggtgtggtggtgaattgttcatgcggtgattcgtcggtgtcaaaggactacgggctgttcctgacttaccctctccagccaggggaaaccctatcgtcggtggctgccgcgaatgggttcgccacgcaggaggatttgctgcagaggtataacccccaggcgaatttcagctctgggacagggatcgtgtttatccctgcgaaagatactagtggaagttatcgtcccctttcctcaagtgcaggactgtcggggggagctattgctggaatttctatagctggagtagttctactaatatttgctatatatttatatattcattttcaaaggagaaagaagttgaagaaagcctcattgctttcatcaccacctgaagcttccatgtacaagccaggagtggttacatctccagcagatggtagttccccatttgcaggtattactgtcgataaatcagtggagttctcttatgcggagcttgctgcggctacagatgattttagcctggcttacaaaatcggaggaggtggctttggagttgtttattatgctgagcttagaggagagaaagctgctatcaagaaaatggatatgcaggcaactaatgaatttcttgctgaactgaaagtcttaacaaatgttcatcacttgaatttggtgcgcttgataggatattgtactgaggtttccttattccttgtttatgaattcattgaaaatggcaacttaagtgagcatatgcgagggttaggtagagatcctctgccatgggccatgagggtgcagattgcccttgattcagccaggggtctagagtatgtccatgagcatactgttccagtgtatatacatcgtgatattaaatcagcaaatatattgatagacaagaactaccgtgcaaaggttgcagattttggattagcaaaactcactgaagtaggtgcatctttgcaaacacggcttgttggcacatttggctacatgcctccagagtatgcacagtatggtgagatttctcctaaggtggacgtttatgcatttggagttgttctgtatgaacttatttcagccaaagacgcaattgttaaaacaggcggttctgtcacggaatcgaagggattgattgctttgtttgaggaggctttcagtaaacctgatcccaaggaagatctgaggaatttagttgatccaaggctcggtcaggactatccaatcgactcggttcttgcgatggcatggcttggaaagagctgcactcaagagaatcctcagcagaggccaaccatgaggcaaattgtagtttcgctgatgacactatcttctgctaccgaagactgggacatcggttccttctttgacaaccaagctcttgccagtctcatgtctggaagataa

SEQ ID NO: 91 - protein, 606 aa, Musa acuminata
MIRSSSITFVLLIVASLLLGRSESSCQTGCNLALGSYYLTSNDNLTYISNLFSAGGANNLLPYNPNITNVNSIKTGGRVNVFFSCDCLNGDFLGHNFSYVVVQGDTYTSIAQTVYKNLTTVDMLSTFNSFPATNVPTGATIGVVVNCSCGDSSVSKDYGLFLTYPLQPGETLSSVAAANGFATQEDLLQRYNPQANFSSGTGIVFIPAKDTSGSYRPLSSSAGLSGGAIAGISIAGVVLLIFAIYLYIHFQRRKKLKKASLLSSPPEASMYKPGVVTSPADGSSPFAGITVDKSVEFSYAELAAATDDFSLAYKIGGGGFGVVYYAELRGEKAAIKKMDMQATNEFLAELKVLTNVHHLNLVRLIGYCTEVSLFLVYEFIENGNLSEHMRGLGRDPLPWAMRVQIALDSARGLEYVHEHTVPVYIHRDIKSANILIDKNYRAKVADFGLAKLTEVGASLQTRLVGTFGYMPPEYAQYGEISPKVDVYAFGVVLYELISAKDAIVKTGGSVTESKGLIALFEEAFSKPDPKEDLRNLVDPRLGQDYPIDSVLAMAWLGKSCTQENPQQRPTMRQIVVSLMTLSSATEDWDIGSFFDNQALASLMSGR

SEQ ID NO: 92 - DNA, 648 bp, synthetic construct
  source 1..648: mol_type = other DNA
atggcggccagcgggggcgccgtccgctgccagcgcatcgggtgcgacgccatgttcaccgacgaggacaacgccgagggctcctgccggtaccacgactcggggcccatcttccacgacgggatgaaggagtggagctgctgcaagcagcgctcccacgacttctcgctcttcctggccatccccggctgcaagaccggcaagcacacgaccgagaagcccgtgacgaacattgtgtccgcgaacacccggaagcccgtccccgtccagagccccgcccagaacaagaacagtgtggactcctgctcacgctgccgccaggggttcttctgctccgaccacggctctcaggggaggccgagcaagccctccgtcgtgatcgagacgaccgacaccgagaagtcaccggcgccggtcaagaagaaggtggggatcaacgagccccagacgtgcaggaacaagggctgcgggaagacgttcaaggagaaggacaaccacgagacggcgtgcgagtaccacccggggccagcggtgttccacgaccgtctgcgcgggtggaagtgctgcgacatccacgtcaaggagttcgacgagttcctttcgatcccgccatgcaccaagggctggcacaacgcggacgccgcctga

SEQ ID NO: 93 - protein, 215 aa, Musa acuminata
MAASGGAVRCQRIGCDAMFTDEDNAEGSCRYHDSGPIFHDGMKEWSCCKQRSHDFSLFLAIPGCKTGKHTTEKPVTNIVSANTRKPVPVQSPAQNKNSVDSCSRCRQGFFCSDHGSQGRPSKPSVVIETTDTEKSPAPVKKKVGINEPQTCRNKGCGKTFKEKDNHETACEYHPGPAVFHDRLRGWKCCDIHVKEFDEFLSIPPCTKGWHNADAA

SEQ ID NO: 94 - DNA, 1680 bp, synthetic construct
  source 1..1680: mol_type = other DNA
atgctctcggggatcaacggcggggtgtggacggaaggaggaggcgatgaagatgacgccgcatcgtggactagagcgaacgcgtccaacgcgtccagcggggtcatggggggccgccgggacgagctcggcctcctcagcttcaagtcgatgctcgacgacgacgacgacaacgactggtacctcgggtctgcggcggcgtccaatcccgtgcctccggcggcctcccaccactccttccaggcattccagacgcaccaggagcttaccgacgtagcattcccctcgaacgtctcgccccacgaggcgctgatgctcccgcccgtggtcaacctcgaccagaaccagcccttcttcacggcgaagtccgccctctcctcgctcttcgtatcggtcagcagcaaccccttcgacaccgggttcggcgtcggctgcgacgccccgggcttcctgcaagctagccaggtgtccaacttcccggtcatgatgaacagggggtgcgggggcggaggcgtcttgggtttcgcggggatgggggcgggcgagcaacttggctgccccgatctgagctcaggagccgcattttcgggggatcacctcctgccctcgtccgggcactgctctggctcgagctcgggagcggccttcggcccgatgggctttgacgggttcgagagctcgccgttcctcaaccggcccaaagtgctccggccgctggagatctttccgcccgtgggggcgcagcccacgctgttccagaaacgcgccgcggctgcgttgaggcagaactcgtcagtcagtggcgagaagggcgggctcctcgggttgtgggagtgggagggggtggtgccagggaaccgggggaaaaccgagctcgaggaggagagcaacaaacggcgcaagggcaacgaggaggacgagatggacgacgggtcgatcgacgcgtcggggctaaactacgacacggacgacgccgccgccgagaacgtaatcggcgaggagaacgcgaacggtgggggggggtgcggcggcagcaatagctacgcgaacagcaccgtaactggcggcggggaccagaagggcaaaaagaaggggctgccggctaagaacctgatggccgagcgccggcggcggaagaagctcaacgaccggctgtacatgctgcggtcggtcgtccccaagatcagcaagatggaccgcgcgtccattcttggcgacgcgattgagtacctgaaggagctcctgcgacgaatcaacgacctccacaacgagctcgagtcgaccccgtcctccagttcggtgcccgtgacctccgccaccagcttccacccgctgacgcccaccctgcccacccttagctgcagggtgaaagaggagctctgcccatcctccgtccctagcccgaacgcccagcccgccagggtggaagtgcgagtcagggagggccgtgcggtaaacatccacatgttctgtgccaggcggcccgggctgctcctctccacgatgcgcgcgctagacggcctcggcatcgacatccagcaggcggtcatcagctgcttcaacgggttcgccatggacgtcttccgggcggagcagtcgaaggagggtcccggcgtgctgcccgaggacatcaaggccgtgttgctgaactctgccgggttcgacaataccgtgagctag

SEQ ID NO: 95 - protein, 559 aa, unidentified
  source 1..559: note = Musa acuminata x balbisiana
MLSGINGGVWTEGGGDEDDAASWTRANASNASSGVMGGRRDELGLLSFKSMLDDDDDNDWYLGSAAASNPVPPAASHHSFQAFQTHQELTDVAFPSNVSPHEALMLPPVVNLDQNQPFFTAKSALSSLFVSVSSNPFDTGFGVGCDAPGFLQASQVSNFPVMMNRGCGGGGVLGFAGMGAGEQLGCPDLSSGAAFSGDHLLPSSGHCSGSSSGAAFGPMGFDGFESSPFLNRPKVLRPLEIFPPVGAQPTLFQKRAAAALRQNSSVSGEKGGLLGLWEWEGVVPGNRGKTELEEESNKRRKGNEEDEMDDGSIDASGLNYDTDDAAAENVIGEENANGGGGCGGSNSYANSTVTGGGDQKGKKKGLPAKNLMAERRRRKKLNDRLYMLRSVVPKISKMDRASILGDAIEYLKELLRRINDLHNELESTPSSSSVPVTSATSFHPLTPTLPTLSCRVKEELCPSSVPSPNAQPARVEVRVREGRAVNIHMFCARRPGLLLSTMRALDGLGIDIQQAVISCFNGFAMDVFRAEQSKEGPGVLPEDIKAVLLNSAGFDNTVS

SEQ ID NO: 96 - DNA, 945 bp, synthetic construct
  source 1..945: mol_type = other DNA
atggaggagaacggaatgaagtcgaagatcctgatcttcggggggaccggctacatcgggaaccacatggtcaagggcagcctcaagctggggcacccgacgtacgtcttcacccggcccaactcgtcgaagaccacgctcctcgacgagttccagtccctgggggcgatcatcgtaaagggcgagctggacgagcacgagaagctggtggagctgatgaagaaggtcgacgtggtcatctcggccttggccttcccccagatcctcgaccagttcaagatcctcgaggcgatcaaggttgccgggaacataaagcgtttcctaccgtcggacttcggggtcgaggaggaccgcatcaacgctctgccgcccttcgaggccctcatcgagcggaagcggatgatccggcgcgcaatcgaggaggccaacattccgtacacctacgtcagtgccaactgcttcgcctcctacttcattaactacctcctccgcccctacgacccgaaggacgagatcaccgtctacggcaccggggaggccaagttcgccatgaactacgagcaggacatcgggctctacacgatcaaggtcgcaacggacccgcgggccctcaaccgggtcgtgatctaccggccgagcacgaacatcattacccagctcgagctgatctcacgatgggagaagaagatagggaagaagttcaagaagattcacgtccccgaggaagagatcgtcgccctcacgaaggaactgccggagccggagaacatccccatcgcaatcctccactgtctgttcatcgacggggccacgatgtcctacgacttcaaggagaacgacgtcgaggcctcgaccctgtaccccgagttgaagttcaccacgatcgacgagctgctcgacatcttcgtgcacgacccgccgccgcccgcctcagcagcgttctag

SEQ ID NO: 97 - protein, 314 aa, Ocimum basilicum
MEENGMKSKILIFGGTGYIGNHMVKGSLKLGHPTYVFTRPNSSKTTLLDEFQSLGAIIVKGELDEHEKLVELMKKVDVVISALAFPQILDQFKILEAIKVAGNIKRFLPSDFGVEEDRINALPPFEALIERKRMIRRAIEEANIPYTYVSANCFASYFINYLLRPYDPKDEITVYGTGEAKFAMNYEQDIGLYTIKVATDPRALNRVVIYRPSTNIITQLELISRWEKKIGKKFKKIHVPEEEIVALTKELPEPENIPIAILHCLFIDGATMSYDFKENDVEASTLYPELKFTTIDELLDIFVHDPPPPASAAF

SEQ ID NO: 98 - DNA, 1800 bp, synthetic construct
  source 1..1800: mol_type = other DNA
atgtccacgagcatcccgctgccgttcttcttctcccacggcgtggtgcacgccaggatcgggcacaggccgatccagccgtccgtgggcctgctctccgcgggcgcgccggccaaggtgtccttccgcaggcagtcctccgcccacaggcacgccgcccacgtggtgagctccgagtccatcaccccggtcaaggaggtgtccagcttcgagccgtccgtgtggggcgacttcttcatcaactacgacccaaagccgctccagaggtccgaggactggatgatggagaaggccgacaagctgaagcaggacgtcaggatcctgttcgagacacacaacgacaccgccaagaagatgcacctggtggacgccgtgcagaggctgggcatcgaccacctgttccaggacgagatcaacaacgccatctccgacatcaaggaatccgagttcaccagctccagcctccacgaggtggccctgcacttcaggctgctcagggagcacggcatctgggtgtccccggacgtgttcgagaagttcaagggcgaggacggccgcttcatcaacaccatcgcggacgagccgcgcgcgctgctgtccctgtacaacgccgcccacctcctggtgcatgacgagccggagctggaggaggccatgagcttcgcccgccaccacctcgagagcatgagggacggctccaggctgaaggccccgctggacaaccagatcaacagggccctgcacctcccgcttccaaggacctacaagagggtggaaatgctgcactacatgctggagtacggccaggaggaggagtgcatcgtggtgctcctggacctggccaagctggagttcaacctcctgcagcacgtgcacctcaaggagctgaaggccttctcccagtggtggaaggacctgtacgggtacgtggagctgagccacgtgagggacagggccgtggagtcctacctgtggagctacgccctgttctacgaggagaacctgaccctgaccaggatgatcctcgccaagatcatcgtgttcatcgtgctgatggacgacacgtacgacgaccacgccaccatcgaggagtgcaggaagctcaacgaggccatccagaggtgggacgagtccgccatctccctgctgccggagtacatgaagaagttctacagggcgctgctgaactacttcagggagacagaggcccaagtggaggcctccgacaagtacagggtgacctgcatgaagaaggagttccagaacctgagcacctactacctccaggagttcgagtggctccaccagaactacaaaccggcgttcaaggagcgcgtggccctgagcaccctgagctccaccgtcccgctgctctgcgtgaccgccgcggtggggcagggcgacgccgtgaccaaggagtccttcgagctgaccaccgtgaggtccagcgccgtcatcgcgtgcgccaagatcatgaggttcatgaacgacatcgccgccttcaagtccggcaggaagaacaagggggacgccgccaacaccgtggagtgctacatcaacgagcacaaggtgacctccgaggtggccctggacaagatcgagtccatgatcgagtccgagtggaggacgctgaaccaggtgaggtgcgaccaccagaagcagttccacgtggtccagcgcgtgatgaacctggccgtggcggtgccgttcttctacgacaagaagaaggacgcgtacaccttctccaagtaccaccaggggaacgtgggcaacctcttcgtgaacccagtgaccctgtga

SEQ ID NO: 99 - protein, 599 aa, Oryza sativa
MSTSIPLPFFFSHGVVHARIGHRPIQPSVGLLSAGAPAKVSFRRQSSAHRHAAHVVSSESITPVKEVSSFEPSVWGDFFINYDPKPLQRSEDWMMEKADKLKQDVRILFETHNDTAKKMHLVDAVQRLGIDHLFQDEINNAISDIKESEFTSSSLHEVALHFRLLREHGIWVSPDVFEKFKGEDGRFINTIADEPRALLSLYNAAHLLVHDEPELEEAMSFARHHLESMRDGSRLKAPLDNQINRALHLPLPRTYKRVEMLHYMLEYGQEEECIVVLLDLAKLEFNLLQHVHLKELKAFSQWWKDLYGYVELSHVRDRAVESYLWSYALFYEENLTLTRMILAKIIVFIVLMDDTYDDHATIEECRKLNEAIQRWDESAISLLPEYMKKFYRALLNYFRETEAQVEASDKYRVTCMKKEFQNLSTYYLQEFEWLHQNYKPAFKERVALSTLSSTVPLLCVTAAVGQGDAVTKESFELTTVRSSAVIACAKIMRFMNDIAAFKSGRKNKGDAANTVECYINEHKVTSEVALDKIESMIESEWRTLNQVRCDHQKQFHVVQRVMNLAVAVPFFYDKKKDAYTFSKYHQGNVGNLFVNPVTL

SEQ ID NO: 100 - DNA, 492 bp, synthetic construct
  source 1..492: mol_type = other DNA
atggcgagcggcagcatcgtcaagaaggaggtcggcgagaaccacgacgtgctcctgttcggggtcaacaacagcgtcaagggggacctggccccgcagcacccgatccaggccacggtgcacaaggaggccaagttctgggcggacaagaagcgcttcggggcggaggccatctacgggtccgcgttcaacatccgcaaggacctcgacgcccagatcctctcgaagttccagaggccccccggcgcgctcccctcgtcgatgctcggctacgaggcgctcaccggctccctggacgacttcgggttcgaggactacctcaacctcccccaggactccgactcgttccacgcgcccgacatgcaccacgggatggaggtccgcctagtgtcggacttcgggcggggcttccacccgtcgagcatgctgcaagtgtggggccttttgcagcagccccacccactgtcctaccacgtggtggacctcatctaa

SEQ ID NO: 101 - protein, 163 aa, Oryza sativa
MASGSIVKKEVGENHDVLLFGVNNSVKGDLAPQHPIQATVHKEAKFWADKKRFGAEAIYGSAFNIRKDLDAQILSKFQRPPGALPSSMLGYEALTGSLDDFGFEDYLNLPQDSDSFHAPDMHHGMEVRLVSDFGRGFHPSSMLQVWGLLQQPHPLSYHVVDLI

SEQ ID NO: 102 - DNA, 2124 bp, synthetic construct
  source 1..2124: mol_type = other DNA
atggccgccgccgccgcgctctccgcggtggcgccggggcagaggccggccgactgcccgtccgagtgcggcggggtggacatcccgtacccgttcggcgtggacaactgctcctggccgggcccggacgatttcaccatcatctgcaagtacagccgcccgtactacaggggggccgagatcgtcaacatttccgtggaggccggggagatgagggtctacagcccggtggtgtcccagtgctacaacagctccaacaccaccgacagcgacggcttcgagttcctcaggctgaacatcaccaacacgccattcctggtggccccggagaggaacgagttcacggccatcggctgtgcgaccctggcctggctgtgggggagggacgacggctcatacctgaccggctgtatcagcacgtgcgcgtccctggcgaccgccgcgaaggaccgcgacccctgcaccggcctgggctgctgccaggtgccgtccatcccggcgaacctctccgtgctgaacatctccctggggaccgggatcgcgaacgtggcctgggaggagagcccgtgctcctacgccttcgtggccgagaagcactggtacaacttcaataggcaggacttcgagcgcgacggcaagtccttcgagcacagggacgagaagatggtggtgccgaccgtgctggactgggccatcaggaagaacgggtcctgcccatccaccggccagggggccccggcctgcaagagcgagcacagcgagtgcgtcaacgccacgaacggcaagggctacctgtgcaactgctccaggggctacgccggcaacccgtacagggacgacggctgcaagaacatcaacgagtgcaaagagccctccatcacgtgctacgggggctccacgtgccaggacacggacggcagctacgagtgcaagtgccagttcggctaccgcggggacggccgcaagaacgaatcccagaaggggaggtgccagccgatcattccggcggcgatcgccaacgccatcgccatcgtctgcatcgtcatcgtgctgctcggcctgttctggctcccgaagaggtggaagcgccgcgtgttcttcgacaacaacgggggccgcctgctcaaggacatggacatcatcgtgtttaccgagaaggagctgaacaagatcaccaataagaagcgcaccaagatcggcgagggcgccttcggcgaggtgtataaggggaaccacaacaaccagccagtggcggtgaaatacagcatcgccaagaatatgacccaaacccactacaaggatgtggtcgagtccatcaaccagaacgtgttccagaccgtgttcaggcagtccaaggtgccgccatccaccccgggccagaacgcggtggtgaacgagatcaaggtgcagctccagatcaggcacccgaacatcgtgcgcctgatcggctgctgcatggagacggaggtcccgatgctcgtgtttgagtttatccccaacgggagcctggagacggtgctgcatggcatcgacaggtgctccctctccctgcagcagcgcctcgacatcgcgatcgggtccgcggaggcgctggcgtacatgcactggcacgggcaccaccagatcatccacggggacatcaagccaggcaacatcctcctcggggacaacctgatgccgaaggtgtccgacttcggctcctccgaactcacgctcaaagtgaagcgcgccgggaagtggaacgtgtacgccgatatgaactacatcgacccggtgtacatcaagacgggcgacttcaccgacaagagcgatgtgtacagcttcggcgtggtgctgctcgagctgatcaccaggaagaaggccaagtacgacgacaggtccctgccggtggagttcgtgagccactacgaggacgaggacaccaggcgcaaaatgtacgaccaggacatgctgccgaccgaggcctcccacccgcactgcatggagtgcctggacaggatggccgatatcgtgctgcgctgcctggagaacgaggtgggcaagcgcccgaccatggccgaagtgctggaggagctgaagaaactgctgccactgctgacgaccacgccggtcgagctcgtgtag

SEQ ID NO: 103 - protein, 707 aa, Oryza sativa
MAAAAALSAVAPGQRPADCPSECGGVDIPYPFGVDNCSWPGPDDFTIICKYSRPYYRGAEIVNISVEAGEMRVYSPVVSQCYNSSNTTDSDGFEFLRLNITNTPFLVAPERNEFTAIGCATLAWLWGRDDGSYLTGCISTCASLATAAKDRDPCTGLGCCQVPSIPANLSVLNISLGTGIANVAWEESPCSYAFVAEKHWYNFNRQDFERDGKSFEHRDEKMVVPTVLDWAIRKNGSCPSTGQGAPACKSEHSECVNATNGKGYLCNCSRGYAGNPYRDDGCKNINECKEPSITCYGGSTCQDTDGSYECKCQFGYRGDGRKNESQKGRCQPIIPAAIANAIAIVCIVIVLLGLFWLPKRWKRRVFFDNNGGRLLKDMDIIVFTEKELNKITNKKRTKIGEGAFGEVYKGNHNNQPVAVKYSIAKNMTQTHYKDVVESINQNVFQTVFRQSKVPPSTPGQNAVVNEIKVQLQIRHPNIVRLIGCCMETEVPMLVFEFIPNGSLETVLHGIDRCSLSLQQRLDIAIGSAEALAYMHWHGHHQIIHGDIKPGNILLGDNLMPKVSDFGSSELTLKVKRAGKWNVYADMNYIDPVYIKTGDFTDKSDVYSFGVVLLELITRKKAKYDDRSLPVEFVSHYEDEDTRRKMYDQDMLPTEASHPHCMECLDRMADIVLRCLENEVGKRPTMAEVLEELKKLLPLLTTTPVELV

SEQ ID NO: 104 - DNA, 435 bp, synthetic construct
  source 1..435: mol_type = other DNA
atggctagtgtctcagctaccatgattagtacctccttcatgccaagaaaaccagctgtgacaagccttaaacccatcccaaacgttggggaagcactgtttgggcttaaatcagcaaatggtggcaaagtcacttgcatggcttcatacaaagtgaaacttatcacacctgacggaccaatagaatttgattgcccagataatgtgtacattcttgatcaagctgaggaagcaggacatgatcttccttattcgtgcagggcaggttcttgctcatcttgtgctggtaaaattgctggtggagctgttgatcaaactgatggcaactttcttgatgatgaccaattagaggagggatgggtgctaacttgtgttgcttatccacagtctgatgttactattgagactcacaaagaggcagaactcgtgggctaa

SEQ ID NO: 105 - protein, 144 aa, synthetic construct
MASVSATMISTSFMPRKPAVTSLKPIPNVGEALFGLKSANGGKVTCMASYKVKLITPDGPIEFDCPDNVYILDQAEEAGHDLPYSCRAGSCSSCAGKIAGGAVDQTDGNFLDDDQLEEGWVLTCVAYPQSDVTIETHKEAELVG

SEQ ID NO: 106 - DNA, 249 bp, synthetic construct; identical to SEQ ID NO: 4
  source 1..249: mol_type = other DNA

SEQ ID NO: 107 - protein, 82 aa, Stellaria media; identical to SEQ ID NO: 5

SEQ ID NO: 108 - DNA, 3906 bp, synthetic construct
  source 1..3906: mol_type = other DNA
atggagatcgccagctcctccggcagcaggaggtacgacgtgttcccctccttccgcggggaggacgtgcgggactccttcctgtcgcacctcctcaaggagctgcgcgggaaggcgatcaccttcatcgacgacgagatcgagcggagcaggtccatcggcccggagctgctgtccgccatcaaggagagcaggatcgcgatcgtgatcttctccaagaactacgcctcctccacctggtgcctgaacgagctggtggagatccacaagtgctacaccaacctgaaccagatggtgatcccaatcttcttccacgtcgacgccagcgaggtgaagaagcagaccggcgagttcggcaaggtgttcgaggaaacctgcaaagccaagtccgaggacgagaagcagtcctggaagcaggccctggccgccgtggccgtgatggccggctacgacctgaggaagtggccgtccgaggccgccatgatcgaggagctggccgaggacgtgctgcggaagaccatgacgccgagcgacgacttcggggacctcgtgggcatcgagaaccacatcgaggccatcaagtccgtgctgtgcctcgagtccaaggaggcccgcatcatggtgggcatctggggccagagcggcatcggcaagtccaccatcggccgcgcgctctacagcaagctctccatccagttccaccacagggcgttcatcacctacaagagcaccagcggcagcgacgtgtccgggatgaagctgcgctgggagaaggagctgctctccgagattctcggccagaaggacatcaagattgagcacttcggcgtggtcgagcagaggctcaagcagcagaaggtgctgatcctgctggacgacgtggactccctggagttcctgaagacgctcgtgggcaaggccgagtggttcggctccggctccaggatcatcgtgattacccaggacaggcagctcctgaaggcccacgagatcgacctgatctacgaggtggagttcccgtccgagcacctcgccctgaccatgctgtgcaggtccgccttcggcaaggactccccaccggacgacttcaaggagctggccttcgaggtggccaagctcgccggcaacctgccgctgggcctgtccgtgctgggctcctccctgaagggcaggaccaaggagtggtggatggagatgatgccgaggttgaggaacgggctgaacggcgacatcatgaagaccctgagggtgagctacgacaggctgcaccagaaggaccaggatatgttcctctacatcgcctgcctgttcaacgggttcgaggtgtcctacgtcaaggacctcctgaaggacaacgtgggcttcaccatgctgacggagaagtccctcatcaggatcacgccggacgggtacatcgagatgcacaacctgctggagaagctgggccgcgagatcgacagggccaagtccaagggcaacccgggcaagaggaggttcctgaccaacttcgaggacatccacgaggtggtgaccgagaagaccggcaccgaaaccctgctgggcatcaggctcccgttcgaggagtacttctccaccaggccgctgctcatcgacaaggagagcttcaaaggcatgcgcaacctccagtacctcgagatcggctactacggggacctcccacagagcctcgtgtacctgccactgaagctgcgcctgctggactgggacgactgcccactgaagtccctgccgtccaccttcaaggccgagtacctggtcaacctcatcatgaagtactccaagctggagaagctgtgggagggcaccctgccgctgggctccctgaaggagatgaacctgaggtactccaacaacctgaaggagatcccggacctgtccctggccatcaacctggagaagctggacctggtgggctgcaagtccctggtgaccctgccgtcctccatccagaatgccaccaagctcatctacctggacatgtccgactgcaagaagctcgaatccttcccgacggacctgaacctggagtccctggagtacctcaacctgacgggctgcccgaacctccgcaacttcccggccatcaagatgggctgctccgacgtggacttcccggagggcaggaacgagatcgtggtggaggactgcttctggaacaaaaacctgccggcgggcctggactacctggactgcctgacgaggtgcatgccgtgcgagttcaggccggaacagctggccttcctgaacgtcagggggtacaagcacgagaagctgtgggaggggatccagagcctgggctccctcgaaggcatggacctcagcgagagcgagaacctgacggagatcccggacctgtccaaggccaccaagctggagagcctcatcctgaacaactgcaagtccctggtgaccctgccgtccaccatcgggaacctgcacaggctcgtgaggctcgagatgaaggagtgtacggggctggaggtgctgccgaccgatgtgaatctgtcctccctggaaaccctggacctgagcggctgcagctccctgaggtccttcccgctgatctccaccaacatcgtctggctgtatctggagaacaccgccatcgaggagatcccgtccaccatcggcaacctgcacaggctggtgaggctcgagatgaaaaagtgtacgggcctggaggtgctgccgaccgacgtgaacctcagctccctcgagacgctcgatctgtccggctgctcctccctgaggtccttcccgctgatctccgagtccatcaagtggctctatctcgaaaataccgccatcgaggagatcccagatctctccaaggccacgaaccttaagaacctgaagctgaacaactgcaaaagcctggtgacgctgccgaccaccattgggaacctccagaagctcgtgtccttcgaaatgaaggagtgcacggggctcgaggtgctgccgatcgacgtgaacctgtccagcctcatgatcctcgatctgtccgggtgcagcagcctgcgcacgttcccgctgatctccactaatatagtctggctctaccttgagaacactgccatcgaggagatcccatccacgataggcaacctgcacaggctggtgaagctggagatgaaggagtgtactggcttggaggtgttgccgaccgacgtgaacctctccagcctgatgatcctcgacctctccggctgctcctccctgcgcaccttcccgctcatcagcaccaggatcgagtgcctctacctgcagaacacggcgatcgaggaggtgccgtgctgcatcgaggacttcaccaggctcaccgtgctgatgatgtactgctgccagaggctgaagaccatctccccgaatatcttccgcctcacgcgcctggagctggccgacttcaccgactgccgcggcgtgatcaaggccctgtccgacgccaccgtggtcgccacgatggaggaccacgtgtcctgtgtgccgctgtccgagaacatcgagtacatctgggacaagctgtaccatctgccgtccaagctgaactttaacgacgtggagttcaagttctgctgcagcaacaggatcaaggagtgcggcgtgcgcctcatgtacgtcagccaggaggagaacaaccagcagaccacaaggtccgagaagcgcatgcgcatgacctccggcacctccgaagaggatatcaatctgccgtacggcctgattgtcgccgacaccggcctggccgccctgaacatggagctgagcctcggccagggcgagccgagctcctccacctccctggagggcgaagccctgtgcgtcgattatatgattacggaggaacaggacaaaggcattccgatcctcttcccggtgagcggcaactga

SEQ ID NO: 109 - protein, 1301 aa, Arabidopsis thaliana
MEIASSSGSRRYDVFPSFRGEDVRDSFLSHLLKELRGKAITFIDDEIERSRSIGPELLSAIKESRIAIVIFSKNYASSTWCLNELVEIHKCYTNLNQMVIPIFFHVDASEVKKQTGEFGKVFEETCKAKSEDEKQSWKQALAAVAVMAGYDLRKWPSEAAMIEELAEDVLRKTMTPSDDFGDLVGIENHIEAIKSVLCLESKEARIMVGIWGQSGIGKSTIGRALYSKLSIQFHHRAFITYKSTSGSDVSGMKLRWEKELLSEILGQKDIKIEHFGVVEQRLKQQKVLILLDDVDSLEFLKTLVGKAEWFGSGSRIIVITQDRQLLKAHEIDLIYEVEFPSEHLALTMLCRSAFGKDSPPDDFKELAFEVAKLAGNLPLGLSVLGSSLKGRTKEWWMEMMPRLRNGLNGDIMKTLRVSYDRLHQKDQDMFLYIACLFNGFEVSYVKDLLKDNVGFTMLTEKSLIRITPDGYIEMHNLLEKLGREIDRAKSKGNPGKRRFLTNFEDIHEVVTEKTGTETLLGIRLPFEEYFSTRPLLIDKESFKGMRNLQYLEIGYYGDLPQSLVYLPLKLRLLDWDDCPLKSLPSTFKAEYLVNLIMKYSKLEKLWEGTLPLGSLKEMNLRYSNNLKEIPDLSLAINLEKLDLVGCKSLVTLPSSIQNATKLIYLDMSDCKKLESFPTDLNLESLEYLNLTGCPNLRNFPAIKMGCSDVDFPEGRNEIVVEDCFWNKNLPAGLDYLDCLTRCMPCEFRPEQLAFLNVRGYKHEKLWEGIQSLGSLEGMDLSESENLTEIPDLSKATKLESLILNNCKSLVTLPSTIGNLHRLVRLEMKECTGLEVLPTDVNLSSLETLDLSGCSSLRSFPLISTNIVWLYLENTAIEEIPSTIGNLHRLVRLEMKKCTGLEVLPTDVNLSSLETLDLSGCSSLRSFPLISESIKWLYLENTAIEEIPDLSKATNLKNLKLNNCKSLVTLPTTIGNLQKLVSFEMKECTGLEVLPIDVNLSSLMILDLSGCSSLRTFPLISTNIVWLYLENTAIEEIPSTIGNLHRLVKLEMKECTGLEVLPTDVNLSSLMILDLSGCSSLRTFPLISTRIECLYLQNTAIEEVPCCIEDFTRLTVLMMYCCQRLKTISPNIFRLTRLELADFTDCRGVIKALSDATVVATMEDHVSCVPLSENIEYIWDKLYHLPSKLNFNDVEFKFCCSNRIKECGVRLMYVSQEENNQQTTRSEKRMRMTSGTSEEDINLPYGLIVADTGLAALNMELSLGQGEPSSSTSLEGEALCVDYMITEEQDKGIPILFPVSGN

SEQ ID NO: 110 - DNA, 1422 bp, synthetic construct
  source 1..1422: mol_type = other DNA
atggtccagccgcacgtgctcctggttaccttcccgacccaggggcacatcaacccctccctccaattcgccaaaaagctgatcaagatgggcatcgaggtgacgttcacgaccagcgtgttcgcccacaggaggatggccaagaccgccacctccacggccccgaaggggctgaacctggcggcgttcagcgacggcttcgacgacgggttcaagagcaacgtcgacgactccaagcgctacatgagcgagatcaggagccgcggcagccaaacccttagggacataatactcaagtccagcgacgagggccgcccggtgacgagcctcgtctatacgctcctgctgccgtgggccgccgaggtcgcccgcgagctgcacatcccgagcgccctgctgtggatccagccggccaccgtgctcgacatctactactactacttcaacggctacgaggacgagatgaagtgctccagcaacgacccgaactggtccatacagctgccgcgcctccccctgctgaagtcccaggacctgccgtccttcctggtgtcctcctcgagcaaggacgacaagtacagcttcgccctcccgaccttcaaggagcagctggacaccctggacggcgaggagaatcccaaggtgctggtgaacacgttcgacgccctggagctggagccgctgaaggccatcggcaagtacaacctgatcgggatcggcccgctgatccccagctccttcctgggcggcaaggacagcctggaatccaggttcgggggggacctgttccaaaaaagcaacgacgactacatggagtggctcaacaccaagccgaagagctccatcgtgtacatcagcttcgggtccctcctcaacctgtcccgcaaccagaaggaggagatcgcgaagggcctcatcgagatcaagcgcccgttcctctgggtgatccgcgaccaggagaacatcaaggaggtggagaaggaggaggagaagctgtcctgcatgatggagctggagaagcagggcaagatcgtgccgtggtgtagccagctggaggtgctgacgcacccgagcctggggtgcttcgtcagccactgcgggtggaacagcacccttgagtccctgagcagcggggtcccggtggtcgcctttccgcactggacggaccagggcacgaacgcgaagtggatcgaggatgtgtggaagacgggcgtgcgcatgcgcgtgaacgaggacggcgtggtggagagcgaagagatcaagcggtgcatcgagatcgtcatggatggcggtgagaagggggaggagatgaggaaaaacgcgcagaagtggaaggagctggcgagggaggccgtgaaggaagggggcagctccgaggtgaacctgaaggccttcgtgcaggaggtggggaagagctgctga

SEQ ID NO: 111 - protein, 473 aa, Solanum sogarandinum
MVQPHVLLVTFPTQGHINPSLQFAKKLIKMGIEVTFTTSVFAHRRMAKTATSTAPKGLNLAAFSDGFDDGFKSNVDDSKRYMSEIRSRGSQTLRDIILKSSDEGRPVTSLVYTLLLPWAAEVARELHIPSALLWIQPATVLDIYYYYFNGYEDEMKCSSNDPNWSIQLPRLPLLKSQDLPSFLVSSSSKDDKYSFALPTFKEQLDTLDGEENPKVLVNTFDALELEPLKAIGKYNLIGIGPLIPSSFLGGKDSLESRFGGDLFQKSNDDYMEWLNTKPKSSIVYISFGSLLNLSRNQKEEIAKGLIEIKRPFLWVIRDQENIKEVEKEEEKLSCMMELEKQGKIVPWCSQLEVLTHPSLGCFVSHCGWNSTLESLSSGVPVVAFPHWTDQGTNAKWIEDVWKTGVRMRVNEDGVVESEEIKRCIEIVMDGGEKGEEMRKNAQKWKELAREAVKEGGSSEVNLKAFVQEVGKSC

SEQ ID NO: 112 - DNA, 3396 bp, synthetic construct
  source 1..3396: mol_type = other DNA
atgagcagcctctcccgcgagctggtcttcctgatcctgcagttcctggacgaggagaagttcaaggaaaccgtgcacaagctggagcaggagagcgggttcttcttcaacatgaagtacttcgaggacgaggtgcacaacggcaactgggacgaggtggagaagtacctgtccggcttcaccaaggtggacgacaaccggtacagcatgaagatcttcttcgagatcaggaagcagaagtacctggaggccctggacaagcacgatcgcccgaaggccgtggatatcctggtgaaggacctgaaggtcttctccaccttcaatgaggagctgtttaaggagatcacccagctgctgaccctcgagaatttccgcgagaatgagcagctgtccaagtatggtgacaccaagagcgccagggccattatgctggtggagctgaagaagctgatcgaagccaatccgctgtttagggataagctgcagttcccgaccctgaggaactccaggctgaggaccctgattaaccagtccctgaactggcagcatcaactgtgcaagaacccgaggccgaatccggacatcaaaaccctgttcgtggaccacagctgcggcccgccgaatggggccagggcgccgtccccggtcaacaatccactgctggggggcatcccgaaggccggggggttcccgccactgggggcccacggcccattccagccaaccgcgtccccggtgccaaccccgctggccggctggatgtccagcccgagctccgtgccgcacccggccgtgtccgccggggccatcgccctgggcgggccgtccatcccggccgcgctgaagcacccgaggacgccgccgaccaacgcctccctggactatccgtccgccgattccgagcacgtctccaagaggaccaggccgatgggcatctccgatgaggtgaacctcggcgtgaacatgctgccgatgtccttttccggccaggcccacggtcacagcccagcattcaaagccccggacgacctgccgaagactgtcgcccgcaccctgagccagggctcctccccgatgtccatggattttcacccgatcaagcaaaccctgctgctggtgggcaccaacgtgggcgacatcggcctgtgggaagtggggtccagggaaaggctggtgcagaaaacgttcaaggtgtgggatctgtccaagtgctccatgccactgcaggccgccctggtgaaggaaccggtcgtgtccgtcaaccgcgtgatctggagcccggatggctccctgtttggcgtggcctactccaggcatatcgtgcagttgtactcctaccatggaggcgaagatatgcgccagcacctcgagatcgatgctcacgttgggggtgtcaacgacatatctttttccacgccaaacaagcaactttgcgtgataacgtgtggtgatgataagacgataaaggtctgggatgctgcgacaggcgttaagagacatacgttcgaggggcacgaggcaccggtctactcggtgtgccctcactacaaggaaaacatccaattcattttttccaccgccctggacggcaagatcaaggcctggctgtatgacaacatgggctcccgcgttgactatgacgcgccggggaggtggtgcaccaccatggcctattccgcagatggcacccgcctgttttcctgcggcaccagcaaggacggggagagcttcatagtggagtggaatgagtccgaaggcgccgtcaagcgcacctatcaaggctttcacaaaaggtccctgggcgtggtccagttcgataccacgaaaaacaggtatctcgccgcgggcgacgacttttccatcaagttttgggatatggatgccgtgcaactgctgactgccatcgatggcgacggcggcctgcaggcatccccgaggatcaggttcaacaaggaggggtccctgctggcggtgtccggcaacgaaaatgtgatcaaaatcatggccaatagcgacgggctgaggctcctgcatacctttgagaacatctcctccgaatccagcaagccagccattaacagcatagcggccgcggccgcggccgctgcgaccagcgccggccatgccgacaggagcgccaatgtggtgtccatccagggcatgaatggagatagcaggaatatggttgacgtcaagccagtcatcaccgaggagagcaacgacaaatccaagatatggaaactgaccgaggtgtccgaaccgtcccaatgtaggtccctgaggctcccggagaacctgcgcgtggccaagatctccaggctcatcttcaccaactccggcaacgccatcctcgccctggcctccaatgccatccatctgctgtggaagtggcaaaggaatgagaggaatgccacgggcaaggccaccgccagcctcccgccgcagcagtggcagccggcgtccggcatcctcatgacaaatgacgtggccgagacgaacccggaggaagccgtgccgtgcttcgccctgtccaagaacgattcctacgtgatgagcgccagcggcggcaagatttccctgttcaacatgatgacttttaaaacgatggcaacctttatgccaccgccgccggcggccaccttcctggcgttccacccgcaggataataatatcattgccataggcatggacgattccaccatccagatttataacgtgcgcgtggacgaggtgaaaagcaaactgaagggccattccaagaggattacgggcctggcgttctccaatgtgctgaacgtcctggtgtcctccggcgcggatgcccaactctgcgtctggaatacggacggctgggaaaagcagcgctccaaagtgctgccgctgccgcaggggaggccgaacagcgccccgagcgacaccagggtgcagtttcaccaagaccaggcccacttcctggtcgtgcacgaaacccagcttgccatctatgaaaccaccaagctcgagtgtatgaagcaatgggccgtgagggagtccctggccccgatcacgcatgccaccttttcctgcgatagccagctggtgtatgcctccttcatggacgcgacggtgtgtgtgttctcctccgccaatctgaggctcaggtgccgcgtgaacccatccgcttacctgccggcctccctgtccaattccaatgttcatccgctcgtcatcgccgcccacccgcaggagcccaatatgttcgccgtgggcctttccgacgggggggtgcacatcttcgagccccttgagtccgaaggcaagtggggcgttgcaccgccggcagagaatggctccgcctccggggccccgaccgcgccgagcgtcggcgcgtccgccagcgaccaaccccagcgctga

SEQ ID NO: 113 - protein, 1131 aa, Arabidopsis thaliana
MSSLSRELVFLILQFLDEEKFKETVHKLEQESGFFFNMKYFEDEVHNGNWDEVEKYLSGFTKVDDNRYSMKIFFEIRKQKYLEALDKHDRPKAVDILVKDLKVFSTFNEELFKEITQLLTLENFRENEQLSKYGDTKSARAIMLVELKKLIEANPLFRDKLQFPTLRNSRLRTLINQSLNWQHQLCKNPRPNPDIKTLFVDHSCGPPNGARAPSPVNNPLLGGIPKAGGFPPLGAHGPFQPTASPVPTPLAGWMSSPSSVPHPAVSAGAIALGGPSIPAALKHPRTPPTNASLDYPSADSEHVSKRTRPMGISDEVNLGVNMLPMSFSGQAHGHSPAFKAPDDLPKTVARTLSQGSSPMSMDFHPIKQTLLLVGTNVGDIGLWEVGSRERLVQKTFKVWDLSKCSMPLQAALVKEPVVSVNRVIWSPDGSLFGVAYSRHIVQLYSYHGGEDMRQHLEIDAHVGGVNDISFSTPNKQLCVITCGDDKTIKVWDAATGVKRHTFEGHEAPVYSVCPHYKENIQFIFSTALDGKIKAWLYDNMGSRVDYDAPGRWCTTMAYSADGTRLFSCGTSKDGESFIVEWNESEGAVKRTYQGFHKRSLGVVQFDTTKNRYLAAGDDFSIKFWDMDAVQLLTAIDGDGGLQASPRIRFNKEGSLLAVSGNENVIKIMANSDGLRLLHTFENISSESSKPAINSIAAAAAAAATSAGHADRSANVVSIQGMNGDSRNMVDVKPVITEESNDKSKIWKLTEVSEPSQCRSLRLPENLRVAKISRLIFTNSGNAILALASNAIHLLWKWQRNERNATGKATASLPPQQWQPASGILMTNDVAETNPEEAVPCFALSKNDSYVMSASGGKISLFNMMTFKTMATFMPPPPAATFLAFHPQDNNIIAIGMDDSTIQIYNVRVDEVKSKLKGHSKRITGLAFSNVLNVLVSSGADAQLCVWNTDGWEKQRSKVLPLPQGRPNSAPSDTRVQFHQDQAHFLVVHETQLAIYETTKLECMKQWAVRESLAPITHATFSCDSQLVYASFMDATVCVFSSANLRLRCRVNPSAYLPASLSNSNVHPLVIAAHPQEPNMFAVGLSDGGVHIFEPLESEGKWGVAPPAENGSASGAPTAPSVGASASDQPQR

SEQ ID NO: 114 - DNA, 1785 bp, synthetic construct
  source 1..1785: mol_type = other DNA
atgatcacgagctccagctcggtccgatccctatgctgccctaagacgagcatcatctccggcaaactcctgccgagcctgctcctgaccaacgtgatcaacgtctcgaacggcacgagctcgcgcgcgtgtgtcagcatgagctccctgccggtgtccaagtccaccgcgtcgtcaatcgcagccccgctcgtccgcgacaacggcagcgcgctcaacttcttcccccaggcgccccaggtggagatcgacgagtcgtcccgcatcatggagctcgtcgaggccacccgccgcaccctgaggaacgagagctccgactcgacggagaagatgcggctgatcgactccctccagcggctgggcctgaaccaccacttcgagcaggacatcaaggagatgctgcaggacttcgccaacgagcacaagaacaccaaccaggacctcttcaccacatcgctccggttccgtctcctgcgccacaacgggttcaacgtcacccccgacgtcttcaacaaattcaccgaggagaacgggaagttcaaggagtccctgggcgaggacaccatcgggatcctcagcctctacgaggccagctacctcggcggcaagggggaggagatcctttcggaggctatgaagttctccgagtccaaactgcgcgagtcgtcggggcacgtggcgccccacatccgccggcagatattccagagccttgagctcccgcgccacctccgcatggcgcgcctggagtcgaggcggtacatcgaggaagactactcgaacgagatcggggccgactccagcctgctcgagctggcgaagctcgacttcaactccgtccaggcgctgcaccagatggagctcaccgagatctcgcgctggtggaagcagctggggctctcggacaagctgccgttcgcgcgggaccgtcccctcgagtgcttcttgtggaccgtggggctcctgccggaaccgaagtacagcggctgtcggatcgagctggccaagaccatcgccgtcttgctggtcatcgacgacatcttcgacacgtacggcagctacgaccagctgatcctcttcaccaacgccatccggcggtgggacctcgacgctatggacgagctgcccgagtacatgaagatctgctacatggccctgtacaacacaaccaacgagatctgctacaaggtccttaaggagaacggctggagcgtgctgccgtacctcgagcgcacgtggatcgacatggtggagggcttcatgttggaggccaagtggctgaacagcggcgagcagccgaacttggaggcctacatcgagaacggggtgacgaccgcgggctcctacatggccctggtgcacctcttcttcctcatcggcgacggggtgaacgacgagaacgtgaagctcctactcgacccgtacccgaagctgttctcctcggccggccggatcttgcggctctgggacgacctcggcacggcgaaggaggagcaggagcgcggagacgtgtcctccagcatccagctgtacatgaaggagaagaacgtgcgcagcgagagcgagggccgggagggcatcgtggagatcatctacaacctctggaaggacatgaacggggagctgatcggctccaacgcgctcccccaggcgatcatcgagacgtccttcaacatggcgcggacgagccaggtcgtgtaccagcacgaggacgacacctacttctctagcgtcgacaactacgtgcagagcctcttcttcaccccggtttcagtatctgtttga

SEQ ID NO: 115 - protein, 594 aa, Valeriana officinalis
MITSSSSVRSLCCPKTSIISGKLLPSLLLTNVINVSNGTSSRACVSMSSLPVSKSTASSIAAPLVRDNGSALNFFPQAPQVEIDESSRIMELVEATRRTLRNESSDSTEKMRLIDSLQRLGLNHHFEQDIKEMLQDFANEHKNTNQDLFTTSLRFRLLRHNGFNVTPDVFNKFTEENGKFKESLGEDTIGILSLYEASYLGGKGEEILSEAMKFSESKLRESSGHVAPHIRRQIFQSLELPRHLRMARLESRRYIEEDYSNEIGADSSLLELAKLDFNSVQALHQMELTEISRWWKQLGLSDKLPFARDRPLECFLWTVGLLPEPKYSGCRIELAKTIAVLLVIDDIFDTYGSYDQLILFTNAIRRWDLDAMDELPEYMKICYMALYNTTNEICYKVLKENGWSVLPYLERTWIDMVEGFMLEAKWLNSGEQPNLEAYIENGVTTAGSYMALVHLFFLIGDGVNDENVKLLLDPYPKLFSSAGRILRLWDDLGTAKEEQERGDVSSSIQLYMKEKNVRSESEGREGIVEIIYNLWKDMNGELIGSNALPQAIIETSFNMARTSQVVYQHEDDTYFSSVDNYVQSLFFTPVSVSV

SEQ ID NO: 116 - DNA, 1992 bp, Zea mays
  source 1..1992: mol_type = genomic DNA
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 117 - DNA, 1413 bp, Oryza sativa
  source 1..1413: mol_type = genomic DNA
agctagcatactcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttaaaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaaaaagaaagaaaaaaaagaaaaagaaaaaacagctggtgggtccgggtcgtgggggccggaaacgcgaggaggatcgcgagccagcgacgaggccggccctccctccgcttccaaagaaacgccccccatcgccactatatacataccccccctctcctcccatccccccaaccctaccaccaccaccaccaccacctccacctcctcccccctcgctgccggacgacgagctcctcccccctccccctccgccgccgccgcgccggtaaccaccccgcccctctcctctttctttctccgttttttttttccgtctccgtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaag

SEQ ID NO: 118 - DNA, 1236 bp, Musa acuminata
  source 1..1236: mol_type = genomic DNA
cctcgaaaggaaaaggtcatgatattttatgtatatatgcatacatcatacatatatataatcaacgcatggtcctatcttggtgcattaattatgttccaagtcatggaacttctgttatcccttcccctcctgacttaaatagcatgcatcatgcgaaaagccaagtgttctttgtttgtgcaagcttcaattaatgatccttgtcctgaagctaatcttggcctgaaactggactgtggacacgaatgattgagtgtacgatgcaaggcacattcccagtgcacataaagcaacaagtgccatagggtggtccttcctttggctgcctccacatcttgacttccagaccaagtagccttgcagcataaaatagcggcgcaaaggggggctgttggtgtgcatgagagaggtggcggtggcttaagatttctttcttgctgttgcccccaccgcccaaatcgccctcaggccaccctctccttctctcctttcccgttcctgtttgatcactgcacgcgcactcgatggctgctaaccccccgtctcttcctttctttacattcctcctttttattctttgtccttccatcgccttctgaactttgtatataagcctggacagcctctaaactagcattttctgtctctcactttctctctttctccttctgcacgcgaagaagcctggctgccggtgagttcctcaacatctcagagtaaattacgctgttccgcagagcttactgtggtcgtaccgtctttctcctcctccactgagagcatgtggaaaccatagctattgtaggtatatgcttctctcttgcagccttgggaaatcgaggtgtggtggtcactgaaactgaaagcagccacatcattggcgtgcactcaaagaaacataggcatgattgaaatttatagtcaatttttccatctgctttctatgttatactcactaagatgatcagatctagaacttcgaaatatattccatacgcttgttgaattccaagtatgggatgttcttaattatataaaagatttagaatgggatataatgtaaatagcattaccttcttgcactgatggtgtaccaggtcattttccaatcaattcctcctatttgctttctagattttggggatctctttgatgagggtctaaatcttgtagggcccgctcatcacctcttcatccatcttaacttggtgcgaggttccaggtgaatccaca

SEQ ID NO: 119 - DNA, 2000 bp, Musa acuminata
  source 1..2000: mol_type = genomic DNA
tgatcagcaggctgtactctcacaacacaatgcagcttctattgcatgagtgacagaactatgacaaatgcaaccctgagattcacatgtagatggtaattgattcggatcaacatcacatttaagaagccacctaacttattttgcagatcgaagagatttggatatatagtgagatgataaattacagagttcgagcatcgttgtcatgctttaaagaatcagcattggagagagagagagagagagagagagagtcctctgtgcagaaagttaaacatctacgtaatgggttctcccgagagcaatccaaacagtacgttattcacggagaatccagccttggatttcggacaaatcggggtttggcttctttacaactgacaagttataatcgagtgaactttgggaaccagccgtcaagcttatcgatgcgctttaccactctctctcatcttgtttcttaatgcgattaatagctgtctgtgagtggcaacggagctttggtattaaggaagcaggacctttcttggagctccctcccatatctagtcctcctctgcttgtgtgtctgcatgttcatggactgcgcatttgatctccgtccaccatggatcatcggcatgactagttacgattactggactcgcacaaagagaggcccgtttgcatctgccgttttctatgcccaggaacaacacaaacggatttaatcaatcatgggcagtgaaacgatcagtaactcttcccagtaatattcaagatgtcattagctttctgacaaagccaagattgacattgtgagataaatacattagattttcctcgtatttcacccactgtgggtgtcttctgtattctctctcgtacatgtcgtcgttggtaaaatatgcacaatagcaaaatatgcacttctgacccaacattcaggagtagtagttatcttttatgcactcatttcagatgttttgctgatagtggtttcagttcatcatccgtggaagaaaaaacaaatggagacatcaccaattacgcaccacctagagacaaagatgagctatataattaattaagattcttctggtccagccctcatatcatcaccaaggtggcaacctaattcataagatcaataaatggtttttggtgatctggaaagctaaataagtcttgcatgttgattttggagggatcatcatctgacattaaaaattgtacacctcgtgcggaacctatatgcatgattgggcaactcgagggcggagtttttgtagctggaatactctggacatgagacgggcgacgtcgatgacgccacctcctcaccgcctattaaacgcgagccacaagccaacttaaccgacgcaaaaatccaacaacaaaatctaaactatcgagcccggattggttccgtgtccgggcgccgccgaccgccacttgctcttgagccatccacgtggctctctcttatttccctcgtttcttaagccggtggtcccctttcactcacgtggcgaaacgggctgtgaatagttaccgtcctcgagaaagttgggttgatcacagtgcttcgcctttaatcggtcatatcaccggactcctgcgtctgtgtgagattactgggagcccctagtgtttgccataccctgcttccattacttccatcaatggaagaaacgacacagccgacacaggcatatagcatccctctcacgtaccggcggtggtccagcatctgacttggaatggtactgcgtggtggggccgacaatgggttgatgaactgcgatgatgatccaccgatgaggatttggtcggtcaggggcggcatattataaccctaaacctcaatccatcggtagcaagattgcacggagagaaccagctctcctgctgctactgttcgtcacccatgccccataaatagctccaagtctccctccttccaagcgcacagcttacaagtgctactacttgtcctctggttcgcaactgcatcg

SEQ ID NO: 120 - DNA, 2000 bp, Musa acuminata; identical to SEQ ID NO: 119
  source 1..2000: mol_type = genomic DNA

SEQ ID NO: 121 - DNA, 1070 bp, Musa acuminata
  source 1..1070: mol_type = genomic DNA
aacgtgattttttttcccccttggagggatgtttttgagtaacatttgagaaattttggtagaaccagtggtatctgttaaatttagtgatcgacccttaaagttgtttgtttgtgaaaaccggatgtgatgtgtttgtcggtgcatcgaagcagatggtattcacctccatactgataagatgatgagttgtgagaatgcctgcaggccatttagtttctcttggccctgtggaactttgtatactgcagttaaaatcttcattatctttttattttattgattgcatcgtcttttcttaacctgtagaagtataaatgatgttacgttttgccggcttcacgatttaatttactgttagaatgtatgagacatgatccatttcgttacttaagcatccactcccgaggctctctcagctgcagggtttcctctgccttgttattggcaaccaaggcatctttgttaacccggcgttttggtcccatcagcttaagaaacaagagctcaacttatgctacgcgtatggttgaatctaatagaaatgtgttatagtcatttgtctgaacattaggttttaacgctagagaagagtaggcatcatcgatagtcacataataataattaacgtcaaatcggatgcatttatttatatttggagctggcgactgccatataagaaaacaattgatccaaaaaaacaatgttatcctatcggaccaaatcaaaagtcgacaaccaaaccacaaaacacaaaggattcccaccaacatatacaaagtcgatggcgtgcttatccagaaagctattgtgatgcgggcttgcatcacaaaatatataaataaaatggaagacattatttggactcacacatcacctaaatacgtgcactataaaattataattctagacatggctgatgagaagactgcgatgttattattattattattattatgattacgtggggggagaggaagttgacaatcacaatggcaggtttcgtgaggacggtggatactgcatcattcttggccgagccttctttctctctctatatatagtgccat

SEQ ID NO: 122 - DNA, 1999 bp, Musa acuminata
  source 1..1999: mol_type = genomic DNA
acgctgctactagtttatcattcgcaacgtagctagagagaaccagaggagaggaaagtggtttttattaaagctttcacgtgagtgagaagtttatatgaaattatataaaattaataatttaaaatatagtaaaattattttttatcctttttattcatgttttttttttcatgtgacaatattcctttcgtaagtataaggatcccaatactactttttaaagtttaatgatcaagatattaatcataattaactataagtgataatatataattattctattaaaaatttataaaaataaaagatatttcatgaaaaaaaattttaaaatatgagtttttttttccagaatttgccctacatcaaatccctataaccaaattcaattaatgctagtaactataccattgttacctgtatctatttatatacgctgttgtatatatcggtaaagtggagaaagggatagtcgtgcattcttttgttattatggagtactgatcagtatggactcctaatcaactgaaaagtaaccttatctccgcaagatggatttatcttcgagtatccctaccaagactaagtgagcatgcctaccaagaactaaacaaaagcaacccaacattatggctggacttgatgctggtaccaaataaattctcaattaacaacgtaaagctgcttttgttatctcgcaagcatatgggttttctttaaggcatgatgagctttagagtgctgcagtgttggccttttggcgcgtgatgctctccttgtagacatgcacattacaacgcaggacagttgaggaagagattaatacaatagcaagcaaccaatcatatattaatatgggatgaatgcagccaagcgactttttcgaataactactagccattatggacgattcgccatatctttaggtagataattactcaattaaatcaacattttacaaaataaaataaagtgttgggaaaagatgactagaaaagtaatagacaccataatttattttctctccttgtatagattaaacagaatatattgaagccatgtatcaagatgaaagaaactataatatttaagtcaatcgagtgagcatcacaaactacaagaaaagaaaatttatgtggaaagctcttcaatgtaaagggaaggaccgtaggcaagctcgtgtagattttttttcactaatcagaatcaataaaatatatacaatcatttttaaatcaactagacgatgtaaaaaatcatcaagcaaaattcttgcctcagaaaataattaatgaggtgaatttcaacaacacgcaaagaagaaaaatcaaaactcactaatagagacattagaaaatagcataagagcaggtttctcaagagcaacaccggtcattttgtaaatcaatatgataagatttatatatcaaaaaattatgacgatctaacttgaattgaccatacaattattcaagatgtatttcccccttttttttcctatttgtgttttcgaaatctataagggtcaaagtcttacctatattaaaccctattcatgggcgggactaccccaacaggaagcacatatcaagcagtacgaagggtttaaggtggctaaaggatgtgaggatgcttaacatataggtactctagaacaaattgcgatatatatcaaaaaatgaccttggatgttatttacgtcattgggtaatggaaatcaattaaatggccacataacatctgaggaaatgtgtctctctcaacttccttcaagatatggctattaatatattgtacaaacgcaatctggtaaagattctgttaatccccatcaatagtgtaatcacgtaagcgagttccgtaagcattgtagtggccttgatttgcccactaatggtggaagatggaagccacccatccaacttagtagaattaagtttaactaaaggaagcactaagcgagctctcctctccactcgaaacttgtataaatccgaca

SEQ ID NO: 123 - DNA, 2000 bp, Musa acuminata
  source 1..2000: mol_type = genomic DNA
tggcatgaggtaacaagtttcgtttccacattagtttcattgtgtttgatggtttagaaacatattttgatcttaaattttcttatattttcctgttataagttatcatacttgtacatgatggaagaatatgggatgtttataagcctctgtttcctttttaaaacttttatctttggtttcttcattctgattgtcgttttaatcggagttggatttgctttattattattattattttctaacaatgtgagcgtagtctttaggatgcaaacttgatatggcatgtttctcttggtggatactttagtcgtagcatgattctgattgatatcggaacaacattctaaggcattactcagtacgattattattttatatgagttttcgttccatctgtacggacttagattactaatttccccttttctttatagatataccgtaattccatgtcattttgattttaaaggggaaatggaataggttagaaagatagtgtggaatttatagtattttcatttcttatttgtattatcatcactatagaattgtttgatgtgtctaaggtttaaaatgaatccagatctagtgtttgagcatgttataagttcatcgtttgactgtcaacttaagttacttaagaaaaatataaggagcacatgtgatcttttttataagatgctcgataaccattcaagatgagtaaaaagttaattcatatgataagtctacatttgattattattgacatgatgtcgtaggcttgtaagaaattattcactttatatatgaacatacgattttatctttgtgagtgaataaagataatccgacggatttatactcatcatcacaaaatagttatgctgattgtaaaaatatacattgaaccgtcaagtattataattattaatagttgagttatccacgtaaaggatctattagattttgttttcgccggtctgcatgaagtgatgaacgagcattattcctttttccactgctccaccgctctctcaagctatgatgtcttcctccaaactctaaagaagaaaggaatcgacaagttttagaaacccaactcgacgagaagacaaaaacgagagtatacttaagttggaaggaatcaacaaacccgattcacaagttgtatatgatcgatggtgagcaaatctcgacctttcattaaggagagaaatgctctctacacctaagaagataaaaggaacaatgacaagattaaacaacacaagttggttctgcatgctgctgattcattatgacctttgcttttgcttgatggtttaaaccacaatctgaaacggtccgtcccaccttattgacaatcccacctcctaaaattattgctttgtgatagagaagtgagtttgctttaatgccctctcaatacaataatatatcatcttaattttttatttatatttttttattaaaatcttttcctaatatttctctctctctctctctctctctctcttagcactaacattaattattttatcgattattttatctttaggtataaaatatctgattattcatatatatatatatatatatatatatatcatcgatatttaattgttcataaataaaaatattttttatcaactctcattctttcggtgtataaaattatttattgtgcccatctttaagatactttaacgatggatgacatatttgtacatgcattccatctcggtcaacacctaccgaaataataaacatgtgagacggctactttcatacttgtaaacacacataaagtgtcaattgggctattttaaactaataaacttggtcaaaaaatgaccctaagagcgctacacaacattgacgtgcaaccactcccactgtgcgtgtgactcagccacctaccgcgaaagccttcttcctcctataaagccccacacggacctcatcgtcttccaccacaactcacaactcacaactcacctcaagaagcttcaccc

SEQ ID NO: 124 - DNA, 1253 bp, Musa acuminata
  source 1..1253: mol_type = genomic DNA
cgagacagctcattgacgaacacaaaaggcatattcgacttccgtgtatagctcaagcgacaaaacactgggtttaatctttttattcgacattctctgtgggatccgcaacacgtatcctaaatcccataagtcgagttaatagaaagaccatttaagctgtctcgtcgctgggtagagacacaggagctgcttgttcttttcctttttctctctgtttatctttgatctcgctactgcaaggatacccgcaaaattttcgtgacagaaatttagcgaaatccattcgaggtcttcctatttaaggcccttccactcgcctcgctccccgtctctctcgtcgccgctctgcacctctcactctccatctctcggggaagcgcagaggggcttcggtgtaaggtcagtcgacttcgtcttccgatctgtgttaagcatgtagaatgccatcgattttggtttattttttaggttttcgcgagttctcttgtaggatctacgttgtattagatgcgtataaattgttctattaactggttcagaaaatgattgttgattcgtatcggtacatcgtggatcgagcgaagatttggtaacttattgaccttcggtgttcagcttctctgttcttgccttttctgcctcattatcttgtttcttgattctttaagcttgcgatgataagatattgtgtttggatattgtgtgaaggattgctcttcctttggtttctgattctggaacaatttgattctcccatatattgcttttatattgagttcctctattaatatctgtttgatgatatttttattcatgatggtcgatcttaaaatatgaaaattaccttatgttgattgttcatcagctgtgaagctgaaatttggaaaagaaaatattcaataactgtttttttaagggaaaatatcgaattattacacctcgaaatatgttagttatgaacaatatgaagtggtcaatgattttacctgattaattcttgtcacataatatcaatacttgctttgaaactttcgaaacagtttcgttgtagtttcttgctggacatagttgcagatttctcagttactcttttgagtgttaaaccagcctttcctgtgtttatggaaggattagtgtatataaatagaagattattttgatgttgaattattgggtattaatttgctagagcatgtgggtggaattatatgttcttcctccttcgatttggttttgcagatatccataac

SEQ ID NO: 125 - DNA, 719 bp, Musa acuminata
  source 1..719: mol_type = genomic DNA
aaccccagaggccacagaggaggaagacgatgggatccaccaagaaagcttcagtgctcctcgttctcctcctctcccttctcctaattgcttctggtaggacttctttttcttttggagaactacaaagttagcagtgaatttggacgactaatcaagagataaaagctgttggaatcacttgccgtgaaatcaaaccgagatctacttgcctgttgtcttcctttatgagattggtgaccaaaaatatctgaatgcattgtgatgtaaagtgttgtaacctggaactgctgttgttgggaatgtcatgcagaggaaatggtagcggtggaaggcaggacgtgcgagtcggcgagcaccaggttcaaggggacgtgcgtgaggagcagcaactgcgccagcgtctgccagggcgagggcttccccgacggcaagtgcgagggcgtccgtcgccggtgcatgtgcaggaagccctgctgaagaagcccctccacctgtttctcatcaccgtcgttactacgttggcgtgcatcttaccatgaccttcctggtttctcctttgttttcttgacctactacttgcaggagactacgtttggagtagccaataagaggagtgtcaacactgcgttgtgctctatgtttgtgtctgtggatcgatgtgtgttgttgcttgttttgggtgaatagtagtggtataaaaatctcttttcagtaaa

SEQ ID NO: 126 - protein, 77 aa, synthetic construct
MGSMKKASVLLVLFLSLLLIASEEMVAVEGRTCESASSRFKGTCVRSSNCASVCQGEGFPDGRCEGLRRRCMCRKPC

SEQ ID NO: 127 - protein, 47 aa, Musa acuminata
RTCESASSRFKGTCVRSSNCASVCQGEGFPDGRCEGLRRRCMCRKPC

SEQ ID NO: 128 - DNA, 599 bp, Musa acuminata
  source 1..599: mol_type = genomic DNA
accgatctcatctctctactatcaccgtgtcgcaatggcgtcctccagaagaatgcttcctccactgcttttgctcttgttccttctcatgacctctggttctttctcctcccaggacttcaaactttcatactgcacgaatatcccggacaacatttaatgtatgtcttgacctttgttttgtgatgtagagatggggacgatggtggtgaatggaagaatatgcgagtctcagagccacaagttcgtcggcccgtgcgtgaggaaagccaactgcgggaacgtgtgccaaacggagggcttccacggaggggtttgccgtgggtcgctgatccaccgcaaatgctactgcaccaagaactgttgaacgcctcaaccggtccatcaataccactgtaatggtcttggaagcggcgttggagactgtcatcctgaaactgctaagaataacatcgatgatcgatgagtagaatgtcgtattataacttcgtgctttgattgcatctttaacgtagttttcctccgtatcatgcagcttctgttctgtgagattgtgtggttcaattcatgaataaatgtcctcttaaacgttgctgata

SEQ ID NO: 129 - protein, 79 aa, synthetic construct
MASSRRMLPPLLLLLFLLMTSEMGTTVVNGRTCESQSHKFVGPCVRKANCGNVCQTEGFHGGVCRGSLIHRKCYCTKNC

SEQ ID NO: 130 - protein, 49 aa, Musa acuminata
RTCESQSHKFVGPCVRKANCGNVCQTEGFHGGVCRGSLIHRKCYCTKNC

SEQ ID NO: 131 - DNA, 1732 bp, Musa acuminata
  source 1..1732: mol_type = genomic DNA
cccccactccggagcctgatatcttctcactccaaacggtcctgtcccttccgagggtgtgaggagaatacacaggccgaaatggcgagactcgtgttcgttctctcgttccttctcttcttgctatcaggtgactctctcttcatctctgttgttgtgctctgctttctgtgtctgtaacatttgtgctctgtttttatggtgctaatttgtttcccctgtcgtgaattctattgtgataagtgacattctgtgctctgcttgcaggggctatggctgcaaccttcaccttgacaaacaactgcgagtacaccgtatggcccggcgtgctttcgagcgctggcacggccccgctgtccataacgggattcgcgttgcagcagggcgagtcccgcagcctcgacgtgcccgctgcgtggtccggccgcttctggggccgcaccctctgcgccgccgacgccagcggcaagttcagctgcggcaccggcgactgcgggtccggcgcggtggagtgctccggcagcggtgctgccccccccgccaccctcgcggagttcacgctcgacggcagcggtgggatggacttctacgacgttagccttgtggacggatacaacctgccgatgctggtggtgccgcagggcggctccggcggtgcctgcagctccacgggctgcctggtggaccttaatggggtctgcccgtcggacctcaaggtggtgctcccgacctccgaagggggcagcgaaagcgtggcgtgccggagcgcgtgcgagaccttcgggtcgccgcagtactgctgcagtggcgactacgggaaccccagcacctgcaagccgtcttcctactcccagttcttcaagaacgcgtgcccccgggcgtacagctacgcctatgacgacgccacttccacttttacctgcgcctccgccaactacctcatcaccttctgccccagcaccacgaggtagtttaaccgtctcttctcccttcattcgctcaactacagtatgttgctggcttcaacgtctcagatgtttcatcactgtaatttacagccagaaatcgtcggactcgaatccggaggcggcaggcattccgtcgagcaacaacagtaccatgatctacagcagcgaggaagtcagccacgccacgcccatggtgccgcgcgtggccgcagccctgcagctggccgtcgccctcgccctcgccgttgtcgccttgtgtctcggcttttagcgcggcggccgcgccgaggccactggcgggcccagggttcacatgccctctgccacttctgggacgcctacgactgccaccaatcacattgtaacgtaaccagagcgagaaggagagggagagacgttaccggtggggacgccaagctgccatccatctcccatcgctcagttccctcgtcctctccaacgctcacataagatcatgaacaatactggatctcgctgcttgcctcgtaattggagtcggcgatgaagaccggctctctctttacgcgagtcttgaagctgacatgcgtcggagctgctattgcttgccttctaatcatcttctcagcggttgggtcattctttgatctctctagttagcttttgggttatattttggtttatttgagtcgctttccgggatggatggggacattatccttacggtaggaattatatgtttttctttctttgttgctaagaaccattaaaatatattatttatt

SEQ ID NO: 132 - protein, 303 aa, synthetic construct
MARLVFVLSFLLFLLSGAMAATFTLTNNCEYTVWPGVLSSAGTAPLSITGFALQQGESRSLDVPAAWSGRFWGRTLCAADASGKFSCGTGDCGSGAVECSGSGAAPPATLAEFTLDGSGGMDFYDVSLVDGYNLPMLVVPQGGSGGACSSTGCLVDLNGVCPSDLKVVLPTSEGGSESVACRSACETFGSPQYCCSGDYGNPNTCKPSSYSQFFKNACPRAYSYAYDDATSTFTCASANYLITFCPSTTSQKSSDSNPEAAGIPSSNNSTMIYSSEEVSHATPTVPRVAVALALAVVALCLGF

SEQ ID NO: 133 - protein, 283 aa, Musa acuminata
ATFTLTNNCEYTVWPGVLSSAGTAPLSITGFALQQGESRSLDVPAAWSGRFWGRTLCAADASGKFSCGTGDCGSGAVECSGSGAAPPATLAEFTLDGSGGMDFYDVSLVDGYNLPMLVVPQGGSGGACSSTGCLVDLNGVCPSDLKVVLPTSEGGSESVACRSACETFGSPQYCCSGDYGNPNTCKPSSYSQFFKNACPRAYSYAYDDATSTFTCASANYLITFCPSTTSQKSSDSNPEAAGIPSSNNSTMIYSSEEVSHATPTVPRVAVALALAVVALCLGF

SEQ ID NO: 134 - DNA, 1050 bp, Musa acuminata
  source 1..1050: mol_type = genomic DNA
catggcttccgccgatgcacgcttacccgatctagcggtagtaaccagccatggcttccgcctcttctctgttctcgtccttcgctttgtttcttgtcatgctcatggttcggaggtcctcggctcagtatccagccatgacgctgacggtggtgaacaactgtccgttcacggtgtggccggcgatccagccgaactccggccacgatgtgctcgagcgcggcgggttcgccctcccgacggtgacccaccgctccttcccggcgcctacccaacactggtccggccgcatctgggcccggaccgggtgcacttacgccaacggtcgcttctcctgcgccaccggtgactgcggcggccgcctcgagtgcggaggcctgggcggcgccgcccccgccacgctagcgcaggtcaccctccaccacggcggccaacaggacctcagctcctacggcgtcagcctggtcgacggattcaacgtggggatgacggtgacgccgcacgaggggaaggggcggtgcccggtggtggggtgccgcgagaaccttctcgccacgtgcccggacgtgctgcagctgcgggctccggcgggaggcggccaagtggtggggtgcaagagcgggtgccaggcgttcggcaccgacgagctctgctgccggaacatgtacaacagcccccgcacgtgccgcgcgagcagctactcggagttcttcaagcacgcgtgccccgccaccttcacctacgcccacgacagcccctcgctcacccacgagtgcgccgccccgcgggagctcaaggtcatcttctgtcactgatccagataaaccactactgtagccaacatcttcctttgcttagatctatgactgtagcgactcatgtaccactgtcgtctctctttctctcgtgttccatctctttggttgctcgtttgcttgttaaaccgtgaatcctctttgtgggctagttgcggaatgcatgctcgtgtgttgaggtttaaggctctcccgtgggaagaagaagaagacatcagagctttgtttcttggga

SEQ ID NO: 135 - protein, 254 aa, synthetic construct
MASASCLFSSFALFLVMLMVRRSSAQYPAMTLTVVNNCPFTVWPAIQPNSGHDVLERGGFALPTVTHRSFPAPTQHWSGRIWARTGCTYANGRFSCATGDCGGRLECGGLGGAAPATLAQVTLHHGGQQDLSSYGVSLVDGFNVGMTVTPHEGKGRCPVVGCRENLLATCPDVLQLRAPAGGGQVVGCKSGCQAFGTDELCCRNMYNSPRTCRASSYSEFFKHACPATFTYAHDSPSLTHECAAPRELKVIFCH

SEQ ID NO: 136 - protein, 229 aa, Musa acuminata
QYPAMTLTVVNNCPFTVWPAIQPNSGHDVLERGGFALPTVTHRSFPAPTQHWSGRIWARTGCTYANGRFSCATGDCGGRLECGGLGGAAPATLAQVTLHHGGQQDLSSYGVSLVDGFNVGMTVTPHEGKGRCPVVGCRENLLATCPDVLQLRAPAGGGQVVGCKSGCQAFGTDELCCRNMYNSPRTCRASSYSEFFKHACPATFTYAHDSPSLTHECAAPRELKVIFCH

SEQ ID NO: 137 - DNA, 1449 bp, Musa acuminata
  source 1..1449: mol_type = genomic DNA
agaagtccaccatgtcttttccaagcctcggtgactgtgaccatgaggagatcgctttcgctgcagcagtgtcgatgttgaaggagttcgacgctttgatcatgtgaggagactcccaattaatgtgtatctcttgtacaaatgctacggttttcttcttcgtctatatatgtccgtacctgacacagtcgacgctcaacccaccaaattaagatatgttactctgacgatgcttacctcgagttgctttcaccgtgtcactccattcccatgagcagtaagaccattttggtagtgggagagcgtggtattgttgcctgctctgatacctgacctcattaacaccggtgataagagtgggaggagagtgatcatcgccaccaccgttaacatcgaagtgaacatggattctaggctctggattttgggggttttgctgtcggttctggccaatgccggtccgtcatcggcggcgacgctggcgttgtacaacaagtgcagggagacggtgtggccgggcatacagcctagcgccggcaaggccgtgctggccaggggcggcttccagctcctccccaaccaggcctactccctccggctcccggcgggttggtccggtcgcgtctggggccgcaggggctgcgccttcgacccggccactgggcgcggccgctgcgccaccggcgactgcgggggcgccctctactgcaacggcctcgggggtgcgccgccggccaccctcgcggagttcacgctggggcaggggcggcagcaggacttctacgacgtgagcctggtggacggctacaacctgggcatgtccatgacgccgtaccgggggagcggtcagtgcgggcgcgccggttgcgtcagcgacctcaacgaggtgtgtccggcggggctggctgtgcgcggcggcaccgacaaccgggtagtggcctgcaagagcgcctgctccgccttcggctccccccggtactgctgcaccggcagcttcggcggcccgcagcagtgcaagcccacggcgtactcgaggctgttcaaggcggcgtgcccccgggcgtactcctacgcctacgacgaccccaccagcatcctcacttgcaccggcgccagctacctcgtcaccttctgccctcaacactaaacctcgcctcctccttcctcagacactacaatttctcctgctacatttcatggatgtcatatgatctctggtctaccactggtaggctttgctttctttagcttcttccacttgggttctgcttcacatagtgtgctgccatggacgttcttgtcataacgagaaggcttgcagtctctaatcgcatgcgttctcttagcaactttgcttgtcttgtgaagagcttagtcgactgtttgatgacatttgatgatctgcatttggatattttgatatgataatacccccttggggagctgcat

SEQ ID NO: 138 - protein, 247 aa, synthetic construct
MDSRLWILGVLLSVLANAGPSSAATLALYNKCRETVWPGIQPSAGKAVLARGGFQLLPNQAYSLRLPAGWSGRVWGRRGCAFDPATGRGRCATGDCGGALYCNGLGGAPPATLAEFTLGQGRQQDFYDVSLVDGYNLGMSMTPFRGSGQCGRAGCVSDLNEVCPAGLAVRGGTDNRVVACKSACSAFGSPRYCCTGSFGGPQQCKPTAYSRLFKAACPRAYSYAYDDPTSILTCTGASYLVTFCPQH

SEQ ID NO: 139 - protein, 224 aa, Musa acuminata
ATLALYNKCRETVWPGIQPSAGKAVLARGGFQLLPNQAYSLRLPAGWSGRVWGRRGCAFDPATGRGRCATGDCGGALYCNGLGGAPPATLAEFTLGQGRQQDFYDVSLVDGYNLGMSMTPFRGSGQCGRAGCVSDLNEVCPAGLAVRGGTDNRVVACKSACSAFGSPRYCCTGSFGGPQQCKPTAYSRLFKAACPRAYSYAYDDPTSILTCTGASYLVTFCPQH

SEQ ID NO: 140 - DNA, 1042 bp, Musa acuminata
  source 1..1042: mol_type = genomic DNA
gacaccggcgggattaataatccatcgttgcacgaatacttacgacacggtcgtcgtcgccgttgcattgccgtctttgatcgcccatggcgcgctccggagctccgctagtgcttgtgctggccctcctcctcctcgtcaccgcaccacatgtgaccatagccatcacgtgcggggaggtcacgtccgccctgagctcatgcatctcctacgccaccggcaaggggccgctgtccaccgcttgctgcagcggggtgaagagcctcaacagcgccgcgaagaccacggcggaccgcaagaccgcgtgcacctgcatcaagtccaccgtgtcccgtctctcgggggcccaagccggtgctattgccggcatcccgggcaaatgtggcgtcagcatcggctaccccatcagcgcctccaccgactgctccaagtgagtacatgggattccactccctttattgcttttcttttatgatgcgaagctggaaaaaccaatctgtattattctttttagtattcgtattcattaatatcaaacttaggctgaataatgagagaacaagaaaaagaaaaaggaaagaaaaaaacacacagacatttaggttcaattcacaaggaattattgtccctctgacaccccacagcatcggtattagtctctcctaaaaagaacaagaaactacttcacatttaaacacgtcatccatccctctatctatatataccttaaaagagaaccctctgtaagtcacataaagattttgaagaatcttctgacgtattctagttttgcaaagaatagaccgctaattcagcttgttatattttgtccgcagggtgaactgagagattctcatggacggtggtttatgtagtgagcttatggtatatatatgtcgaataaaggtggagtactgtgacgggcactcctcgtcaccgctctacccctctgtgcttgtgagcctccgccgctagtggggtgctttttgtagcagtgttgtcatagacatatctattgagattacatatcgcttatccatcc

SEQ ID NO: 141 - protein, 117 aa, synthetic construct
MARSGAPLVLVLALLLLVTAPHVTLAITCGQVTSALSSCISYATGKGPLSTACCSGVKSLNSAAKTTADRKTACTCIKSTVSRLSGAQAGAIAGIPGKCGVSIGYPISASTDCSKVK

SEQ ID NO: 142 - protein, 91 aa, Musa acuminata
ITCGQVTSALSSCISYATGKGPLSTACCSGVKSLNSAAKTTADRKTACTCIKSTVSRLSGAQAGAIAGIPGKCGVSIGYPISASTDCSKVK

SEQ ID NO: 143 - DNA, 870 bp, Musa acuminata
  source 1..870: mol_type = genomic DNA
gcaaccccccactccatctgcagtaagctcacaccacgcgtctgcatccggtgctgcacatggctcgctccgctctgcatccggtggccttggtggtggcaacgaccgtggtgctactggcggccgcaccccgtggcggcgaggctataacctgctcccaggtgtacggggacctgatatcgtgcgtggggtacctgcagggcggccccatcaagcagcagtgctgcagtgggatcaagtcgttgatagcggcggcgcggacgacgaaggaccgccgcaccgcgtgcgggtgcatcaagacggccgccgcggggctgtcggggatcgactacgggcgggtcagccaactgcccggccagtgcggcatctccgtttcttacaagatcagccccaacgtggactgctccaagtaagtaacatacatatgtacagacccaccgctcgagtccttctcttcgtgcgctttggtttggttttatgatcatataaacaataaaattgctgtaaaaatcctttttttttttcttacaaaaaaaatcccattaagatcaaaattatatataattgtttgctgtgggcttttctgtgttgcaggataaactgagagaccaaaggttggaagccatcgggcctgtgtttttctcgtttatgcgcgagagaagaacaacaaggagcggtgctcctccatcccatcccatcccatcccattccatcccaaccaataaagaccctttgtttcttcagtgccgtgtgctgctcctctcgagtcggacgtgtacttatgaagcaagctatttagggtgttgaactctgtggtgcttaataatggtgaattatatgaacatcaatacgtgttcttttctttagata

SEQ ID NO: 144 - protein, 119 aa, synthetic construct
MARSALHPVALVVATTVVLLAAAPRGGEAITCSQVYGDLISCVGYLQGGPIKQQCCSGIKSLIAAARTTKDRRTACGCIKTAAAGLSGIDYGRVSQLPGQCGISVSYKISPNVDCSKIN

SEQ ID NO: 145 - protein, 90 aa, Musa acuminata
ITCSQVYGDLISCVGYLQGGPIKQQCCSGIKSLIAAARTTKDRRTACGCIKTAAAGLSGIDYGRVSQLPGQCGISVSYKISPNVDCSKIN

SEQ ID NO: 146 - DNA, 902 bp, Musa acuminata
  source 1..902: mol_type = genomic DNA
atagggttaaaatgactggggttttccttccttgctgtctataaattgatatgcagccccccactacatatgcagtaagctcaagccacccgtctgcatccggtgctgcacatggctcgctccgctctgcatccggtggccttggtggtggcaacgacggtggtgctactggcggccgcaccccgtggtggcgaggctataacctgctcccaggtgtacggggaccttatatcgtgcgtggggtacctgcagggcggcccaatcaagcagcagtgctgtagcgggatcaagtcgttgatagcggcggcgcggacgacgcaggaccgccgcaccgcgtgcgggtgcatcaagacggccgctgcgggtctgtcggggatcgactacgggcgggtcagccaactgcccggccagtgcggcatctccgtttcttacaagatcagccccaacgtggactgctccaagtaagtaacatacatatgtacataccccccgctcgggtccttctcttcgtgcgctttggtttggttttatgatcatataaacaataaaattgctgtaaaacttccttttttttttgcaaaagaaatcccattaagatcaaaattgtatataattatttgctgtgggcttttctgtgttgcaggataaactgagagaccaaaggttggaagccatcgggcctgtgtttttctcgtttatgcgcgagagaagaaaaacaaggagcggtgctcctccatcccatcccaaccaataaagaccctttgtttcttcagtgccgtgtgctggtcctctcgagtcggacgtgtacttatgaagcaagctatttagggtgttgaactcttgtggtgcttaataatggtgaattatatgaacatcaatacgtgttcttttctttagataca

SEQ ID NO: 147 - protein, 119 aa, synthetic construct
MARSALHPVALVVATTVVLLAAAPRGGEAITCSQVYGDLISCVGYLQGGPIKQQCCSGIKSLIAAARTTQDRRTACGCIKTAAAGLSGIDYGRVSQLPGQCGISVSYKISPNVDCSKIN

SEQ ID NO: 148 - protein, 90 aa, Musa acuminata
ITCSQVYGDLISCVGYLQGGPIKQQCCSGIKSLIAAARTTQDRRTACGCIKTAAAGLSGIDYGRVSQLPGQCGISVSYKISPNVDCSKIN

SEQ ID NO: 149 - DNA, 447 bp, Musa acuminata
  source 1..447: mol_type = genomic DNA
agctcgatcctccacttgtgccatcgtctcatctcctcctgctcgctactaattaagcttcttccaagatgaagtcctcggttgtgttcttctgcgtgatcgcggccctgctcctcctccgcgccccgacatcggtggaagcggcgacgtgcaacccaacggagctgacgccctgcgcggcggccatcttcaactcttcgccgcccagcagtctgtgctgctccaagctcaagtctcaggcgccttgcttctgtcagtaccagaagaaccccagcttgagcagatacatcagcgggggacggaaggtggccgcagcttgtgccgtaccagttccaagctgctgactccgctcgtaccctccaaggatcttatatatgcctactatatccatgcatcgtggtcatgaataagaaacaccgtcagcggtcagtgcccgcgtgacactca

SEQ ID NO: 150 - protein, 91 aa, synthetic construct
MKSSVVFFCVIAALLLLRAPTSVEAATCNPTELTPCAAAILKSSPPSSLCCSKLKSQAPCFCQYQKNPSLSRYISGGRKVAAACGVPVPSC

SEQ ID NO: 151 - protein, 66 aa, Musa acuminata
ATCNPTELTPCAAAILKSSPPSSLCCSKLKSQAPCFCQYQKNPSLSRYISGGRKVAAACGVPVPSC

SEQ ID NO: 152 - DNA, 716 bp, Musa acuminata
  source 1..716: mol_type = genomic DNA
aaccccagaggccacagaggaggaagacgatgggattcaccaagaaagcttcagtgctcattctcctcctctcccttctcctaattgcttctggtaggacttctttttcttttggagaactacaaagttagcagtgaatttggacgactaatcaagagataaaagctgttggaatcacttgccgtgaaatcaaaccaagatctacttgcctgttgtcttcctttatgagattggtgaccaaaaatatctgaatgcattgtgatgtaaagtgttgtaacctggaactgctgttgttgggaatgtcatgcagaggaaatggtagcggtggaaggcaggacgtgcgagtcggcgagcaccaggttcaaggggacgtgcgtgaggagcagcaattgcgccagcgtctgccagggcgagggcttccccgacggcaagtgcgagggcgtccgtcgccggtgcatgtgcaggaagccctgctgaagaagcccctccacctgtttctcatcaccgtcgttactacgttggcgtgcatcttaccatgaccttcctggtttctcctttgttttcttgacctactacttgcaggagactacgtttggagtagccaataagaggagtgtcaacactgcgttgtgctctatgtttgtgtctgtggatcgatttgtgttgttgcttcttttgggtgaatcgtagtggtataaaaatctcttttcagtaaa

SEQ ID NO: 153 - protein, 77 aa, synthetic construct
MGFTKKASVLLLLLLSLLLIASEEMVAVEGRTCESASTRFKGTCVRSSNCASVCQGEGFPDGKCEGVRRRCMCRKPC

SEQ ID NO: 154 - protein, 47 aa, Musa acuminata
RTCESASTRFKGTCVRSSNCASVCQGEGFPDGKCEGVRRRCMCRKPC

SEQ ID NO: 155 - DNA, 703 bp, Musa acuminata
  source 1..703: mol_type = genomic DNA
ttggaagagctcagctctctggtggagaagatggcctcctcgaccgccctctccctcctcctcttccctctgctcttcgccctctcccatgcggccaccttcgagatcgtcaaccaatgctcctacaccgtctgggcagccgccgtccccggcggcggccgccagctcgactacggccagagctggaccaacgaagtgaacccgggcactgcaagctgccgcgtctgggcccgcaccggctgctccttcgacagcagcggccatggcagctgcgagaccggcgactgcggcggcctgctcgagtgccaagcctacggctccccgcccaacacgctcgccgagttctcgctcaaccaggacaacaacaacgacttcatcgacatctccctcgtcgacggcttcaacgtgcccatggacttcagccccaccaccgggggctgccatgggatccggtgcgccgccgacatcaacgggcagtgccccagcaagctgaaagcgccgggcggctgcaacaacccctgcaccgtgttcaagaccgacgagtactgctgcaactccggcagctgcggcccgaccaactactccatgttcttcaagaatctgtgcccggatgcttacagctaccctaaggatgacgccaccagcgtcttcacctgccccggcgggacgaactacaaggtggtcttctgccccacgtcgtagg

SEQ ID NO: 156 - protein, 223 aa, synthetic construct
MASSTALSLLLFPLLFALSHAATFEIANQCSDTVWAAAVPGGGRQLDSGQSWTNEVSPGTAGCRVWARTGCSFDGSGHGSCETGDCGGLLECQAYGSPPNTLAEFSLNQDNNNDFIDISLVDGFNVPMDFSPTTGGCHGIRCAADINGQCPSELKAPGGCNNPCTVFKTEEYCCNSGSCGPTNYSMFFKNLCPDAYSYPQDGPTSLFTCPGGTNYRVVFCPTS

SEQ ID NO: 157 - protein, 202 aa, Musa acuminata
ATFEIANQCSDTVWAAAVPGGGRQLDSGQSWTNEVSPGTAGCRVWARTGCSFDGSGHGSCETGDCGGLLECQAYGSPPNTLAEFSLNQDNNNDFIDISLVDGFNVPMDFSPTTGGCHGIRCAADINGQCPSELKAPGGCNNPCTVFKTEEYCCNSGSCGPTNYSMFFKNLCPDAYSYPQDGPTSLFTCPGGTNYRVVFCPTS

SEQ ID NO: 158 - DNA, 1633 bp, Musa acuminata
  source 1..1633: mol_type = genomic DNA
tcccagccgaagagacaaacacaggcaaaaaaggtgtgagggaattgatgatggaggtgtgtcggtgaagggtggtggcaatagcggtggagaagctgctctccgccttataaatgcacagctcgggttgcaacggctttcttgtgggttcgtctgtaatggagagatctactcaggctcttctcttcctgatcctcctcgtttgctgtgctacaatggcagcatcagccaaggttaaatctcccccccccttcctctctctctttctctccttttgtagattacacagcaacggcatctgatgaaggctgcaattttgcctatgcgtttgacatgtatgtaggatctcccagctgaggagttcggtgtgagcttggcagaaaaaagggtcggtaaaatagcaacttagttgtcattccatataatgtttgcattgtcgaagggagtgagagattctgtgtataaggcctaactggtgtcttttcacttggttggcagcataaacataagcattctgggttttctgaagcaggtatggttttagaaatggcagctatgtttctgattgttctctgttcttacaaactgctgcatcagatgcaacatgtttgatccatttatgaagtgcttgtacttgcatttgttagaactggccttaaatgtcctgagctactttgatcttatgccaaccctgtcaggttaacatgaaaccttcaatggcaactggagaacagttccattgtttcatggagtatcttctcctgtattttcatcaaccatcatgcatgattctatcacatcaaccaaaagacaaatctgtgatgacagagatagatccagtcttggtaagcaaattgactagagagaaagttaataggaatggaaagctacaagtacatcaacaatggatttctccttgccatcacatctttttggtcaacgctaaataagagtgttctgctcatcaacaacatatatctccttgcttggctgacttaatctaatccgatgacaatacttttgtgaagttcttatagaaatatagagaggaaaaatctttgttatgttgcaacttatattagtcataagctctaccaggaaaagaaaattaaatctattaatgcttgtaattagcgatgtttgccagattggcaatctctcattttctcatccttggatatgattttgcagagtgcccaggtgcctgtcaatatagatgctccaagactgcctacaagaagccctgcttgttcttctgccagaagtgctgcttcaagtgcaggtgtgttcctccaggaacctatgcacacaaggaggtttgcccatgctacaataactggaagaccaagagaggaggtcccaagtgcccatgagctcctctgcttgaagatttcagacttcttctctaaatatattatgattattgaagtagacaagcttatttctaccaatgtatgtccttaattgtgtgacagcatcagtaacataattgttctaagccctgatactaggctgctggtgcaactctaatgagattagatcttcaaatctgtggctaccatttgtattggtttaatattgtcaccatctgaagatatctgatccatttaaattctatgttcagttcattcaa

SEQ ID NO: 159 - protein, 126 aa, synthetic construct
MHSSGCNGFLVGSSVMERSTQALLFLILLVCCAAMAASAKDLPAEEFGVSLAEKGHKHKHSGFSEAECPGACQYRCSKTAYKKPCLFFCQKCCFKCRCVPPGTYAHKEVCPCYNNWKTKRGGPKCP

SEQ ID NO: 160 - protein, 87 aa, Musa acuminata
KDLPAEEFGVSLAEKGHKHKHSGFSEAECPGACQYRCSKTAYKKPCLFFCQKCCFKCRCVPPGTYAHKEVCPCYNNWKTKRGGPKCP

SEQ ID NO: 161 - DNA, 595 bp, Musa acuminata
  source 1..595: mol_type = genomic DNA
ctctccacgctgacaccccatccacgcttctctctctgttttcccctccgatctttcttactatctcgcttgaagatgaagctcttctctgccgccaccctcctcctgctgctcgttttgacttcctcctacctccgtggttccatggctgcagactcaggtaaacacttgtacccgtcgccatggcaagatcctcgtaatgtgtcatcaagatggcattgacgcgtgttgtttcttggatgtcgtttttctgtgtcagcctattgcaaatccaagtgcaaggtgaggtgcgccaaggccggagtgatggaccggtgcctgtactactgcggaatgtgctgcgaggagtgcaagtgtgtgccttccgggacttacgggaacaaggatgagtgcccttgctacagggataaggtcaccaaggacgacaagaagaagtccaagtgcccatgactggtctctcttagcgaaagcactcaactcattgtactcttctctttttttcgtggggaatgtactggtacgagaagatgtcatcctttgtctggaataaagtcatttgcaatccaggaaaactaagaacaagaagattgatcac

SEQ ID NO: 162 - protein, 91 aa, synthetic construct
MKLFSAATLLLLLVLTSSYLRGSMAADSAYCKSKCKVRCAKAGVMDRCLYYCGMCCEECKCVPSGTYGNKDECPCYRDKVTKDDKKKSKCP

SEQ ID NO: 163 - protein, 66 aa, Musa acuminata
ADSAYCKSKCKVRCAKAGVMDRCLYYCGMCCEECKCVPSGTYGNKDECPCYRDKVTKDDKKKSKCP

SEQ ID NO: 164 - DNA, 902 bp, Musa acuminata
  source 1..902: mol_type = genomic DNA
ccttggtctgttatacttcctctctcatggcgaagtcttccatcgcctgcgcagtgcttctcctgttacttctggtggaggtgagtccatgtgtgtgattataacatatacttgggtggggataatccacaatttaaaggaacttgcaatctcttttaagacttgcagcaaaattatgaacatggaaaactgatgtagccaaaaattgagaacattttttgtttgctttgggtatatatacaagcgcaacaaaatttgtctactgcactctgaagccggatctatgaagtcatgattggtgttaacatctgtggtagtgtttgggtgtctctgctctttgattgagaactggagaataatgttgccgttcaaatgcactttattatgtgattctgcacttgtaatacttacctctctcgtgctgtgttctgcagatggatatctcagctgtcgcagaaagagaaatcagtaagtcaatgcacatttctagcatatagagagatcatcaagtaacacaatcagatgatcagtgacctcgaatcacggttggtattgctgcagattgtgaggcgaagtgttcgcaccgttgctccaaggcatcaaggcacaagatgtgcatgagagcttgcatcgactgctgtcacaagtgccactgcgtcccgcccggtacatacggccacaaagaggaatgcccctgttatgctcacatgaagactcgccatggtaaacccaaatgcccttagaagctaaacccacataatcctataactagcaatattgctctctaatctcgaaataaaagaaccgtactgttctatgtgctacttactgagtgccacttcaatgtgtgatcctaatctgtagccctaaataattagtaaagtctggtcttatatactcta

SEQ ID NO: 165 - protein, 89 aa, synthetic construct
MAKSSIACAVLLLLLLVEMDISAVAEREINCEAKCSHRCSKASRHKMCMRACVDCCHKCHCVPPGTYGHKEECPCYAHMKTRHGKPKCP

SEQ ID NO: 166 - protein, 64 aa, Musa acuminata
EREINCEAKCSHRCSKASRHKMCMRACVDCCHKCHCVPPGTYGHKEECPCYAHMKTRHGKPKCP

SEQ ID NO: 167 - DNA, 225 bp, Musa acuminata
  source 1..225: mol_type = genomic DNA
atgaagacagaagtttctgtggtgttaagttccagcttaatcatatgcagttttgatgcagaggaggagggcgtggaggcgaagatctgcaggttgcccagtataggtttcagagggccatgcacgtccgataagaactgtgccaaggtttgccggagggaaggcttcgacagcggcgactgcgaggggtacaccatgggatgcctctgcaacaagcactgctga

SEQ ID NO: 168 - protein, 74 aa, synthetic construct
MKTEVSVVLSSSLIICSFDAEEESVEAKICRLPSIGFRGPCTSDKNCAKVCRREGFDSGDCEGYTMGCLCNKHC

SEQ ID NO: 169 - protein, 47 aa, Musa acuminata
KICRLPSIGFRGPCTSDKNCAKVCRREGFDSGDCEGYTMGCLCNKHC

SEQ ID NO: 170 - DNA, 933 bp, Musa acuminata
  source 1..933: mol_type = genomic DNA
ccctcggatctgcacaacacagagcacgaagcctaagactcgcagttagatggcttcgtccagcattctctccctctccttcttcttcttcttcttcttctcccttcttgtcaccctctcccatgccgccaccttcgagatcgtgaaccgatgctcttacacagtgtgggccgcggccgtgcctggcggcggacgccagctcaaccaggggcagtcgtggaccatcaacgtgaacgcgggaaccaccggcggccgcatctggggccgcaccggctgctccttcgacgggagcggacgcgggcggtgccagacgggcgactgcggcggcgtgctgtcatgcacggcgtacggcaacccgcccaacaccctggcggagttcgcgctcaaccagttcaacaacctcgacttcttcgacatctccctggtcgacggcttcaacgtgccaatggacttcagccccacgtcaggcggctgccgcggcatccggtgcgcggcggacatcaacgggcagtgccccggggcgctgaaggcgccgggcggctgcaacaacccctgcaccgtgttcaagacggaccagtactgttgcaactccggcagctgcagcccgaccgactactcccggttcttcaagagaaactgccccgacgcctacagctatcccaaggacgatcagacgagcaccttcacctgccccggcggcaccaactacagggttgtcttctgcccttgagatcactgacgccactaataagccgccctcgtgcaggcaaccacgcatatagatatatatactagtctcctacaatacaaataagatgtcaagagtgatggaaggtattagccttctcttgggctaatactgttgagtgatgtaatagaagtggtgcaaccaactaataagatgcataatatcatcacagcatgagtgctttt

SEQ ID NO: 171 - protein, 226 aa, synthetic construct; identical to SEQ ID NO: 67

SEQ ID NO: 172 - protein, 199 aa, Musa acuminata
TFEIVNRCSYTVWAAAVPGGGRQLNQGQSWTINVNAGTTGGRIWGRTGCSFDGSGRGRCQTGDCGGVLSCTAYGSPPNTLAEFALNQFNNLDFFDISLVDGFNVPMDFSPTSGGCRGIRCAADINGQCPGALKAPGGCNNPCTVFKTDQYCCNSGSCSPTDYSRFFKRNCPDAYSYPKDDQTSTFTCPGGTNYRVVFCP

SEQ ID NO: 173 - DNA, 773 bp, Musa acuminata
  source 1..773: mol_type = genomic DNA
tcggatctgcacaacacagagcacgaagcctaagactcgccgttagatggcttcgtcaagcattctctccctctccttcttctcccttcttgtcaccctctcccatgccgccaccttcgagatcgtgaaccgatgctctgacacagtgtgggccgcggccgtgcccggcggcggacgccagctcaaccacgggcagtcgtggaccatcaacgtgaaagcgggaacgaccggcggccgcatctggggccgcaccggctgctccttcgacgggagcggacgcgggcggtgccagacgggcgactgcggcggcgtgctgtcatgcacggcgtacggtagcccgcccaacaccctggcggagttcgcgctcaaccagtacagcaaccacgactacgtggacatctccctggtcgacggcttcaacgtgcctatggacttcagccccacgtcgggcggctgccgcggcatgcggtgcacggcggacatcaacaagaagtgccccacggcactgaaggcgccgggcggctgcaacaacccctgcaccgtgttcaagacggaccagtactgctgcaactccggcagctgccagccgaccgagtactccaagttcttcaagagcaactgccccgacgcctacagctatccgaaggacgatccgacgagcctcgtcgcctgccccggcggcaccaactacagggttgtcttctgcccttgagatcaccgacgccactaataagcctccctcgtgtaggcaacaacgcagatatatatatata

SEQ ID NO: 174 - protein, 221 aa, synthetic construct
MASSSILSLSFFSLLVTLSHAANFEIVNRCSYTVWAAAVPGGGRQLNQGQSWTINVKAGTTGGRIWGRTGCSFDGSGHGRCQTGDCGGLLACTAYGSPPNTLAEFALNQYSNQDYVDISLVDGFNVPMDFSPTSGGCRGMRCTADINKKCPTALKAPGGCNNPCTVFKTDQYCCNSGSCQPTEYSKFFKSNCPDAYSYPKDDPTSLVACPGGTNYRVVFCP

SEQ ID NO: 175 - protein, 199 aa, Musa acuminata
NFEIVNRCSYTVWAAAVPGGGRQLNQGQSWTINVKAGTTGGRIWGRTGCSFDGSGHGRCQTGDCGGLLACTAYGSPPNTLAEFALNQYSNQDYVDISLVDGFNVPMDFSPTSGGCRGMRCTADINKKCPTALKAPGGCNNPCTVFKTDQYCCNSGSCQPTEYSKFFKSNCPDAYSYPKDDPTSLVACPGGTNYRVVFCP

SEQ ID NO: 176 - DNA, 961 bp, Musa acuminata
  source 1..961: mol_type = genomic DNA
tggccttctctggtgcagcaatggcatcgctcggcggcctctccttcctcctcctccccctgctcttcgtcgtctcccatgcagctaccttcgacatcgtcaaccaatgctccttcactgtctgggccgccgccgtccccggaggcggccgccagctcgacccgagccagacctggaccatcaacgtcaatcccggcaccacaggcggccgcgtctgggcccgcaccgactgctccttcgatggaagcggcagcggtagctgccagacaggcgactgcggcggcctcctggagtgccaaggctacggcacgccgcccaatacgctggccgagttcgccctcaaccagttccagaacttggacttcgtcgacatctccctcgtcgacggcttcaacgtgcccatggacttcagccccacctcgggggactgccggggcatacggtgctccgccgacatcaacgggcagtgccccgcggatctgagggcgccgggcggctgcaacaacccttgcaccgtgttcaaaactgatgagtactgctgcacctccggcagctgcgggccgacaaactactccatgttcttcaagaacaactgccccgatgcttacagctaccccaaggacgacgcaaccagcaccttcacctgccccggtgggactaattacagggttgtcttctgcccttaattatcacgtgggaggagaaggaggtgacggcggacgtacaaagaatctactcgtagataaaagaataataagccaattacccagtaataacaacgaataagagggtgtctgtattgacatccgaattgatgagtgctttgttgcacaagtgatatatctcatatccctatgttgtacgtcatttgatgttatgaaagtttctatagaagtcgacaataggatctcttgttggcatcagactcgagaaaaaaataatatcacaacatttagatctt

SEQ ID NO: 177 - protein, 221 aa, synthetic construct
MASLGGLSFLLLPLLFVVSHAATFDIVNQCSFTVWAAAVPGGGRQLDPSQTWSINVNPGTTGGRVWARTDCSFDGSGSGSCQTGDCGGLLECQGYGTPPNTLAEFALNQFQNMDFIDISLVDGFNVPMDFSPTSGDCRGIRCSADINGQCPAELRAPGGCNNPCTVFKTDEYCCTSGSCGPTNYSMFFKNNCPDAYSYPKDDATSTFTCPGGTNYRVVFCP

SEQ ID NO: 178 - protein, 200 aa, Musa acuminata
ATFDIVNQCSFTVWAAAVPGGGRQLDPSQTWSINVNPGTTGGRVWARTDCSFDGSGSGSCQTGDCGGLLECQGYGTPPNTLAEFALNQFQNMDFIDISLVDGFNVPMDFSPTSGDCRGIRCSADINGQCPAELRAPGGCNNPCTVFKTDEYCCTSGSCGPTNYSMFFKNNCPDAYSYPKDDATSTFTCPGGTNYRVVFCP

SEQ ID NO: 179 - DNA, 732 bp, Musa acuminata
  source 1..732: mol_type = genomic DNA
cctatataagcagcggcagctcttctcatcttgctcaccacctaaccggcacggtactgctacaccatggctcttaagctggccatgtttgtctttgcatctctactcgtcatcaccaccagagtaagccagccatctgagttctactcttcggacgaagggaaccgcgcgtagcgcgtctgatgaacacgtgttcgatgttgcaggtctcgtccgacgcaaaggacgcgttcatggaggcgggctatgccaaggctcctgtggtcgctcctgttccggctcctcccgcacctcgcatcatcaaggacaccaaaggtgaggagctcttgtcgtttggttctgcgtaggtcattggcaagcgtcatcgacgtgctttgctggtggtgcagagtgcggcgacgcttgcaaggagcggtgcaagctgcactcgcggcagaacgtgtgctctcgagcctgcatcacctgctgcagcttctgcaagtgcgtgccgccggggacctatggccacacggagctgtgcggcaagtgctataccgactggaagacgcacgggaaccggaccaagtgcccctgaacgccggcaccgtctgctgcagctccgcctttctttccgcgaccaagaagctagaagatcactctgttcttttggctctgttttgatcgagacacgagtgttcgtctcatgttttcgttggcctggaggagaataaatcgtgttgtgttcatgctcta

SEQ ID NO: 180 - protein, 116 aa, synthetic construct
MALKLAVFVFASLLVITTRVSSDAKDAFMEAGYAKAPVVAPVPAPPAPRIIKDTKECGDACKDRCKLHSRQNVCSRACITCCSVCKCVPPGTYGHTELCGKCYTDWKTHGNRTKCP

SEQ ID NO: 181 - protein, 92 aa, Musa acuminata
KDAFMEAGYAKAPVVAPVPAPPAPRIIKDTKECGDACKDRCKLHSRQNVCSRACITCCSVCKCVPPGTYGHTELCGKCYTDWKTHGNRTKCP

SEQ ID NO: 182 - DNA, 779 bp, Musa acuminata
  source 1..779: mol_type = genomic DNA
cttcatatgcatctaacttcccctgctctctctcactctctcttcggctatggaggattctcggagaatgcttccagccatcgttctgctcttcctgctactgatctcctctggtgctctcctgttctgctttcttctccccatacatgttgcagttaggtgttatcaggatccaactagaacagtaaaacacatagcatgttagcagtctgaaactaatagaaacaaggattctaagcaataagacatgcaagtatcaagcaggcccatgtaactctcttatttcttaatatattttccataaaagtcaaggttctgttgcatcaaggagtcttcttgcttctagtaaactgttgtgtgagtgttcatatactaataatgcttttattctcactgtggaagacatggggatgacatcagtggaagcgaggaagtgcgagtcggcgagccacaagttcaaggggctctgctttcgggccagcaactgtgcaaatgtctgcaagacagaaggcttccatggaggccattgcaggggcctccgtcgccgatgcttctgcaccaagcattgcaactctgctacatgagcacaacatggtgaaaatgatgtcgatgatggtggctaggttattggtggtgtctcgacaataaattatgttaagagtccatcttcctttcttatgccaagagtttgctgttaaagctgccttgatcttgtccatggtttccactttgtttgaatcttggatcttcatgagaataaagtcttgtggttactatca

SEQ ID NO: 183 - protein, 81 aa, synthetic construct
MEDSRRMLPAIVLLFLLLITSDMGMTSVEARKCESASHKFKGLCFRASNCANVCKTEGFHGGHCRGLRRRCFCTKHCNSAT

SEQ ID NO: 184 - protein, 51 aa, Musa acuminata
RKCESASHKFKGLCFRASNCANVCKTEGFHGGHCRGLRRRCFCTKHCNSAT

SEQ ID NO: 185 - DNA, 1051 bp, Musa acuminata
  source 1..1051: mol_type = genomic DNA
aacctgaaggaggaggctaccttatcactttcccttgtcatctctactagcttgtagccatgacagtggaaaacagagtggccattcttctatgcatggtggtgctgcattgtttggtcatccaagcccaggtctttccctctctctctctctctctctcactctcacacacacacacacacatacacacattttaggtttatgattgttttggtggatactgtaatggtagttcaacatgacagtgaatcttttgggtctgctaaggatcaaaaacgaaacaccattgacatgattaacctgtcacttaaagaagttgattgacacttgaaacatagctgcagtagtttacaggtggtgacgcgaaggttcttattggtggtgcagggtaggatttctctggctcccactgcccaaccgggaaatcctctcggcccaactcctcagccgatcaaattccccatggtaaagccaaacatttctgttaatctatggtccaatcactgccattgctgttgttccagtgaatgggtgcatactaaacttggacagccttgaactgattgcagtatggagccacaccagggagcctccaacctcaaggtacggaagagtctccccttcttgattccagcttcagcctacttgcaatgccaagtcatttctgtcactaatgccgcagaatgctcgggccgctgcgccggtcggtgctccgccaccgcctacaagaagccctgcatgttcttctgccagaagtgctgcgccaagtgcctctgcgtgccgcccgggacgtatggcaacaagcaagtctgcccctgctacaacaactggaagacgaagagaggcggtcccaagtgcccctgagatgacatatgcccgaatgctgtgcacccactgcttctgagtctgtatgtgctttgcatgcctccgtgggtaataaaggtggctttgagactgacgagtgttcatgcaacagtacttcaatgaggagacctccaccatgaacttctcatccagttaatcttgcttatgaactgatatttgatcctac

SEQ ID NO: 186 - protein, 121 aa, synthetic construct
MTVENRVAILLCMVVLHCLVIQAQGRISLAPTAQPGNPLGPTPQPIKFPMYGATPGSLQPQECSGRCAGRCSATAYKKPCMFFCQKCCAKCLCVPPGTYGNKQVCPCYNNWKTKRGGPKCP

SEQ ID NO: 187 - protein, 96 aa, Musa acuminata
RISLAPTAQPGNPLGPTPQPIKFPMYGATPGSLQPQECSGRCAGRCSATAYKKPCMFFCQKCCAKCLCVPPGTYGNKQVCPCYNNWKTKRGGPKCP

SEQ ID NO: 188 - DNA, 1326 bp, Musa acuminata
  source 1..1326: mol_type = genomic DNA
ttcacttcggctcatcatcgcttctctcttccaccaactcgccatggctatgcgttggaggcagcttctctacaccgtgcttctcttctgtgttctggttgatcaggtaacaccctccctcttcgttggatcagatctagtctcggaattttccttcctgctcataatccattcgaaatgcttgcaggctcatttgcgcaaggcggtcgttgaggatgaacctgtaatctaacttgcatgagctacttccatgtcttcttcttcttcttcttcttccatcctctgttttgatctaactgttgtagtggcatcgtttcttgcattccacagcctgtttcaggaggaggggatggaaagccgccggttcccgtgataggtgagcactaatcctcttagccttttgcttccttgatcaccatactgctgcatgactcccatttccgaagaaattagtgtaatatgatgctgatttggttggattttgagtcgatgacaagaagaaggcaacatattcgacgaatcacacaaagaggtgaaaatggttctgagaaccataaaagagcctcaaactaaaacgttcatggtagcattggatgctgaaacgttcacgtggatgtcatcccactgagaaggcaaaagcgttaaagtggccgaggaaaaaggccacccaaagcagagaacacctataataaatatacgatggcagaacgggccatgggttggtcaccatcatcatcatttacctttcaccactacggtctcttcctttaagagaacgtacgtatctttagcagccgtgtcttcttgcagacccatctgccaaaactggacgcgtacgtttcctttgagctgtgcttactatctagttggagaagccaagaagaagcactgcagctcttgtcgagagctctccatctgttggtaatggttgtgtcggctttacacagtgcatgagaatgacttgcagattgcgccgggctgtgcggggtgagatgcagccggcactcgaggccgaacctgtgcaagcgggcgtgcgggacgtgctgcttcaggtgcaagtgcgtgccgccgggaacctccggcaaccgcgagatgtgtggcgcctgctacaccaacatgaccacccacggcaatcgcaccaagtgcccctagttgcagacccacctcctcctcggagccgcgtgtctcttccatggtctcgtctcctatgtagtgcacaaagctgtgtgtctctcatcgtctcttgctgtttgcgtctgcatctgtgtctttgcatgcatctatgtgagatactagctacgtgtgagcgttaataatgcacttccc

SEQ ID NO: 189 - protein, 109 aa, synthetic construct
MAMRWRQLLFAVLLFCVLVDQAHLRKAVVEDEPPVSGGGDGKPPVPVIDCAGLCGVRCSRHSRPNLCKRACGTCCFRCKCVPPGTSGNREMCGACYTNMTTHGNRTKCP

SEQ ID NO: 190 - protein, 85 aa, Musa acuminata
RKAVVEDEPPVSGGGDGKPPVPVIDCAGLCGVRCSRHSRPNLCKRACGTCCFRCKCVPPGTSGNREMCGACYTNMTTHGNRTKCP

SEQ ID NO: 191 - DNA, 505 bp, Musa acuminata
  source 1..505: mol_type = genomic DNA
tcctcatcatctgacctaatcacagtgatcgcacacagcatgaagccgatactgctgctctttctcgtctccgcgctcctcttatccgcggcggcgccgacggcgtcggtaacctgcaaccccctggagctcagcccgtgctcgtcggcggtcctgtcgggggctcggccgtcggccgcctgctgcgccaagttgaaggaacagcagccttgcttttgtcagtacaagaagaacccgagcttgaaggactacgtcaactccgacaacggtaagaaggcactcaaagcttgcggggtgcccatacccagctgctgaatgcttcaagagtgaccatcttctccgtggggagcgtgatcgccatccatgcaagatccttccatgcctgcgctgtgctatgtgtgtacaataagctagacattgaccaatgtctagagacttaatgctgttcttccgactacctttctaagtggtgacacttttaattcgatggatctttaaatgctta

SEQ ID NO: 192 - protein, 91 aa, synthetic construct
MKPILLLFLVSALLLSAAVPTASVTCNPLELSPCSSAVLSGARPSAACCAKLKEQQPCFCQYKKNPSLKDYVNSDNGKKALKACGVPIPSC

SEQ ID NO: 193 - protein, 68 aa, Musa acuminata
VTCNPLELSPCSSAVLSGARPSAACCAKLKEQQPCFCQYKKNPSLKDYVNSDNGKKALKACGVPIPSC

SEQ ID NO: 194 - DNA, 785 bp, Musa acuminata
  source 1..785: mol_type = genomic DNA
ttgggatgacaatatgagcttgtttcacctccttgcagcctctcctgatctcaatggcttcatcaaccattctctccttcctcctcttcgctctcctcctcgccctctcccgtgccgccactttcgagatcgtcaacgggtgccctttcaccgtctgggctgcagccgtgccaggtggcggccgccagcttgaccaaggccagacctgggtcctcgatgtgaaccccgggacgaccggcggccgcatctgggctcgcaccggctgctccttcgacggcagcggcagtggaagttgccagaccggggactgcggcggcctcttggagtgcgaggcgtacggcagcccacccaacaccctcgcggagttctcgctcaaccagttcaacaacatggacttcttcgacgtctccctccttgacggcttcaacgtgcccatggagttcagtagctgcatggcgatccggtgctcggcggacatcaatgggcggtgccccgccgagctgaggaccccgggcggctgcaacaatccatgcacggtgttcaagacggacgagtactgctgcacctccagcagctgcgggccgaccacctactccaagttcttcaagagcaactgccccgacgcctacagctacccgaaggacgacgccacctcgaccttcacttgccccggtaacggcgactacaaggtcattttctgcccctccgtctcgtcgttgccggccgatgggtaccccacgaggttcggccggatcgctgtgagttttgtgatcccaaaggcttca

SEQ ID NO: 195 - protein, 241 aa, synthetic construct
MASSTILSFLLFALLLALSRAATFEIVNWCPFTVWAAAVPGGGRQLDRGQTWVLDVNPGTTGGRIWARTGCSFDGSGSGSCQTGDCGGVLACQGYGSPPNTLAEFALNQFDNLDFLDVSLVDGFNVPMEFSPITGNCSAIRCLADIKGQCPAELWTPGGCNNPCTVFKTDEYCCTSGSCGPTNYSNFFKSNCPDAYSYPKDDATSTFTCPGNGDYKVIFCPSDGYPTRFGRTAVRFVIPRA

SEQ ID NO: 196 - protein, 220 aa, Musa acuminata
ATFEIVNWCPFTVWAAAVPGGGRQLDRGQTWVLDVNPGTTGGRIWARTGCSFDGSGSGSCQTGDCGGVLACQGYGSPPNTLAEFALNQFDNLDFLDVSLVDGFNVPMEFSPITGNCSAIRCLADIKGQCPAELWTPGGCNNPCTVFKTDEYCCTSGSCGPTNYSNFFKSNCPDAYSYPKDDATSTFTCPGNGDYKVIFCPSDGYPTRFGRTAVRFVIPRA

SEQ ID NO: 197 - DNA, 860 bp, Musa acuminata
  source 1..860: mol_type = genomic DNA
atgcttgagaaggaagtgtccactttgtttcttatcgactgatctgtctaccactcacatgcaagttcatccagtaccacagaggtacagtctccttggctctatttaggtggaagcctcgcagattccacaagcgcactccacactctcggcttctcatcaccttcctcttctcgttgcaatggcagcacggcttccctgcactttgcttctggtgctcctcctcacactctctttcaccgaggtgcgttgatcaaactctcatctgcatgcactttcagtaatctcgcaacattgctggtgttgtacactgacagtagtgtctttgtgtttctctttcggccctctagagtgtcagaggtggttctctcaagccttcaggtactgctacgttctgaactcatccattgtgtttgtgttggtgtggtcctgtggccggaggtgttacaacctgtgggtggtcattgcagaatgcaataacaagtgcgagttccggtgctcggcgacgtcgcacaagaagccatgcttgttcttctgcaagaagtgctgcgccgcgtgcctgtgcgtgccgcctggcacctacggcaacaaggaggcgtgcccctgctacaacaactggaagacgaaggaaggtggccccaagtgtccctagttttgaatagttgggacgcaagatgatggaagccaaacccacttccttttttctcactatgatttgccagtcattccatctgtgtttttaatttgctaaagaggagagattcataatataagtgaatcccctctactatcattgccaatatttggcaaagttgatatgagctaaataaacttcaactccaccactggtttgagaagaa

SEQ ID NO: 198 - protein, 91 aa, synthetic construct; identical to SEQ ID NO: 81

SEQ ID NO: 199 - protein, 66 aa, Musa acuminata
GSLKPSECNNKCDFRCSATSHKKPCLFFCKECCATCLCVPPGTYGNKEACPCYNNWKTKEGGPKCP

SEQ ID NO: 200 - DNA, 588 bp, Musa acuminata
  source 1..588: mol_type = genomic DNA
tcgcttcgaccacataccaccgtgtgagcattccgtagcctggtccagatatgaagcccctccacgccatcgtagctcttctgctcatcctcttcaccgcttcctatctgcaagccaccatggcaggctcaggtagatataatcaaacacaagtcttctgtgtcaccatttctcatgcatggattttgatcgtttcatgtcgttgttttgggtttcagcgttctgcgggtccaagtgcaaggtgaggtgctccaaggcgagcgttaaggaccgatgcctcaagtactgcgggctgtgctgcgagcagtgccggtgcgtgccatcggggacctacgggcacaaggacgagtgcccctgttacagggacaagtacaccggcagcgggaagaggaggagacccaaatgcccctgatcgaacaccttgaggtcatccggcgtcgaatggtgtgtgttttatagcgtatatgaatgagcgtgggggtcgcatgtatcgacgtgactatgggcagtggtgaaaataaatgttgtgctactcggtttactactatgtttgatgggagagagagagagagagagagagagagagag

SEQ ID NO: 201 - protein, 91 aa, synthetic construct
MKPLHAIVALLLILFTSSYLQATMAGSAFCGSKCKVRCSKASVKDRCLKYCGLCCEQCRCVPSGTYGHKDECPCYRDKYTGSGKRRRPKCP

SEQ ID NO: 202 - protein, 66 aa, Musa acuminata
GSAFCGSKCKVRCSKASVKDRCLKYCGLCCEQCRCVPSGTYGHKDECPCYRDKYTGSGKRRRPKCP

SEQ ID NO: 203 - DNA, 377 bp, Musa acuminata
  source 1..377: mol_type = genomic DNA
atggaggtgaaggcaacagcatgtgctgtcgtcatgtttctgcttttctctgcctctcgtaagtagtgttgtctggataagtgatgtgcatgttcaaatcttttatgatgaatgtttcagcttcatccattatgatctggtcctgattagctatgaagatgacgatggtaataacgtacgtctggaaatgaaaactggtcgcagagctgatgatgccgggagtggaagcaagagtgtgcaggtacccgagccgctccttccgggggctatgcttctccgacaccaactgcgcccatgtctgccagaccgagggcttcaacggtgggcattgccacggtctccggcgccggtgcatgtgcctgaggagaggctgctga

SEQ ID NO: 204 - protein, 76 aa, synthetic construct
MEVKATACALLMFLLFSASQLMMPGVEARVCRYASRSFRGLCFSDTNCAHVCQTEGFNGGHCHGLRRRCMCLRRGC

SEQ ID NO: 205 - protein, 48 aa, Musa acuminata
RVCRYASRSFRGLCFSDTNCAHVCQTEGFNGGHCHGLRRRCMCLRRGC

SEQ ID NO: 206 - DNA, 851 bp, Musa acuminata
  source 1..851: mol_type = genomic DNA
acagacacacggagacaagaagagggacgtagccatggcttcctccagtgctctcctggcagtggcgctcgcctgccttctcctcgccgcgccgaacacggcgcaagccgcgatcggctgcggccagataatctcctacctcattccctgccttggctacgcccagggcacggggcctctcacagaaggatgctgcagcggggtgagggcactcaacggcgccgcccagaccacaccggaccgccaggccacctgcaactgcctcaagagatccgccgccggcgtctcggggattcagcccggtctcatcgccgggatcccgagcaagtgcggcatcaatgtcccctaccgcatcagcccctccaccgactgctctcggcaagtgcctgatcaccatgccaaacattaccgatcatacacaaagacctaaaacaactgagagctgccttagttgacagctcggacaagtcttgcatggaataaactctaataacacatcatgcgtgactcgaccaaactccaaaatcgtactctcccagcgaaggctaaaccttgcacgcacatctacaagaaactgtgtgggtttgcatcaatactaagctttcttatttgatcttttgcagggtgaggtgagctgctgatttaacctgaagaaggcgtttaaggtagtgagttctatctaataaggtgggactgttgtctgcaccgtgtcatcaatttgtgtgggagcgctctcagcattattgtagctttgatctctgtgctgctgtcaatgctactgttccagtgggttgggacttgtagcaccgttactatctatataatatgagttccattaaacaaaagattga

SEQ ID NO: 207 - protein, 117 aa, synthetic construct
MASSSALLAVALTCLLLAAPHTAQAAIGCGQIISYLIPCLGYAQGTGPLTDGCCSGVRALNGAAQTTPDRQATCNCLKRSAAGVSGIQPGLIAGIPSKCGINVPYRISPSTDCSRVR

SEQ ID NO: 208 - protein, 92 aa, Musa acuminata
AIGCGQIISYLIPCLGYAQGTGPLTDGCCSGVRALNGAAQTTPDRQATCNCLKRSAAGVSGIQPGLIAGIPSKCGINVPYRISPSTDCSRVR

SEQ ID NO: 209 - DNA, 599 bp, Musa acuminata
  source 1..599: mol_type = genomic DNA
accgatctcatctctctactatcaccgtgtcgcaatggcgtcctccagaagaatgcttcctccactgcttttgctcttgttccttctcatgacctctggttctttctcctcccaggacttcaaatttacatactgcacgaatatcccggacaacatttaatgtatgtcttgacctttgttttgtgatgtagagatggggacgacggtggtgaatggaagaacatgcgagtctcagagccacaagttcgtcggcccgtgcgtgaggaaagccaactgcgggaacgtgtgccaaacggagggcttccacggaggggtttgccgtgggtcgctgatccaccgcaaatgctactgcaccaagaactgttgaacgcctcaaccggtccatcaataccactgtaatggtgttggaagcggcgttggagactgtcatcctgaaactgctaagaataacatcgatgatcgatgagtagaatgtcgtattataacttcgtgctttgattgcatctttaacgtagttttcctccgtatcatgcagcttctgttctgtgagattgtgtggttcaattcatgaataaatgtcctcttaaacgttgctgata

SEQ ID NO: 210 - protein, 79 aa, synthetic construct
MASSRRMLPSLLLLLFLLMTSEMGTTVVNARTCESQSHKFVGPCVREANCANVCHTEGFHGGVCRGTLIHRKCYCTKNC

SEQ ID NO: 211 - protein, 49 aa, Musa acuminata
RTCESQSHKFVGPCVREANCANVCHTEGFHGGVCRGTLIHRKCYCTKNC

SEQ ID NO: 212 - DNA, 178 bp, Cauliflower mosaic virus
  source 1..178: mol_type = genomic DNA
tttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcctg

SEQ ID NO: 213 - DNA, 253 bp, synthetic construct
  source 1..253: mol_type = other DNA
gatcgttcaaacatttggcaataaagtttcttaagattgaatcctgttgccggtcttgcgacgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcccggtgtcatctatgttactagatc

SEQ ID NO: 214 - DNA, 393 bp, synthetic construct
  source 1..393: mol_type = other DNA
caataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggcccgggaataaaatatctttattttcattacatctgtgtgttggttttttgtgtgaatcgatagtactaacatacgctctccatcaaaacaaaacgaaacaaaacaaactagcaaaataggctgtccccagtgcaagtgcagtgccagaacattt

SEQ ID NO: 215 - DNA, 234 bp, Musa acuminata
  source 1..234: mol_type = genomic DNA
atgggatcgatgaagaaggcttcggtcctcctcgtgctcttcctctcccttctcttaattgcttctgaggagatggtagcagtggaaggcaggacgtgcgagtcggcgagctccaggttcaaggggacgtgcgtgaggagcagcaactgcgccagcgtctgccagggcgagggcttccccgacggcaggtgcgagggcctccgtcgccggtgcatgtgccggaagccctgctga

SEQ ID NO: 216 - DNA, 414 bp, Musa acuminata
  source 1..414: mol_type = genomic DNA
atgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaaaagagaggccgaagctatgaggacctgcgaatcagcctcctcacgtttcaagggaacctgtgttaggtcttctaattgcgcttcagtctgtcagggcgagggattccccgacggacgttgtgagggtttgagaagaaggtgcatgtgtagaaaaccctgttga

SEQ ID NO: 217 - DNA, 240 bp, Musa acuminata
  source 1..240: mol_type = genomic DNA
atggcgtcctccagacgaatgcttccttcactgcttttgctcttgttccttctcatgacctctgagatggggacgacggtggtgaatgcaagaacatgcgagtctcagagccacaagttcgtcggcccgtgcgtgagggaagccaactgcgctaacgtgtgccatacggagggcttccacggaggggtttgccgtgggacgctgatccaccgcaaatgctactgcaccaagaactgttga

SEQ ID NO: 218 - DNA, 420 bp, Musa acuminata
  source 1..420: mol_type = genomic DNA
atgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaaaagagaggccgaagctatgaggacctgcgagtctcaatcccataaattcgtaggcccctgcgtaagagaagccaactgtgccaatgtgtgccacactgaaggatttcatggaggagtgtgtaggggaacgcttatccaccgtaaatgttactgcacgaagaattgttga

SEQ ID NO: 219 - DNA, 500 bp, Musa acuminata
  source 1..500: mol_type = genomic DNA
gatatgattatatacacatccatagtatagtatgaactctccaataagagagatttcatatgttgtcacaattaggaatctatataattgtttcatgaataaataaaatactcatgtcttatcactaacatataataaaatatttattctatatttaggtcttcgaaaagataggaattgactgtaaagtaaatgcaactgttagaattcactttcttagttatctgttctttatttatctgttagaagtcactttcctcccactacgttgcaatggatgttgttgacagtattcaccgacaacatcgagataaactggtgtttcctgcataatttatgattcctgccaccaacaatcacttcgtcaagtaaatatttatcttttagttgaccgactgggtcattgtttatcctcaaattcatggatcaagatttacaaggggagcgaccttgaatggatctttgaggtaattcccactatttgccatcagagaggaaga

SEQ ID NO: 220 - DNA, 241 bp, synthetic construct
  source 1..241: mol_type = other DNA
cactttcctcccgctacgttgcaatggatgttgttgacagtattcaccgacaacatcgagataaactggtgtttcctgcataatttatgattcctgccaccatcgtcaagtaaatatttatctttgagttgaccgactgggtcattgtttatcctcaaattcatggatcaagatttacaaggggagcgaccttgaatggatctttgaggtgattccctctatttgccatcagagaagaaga

SEQ ID NO: 221 - DNA, 239 bp, synthetic construct
  source 1..239: mol_type = other DNA
cactttcctccctctacgttgcaatggatgttgttgacagtattcaccgacaacatcaagataaactggtgtttcctgcataatttatgattcctgccactcgtcaagtaaatatttatctttgagttgaccgactggatcattatttatcctcaaattcatggatcaagatttacaaggggagcgaccttgaatggatctttgaggtgattccctctatttgccatcagagaagaaga

SEQ ID NO: 222 - DNA, 241 bp, synthetic construct
  source 1..241: mol_type = other DNA
cactttcctcccactacgttgcaatggatgttgttgacagtattcaccgacaacatcgagataaactggtgtttcctgcataatttatgattcctgccaccaacgtcaagtaaatatttatcttttagttgaccgactgggtcattgtttatcctcaaattcatggatcaagatttacaaggggagcgaccttgaatggatctttgaggtgattccctctatttgccatcagagaagaaga

SEQ ID NO: 223 - DNA, 13578 bp, synthetic construct
  source 1..13578: mol_type = other DNA
gtttacccgccaatatatcctgtcaaacactgatagtttaattcccaaataataaaaaagccggattaataatctggctttttatattctctctgcagcaccctgcaggggtttggccacgcagcacaccatccacctcatacggtctctagcgcaagagcgcaggttcgcattaatgctttaattaggcgattcccgatacgtagtctcacgttacaattagtactaaaataaacgtaaaccgaacttaatctcctcattatgtgccatgttttaaatcatttgttttgcggttattacgcttacttttttctacatctttattttataaaatagatcatgaaatagcatgatgaagggaggtttataggtccctcgcctcgttttactctatgagcccatcttatctttaatcatattccatagtccataccatagcacatacagtagttatatgctgcagaagagatccaacaaaacattcacaatggattatagaaacatttgtttattcattataatgagatcttacattcatttaatattagaaaaagccacaaattcataacacaacaagccaagaaaaaaacacaaacttaagcacacaagctttttatttgacacaccaaatatttcatcttcatcttcaagctcaagggcagaagacaaccctgtagttggtgccgccggggcaagtgaaggtgctcgtctgatcgtccttgggatagctgtaggcgtcggggcagtttctcttgaagaaccgggagtagtcggtcgggctgcagctgccggagttgcaacagtactggtccgtcttgaacacggtgcaggggttgttgcagccgcccggcgccttcagcgccccggggcactgcccgttgatgtccgccgcgcaccggatgccgcggcagccgcctgacgtggggctgaagtccattggcacgttgaagccgtcgaccagggagatgtcgaagaagtcgaggttgttgaactggttgagcgcgaactccgccagggtgttgggcgggctgccgtacgccgtgcatgacagcacgccgccgcagtcgcccgtctggcaccgcccgcgtccgctcccgtcgaaggagcagccggtgcggccccagatgcggccgccggtggttcccgcgttcacgttgatggtccacgactgcccctggttgagctggcgtccgccgccaggcacggccgcggcccacactgtgtaagagcatcggttcacgatctcgaaggtggcggcatgggagagggtgacaagaagggagaagaagaagaagaagaagaaggagagggagagaatgctggacgaagccattaagcttagctcttacctgttttcgtcgtcttggcttttcttcttcctggccacgtgcctgaatacttcgccgccttgactacttctaggctacttgctctctttctctctctaggactatctctctgagattttgctcccttaacaatgagggaggggctaagtatttatagactgacgggtgagtggacatttcccaaactacccttacttatttcgtaagcccttacgtcattgctccattattggagtctgaagatgccttcacatggtggacccccacctgtcggccacgaatcttatttgttcgtcagaaaaagccaaaccgactgcacagtttttccatcgtggtagggaccactttggtattgattaaaggcagccgacctaacctttgtcagacgcattatttcacgcgttttcttttacagattccatcttcatttgtgggacagtatgtctgccactcctgacggagctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagaatggcagcacggcttccctgcactttgcttctggtgctcctcctcacactctctttgaccgagagtgtcagaagtggttctctcaagccttcagaatgcaataacaagtgcgacttccggtgctcggcgacgtcgcacaagaagccatgcttgttcttctgcaaggagtgctgcgccacgtgcctgtgcgtgccgcctggcacctacggcaacaaggaggcgtgcccctgctacaacaactggaagacgaaggaaggtggccccaagtgtccctaggcttacgggaattaaacctataaacataaatataaataatatatataaacctaagtgtctaagttccataaattaagctgtagtctctggcttaaaacatgttaggtttgtttatacaagtagttggatgtttggagtacttcggtcttttgcgtaccatcaatatttaagaactaagttagttatgttccgtaacttatgggctcttaattaaactatatctgcacaaaattatatatatatcaaatgtgatggtatgtggactataaaaagatatggttgagaaccacaaactttgaaacttcgaataatatattgccagtgacagtcttgttgatttgttatagcaagtcctattttcttaatcattgctttgttttaacgtacctagatttcataacttttgtctttgtctcaagctgaacctaatgatgatagtaatattaacttattgtataggggtatttcataggataaaaaatgatgtgcaattacgtgtagaccaaatattacttgatgacagatggataaatattttaaaactcgcttctgggagtgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagctttcgcagatctgtcgatcgaccaacaatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacacatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagctttttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcctgcacgctctcctgagaccgaattccaaacgcgaagtaatcttttcggttttaaagaaaaagggcagggtggtgacaccttgcccgtttttttgccggaccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtccccccttcagaagttcctatactttctagagaataggaacttccctatagagtcgaataagggcgacaccccctaattagcccgggcgaaaggcccagtctttcgactgagcctttcgttttatttgatgcctggcagttccctactctcgcatggggagtccccacactaccatcggcgctacggcgtttcacttctgagttcggcatggggtcaggtgggaccaccgcgctactgccgccaggcaaacaaggggtgttatgagccatattcaggtataaatgggctcgcgataatgttcagaattggttaattggttgtaacactgacccctatttgtttatttttctaaatacattcaaatatgtatccgctcatgagacaataaccctgataaatgcttcaataatattgaaaaaggaagaatatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaaaagcttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgaggtcccgcagccgaacgaccgagcgcagcggcgagagtagggaactgccaggcatcctgggcggttctgataacgagtaatcgttaatccgcaaataacgtaaaaacccgcttcggcgggtttttttatggggggagtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatgcatttagaataccttttgtgtcgcccttattcgactccctatagaagttcctattctctagaaagtataggaacttcccttcattttccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagacgttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttag

SEQ ID NO: 224 - DNA, 54497 bp, Musa acuminata
  source 1..54497: mol_type = genomic DNA
cctccttccgcccccgcgcgcgccccccgggcccccacaccctaatttacctctacttttcccgcactgagtcgctcttctgttctttctttttcctcctttttcttcggccccttcctgcttcttgggccgctttgcgtagctctggttcttcctccgtctttgcatgcacccagagatgtctcgaccggtctccctttttatcacataatcatttcttttcgtttgttgtctctctccgttccccaaatctcttccttttaaatccgtcttggattgactgctaaatatctcactcttgtagcagggcgacgaagatctccttcggcagctgcaggaaaccctagattccgatcccgataatccgtcgcaccattataacatcgtacgctccgattattacattttctctctcagtccggctaccttccttcccctttgatttttatttgttttaaattgtgcagaacaagtatgtggacttctccgtgtctattttattttattttgtttctatttctttagggcctcttcttgtggaaaaagggcgaagcagtagatgatcaagctgaccaatcgaagcaattaagggagagagcagccgagcattttttggcctctgcgaagctgaaccctagtgacggcgtcgcattccgctttctggggcattactacagcacggtttccgtcgatgcacagaggtctgcaaagtgctaccagagggctgtcgcccttaatccccatgattctgaagctggcgtgagttgtttttgttcccacttattgaacttgtgtcaggcttgtaaactgttatactgagtattaccgcgaaacgtttgttttatttgttagccttttatttatgtatgcaggaggcgctgtgtgatctgctggatggagaaggaaaggaaagcttggagattgcggtgtgtagagaagcatctgagaagtctcccagagctttctgggcattccagagattgggatacttgcaggttttaatgacaattgctctttgatttctcttgtatattattacgtggttgtcattgtgtgccatgtgttgctggaagctatgttactatgttggtgttatacttatttgtcacataattatagaggaaaagaaagattccacatatgttttatttgagtgaaaaaagttgcactggctgcaacaaagttttatggatatattcatgctgtatcataaatttcaggtccaccagaggagatggtctgaggcagtacagagccttcagcatgctatacgaggattcccggcatgtgctgacttgtgggaagtgtgttatttgacaagtcattctagctctatatttgactttttaattttaaatatccctgattgttaattctgttatttcaggcgcttggtcttgcctaccatcgcttgggcatgtttactgcagctgttaaggtgtggtccctattttctgatatatctgtctgttaaatctgttgcttcctgaaattataccaacgttaaatctctaggtgctttggtgcccctgataatgtgggaaaaaaaatttatgatcaatttccccaattctgctgtgtaattgttatcgtgctgacattcatttgccttgcaacagtcgtatgggcgagccattgagttggagaactcaagaatatttgcattggtggagagtgggcatattcagctaatgcttggttctttcagaaaagtaagtcttctgctttttggttagtcttttgtggaaaagtgaggcttggaacactggtctaaactttactgctacttaggttttgtaataagtttgtatgtccaggaccacattattttggtgaaatatgtgaagtgatgaccacttttgtgaatcttactttgaattttctattaaagtttttctggttcattcatttttttaaaaattgtgcaattaacatttttgagctctattttatttcctatttctaggtacaagaaaactgtatccttactggttacctaggtaagtggccattgccaatggctttttacctcatggttggatttgttctgcccagatagcactcagtactgagatgaagcaattgaggttagcctatgtggctgccaatcatttcttgtcaatgtaatggagttgcttttgttggtaagctcttggcatttgacagagctcgtctccacttggtagggagggatagtagagagaagcagattctgttaagtgaactgtcattcaggttacattggaatcaactggcagttggttggtggggcctttaggtggattccattaattaggtgatagcaagttgcaaagcccactttgaaaagtcaaaaattacatgttttctagcaaatcccattaaaaggcttggactacactcattccttgtatctagagaaagaggtgtgttgttgttctcctactataagagagtatcatgatttttccttatactagagagaagatgtgaatccttttgccatagtcaaatccttataaccttgcccatggatgttttccttagattacttatgggttttaccatgttaaatatgttttgtgtcgaatatttttgtttttcttgatttcaactttagttgtagatattgtttcatcttaacactgttaatgttgggcccaagccataaaaaaggttgagggttgtgttaggtcattgatagcaagcagtgtgaaactatgccagatctcatgatgacggcagaagatccactagtgaagtcattgggtgctcgaatgctcgcctaggcgcctaggcgaggggaggcatggctcgagtgcttgcctattaggttggcagggcgactttaattaggtgcggtctaggcgcttgcctgggcccaagcgttggttgagccgggcgttcgcctggttcaactcatgctaggtcgagcttgagttcgagcttgagtcgaaccaaatcacactttggtttggttgaactaggtaatgcataattgccaacaccccatgcgagctgtgacctaatgcaaaactctctctttcacactgtgtttctgcatgacggtgaacttcatcccatcaatgaacgacgatagcaatgatgtcttcctccaatggtagtggtgatgtcttcctccaacggcagcgacaacttcttcctccactggcaatgactatgttgtcctccaacgacagtgatagcagtgaatgtgatgtcttcatccgacagtggcttcttcccctcccctctcgtatatctctttcattctccctcctccctcctccctcctccctcctccctcctacctttggcctgttcaccatggcccctcgtctccctcctccccctcgtcgtagccccccttccttgatccctcttccctctttgttgctacttctcccctcttccccttcctcttccctccttccctctctatgcgaccaatgtatttcagtgatttaaaaaggcgctcgggcgctcgcctaggcgctcgggcgaggcaaggcgaggcccgagcgcctcgcttcacttccaggcggcgcgcttcaaagaggcaccgcttgggcgctcgcccgagcccaggcgctgggcgcttcgggcgagcgcctgggttaaaccaggcgactgaaccagtgttttaggtctggttcggtctccggtgctttagttggttcaatcgaaccaactaaaggattgaaccaactaaagcaccgatatcagctatcgctgcccaaccctaaccctgctcgttgccgctgccgctgtcgccgctcgtcgctgtcgctgctcgctaccgctgtcgctgctcgctaccgctgtcgctgctcgctaccgctgtcgctgttgctgctcccgctcccgctgtcgctgccgcttcctcttttctcagttagcacccccttacactcctcaacattgttctccttctgtatactattaacagtatactaataatagtatttgtatttattagattaataatatattattttgattttgtatcttagatttttttaatttaatagcatatttttatttaaaattttaaataattatattatatatctttatattttagcgtctcgcttcgctcgggcgagcgcctagcgcctcgggcgtttttggaccttggcgcctagcactttttaaatcactgatgtatttgtttattgttgcctttgattcctatggcttcattgattaagctctactgtcatccctactattacttagaaaaaagaaaaaagaactagatcaagtgcttatacaccttcagccgatggaagcgggtattcactgcgctattcatctagattgacccctttacagaactgaaagagagatcaatgattttagccaactcaaacaatcaccgacaacatcttttccctctttatttgccatcatagtttcccccatccattgatgtgttattttagttttagtttatttttattaatcatgatatataatttttaaattataatatttagaagttccttgctttgctcaggtgagtgcctagcaccttgggcattttgggatggtggtgtttttggtgcctagcacttttaattaaggattttaatttcaaatgatgccgtatgtaccgggtggtacgtatcggtctgccagcagactggtacgtagatcggtatgcgtgggcggtaccattgattggggttgtttccaccccgtcactgctcgaaatcggtcggtaacggtcgatttcgatcgtcatcgcttgctactgggcggtattagccgagggagaagaagagggagaagaaaagggagacctggagatccgatgctgctctcccttgacgatcccgatctgtcgccgccctccctcaccggacgccacaaatgaaatgtcgcctcctcgtttgaggcgatgaggccaccatgttgtcggcgacttctcctcatctgcgtggggagaagaaatgaggcaaccttgcctcgatgacaatacgcttttctttattttttaactattatatatatatatatatatatatatctcgagcggtacgctcgtaccatactataccgagctgagctcgatacttcggtatggtacgaaattacgagtcttgcttttaacaatacaaaaatctacatagccaaccccaaatatgtgggacgtatggttttcttgttgttatatattgtttcatcatatgaagtttctaacttatggtattagagctcgattgcagggttattgttcattgtgaatagtgggagttgatttgagttagaagtatatcaacttttgtataattataacaaattttgaaatagaagttcaactggtgtaatttctcccttgaaacatgatgatgaaggcaattttgaaatagaagtatatcaacttatgtataatgataacaaagttttcaacaagtgtaatttctaccttgaaagatgaagacaaaggcaattttgaggaaggacatagtagatataagttcaaaaagtaagaagaatgtcaagtgctataatcatggcaagcgcggacacataacaagtgttggtataatgagaagaatgcatataatggaaaaagtcctcagtcactaaatacccaagcatgtataccaattatatttgatgatgctgaagcattatacaatgaaggaactatagtcttggaaggtaaaaaacagtttgatgcccgactagtagatttagcaacaacttgacatgacttttataagagaatggtttcaacaatatggaatggacctaatgaaggaatacatttttgggtgataatcatgccttggagattgttggtattggcacaatttatactattaaaagggcacgagacatgaagtgtttgaggaaatgagcttagttgccagatatatgaaaaaaaatggatcatgaagtttgtcaaaggcgtgcttgttgttataaaggctattaagtttgctataaatctatatatgcttttggtcctttatattggttcttcctggtcataaattggtaaatatattgtgagcattgtgttagaaagtaagccttcgaggcacgaatggaggataagattcggacgctttttaccgaacttagattgggtcggccactaagcttgaagaaatcacatcaaggagagagctttgcccaatcgcaccaagctcgaagagatgacttccaagggaggggaggctctatgaccgacctcaactatccacgcatgagggtggacttccctagatgggaagaaggagacccaattggttggatctcgtgcgcggagcgatattttcggtaccacaaaatcgcggacgcatctatggtgaaaattgcggctatacatcttgaagggaatgccatacaatggtttgactggtttgaacacactcatggaatcctctaatggcgacaattcaaaaaaggactactaatccgcttcggaccaactgattacgagaacattaacggacaactagcaaagatccgataaaccttcaccattcaggagtaccaaaccaggtttgaaaggttatctaatcaaactcatgattagtctcaaaaacaactattggggacctttattgagggcttgaagccggagatccgaggagaagttaaagcgcgacaatcgtacactcttatggtagccatctctttcgcacgacattaagaggaacgattgaactgtgaagctcggaggactagggtcgctcctcgaccagcaatactgaagccctcaacccccctactgtcgacggagtccctgcactaaagaggttgacaagagaagagctttgggagcgatatgcgaaggggttatgttggcattacgacaagccgtggagccgtgagcatcgctatagtaaagggagacttcttatgattgaaccagtagaagaagaggtcattgaacatccaaaagagagccttgaacatgaagaagatgtaaaagaagagccataaccgaccgaagttatggcacatgcactagccggctactcaaacccgcaaatgatgaaagttggaggccttctcaaacaacaaccgattattgttttcatcgacacgggcagtactaataacttcctaaatagtaaggttgctgtccggatggccgtacctgtcgagaattgcagcaggtttgatgttaaggtcgctgacggatggattttgaagtatgatcataggcgcccgcgagtgaaactattgctgcaggaccaagagataattgcatatttcttcctcctccctcttgatgatcatgaggccatgcttagaattaaatggttgacgacattaggtgatgtttcttggaattttatgcaactaattatgaaattttacagtaaggagaaacatgtgatactgtacgagaaacatgggggcgatgtaacgacgatttgcacacaacaaatggataaggttttgcataaagcatgcagcggctttttggtacaacttgagcagcaaactaagggagagccaatagaatttgaagatccaaacctacttcctttgtttgctgaattttcagatatatttgacgaaccgcacaacctacctcttacccgtcggcatgatcattatataacgattcttccaagcaaacttctagcaaatactcggccatatcagtatctacatctccagaaggatgaaatagaaaggattgtaaaagagatgctcgaaacaggagttattcggccaagttgcagcctctactcttcaccggtgctacttgtacgcaagaaggacggaacatgacgaatatacgttgattaccgagctctcaatagcataaccattaaggataaataccctattcttgtcacgacccgagtctgatgagtcaattggccaataactccattttggtccttcaaccacggaccaaaatgcttaagcgggagttagcgtagtacactcatcaggcccatataagctaatcatacacattgcccacttccgatgtgggactattgagatgttacaatatccccaactcaattagcttgacgtcctcgtcaaggctcgacataacccagatcgaaccctagcatagtgcatgtgaggtttaactcagtggtggctccacgccgtgatgagttctcgactccatccacgggtagccctttcctactcgagccctctcaccctgcccaaatgctaggtcggctctgataccaaatgtcacgacccgagtctgatgagtcaactgaccaataactccattttggtccttcaaccacggaccaaaatgcttaagtgggagttaacgtagtacacttatcagacccatataagctaatcatacacattgcccacttccgatgtgggactaatgggatgttacaatatccccaactcaattagcttgcgtcctcgtcaaggcccgacataacccagatcgaaccctagcatagtgcatgtaaggtttaactcagtggtggctccatgccgtgatgagttctcgactccatccacgggtagccctttcctactcgagccctctcaccctgcccaaatgctaggtcggctttgataccaaatgtcacgacccgagtctgatgagtcaactggccaataactccattttggtccttcaaccacggaccaaaatgcttaagcgagagttaacgtagtacactcatcaggcccatataagctaatcatacacattgcccacttccgatgtgggactaatgggatgttacaatatccccaactcaattagcttgacgtcctcgtcaaggcccgacagagcctagatcgaaccctagcatagtgcatgtgaggtttaactcagtggtggctccacgccgtgatgagttctcgactccatccacgggtagctctttcctactcgagtcctctcaccctgcccaaatgctaggtcggctctgataccaaatgtcacgacccgagtctgatgagtcaactggccaataactctattttggtccttcaaccacggaccaaaatgcttaagcgggagttaacgtagtacactcatcaggcccatataagctaatcatacacattgcccacttccgatgtgggactaatgggatgttacaattctagtagtagatgaattgctagatgaaaggaagcataaatcttcagaaagctggaccttcgatccgggtatcatcaaatacgagtgtgcgaaaaagacatactgaaaatcgcctttcgaacacacaacgaccactatgaatttttgctttctttaacaaaaggtggagtatcttgggcatatcatatcagaggaaggtgttatggtggacccctccaaaatagaagcaatgcagaactggccgaccccgaggaacataaaattgctacatgactttttgggcttaataggttactcccgcaagttcgtgaaaaactatggaaagatcagtgcaccacttccttactgaaaaaagatgtcttctaatggtcggacaaagcctccgttgccttcgacaaacttaaggtagccatgacgacgacaccggtgctagcactactagaattcagccgacccctcattattgaggccggcgcatctggagtcagaattggagccattctcattcaagatggtcgaccactcgcatacactagcaaggcattgtctccctcccatcaaaatatgtcaacatatgataaggagatgttcgccattatgcacgcagcaacgaggtggagaccctacttgatcggtcgacgatttcaaattaaaactaacaataaatgcctcaagtactttttggagcaaaagatatcatcccctgaggagcaacaatgggtaacaaaacttcttggatttgattatgaaataatttacaaaaaagggaaagagaatgttgttgtagatgcgcttttgctactacccgagcaagctgaattttcggccgtttcactttcgaccagtgacttccttgaggatattaagatcgaatggcaggaagattcggagatcagtaagatcataaaaaaattggagttagcactaagctccatggctcattacaattgggactcaaaagaattacactataagggacgcattgtgcttgtgacaaattctacttgtatcttcacgagcagattttggacaaagttattccatatgcaagatactaaattaaaaaggagtacgacatatcacccacaaaccgacgaccagacgaaacttgtaaataggtgcttagagatagcccaaagctacccaccaaatatgactatccaaggagaacttcagatctaaacaagtgccgttattaatcgacggatcgtgactcgacgacgatgacccactactgaagttctaatatagtgggcgaacctactaacagaagatgccacttgggagaactatgacgacttgaagatcaaattcctaaaattcatgaatcgtcagcctcgaggacaaggttgatttgaagagggcgggtctattaagactctagctaggagagtcctaattgaaagggacattcatgtaaaacctacctaagccgacccttattaaagaggtgaagaggtcggctagggttaggaggttgtttcttagagaagaattaggagttgtaaaggaataggagtcttgagtaggagtcctattaggagttggttagaagtatgagtcttgagtaggagtcatattaggagttcgggtttagaagccctataaatagccatgtattcctcctcttttcataagcaatagatgaatcttttctgcagcctttgagcagcaactccaagggaggaacccctatagagttccaagaaggccgatcccctaaagagatcaaccccaagtttagaatctacaagggttctatcatgtttatagccgtcatacaacacccttctatcataatgccaaggtcttcccaacagcaaatgacaagcatccataggagcaacatcacatcatacttcgtctttataatatttgctaatagaaaacgaaactaaacatcttttagttaccttgatgtcatttcctttttgcaaccaacataattgatacagatgtggatgagggattgtatccaactttagcttctgcaccatctccaaagaaaccacgttcttaaaattgccgctatcgatgatcaccaagcataccttgccaagagaagtgcatttagtgtgaaaaatgttttttcgcacccaactttcatcatcggccacataagacacttgcacgctacgttacaatacaatagacaaaccatgatctgtataagtaacctcttcctcatcatcatattttggttcgtcgtcggcttcatcttctcctccaccactgtgatctttaacaaaagttacaattcttctatttggacaatctgaagcaatatacccaaaatcatgacatttgaagcattttcggccactttgggatagaagaatttgatgtttttttgttgccagcagattcttcatccttttcttgcacttttgatattaccttgctttagacagtaggcttggaacttcctccttttgtatatccttcttttgagccgtaccgagaactcttttcaaacttctgttgcttttcaactttcaatgccagcttactcacatcatttaaagaccagtatggctgtagttgaaccactttagcaatctcatatttcagacctcccaagtatcttgcaatggtttgctctttcggttccacaaactctccttttaatatcagattattgaattctgcagtgtactccttcacattaagatctttttgcttgaaatcatggattttgagaaagatttcttgcctataattgtgaggtaaatattttctttttagctcccttttcattttttcccatgtcacgatcttactcttcccctcacattctctttgtttcttcagattttcccactaaaaagatgcatttcgtctaagttttagtgccacaagtttgaccttcttttgttctggtggttcatgaaaatcgaaaattctttctactgtattaagccaattgataaagtcatcaacatttattttaccttcaaactatggaatctccaatctgagattatatttatcctgccactcgtcttagactctatttcttgtccgacatcttcttgactcccttgggagagaaggtttatcttcattaaaagtatattcatggacatcaattttatgcttgttttatctactttgaagttcttcgattatttcattcttttcttgtagactacgcagtaatcttcgtagctgcagcctcaacgactcgacattatcttcatagtcgctaccttcaccaactatatcttttccattccgccttgccatgatctctagcctttagctctgataccaaactaataccaccgtgaagcagaatgggggtaaagtataatagaactcgaaagaaaatcgatagtacattttttttttttgggtggaaaatgtacgaactcaataaaatcgaaaaacagcttaccacccagttcaaaatcacaaagggatatagaaagttcactaccctaaggaagttaaacaaagatacagaactgaaaataaaagactcaccacttaaagaagcatccaagagatacagagtttaagcgagaacttcaagagagcttctgcaaaatatcatgagttcctaaagttttttctaagccctaaacaccaacataagtactagtgcatgaaacaacccttcatgcatagggaatttcgaaattaagtgttcacagttgctaaacaactcttttaatgcattccttggtcatgaagaaatgttcacagctgctaagcaactcctttaatgcattccttggtaatgaagaaaagcaccttgaaacagctttaactttatgtagggaatatgctgatgagctgttatatttgagtgggttgagttgaagattatgaggcatcattgttggctgaaaaaaataccatatcataatcagattgtagtaaattagacacccccgagtcatataatgcctaagaaagaacaaagttggatccaaaatccatgaaatgtttttcttttgggatattctaagagagtcaaggatatcatttattggatcccacttgtcacaaggtatcaacggtgattttatctttgtagaagatgaaatgaaaccacaaagagatgaaaatgatagcataatgaaagtcaatattgaaactatgaacattctcaaccggaaaattcaatttccttttaaggagcagagcatggtgaataaaaattaattgtatttgaggttccaaaagtacgatggccaactcctaaaagaaaaccaccacttggcactcagaatatatgatggagagtaatataacattactgtcttttgatagagaataaagaagcatcaacatttcataaagttactaatagctcatatgcttctatgtgaatggcactagtgtaggataaaattaatagagaataaaaaggcatcaactttttataaagctactagttgctcaggtgcttctatgtgaatggcaataatgtaggataaaatggaagctcttgcaaaaataagacatgggatcttgtacatttaccccttaaagctcacttttcatattctatttatcaattttagaattgccatttccatgttgcttaagtttttactagaccgtagacttgtcaacattggttggcttcgtcatgacatctgctatgctcttcaaagattgactattatgcatatcccacattccttgactactttttgaacaaagcgatactgaactcctatgtgctttgaataggaatgaaatgcaaggcactgcctctattgtagatcaggctacaacaggttgtaatctagaaaccaagctcactctcctgaagtgtaaacacagctaattgtggattttctcttgtcaagaggtcacttaaaaattttgaattaatatatcctctggccgtaaatctgaccctctataacataatggaacatttatggtccttttgatgtatcataggatcatcttaacaatgtctaaatagaatttgccatgaaccaattgtccatatcaattgtcgtaagtcacctacttgtgcaattttaattcttgtacatactatagcaaaattaagccatatagacctcaagacattttccatactctttgcttcatttcctggacatatctaagatcattcaaaattggttcacaatcttgcacgtttagcgtcacaaattttctttaaaaagttcgtctcaaaaagttataccttcttgtcttttttgtgtcggtgaacttgcatcatatagtattatttgttggttccaaatccttcatacgaaactccttggctaatagtgtctttaattcttttaattctttaactcgatctgttttggggcctaccactcaatgtaccatagcaatgtaatgaaatcattattatcttcaaacctcatagtatgtacaatggcttgaagcaagtctattgtatttatagctaagaatgaagcaacaaaatctctttgtaccaccacctcaatgcctaatttagactataaatggatttttttgacttgcaaaccaagttctctatcttttttaccaaatccttagggttggagcatgtaaatttcttctgaagaaatattgcttttacatctaattgctctaaatataaatcaaatttagcatatatatccaagattaatttaaccattgtaagtgactagtgttgacggtagttgtcgagcaaacctttagcatgggaggatggatcctacatgaaaccggatcgagtatgaccctcgaatcagtggacgctcaagcctaccttgacgattggacaaaggcagcgaaacgaaaacaagagatcattagcaaagaaaacaacattcccttcgacctatcaatcgaagggactatcaaaattggaatgtccggcatcgacggtgatgactattaaatttttataattttactcaattttatatcaagtttcttttgttgtccttacacatttatgctcattaaagtcgttatggatgtacatcaatttttttaaaatttaaaaataaaattatgattttttaatagtataaattattttttaaaacaataaaaaattattggcttaaactagaatcaaacccggaatcggaattgctggttcccattctagttctaaaattgtcttatgctagttcggtttcgatttctagtttctaccgaactgaaatcgatggttttagaatcgttgtcaggtctagctataacggtagcaaaagctacaagtatgataaaatccaaggcatatcatagtatatatggaaactagatactacggaggatggtcaacatggacaatttttaaattgttaatattaggaaaagaaagagtagagatttgatttcactaagtttataaaggatgaagatcaaaagatacttgatagatgtggaggccaaggtttgatgtaccgaagcataccgcttggtacgggaggtacataccggttcgataaaggattggtacgggcagtacatcagtataccccagtgtaccatatgttggtatgcttggtacaactcggtatggcttgatacatatcgtattgacagctggtcggtatatcggtacggactggtaatacgaaccacgatggagacaaaggaaaaataaagaaataattataataaaatggtttattgtattatatagatttaagctttagagataatagtgttgagttcaaaaaacatatatttatacgtgaaatcagaacaaatgagataaaagaagctttaagcagattgaaaatgtgtttatagtagttaatgagacatgatggttttacccaaggttcttaattttagcccgtaatgttgtatcgagctgtcatggccttagttggaattgcctaaggcgtgaggcacccttgcggtaatgacgcgaacttagcttgcgttgcctaagttgcgagggacccttgcggcaaagacgcgaacttagcttgcgttgcctaagtcgcgcttggcccttgcgatttgcgtccgcaaagattagcccacttgcaacctctcgcaggtcccgaaggacctgtaaaaaaagaaagttgattagtacgaagaacgagcgacggacaagtcccgacgtctcacgaaaaggggaagctttacaagcaatttagcgagcaccttgcgtgcacaagagaaaagagggagagagggaaaacaaggactttagaaggttgaacgaacagttgcaagtccacaaacaattgctcatcgggtgccgggcgccgggcgggacaacaagttctcgtcaaggtaacgtgcgaacttacgaaagattgttcaacgctcgacactataccgaagccccatccagccttgtgccacctgggtggttccaaggggttgaaatggctgacgttttgtgagcggaggcggactgtagaaaatagctcgtggcacacgtaaacggagctgttttgggctgttttggggctgttttgctcagttcggtgagcgatcgctttgtagccctgcaacttgttcgaacttacatttttataagcaaaatgactcaaaaccaagacaaaacatgctgccaagcagctatacatgaagatgagagcgacgaacggttcgttgaacggagttgttgcggctacgtgacggccatttgtgacagagccctcttcggtatgatacgtaccaaggtatactgacggtacacctacaaagagctgaaaaacatccaaaaatacttggaggtaacggtccaaattatcagggtgtatcgatcggtacacctcggtaatggtcgaaatctaaggtgtatcgaccagtataccttggtaacgatcgaaatatggtgagtactgattggtacacctcgataataatcggatttcaatcgttattgcccaatacagatctcgtatcgcccgatacagggtcaaaacaccgttatcgcccaatagagagcggtccacataccagaatcctttggaccggtacgtaccacttgtaccaggcggtacacatcgaaattaagaatcttggttttacctttgaggtttatagtagttcatgaaagcaaggtttgctgtaccggactgtaccgcccggtacgggcggtacgtaccggtccgacaagccagcggtacgcggaccgccccgtaccgtaccgagtactgtagcagtgtacagtagcactgtagcagtgtacagtgctcggtacacctgggtgtaccgaggggtataccataccgtaccggtaccgtgcccgggtcgaaacgccggtacggtacggtacgacgaaccttgcatgaaagcatgatatagtttagataactagtttattaaacaagattttgaaaattaaaaaaatgcttgatgagtaaagagctttttgttattaatttatgataataaggagatattcaaagctattgtaattatataggagctaaaataataaatcacactattaaattatgagacatgatgaatgaaggtagaataagataaaagatagtatttttgagaatcattttgattttgtaaccaaaagattaactaataaaagctattaattgataagattaaggattttaattttgaataatacagcccataccgggaggtatgtaccgcgctgctggcggaccggtacatggaccgtcctgtcacagacaaaattctaaacaggatgtttgatgtaatgcttatgtgtgtccgtgttttttggtatgttcatgctttgcacaacatgtagagggacggtcgaaggcttaacgaccccattttagttgggtttggtggtcgttttaggcttgtaaataaaggttgtgtcatgtggacacttgcgagagatattcggtgtgcgatcgttcagagcttatgaagtctgtttgtaatttgcattagttttgaagtgttttatgaaatgtttgtttgtggattccgagtgagacactttttctaacccgttttctcttttgtaggtcctaagggaccatgggaggtttcgaggaggctgacctttgcggacggacacgcaagggtgccgcacgacttaggaaaaaacagctaagtccgtgacagatggtatcagagttggacaaacactcatagaaacacttggtatgtaaaatgtgggggacctagcggggctgcgttgagggtagttagcacgcgcgaccgtttgggggcaaaacgggcatggaaatgtagggaaaaaggagtcgttcagaggagcgagcatctgagattgacattcagaggaatggctaacccttcgcgcaagaagcacaacgaggataagcaagctgggaagaatgtgtagcgcataaaggttggggctgagtttgagctacggctcaacgttagtaaccctacttgatggtgttcaaggtaagcgaggtgcttggtaaaggatgagaccatgtaaggtggaatgagttattcaacgaatgaaagagttatgcaaagctcatagaggtgaggggaattgctaactcaaagaattcggtactcatgcaaatgcttgtgtgcgaatgatggagtgttcgtggctatcccaagacgatcgaaacttggcgtcatagagcattgaaactttctcttcagcatgagaaggatacgtccgtagaaggctgaagtgtgcagcgagttcagcatgttgctatgcctcgaggggtgtagcgggggctgtattgatatggagtcgcaatctagcaagtgcgtttgcggtaggcagaacaatgcacagtttgttcagcaaggcaaagtagtcaaggggatggtggtctccgaaactttcaaagagaaggaaatgaagagagaagttgctcgaacgggacagatatccatgagggataagtctcggctctccaaagggagaaccatgtgcaacagatcgcatatgttgaggaggaggaccacaaaaacaaatttacaaagcttaacggaccaagcgagcggcaaggagtcgtcgcatgatctcacttgagataatgcattgggggaatgcattgtgagatcgagtggggaagcgacccaagacaacacaaatgaaggcacacttggagtcgatatggagatcagactcaatggagggttgacctgtggaatggtgggtgtaagagccaccatcagctcaatgcaaaacgaggagcggagcaacttgggtgtaacttggcgaagtacttaagccgcatgaagggagctggcatagaagatggaacatggagcggaggcacagagctttccttggacagaggtcaaggacatgaactcttgcagagacaagagcgggatcatgtcgttccgtggattactcatcttacatggtgccaaagacgaagggagcttctgggcacatgcatcttacctcagagaagtatttgacggaggaactgaggcgactcaacttgcggaggcgaagttgggttcagaagaccttggcatggggcaagaggacacggaggcgggtactcttgaagaatatgccacagtgttgccattcaagttgctatgaagggagtggtgcacaacggagattgtgttggtaggggcagaggcttaggatccagataatggtgcacaaatttcagcgaagtcaatggacttcgggaggtactaggcgacggactatcctagagcggtgtttcatctaggtgtgacccgagggcgggtgaaggaaggtcgattgccaaaggagcgaacacaatcgaaggtagaagaggccacgttgtgttggcagaggccacacatggagggatcacaatccgagttcatcccacaaggattagaatgtaatgaagatgtcaccaggaggcgacatggtgtagcggatcgtggtggaatagttcgtggcaatgcgatacacacgaatcttgtcccgtgagggattatatcatacggaggtatgatcgggagctactgggagctccactttggtgaacaacacgatggaaagaagggctatggatttaaggagtaaaagccatgataccgtagaggcgggtcttccgtgcgtgcatcgaattttgcatcggattaaagtcttggtcatcagcatatgggggctgtgtaccaccgagggaaaagttcgaatgcaagtaccattgagtcccatgggagggacttgatcatgtagtgatcgaagcagctagagagttggactactctagagcatattcgcttacgggagcctgacaagttaaaggacaaggctgagtaagcgaacgttgctaccaaggaagctaaagagaacagaatcggtgcgaaccccgcaacatgatggcaggggccatacatgggagttgcaatctgtctttccatcgactaaggggaactgcttggagaacacgaaggtgttgaaacagggggtcgaaaggggcgatgaagcgacgacgagtccaaagggacttagctacccaaaacaaagcatcagttagaatggaggtggactcggaggagtgctacatagatatatctaccgatcgtgaagataaggatatagatgtgaggcgacagatagtagggccatgggcatggcaacgccttggtacaacagaggtgggactttcatgaagtcatcgatcccttgctctcacggagggagagcacttggtcgtgaaaggggctgaggaggtggagcatgcagaggcaaactccaagtatcgagacaaggctgaagggcagaggccaaggaacttcgtaagaccggtgtcatcgagctttttatcaagatagccgaaagtgaaggacttcgggtcgtgcaagagtgcatgaccaaggaacgaaataggcagtatgcggtgttgtacctttgctactcagtggagtaggcggcaaggttgatggagaaggcggtacaatcctagaggcgaccaaagctacaagagacttgctccaagttgtggtgaaaacttcttacattccagaagttcgatggcattgagaaggtgaatcacagtaattaactcaatgcaaggagtgcaaacatttcaagtacttcagaagtgtgagcaaagagcaggcgaaggccagtaaccagctcgatgcatggagtataacattcaaggagacaacgaagtcaagtaacctttgccttctcaacccttaagagaatgggcgaaaccgagtacccaaattttcttatctatccagcagaggggctctacaaatgttcaaagaccattcgaagaaactaaatggaagacaatagttgtcaaattcaccaatggtgatcagtgctactgagagtagattacccgcttcatttcctaagagaatactaatcgaaagtggaagtgatgcgaacctacttagaactgacaactaagtaaaagaagagtcgatgggcaaattttgtggaggaaggatccaaaaacttcaaaaagttcaagcagcatgaggcatttaagagactggcgcatagcaaggatggtcttttccttcatctgaaggatctgcaggaaccaatagggatcaacacaacttagccaaccccacattagagccaagaggtgcattgcagctggagcagaagattgaagactcagcaaaggcgaggagttgtagtgtcggcaaaggcttcgacgaagacgtcgaaggaataagtctaggagaatgtcacagacaaaattctaaacaggatgtttgatgtaatgcttatgtatgccgtgtcttttggtatgttcatactttgcacagcatgtagagggacggtcgaaggcttaatagccctattttagttgagtttggtggttgttttaggcttgtaaataaatgttatgtcatgtggacacttgtgagagatattcggtttgtagtggaccattttggatcatttgttatgcgaccgtttagagcttgtgaagtatgtttgtaatttaccttggttatgaagtattttctgaaatgcttgcttgtggatcctgagtaaggcgctttctctaactcgttttctcttttgtaggtcctaagggaccatggtaggtttcggggaggttgacctttgcggatggacacgtaagggtgccgcacgacttaggcaaaaccaccgtgacagtccgctaccgtcgattggggttgttttcgccccgttatcgtcctaaattaggcggtggtattaaccgaggtagaagaaagaagagtattaattgagggagaagaagaggagaacctgaagatcgacatcgctctccctcctcgtttgtcggtgacttctcatccacacggggagaagaaacggtttcttttccctacttttccgcacaggcaaaaaagaggcgacatcatagaaaataatttttttttcttatatatatatgtatatatacatatttcttctccccacacgccaaaaagggtgatgagacagtcacccttttcttttttcttatatatatatatatgtatatatataaaacgaggcgacgttgcattggttttctactcgtgtggggagaagaaatcgtcaccctttttttttacttatatatatatatatacacacaccaaacggtataccgaaatgtatcgctcgatatatagtatcgtaccgtatcgagcgaatcttgaaactccggtatgatacgaaatttcaatccttggataagataactaatggaaatatgtagagaattaaaaaatcacatataaatttattgactttgaaaaagcttttgataggcaggtagggagctcctaaagatttgttatgattcatctaaaaagaaaggcaaaactactaattgtattgatgttatgaaaaatgcataatggtgtagttactggtattaaacctgtagagaatgtatttagcaaatttcctatcattatatcaatgattaactttatgccattaagtttttatattggtaatggaggaacttattagtcatgtatataatagacttccttactgtatgttcttttgctaacaatatcattttagtaatgaaagtttagatgaaattaatattaaatttgagttttggagattgaacctatcaaatgcaatattagttatactaagagaacaattaactatgtagttataatcattgagcaagaagcttttgtaaccaaagtatttaggttttttggaactattattaacttggaaggagagattcaagaagatattgttcataaagtagaagtggataattaaagtgaaaaggagcatcaagagttgtgtgatcataagacgtctcatggtttaaaagaaaaatttgataaaacaatatctagtctatcatgctttttggatctaactgctgaacggctacgggaaaaaaaaaatttatatagctgagatgagaatgtctagtataaggaaaaatataaaaatattttttttgtgaataataagatatagctgtgataaaacataaaatgagagaaagatcatttaagataatattgacttgttcaaagaagatatatagatgctatggttgcacaagatgagttgattgatattactagtgtgagttgacatagtgagagatgaaaagattttaataaaaataatataaatatatttatctgctctttaattgaatcagagatttatgtagctgaccccaaatagtttggaacatgctatttaaatgctcttgtatagagctcaatggtgggaaagacatgtagctggcctcaaatagtttggaacattatagcttgttattgttgttggtggtgatggtattcctcgaggatattgttgtgatgcttctgcatgagactattcataacacctggtgcaggtgtgcaattgaaattttagttcgtactgcttagcttaatgcaaattttgatgagttagtctttattccaacctaccttggagaattctgtttttttctttttagcgaaatctatggttacagggagttgaacaatttcgatgtgctatggaacttgcaccacacaatatatctgcacaccttgggcttgcttctgggctgcttgattggtcaaaagattgcattaaatctggagcatttggttgggcagctgatctcttacaagtgagttgtcaggactgtttcaaagcttaccccctctcaaggcttgagtgctttatatgattatatgtgcatgctggtggactataggaagcatcagaagctgcaaaagtcagtacctgtttgtctggaaatttatattctgcttggaagttgcatggggacatcaaggtatatgtcgtaattaataaattttattgttttatgggttttgttttttatgtactttgcatctttagacttgtgcctattgaaactcaaatgcttaatatgaatgtaccatttagttccttgtgtgatggtaactgcttgttgtagtttttggtgttggaaatatgaatgtaccatttagtttttgcattgtttagttatcaaggggctcaatgttcctaggattatttatgtctaaggtttttattcttcttcagttcttctatgcttcttccccttcacattagctctttgtcttgttcttatgttgtttgacttttttaagcaggagttacatgcgaacagaattgaagctccacatttttggtgccttatacttgtcaaacttctattaatgtttgataagttaaattagaacttcaacgtaaactcttaattaattaagacaatgaaataaaaaaagacaaaaatgaaatcaaagtcttatggtcatcatgcttgtttgatatctctattaatcatacactatgacctaatctttgaggtggaaacgcagcagaaattaaatccaaataataactatttcaaattcacaagagatttgctctcttacctttctattgtccaataacactagcttctttttccataagtgggaaccataaaggcacttgccttcttgcaataagtttcccatttccctcttttttgtgcatccatcaagagagcaatagtaagcaaggagatctatgcccttacataatttgccagcccaataattagcatacagaaatcccatgcttggccagcttgatagttgtcagtagatccatgttatgaggattgtgcttttagacattgccctaactttttaatgggcacataacttttttcatcttgacatcatactactatagtatgtccaagtggtatgtgctgattggcaaaatcagggttcaccttattggttcatatcggtgtatcgaacagtcaacgtatcgttctgcatggatgtaccatttgttggtatggcaaggcatactgatggcatggaaaaatgatcgaaaatagttccaaaaattcttgcaaaataaaaaaaagttaaattagggtttttaagatggaaataaatgtaagtttagtataattgtagcaaaacaatctaaattaggaaagaataatagtagatatcttttttaggctttaaggagtagcatcatggtatgtttcgcactatccttttgttgatattgaattattgacaaagaaatgatttgaattgttagattatttgttaaacaacttaaactttaagattcaaatgaatcaagtaatcgatcgatggatatacttggttctaccatcaaaagaatgacaggactccatgagcggtgagtcggggttctcgaccctatatatctgtatatcaatttgatatatttgtcggacctaatcttaaaattgatctcatgacatagtatattgatacaccgtatgtcattggtgcatcaatgtggtgatggttatagtttgattatcgtgaaccacatatgttcgagacggctcttgaggcaagtacgtttgtgacatgtattgtgacaagtacgatctgttactttgtatcataagatcgatcaccatactattgttcagagaagaatgaccaattatcacgtcaagctctatgatttgaatcttgggtggcatgtgtaagatattactcctcggtctatacaccatcctcaaattatgtagagagggccatcgactcctcgacatcgccgccatcatcagttgagacactactgttcaagtcgctgtcatgtctccagaccaaatggattgctaaatgcgattgagaagctacatactgttaattctccctaattagcctcctatgcttgatccaatccacaaatcgcagctttgtggagttcaagagagtgcaaatgagagatatgtgagtgaaaaagtgattgggagagtagatgagttagggcaagtgcaaggggagagaatgagggggtagaaagggtttaagaaccttagatctggttcaatttattaaattggttgtgaaatgttgcagttttgtggtatcatgttatgttaagcttcaatttatcaaatgacttatttttcactatctggcatgttaatacagaactctgaagtctgaattacttttttatgcattttttgttgatattagattgcatatgcaaaatgctatccatggggaactggaaggattggatatgaaatagatgaggatctcttgaaatcttccatcatcagctggaaaaagacatgctattcagctgcaattagtgctaaacactcgtatcagcgggctttacacttggctccatggcaggctaatatttacactgatattgcaatctctgttgattttattggctatctagaagagagaaacaacaattatttggaaatttggtaagctatttaaataaaactttggttatcgcatctgaatataagaatcaggctaaattgattctgtatttattaggcagctacctgagaggatggccctaggtggcttgttgctggaaggagttaataaggagttctgggttttgttgggatgtctggctagcagtgatgctctgaagcaacatgctttgattagagcgttgcagttggatgtgtctctttcctcatcatgggcatatctgggaacggtaatgatgaaagcattcatgtattgccaagtcttgaaacataataatctgattttgttagtttgcagctctatagaaactcgggagaaaagcaattggcatgtcaagcatttgatcgtgcaagaagcatagatccttctttggcattaccatgggctggcatgtcagctagttatcaggatgggtatataatgaatatttctcaagacccaaatatcatgtttcttaggcttttgacatttagcaaattatttccattgacaaatattatcttaggctttgctcgacaaatgaagcttttgaaagctgtctgtgggctgtgcagacactgcctgtaagtgcctttctctccaatactctattgcctttctcttttctgagcacattgaagttttgcatgtgcaatttaatcaagggttttcataatgaagtatttatttgattttgattattgattttgttataatttcataattaatcgacagacaagctctgaaaaaatgaatttcattataatgtatttatgtgactactaaaaaactgattttttcttgaaaaaattgcgattttgagatttctgtagtttggttttttaattatatgttgaaataaattactttacattcaaaataatcaagccactgcagatagacttctttatttgttggttatccagatattttacacacttgccatttgaaaattgaatataatcaccacttatacattgtaattccacttatttgtaggctgttgttttagctatatggtttcctgattgtgcttatgatctgggtgcagcttgcagaatttcaagttggtctgaaataaattatttacactcaaaataatcaagccactgcagataggcttctttatttgtttgttttccagatattttacacaattgtcatttgaaaattgaatattatcaccttttgtacattgtaattccacttatttgtaggctgttgttttagctatatagtttcctgattgtgcttatgatttggttgcagcttgctgaatttcaagttggtctttgtgcacttgcagttctttctggtcatctgtcatccccgctggtattgacattttggttaattattgtgttcgcttatctaaagccacaactatttgccttaacggttaaggcttaaccatgtttgtttcttgacttgctgaaagagaagcacaaattgttcaagcatttcagcacgctaaaatattgagagatatgtatatatatatatatatatatatatatatatatatatatatatatatatatctatatatatttatatttatatatatatatatatattgtaatttcttattaaaatgtttaaggcatgctgtgtggtcatactagaatttgccaattgtgaatgccaaataagtgaatctgtgatgatattttttttaaacagatagtgtctagcttgaatgtagtttgggttgtggatatctaatttgaacttttgttctcatctaagttgacagatttgagacatgagatagtcacatttataaccagtttttggtcactgttctgtggatgttgacacttgagaaggattttatggcatctgattcgatatagatttgtctacagcctatgcatcagagaagaatttttttcttaataatttgatattttattgcgtgttatgggtaggtccaagtgtgaaagaatataaatttataagtttcataaactaaaatggtccagatttagtgttattaaatcaattggtttatagaatccattattagagtttgacatgagggtcaagtttgtaagatccgtaagatcagtatgttatcggctagtgatgacaaaattagaattgataggacacatcgtagtggattttcattaataatttgataatttattgcgtgttatgggtatgtctgactgtgaaaagttagaatataaatttataagtttcataaaccaaaatgttccagatttagtgttatcacatccattggtttatagaatccattattagagtttgacatgatggtcaagttcgtaagatcagtatggtttcggctagtgacgacaaaattagaattgataggacccatcgtagtggatctaaaggatcatgattggccttgaatggggtgccggttatggatccgggtatatcagaatcagatgatactgtaaactctcaatgtagtcataaattcacggaaaacataattttaaccaggttgatgcaagaaactaaggtaacactgattttgaagttagataagactagttgttctatacttggttaggtgagaaaagatgttagtaacattcatcagttgatggatcactttggtttttgtttttaacagttgtagactacattgtcattccatatgtatatgcctgtcatgtttgttaattaggttttcagaatatcatgcatttgatttgcaggtaattggagccatagggcaggccgttcaaagggctccgtacttacccgagtcccataatttacatggcttagtatgtgaatcacggactgattatcagtctgcaattgttgcttatcagaaggcacgatgtgctttgagaatgtttcccaacttcaagcatgatcttcaatcttccttcactgatgtatcagttaaccttgctagatcactttgtaaggtaaattatgtttagttttggttacattgaatgcctttagtccacagaataaaaaatcatttacttattcatttttgtagaagcttttctaaaatgccttttcaattttatttccttccctccaacccccaactcctcatgccatctccatgtatacatatgcatcattgatggagcatttttgcgtatctttatgctctccacattctttattttttgcaggccggacatgcaattgatgctgcacaagagtgtgaaaatttgaagaaagaaggtaaggccttgaacttattttctacacatgttctaatgtggggaaatttttttctgcaaatttcatgtgaatcatagatataaataatgtgtcaattttacttctggggaaataactagtagcagagcaggctttaatgatatttacatgtacttttaatgtcattaatatttcaggattccttgatagtaagggcttgcaaatatatgctgttgcattgtggaaacttggacaatacaacttagctcttactgtggcgagaaagttggctgagaatgtctcaaccatgaaacagacatgtgcagctgcttcccttgggttgatatgcacactaatgtacaacatttcaggatatgactctgtagttagaacaattagaaaatttccaagtgagtttttgcaaaatacaagaatgagtctcatcgtctgtgcattaaatgctttagacacaaataaacaactgcagtcgcttctgccaaccatttctcaagctgctgcttctcatggtattgccattgaaatacattccattaccgctattaacaaattggtaagagtattcttctctagtcacaagtctcagtatgtagttttttcttctctgtactcttttgaagcaggatagtataaatgttcattgttgagtttaactttgcagataactcaggagtctcctcaaactgtagccattgacaggggagtagattacctaagaaaggcccttcatatgtatcctaacagtcttttaataaggtaatgagtgttagcatcctatatctgatcctcctcttatgacaacaatttctaccagtgccctaaaagctttcatgaggaatcctattgctgtttatgcaatcttggttttctgtaattattagtctaacatgcttttatgtggcttagcttttagtgttatgtatgacttgtgctgctttttatttacaagttctgcatcctttctgcattgtctaattgttgcacaattcaaaatctgtaaccaagtagatgatccacctgctagccttgagtgcttaaagatgaaaccaagaaatgctatattgaagcttgtcatatcagaattttgaatagattctagcgtggataaaagaaaatcacatgtaaaaactgaatacaccaacttgcaacttttatcaagctgaaagtttcaaaagtttgaatctctggaacctgtaaaagcaataagaagacagagggaaaaaatgatagaacccttgcagattctaaacttggggttgatctctttaggggatcagcctccttggaactctataggggttcctccctccatgttgctgctcaaaggttgcagaaaagattcatctattgcttaagaaaagaggaggaatacatggctatttatagggcttctaaaccctaactcttaataggactcctacttaagtctcttacttctaaccaacgcctaataggactcctactcaagactcatattcccttacaactcctaattcttctctaagaaaaaacctcataaccctagccggcctcttcatctttttaataggggtcggcttaggtaggttttacatgaatgtccctttcaattaggactcttctagctagagtcctaacagacccgccctcttcaaatcagtcttgtcctcaaggctgagattccatgaacttagggaaccaggtcttcagcttctcatatggttcccatgtgacgtcttcaagtggtaaattattccaatgcactagtacttcagtagagggacgtcggcgacgcatcacgatcctgcgatcgaggatggcttgtggttgagcatgaataactccatccttagtggtgttgggctgttggatctggggtgactcgtgttctcccaatttcggcttcaggcatgacacgtggaagacatgatgaattttggcatcctcaggtaatttaagtctatacgccattgctccaatacgctctgtgatctaatagggcccaaaaaatcgtggagatagcttcatggaggcttgagtgttgatggagagttgcttgtatggttgtaggcgaagataaacctaatctcctactgaaaattctctttcgcttcgtcgtgtgtcggcttgctgcttcattctggcttgagctgtagagagattatcttttaacagttgtaggagtttgtccctatcaatcaattcttgtttaacctgatctaccttggccgagccaattacatactttggaatcacaggggtcggtcgaccatacaatgcttcataaggggcacattttgtagataaatgatatgtagtattataccaccatttggcccagggaagccattttgcccactcttttggtcgatcgctagcgaaacaccgaaggtacgtctctaaacacctgtttaccacctctgtttggccgtcagtttgtggatgatacgctgtgctcatcttgagtttggtgccttgtaactggaataactcggtccaaaatttactagtgaagatcctatcacgatcacttacaatggaccttggcatcctgtgcagtttaacaatattttttatgaaaacttgggcaatactagcaggagtgtagggacttctcacagcacaaaaatgagcatatttcataagtcgatcaaccaccacgagaattgtgcttttacctttggcaggtggcagcccttcaatgaagtccatggagatatcagtccacatcgagttcggtataggtagtggttgtagtttccctggatttgccacagtttcacccttgtgctgttgacatacatcacattgtgccatatattcagcaataatttttttcatccctctccaataaaaattttgctttactcttttgtaggttcttaggaatccagattgccctgctgaaggtatggagtgcatttcatgcaggatgattgtgatacaaggggtgtcaggtataagcacaatacgacctttgtagcgtagatccctcgaatcccaagtgaagtgggctattgcacttggatcctcctcctttataatgttgctgatctctggatccttcttccattcttccctaatgtccacgaggagactagtggtaggaagggagatggctgaaaccttagcttgcttaggtagccatgagagtgcatctgcaacaacgttttcttttccctttttgaaataatttcataatcaaatccaagaagtttggttacccatttttgttgctcaggggatgatatcttttgctccaaaaaatacttgaggcttctatggtcggttttaatttaaaattgccggccaatcaggtagggtctccacctcattactgcatgtacaatggcgagcatctccttatcatatactgacatgttttgatgggaggaagataatgccttgctagtgtatgcgagtggtcgaccatcttgcatgagaacgactccaattccgactccagatgcgtcgatctcaatgatgaagggtcggctgaaatccggtagcgctagcaccggcgtcgttgtcatggctaccttaagtttgtcgaaggcaacagaggctttgtccaaccattggaaaacatctttttttagtagagaagtgagtggtgcactgatcttcccatagtccttaacaaattttcggtagttgcctgttagtccaaggaacccgcgtagtaatttcacgtttgtacgtttcggccagccttgcattgtctcaatttttgttgggtctaccgccactccttccgatattatgtgcccaaggtactctactttttgctggagaaagctacactttggttgcttaacaaaaagagtattctcccgaaggttcgtcagaacaatccgtaaatgctgaaagtgagtctcaagagaagggctgtaaatgagaatatcatcgaaaaatacaagcacaaatttgcgaagaaagctccgaaaaatatcgttcatgagactttgaaaggttgaaggagcattagtgagtccaaaaggcattaccaagaattcataatgaccatcatgtgtgcggaatgcagtttttggaatgtcatcttcacatacccgaatttggtggtaaccggatcggaggtccaacttcgtgaagacttgagctccttttaattcatcaagaagttcatccaccactggtattgggtacttgtccttgacggtgatgccatttaaagctcggtagtcgatgcacatccgccaagttccgtccttcttgcgtacaagtagcactggtgaggaataggggctacaacttggtcgaatcacccctgtttcaagcatctcctttacgatcttttcaatttaatctttttagaggtgaggatattggtacggtcgagtgctcgctggtggcttgcccggaaggattggaattcgatggtcatgttgtcgagaaggaggaagaccgcatggttcagcaaaaatgtcggcaaattcagtaagtaattgggcaagattttgatcttcaatttctattttcttctcttctagttgtggctgaagatgcatcaaaaagccaccatttaccttatgcaaaactttctccattcgttgggtcgaaacagtggtaacgttgcttccgcgcttcctgcgtaggatgatctgtttgcccttacagtagaatttcataattaatttagaaaagttccaagagacatcacctagtgtagtcagccattctataccaagcactgccttatagtcatcgattggtaggaggaagaaatcggtgataatttcttggtcttgcagtaatagtttcacttgcgggtatctttggtcgcactttaggattttgccgtcggcaacctttacatcgaacttgctgcaaccttcaatatgcagtgccatccgagcatcaaccttactattcaggaagttattagtgctacccgtgtcgatgagaacagtgatcggctgttgtttgagaaggcctccaactttcatcgtttgtgggtttgagtagccggctagtgcatgtatcgtaatgtcggtcggttgtggctcttcttccatatcttctatttcatattcaaggctctcttctggatgttcaatgacctcttcttctattggttcaatcataagaagtctcccttttctacagcgatgcttgtggctccacggctcgtcacaatgccaacataaccccttcgcagatcgctcccgaagttcttctcttgttaaccttttcggtgcagggactcggttgatagtagggggggctgagggctttagtattgtaggtcgaggagtgatcctagtcttccgggcttcatggttcaatcgctcctcttgaagttgtgcaaaagagatggctgccataagcatgtatggttgtcgcgccttaatttctctctggatctctggctttaagccctcaataaaggtccccaatagctgtttttcagaccaatcacgagtttgattagatatcctttcaaacctggtttggtactcctgaatggtggaggtttgtcggatctttgctagttgtccgtcaatgttctcgtaataagttggtccgaagcggatcagcggtccttctctgaattgtcgccatgaaaggactccataagtgtgttcaaaccagtcaagccactgtatgtcatccccttcaagatgtattgctgcaatttccaccatggatgcatccgcagttttatggtaccgaaaatatcactccgcgcgcgagatccaaccaattgggtctccttcttcccatctagggaagtccactctcatgtgcgtatagttggggtcggtcatagagcctcccctctcttggaagtcatctctttgggcatgatgtgattggttagagctctctccttgatgtgatttcttcgggcttggtggtcggcccaaactgaattcggtaaggagagtccgaatcttatccttcatttgtgcctcgaaggcttcaaatttagcattgattgcctcctcagatgccatagtatatgactccaaatctgtgatgttaagatctctcttttgttgacgggttaaaggcatgtataggttgagagaagtgatggttgaaagggttggtggattgatagatgtatgctgcggtttaggtttgttttgtggcttttttgggtgcagtttgagggtgtggtttggtggagttttagggctgtggatgaaagttttcgatctatggtggatagcagcaaaggtttgatagaaatcagtgaaagtttgttgcagaattgtgttgtcttgtaagagcagaattgtcgtcgaagaaacaacggtttttcgacgaaatctccaccaaaaatttgagagatttgaggagagaattgacagtaatatgttagtttatatgacagagaaaatatagtatcaagatcaaacaaacagtgctatgcggctgaaattctgcaatgtatctttcgttgtagagatgaaaactttgtgacgaaaagtttatgcagcaatataggaacaatttttttttttttggattttcggatagggataactaaattgcagcaacagtaaggtaggaaaccagaacctgttgtaattcacgggatgatccgcggtggtggagatgatggcggaatttggcgacgatcttttaagcaattatgggaattgctttgggcggttgtggagggctgatggatggctatggaagggcagcgagatgccaagaacgctgctctgataccaggtgatagaacccttgcagattctaaatttggggttgatctctttaggggatcggcctccttggaactctataggggttcctctctccaagttgctgttcaaaggctgcagaaaaggttcatctattgcttaagaaaagaggaggaatacatgactatttatagggcttctaaaccctaactcctaataggactcctacttaagtctcttacttataaccaactcctaataggactcctactcaagactcctattcccttacaactcataattcttctctaagaaaaaacctcctaaccctagccggtctcttcatctctttaataggggtcggcttaggtaggttttacatgaatgtccctctcaattaggattcttcttgctagagtcctaacaaaaaatgagataaacaaagatacaattcaaatagaaagaaattttccttacaagttaatcagtagagtcatctacttccagtgtgacaatatgttgcccacatgtttaagctcaagactggatggcgtcatgacatatgtagatccaatgtctattgcctacatgtcatgtgccactgaacttttggcatgcgaccagcagcagttgcatttaaaatgagacaatttttaaggattttttatttatgtgtttcctgacctcttcttttgttggcctaaaatgttgcaaaaaaaaaaaaaggcagtctcttaataattgtctctgtcattattcaagcctgttcataaacaaaggctcatctatggtgcacagcactaacactattctggcgattactctattgttaagccttttaagttctctcctaattttgtcatgtgtgactcaaatatatggtcaagatcataaattagtatagtaatactgtttcaatatacctgtatcttgtaacttcctaaattaggttactagtcctataatttaacaaatctgtacttacaaccttactatctctgttgtgtgtcaaaatgcaaaagttggttctactataattacaagactagcagaccaaattaaggatttttggattcattttgatccagtctaagaatttgaactgccctaaataatttggtacttgatgtatttttatgtcagtgttgtcatttgtcacatagaaatcattgccaagtttgtgatttgctcaaaggaacaacattgttatcacataaaagttaccttcctattttaattaaaatgaaggtttcttcatcgtaattaagttatgaatgaaatttatataattgatgttactgttttcaaatcagagtttttagggtgggaacttgggtggtatggattggtttgttttcaggttaacttaaaagtcctcatgttgcttcttgtagcctttctttcagttaagataatgtatttaataagtttaatatttttcctttgcctttgtaatcgaggaactaatgcatttaattgactttcagttggtgcattttctttagaatttatatcctttctgtggctagttgtttcatgaagttttccatgtgtattggctgaaactgatgcatgagagattgaccatatatttccgtttggaaaaggagtacctcttattgttgtgaatttataataatatatcttttgcagcgatttaaaaaggcgctcgggcactcgcctaagcgctcgggcgaggcgaggcgaggcccgagcgcctcgctaatcttccaggcgatgcgcttcaaagaggcgccgcttgggcgctcgcccgagcccaggcgctgggcgcttcggccgagcgcctgggttaaccaaggtgaccgaaccaggattttaggtctggttcggtcctgattcggtctccggtggttagttggttcaatcgaaccaactaaaacctatatcagtgacaacccaaccctaaccctcgttgccgctaccgctcccgatcccgatctcgatcccgctgctcgccgctgtcgctgcttggaaacgttgccgttgtcgccgctcacgcctcccgctgctcgccgctcccgctcccgctcccgctgccgctatcgcttctcgcaaacgctatcgttgtcgccgctcgtgcctcccgctgctcgccgctcctgctcccgctatcgctatcgttgtcaccgttcgccgctgccgctgtcgttgccactgcctccttacactccgctaccgctgctgcttcctcttttcttagtcagtaggctcagcacccccttacacttccttcttctccttctgttaacagtatactgtatactgttaatattattaggtttatttgaaattattaatttttaatattgttaatagattaataatatattattttgattttaatactgttaacttttatttatttgaaattattgctagatttcaagataaatgacaagtggacagagcaactcaatagagtctccaatgacataaaaaaaagattcaatgaagatgatttatgtgaaaataacgataatattgattatgatgaatgactttgatataaaattttattgttttgaatcttgaaaccttttgttaatgtgacattgtgatttggtgtcttagattttcttaatttaataacatattttaatttaaaattttaaataattatatttattaattatattatatatttttatgttttagtgcctcgcttcgttcgggcgagcgcttgggcgagcgcctagcgtctcgagcgtttttggaccttggcgtcttttggcgcctagcgctttttaaatcactgatcttttgtaaaggaagcatccactatgaactagacaaacaacatcagctgttcaaatttttatgtttcatgcgtttgcacattttgctattaaaaggtgatcgttgtacttgttttgcatgtcacagaagccatcttggtttgcttctactgtctagtggagattggatggctcttcagaaagcagccaggtgtgctgtcattccaactggtcaccagcaccctgtcaagaaaggatttagactaccatatgaaattcatggtgctatggcagttgcttgcggttccatctgttccaccaacccgaagtcttctttccctacctgcaatgatgggcttatgcatggagctagacacttgcacttgcttcaaaggtattattttatattttcttggtaattttcaaaactactctgggctggcaagatatgacagggttgtacattctgtgttgtctttttcagatggttgcatcaagaaccatggaaccaaaatgctcgttacttacttgctcttaatgttttccagaaagctcatgaagagaacttcccccaacaactttgcattatttcaaaacggttggttttagatgctctctctacagaagagtttctgggggacaataagctatctcagtctcggagtctcctactcctctgtgcatcagaaataagtttgcagagtggtgactctgttggatgcatacatcatgcatctaatgcattaggtcttcttcctaccaatagtgatatgttttttgcacacttgcaattatgccgtggatatgcagtgcaggaggactattcaaaccttaggaatgagtacaccaagtgcttgcagatgaagaccattcatccgatttgttggatattactaaaatattttgaatctagatacagtttgcagaataatttggacataatacataccaactttcaggcatgtgcagccaggaaaggaagttcctccaacaattggtctgctaactttgaattagtttgtgctcaatgttatttgtgggatcaggactacttccacgcagagcaaaccttggcacgtgcttgtgtagcaaatatggacagctgccttctactttgtcatggtattaattctacactgcatgtcaagaatttcaattaattttttatcaacatgatatgctttccccatatttgttcatgctgttttggtaatgtgatgtcagcattttgtactcgcatctatgtcctattacatcacggaggtactagtctgttgggattttctgataaatatcaagcatttgtcacatcagaaaatattttgtgcttggtgatctttgttttttcataaaaacaaaaggaatgaatgattgtaattgatttgttgcatatttgtgatgacctgctgatactgattagtttattgtgccatctcaatcatttatcttgcttcaatgtggtttaatcttttgtgacaaaaatactttcaaatgtagaactctttcacctaaaaaactaagggttatggaagaacaatgtcttgcaatagtattaattactggtaggattaaagtactatttactggtaggattaaaaatgttctatcatgttataacttactagtgacgatggagcaacatgaccaggttcgagcccgaggttggagtgggtcaattgactcgggaagttttgaggttgtcggtgagaactctattgagaaggattcgaatgctgatgttgtatttgttgctcccgaacaccatctgggatcctacaagaagatcggtgtcggggaagctcgacacagtccctccaacattcaagtaagcattctccaaagtgtggaagaatgtgggatgtggaagagaggagagaatgagagtggctatttaaacctgagggtctgtagctatcagtggttaaggtggatagggcaattgctaagggggcacggagatggtggatgaggtggcaaatggcagtgaagtggcatggctgcctactgaaattgcctaactatttgccaaggtaattgtgtaactacctgccttggtggtgttctcatctgctgagatggtgtaattgcctctttgggtagttagatggtgcaactgtccactaaggtggctttgaaaggacactttatcctctatcatttgtttcctattgtgcttcagcaaagaaaattttcacaatggagcatgatgtttgcttttggacaccaaacacctttgggagagcaagatggtcatggtccccgtgttgtcatctttgctggagttgattctttcacagtccagacttgtccttgtttgctgtgagtcttgttgccctgactgacttacccacatttggtggggaatccctagcaaaactacccacatcaggtagtgaatccctctaatcctgatcgcattagagaaagggtcttctgaatgttgacctatccacatcggacaaggtggcttcccaggcttgctgacttgcccatgttgagaaagaaatctcgttgagctggccgacttgcccacatcaggcgagatgacttctgtgttgtgtcgatctacccatattgggcaagaggactcttggaactccgatctgcccacatcgggcaagaggatttgtggaagctactaatctgcgtacatcaggtagtggagcttctgagcccaaccgacttacccacttcaggtaggtgggtctttagagttgcccgacctacccacatcgggtagggaaggcttccaaggtggccgacctacccacatcgagtatcttcaagatagtttgacctacatgcattgggctgagggaaccttaagcatggttgacatacccccagagagtggggattcctccctcttgattgacctacccacacggggtaggtgtcctttgaagcacttgacctacccacgttgggtagaggtcctctaaagttgttgacgtatccacattagatagggttcctcctaagctgtagacctacatacattgggtagggctcctacgaagttgcagacctacccacattgggtagggactctaaagatggttggcctacccacaataggtagggaagtctctagagacagttgacctacccacatctagtagtgggggtctctagagctttgattgcagtcgacgtatctatatcgggtaggagagtttttgtgcttggttgacctacccacattaggtaatggagcttcccttcttgctcacatgctcatgtcacgcaaggaagtcttcgatttggtcaacctacccacatcaggtttttgtttccttttttcttttggggggatgggtgggggggtatataggggagtcttcatagcctaatgatgcattagatgcttaggatgctcatgcgtatgtttgcaaaaggcgtattgtgcaaggaattcttcagtttcgttgacttacccacatcaggttttggggtagtcttcatagcttgatgatgcatcaaatgtttaagatgcacatgttcgtgatgggcacatcgtgcattggtgatgcttttgtccgcgaagaacacattagatgttgtagatgcttatgtctatggaagatacattatgcactggtgataagctcgtgaagggtgcattaggaaattggtaagcttctgcttgtgaacgacacattgggcgcttgagattcttttgccccaaagaggtgcattgagtgcttgagatgcttctacccacgtaggtgtatcagatgtttggcatgcttctgttcacgagggatacatcgggtgcttgagatacttctgcctacgagggatgcattgggcgcttaagatgcttctgcccacaggtgcacatataatgcttaggatgcttctgctcatgaaggtgcattgagtgcttgggatgcttctatccatgagggcacattcggtgcttgggatgcttttacctacgagggcatatcgggcgcttgggatacttttgcccatgaagggtgcatcaggaggtttggatgattttgctcacaaagagtgtataagatgcttgggatgcttttgcccatgagggatacatcggatgcttgagatgcttcttctcatgagggcacatcgagcgtttgagatgcttctggctatgaggggtgcgttgagtgcttgaaatgcttttgtctatgagtggtgcatgaggcgcttgagatgcttctgtcgatgagggtgcatcgggcgcttgggatgcttttgcctatgaaagacacattagatgctttagatgcttatgtcagtgaaagacattatgtaaagaagtctttagtttggtcgacctacccacatcgggttttggggggtcttcagtagctcgatgatgcattagatgtttaagatgctcatgctcgtgatgggcacattatgcattggtgatgcttatgttcgcgaaggacacattagatgttgtagatgcttatgtttgcgaaagacacgtgcattggtgatgcttatgtccgtgaagggtgcatcaggagcttggtaaacttttgctcgtgaacgacacattgggcgcttgagatgcttttgtccaaaaggggtttattgagtgcttgagatgcttctactcatgagggacacatcgagtgtttgagatgtttctgcccataagggcacatcaggtgcttgagatatttctacccacaaggggcacatataacacttgagatgcttctgctagtgagggtgcatcgagtgcttgtgatgcttctgtctacgagggcacatcgggtgtttcgaatgcttttgcctatgctagcatatcgggcgcttgggatgcttctgctcatgaagggtgtgtatcaagaggttgagatgattttgctcacaaagagtgtaaatatgcttaggatatttctacccacgaggggtgcatcgggtgcttgagatgcttcttctcacgattgtacgtcgagcatttggtatgcttctggccacgaggggcacatcgggtgcttgaaatgcttctgtccacaaggggtgcatggggcgcttgagatacttctgtccatgaggggtgcatcagatgtttgggatgcttctgtcgatgagggtgcattggatccatcggtcgcttgggacgcttttgctcatgaaggacatattagacacttgggatgctttagccttaaggggcgaatctggtgtttgagatgcttctgctcatgagggtccatcgggcgttttgtcacgaccttagctagaattgcctaaggcatgaggcacccttgcgacaaagacgtgaacttagcttaagttgcctaagttgtgacgcacccttgcgccaacttctcggacttagctgggattgcctaagtcttgaggcgcccttacaacatatgcgtccgcaaaggttcagcctagttgcaacctcgtacaggtcccgaaggacttgtaaaacagaaagttgattagattgaaaacgagcgacggacaagtcccgacgtctcgtgaagagggaagctttacaagcaattcagcaagcaccttgtgtgcacaggagaaaagaggaggaaaacaaggactttagaaggtagaacgaacagttgcaagttcacaaacaattgctgaccaggtggcgggcgcgaagacaagttcccgtcaagaaaacgtgcgaacttgtgaagattgttcaacgcccaactatatatcgaagccccatccagccctgcaccacccggggggttccaaggtgctgggatggttgacatttcgcgcgttgcagcgggctgcaaaaaacagcctgtggcacgtgaaaatggagccattttggggcttttggcctggcgcagtgagcaatcgcactgtgcgctgcaacctgttcgaacatgtatttttacaagtaaaaacacacaactaaggcaaaacaaactgccatgtctctgttcaagcatgcaaaaccaacgaacggtttgttgaacgaagttattgtgagtgcgtgacgatcattcgtgacattctcccccacttaaactgtcgacgccctcgtcgatgcttgttgatagttgttgatgattacttcttcatgtcgtagggcgtcttcgagctcccaactggcttcagttcggggaagctttcgccacttcaccaagtgctcggtctactcagctccattggatagctttatcttgcgatccgctagaatggtttcaactcgtttctcgtaggaggctcaggtagggggtagttgagttggaacacttcgggaagcatcttgcggatctgagtggtaggctttcaagttgctggtgtgaaaatcgttgtgaattttaagctacgccggtagctgcaacctgtaggagacgttgcccaccttactaataattggaaagggcccttcatacttgcgcaccaatcctttgtgtactttgttcctaaagaattggagtgatgttggttggagcttcaccaataccaaatcgtcgactttaaactcttgcgatcgccttcccaagtcggcccacttcttcatctttttgccgccttctccaagtaagcccgcgcaatatctgcatttcggtgccattcttttgtaaagtgatatgctgatggactactcctagtgtatccgatagccaaggtgtgaggagtcaacgattgttgtcctgtaatgatctcgaaggggctcttgttggacgcagagctctgttgcaagttgtaggagaattgggctatgtccaacatcttcacctaatctcgttggttggcacccacgtagtgtcgaagatattgctccaggagtgagtttatcatttcggtttggccatccatctaggggtggaggcttgtggagaagtataacttagaccccaacaatttgaatagctctatccagaatcgtcctaggaaccaagcgtctcgatcactgatgatattgtgcgggactccccaatacttcaccacattcttcatcatcagtttggccgcctcttcagttgaatagtgtaggggtgtagcaatgaaagttgcatactttgaaaatcgatcgaccaccacgagtatcgatctgagtccccctactgtcggcaagcttaatatgaaatccaaggaaatgctctcccacgacctttcttgtatgggcaacaactccaaaagtcccaccggcttccgttgctccactttatcttgttggcaagtgaggcacattcgaacatattcctccatatcagtacccatcttcggccagtagaaggccctctccacgagagccaaggttctgtgaatgctcagatgtctagcccaaagggaatcgtgacactctcttaagagctcatgcctcaaattgtttactcgagggacataaaccctattcccttttgtgtaaaggagtccctcctagacccaaaatcgtcgtgccttgccttttttgatgagctgcatcagggttactgcttggggatcactgtacaatccatccctgatcctggaaaggaagttggagtgcaactgatttgcttggcctctgccctcaaattgtacagcattcacacgctccactttccgactcaatgtatcggccacgacatttgccttttcgggcgtgtactctattgccatatcaaacttagccaggaagtccggccatcgtgcttgctttggggagagtttctttttagtttggaaatagcacaaagcaatattgtctgtcctcagcacaaatcacgatccaagaaggtagtgtcgctaaactcgtagacagtggaccaccgttgtcatctccttctcgttcaccggatatcgtcgctcggtttcattgagcttacaactctcgtaggccatcggatgaccctcctgtatgaatactcccccaatagcgaagtctgaagcatctgtatggacttcgaagggctccccatagttcggcaatttgagcactggttcttccaacacaacagccttcagatcttgcaatgcagcttcacatttattagaccacctccaaggctgctccttcagcaactctgtcagtggagttgcacgcttcgaatatcctaatatgaagcgtcgatagtagttgacgaaaccaaggaaggatctcagctctagcaccttctttggagttcgccattccgcaatagcttgcacctttgatttatccatctgaatggagccatcaccgattcgatgccctaagaataagatcttcgtctgagcaaagtagaacttctctcttttcacggacaaagtgttctctatgagaaccttgaaaattgtccgaaggtgctcgacatgctcctcgagcgtttggctatagacgacgatatcatctaattagacgaccacaaacttatccaaatattccttgaatagttggttcataagagtgcaaaacgtggtcggagcgttggttaaaccgaaaggcatcaccaagaactcaaacgctccatacctggtcacgcaggtagtcttcgcttcgtcgccttcagcaatgtgcacctgctagtacccagaccggaggtcaagtttggagaaatacttggctttgcccaattggtcgaacaagtccgcgatgagcgggatgggatacttgttcttcaccatctctttgttgagggctcgataatcaatgcatagtcggaggctcccatcttgtttcttttggaagagaattggagctccgaatggtgctttagaactgcggatgagaccaccgcttagtagttcacctagctgctttttgagctctactaactttggagggggcatacggtagggtggtctcgctagaggcttcactcctggctccagttcgatatgatgatccacgcctctgtgtggtggcagtctttggcaactcgggtggcataacgtctgtgaactccttcagaatgtttgccaccacagtagtttcatgaatgaccttctcgtcgagtagctctagcttcatagtagccacgaatgttaattcaccttttcgtacccctttcttcagttgtaatgctgatatctattgggggtcctttgttcctctccgagagacgggaaccatacaggggtcgtcacctcccatcatacatagggagtttaggaatgacattggcaccaacttggctgcgtacataaactccattccaagaatcacttggaagtcgtccagtggcaccgccatcatgtttgtgctcccactccatgttccgatcttgatgggaactccctttgccaacctagagattcgcttggcctccgagttcatcgccttcattcgacttgggttcttatccaagatcagcccaagtcgctctgcttctcgatcaacaataaagttgtgggtagctcccgtgtccaccattgcacgggttgtttggccattgagcttaatgtccatgtacatgagctcgctattccctgctttcaatggctttgtctttatgttttcccccatttgaccctgcaatgcatttaacaaacgcattgctcccactcggggaccctgcgactcttcatcgtcgctgctagattttgaactgcttgaactaagggcgatgactttgcccttgtctgatttgggggggatggatggaagctgttaaagcattgagtgcctgtttctgtgggcactccctcaccatgtgcggccctccgcacaagaagcacccgccagtttttggggccttgccttttgagcttggccctttgtgggaactcttcttcctttgttcgcctttgggctccttccctcgagaatattttggagggcgatttcctaaagatggtctccttttcctcgggtcctcaaaggaaacaaagtcggtgagcctttctgctgtccaattgccccgatcacatcggtgacattccttcgatgcagtttttgttgagcccatggcttcaaaccatcgaggaaacggaataacttatccttcttggacatgtcctgtatgtccagcattagcgcagaaaattgcttcacgtagtctcggatggaagtactttggcggagttgtctcaacttcctccttgcgacgaactctgtgttctcgggtagaaactgagttctcaactctcgctttaagtcctcccatgtgtccacccgacgtcgaccttgttggatttcctcccgacgggttcgccaccaaagttttgcatcccaattcaaatacatggttgctatcgaaactttggtttcttaatcaggcctcgtagcttgaaaatattgttccatgtcgaataggaaattctcgagctctttagcatctcgggtacctccataacaatgcagctcgggtgccctcaagttttgtggtggcgcaacgcgggtgttgcttcctcccgcattcaatgccctggtgagcatggtcaccttggaagtgagttctgctacgacttcctgcagatcttgcacggagtctttggtgtcgtccgacagtcgatcgactagggcctcgaccttgtcgatttgggattcaacttcttcttgcgagctctctaccccaagaagccttcgctggccttggtagagttcctccaagctcgcttcaagaacatccaagtgggtttccgcagcttgagtctctccttatggctcttctacccggttgacgcaatagattgcgcctccgctcgtgaagagtagccaatttctcgctcatcatgtttgctgttacgatcctccaaagcgtttccaacgacataagagcaagtttgcacttgcagcccacctgcgactgcttggggcaaaggttcgacttgccccgccttgctagattcgccacgatgctttgccatagtgaaattgcgaagttccttcactgcttgctcgaattgcttgcccgctctgataccacaatgtcacggccttagctagaattacctaagacgtgaggcacctttgcgacaaagacgcgaacttagtttgagttgcctaagtcgcgatgcacccttgcgccaacttctcggatttagttgggattgcctaagtcttgaggcgcccttgcgacatatgtgtccgcaaaggttcagcctagttgcaacctcgtacatgtcccgaaggacctgtaaaatagaaagttgattagattgaaaacgagcgacggacaagtcctggcgtcttgtgaagagggaagctttacaagcaattcagtaagcatcttgtttgcataggagaaaagagggcaaggaggaaaacaaggactttagaaggtagaacgaacagttgcaagttcacaaacaactgctcatcgcgtgctatgcgcgaaggcaagttcccatcaagaaaacgtgcaaacttgtgaagattgtttaacgcccgactatataccgaagccccatccagccctatgccacccagggggttccagggtgctgagatggttgacatttcgcgcgttgtagcgtactgtagaaaacagcctgcggcatgcaaaaacggatccattttggggctttttggcccagcgcagtgagcagtcgcactgtgcgctgcaacctgttcgaacatgtatttttacaagcaaaaacacataaaaccaaggcaaaataggctatcatgtctctgttcaagcatgcaaaagcgacgaacggttcattgaacgaagttattacgagtgcgcgacgaccgttcgtgatagtttgggatgtttctagccacgaggggtgcattaggtgcttgaaatgcttctactcatgaggggcgcatcgagcgcttgggatgtttttaactataaagggtacattgggtgcttgagatgcttttgcccatgagggtgcatttagcgctagtgatacttatgcccacgaagggtgcatcgggcacttgagacgcttatgtccacgaggggcacatcgaatgcttgggatgtttttgccacgaggggtccatcgggcgcttgaaattattttgcccgagagaagggtagacccaacctcatgcttttcaagcaatttgacaatcggatgatgacgaatatgacacgagtcacttgccaattgctcccccttgagtctaacttctctttgcttccacattggtcaagatagagaagtgaagatcaagatgaaagacttcaatgacaatcctaaagcttcactcgaggcttcatcccattaggagaaggaaactggagatgtagggaatgaagctgcgagtggcttgacaaggtgtaacgtttgcgcttagaatgaaaatggattgtcctaaagaaaaaatttaggtgataagccctttggcatgtaacaagtaatatgtgaatataaggttgcttttctcgaggtgcccccatcctatgaattcctcgatctgctccttcaggtcatgttagtcctcaatgtcatgcccatggtctatgtgaaatctgcattacttacttttgcctcaccttacttaggatgatcatgttggacgagggtcgttcagcactcccttttctttaaggaaaacttttgttttcaaggcgtttaagggagtgaagtctgcctcacttaagagggtccttccctagcaggctcagactttcgggttggcggttattgaggtcgtgatagttcttcatccctcacttgttttcaccttaagtcctttttactacttgaggcaagaagctctatagctatgtgttgatttgcttgctgtaagagattgggaaaggtggttcgttgaaccacataaactgagattttcaccacataaaggtggttcgttgaaccacataaacttttgttgatttgcttgctgtaagagattttcaccttgatgttcgaccatataaactgagaaggttgagaccttgcatgaagttctaaacgatcaatgggtgcacatggtcacctgatgtgcctcggtttcatatctagagacgtaattggccaactactctccttgttagattgtcaggagcgatgtcacagtacgcatcaatcattagtcattggcaatgtgcgccccgaactacttcacaagcctgtcgaatgtggatagggaaataggagatagggtgataaaaccattcttgagtggttccccgaagcattgtcgagaacaatcgacacatcaaggcatctagtgtggcatagagtcacttgtgctctaaatgatgccaagtgctccttggggttgacactaccatcaaatgtctccaaggtgggtaatttgaatccttgagggatttgctcctcttgaattcttaggatgaagggagaccatccgtatggttgcatcttatctccctctttggcaaattgaaactctcggcgcatgctctctagcctcaatccatctcttgcagttaggcatgtagagtggcattagtgtcgtacatggaagcttcggacttgagtctgcccgatcccttgccgtcccccataggagatggcgatatgaccacatgaacaagttgcagcatcgatctgctacggtgtcccatctttacttgctagctgtaaggcacctagagctgtggggtttcagagcactcagaatcgtgttaggttgcaaagtggtctggttttaggtgaaggtgagttgcatcagttgtggaatgaagggaatgatggactgcatcatctccaaaaaggtttggatctgctaaaatgacctcaggaaagcttctgttgacattgtcataggtgggggctcgctcaattgtgagtaaaaaattaaatagccaaaaagtcgatgatatttagtggcatttgttaggtcttgaagcggcaacaaaagagaatgcaggttgagctgatctcaaacttgtgaaggtggggaagtggttggccccaagacactcctgaaccttcttgggaagattatagctctgcatcatatctcaggccctcttttagcacccaaaatgttcacctcaatgtttgatcactcgacatgtggcgatgatggagcgacgcaactaggttcggttccaaggtgagagtggatcaatcaacccgggaagttaggaggttgtagctgaggacctaccaagaaggatatggatgtcgatctgaatgctgatgtggtttctgttactcccgaatgtcatgcaagaagatcgggttggggaagcctaacttgatccctctgacactcaagttagagaaccatttagagtagaagaatgtagaatgcggaagaaagagaacagaatgaggggctatttataccaggggtctgtagttgtcagtggttagggcagttgccaaggggttataaccgcctttcgagaaggtggatgaggtggcctaactattgccttggtggcaatgaagtggcacagctgcttgctgaaatagtgtatctgcatgccgaggtggtgttcttgtctgctgcggtggcataactgcccatttaggtagttaggtgttgcaactgtctgttgaggtggcagcttcagtggttgggagacgcctgactgctgttgaggtagcagaccctgttgctgcggtacttctagttgttgaggagttggtgttcatgtgtaagctttgaaatgttacttttatcctcaatcattacttgttctagttgtcctagtggagctgggcaattttgatatatgataccataaaatatagtgtaaccatgtagtttccgattgatacacaccattttgttttttgttagaaaacatttggttcatatcttggttgaactatatgatgtgacaggtgccatctgtatggaacttgttaggcagcaggcaggtcttcagtttctgtctcgtgcaattagcagcctgacaaaagctcagaagtgttcgcctatgttcttgcccattgtttcactgttgttggctcaagcagaagcaagccttggagcaaaagccaagtgggagaagaaccttagccttgaatggtttgcctggccagcaggttctgaacaaaactctagatgcagaaattgttccagtcgtttgcttgtcttttgttgacttcatttttgttgtcacagaaaggaggccagctgagttgtatttccaaatgcacttgcttgcaaggcaatccagtacagtctccaaccaagattctggcgtagggtcgactcaaagcccagagaggtggatactacgagccatacatttgaacccctcatgtttgagatactggagaatacttcacaagtgttacagctaggtaattgacttgcttatccagacatgtgcttcgtggttgtatcagctggctttagcctcgttcattttagtggttaatatttcgaatctgatgatcactgtccaacaaaattcgtgtacttttatgtgtaatccgatgagaatgtgtattgcaaagtggcaa